(19) United States
(12) Patent Application Publication
(10) Pub. No.: US 2026/0146035 A1
(43) Pub. Date: May 28, 2026

(54) FUSED BICYCLIC COMPOUNDS AND THEIR USE AS MER AND AXL INHIBITORS

(71) Applicant: DONG-A ST CO., LTD., Seoul (KR)

(72) Inventors: Richard F. CLARK, Gurnee, IL (US); Zhiqin JI, Libertyville, IL (US); Yujia DAI, Gurnee, IL (US); Roberto M. RISI, Pleasant Prairie, WI (US); Julie M. MIYASHIRO, Morton Grove, IL (US); Yiyun YU, Grayslake, IL (US); Guidong ZHU, Gurnee, IL (US); Patrick B. BRADY, Grayslake, IL (US); Jane GONG, Wheeling, IL (US); Robin R. FREY, Libertyville, IL (US); Thomas D. PENNING, Elmhurst, IL (US)

(21) Appl. No.: 19/122,220

(22) PCT Filed: Oct. 20, 2023
(86) PCT No.: PCT/IB2023/060602

§ 371 Date: Apr. 17, 2025

Related U.S. Application Data
(60) Provisional application No. 63/380,328, filed on Oct. 20, 2022.

(51) Int. Cl. C07D 401/14; A61K 31/4439; A61K 31/444; A61K 31/4545; A61K 31/4709; A61K 31/4747; A61K 31/496; A61K 31/497; A61K 31/4985; A61P 35/00; C07D 213/81; C07D 213/82; C07D 401/04; C07D 405/12; C07D 405/14; C07D 417/14; C07D 471/04; C07D 487/04

(52) U.S. Cl. CPC C07D 401/14; A61K 31/4439; A61K 31/444; A61K 31/4545; A61K 31/4709; A61K 31/4747; A61K 31/496; A61K 31/497; A61K 31/4985; A61P 35/00; C07D 213/81; C07D 213/82; C07D 401/04; C07D 405/12; C07D 405/14; C07D 417/14; C07D 471/04; C07D 487/04

(57) ABSTRACT

Compounds of formula I

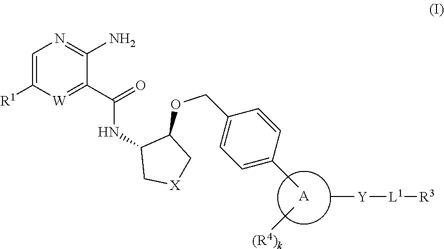

and a pharmaceutically acceptable salt thereof, wherein ring A, W, X, Y, L¹, R¹, R³, and R⁴, are as defined in the specification, are useful for treating or preventing cancer or an immune-related disease or conditions. Also described are pharmaceutical compositions of compounds of formula (I), and methods for using such compounds and compositions.

### TECHNICAL FIELD

Compounds of formula (I) and methods for inhibiting Mer and Axl kinases are disclosed. Additionally, the present disclosure relates to compositions containing compounds of the present disclosure and methods for their use.

### BACKGROUND ART

Receptor tyrosine kinases (RTKs) are enzymes that can phosphorylate specific tyrosine residues in target proteins, using ATP and share a highly conserved catalytic domain. The conservation makes it hard to develop a selective tyrosine kinase inhibitor (TKI). The TAM receptor family consists of Tyro3, Axl, and Mer, which play important roles in cancer, hemostasis and inflammation. Mer is, especially, a key regulator of macrophage and dendritic cells activation. Mer activation promotes apoptotic cell clearance by macrophages and additional cell types such as retinal pigmented epithelial cells. Moreover, Mer is abnormally overexpressed in human cancers such as AML, ALL, lung cancer, glioma, melanoma, prostate cancer, schwannoma, mantle cell lymphoma and rhabdomyoscaroma. Axl is more broadly expressed in a variety of cell types and tissues and participate in diverse cellular processes such as angiogenesis and tumorigenesis. Axl overexpression is critical for the development, growth, and spread of tumors, including proliferation, invasiveness and immune modulation. Axl has been implicated in chemoresistance as well as resistance to targeted therapy of oncogenic drivers, such as BRAF and EGFR. Its overexpression in resistant tumors correlated with poor survival in a number of aggressive tumors including triple-negative breast cancer, acute myeloid leukemia, non-small-cell lung cancer, pancreatic cancer and ovarian cancer. Much less is known about Tyro3 but its main function is involved in innate immune response, platelet aggregation and neuronal signaling.

Since ATP-binding site is similar for all protein kinases, it is challenging to find an inhibitor that is specific for Mer and Axl kinases. Compound-52, a 2,6,9-trisubstituted purine that competitively binds to the ATP binding pocket, was actually the first molecule that was found to be successful in inhibiting Mer (J Struct Biol. 2009 February; 165(2): 88-96). This inhibitor has, however, limited potency and lack of selectivity. Several compounds have been discovered mostly by modifying Compound-52 including UNC-569, UNC-1062, and UNC-2025 (ACS Med Chem Lett. 2012 Feb. 9; 3(2):129-134, Eur J Med Chem. 2013 July; 65:83-93, J Med Chem. 2014 Aug. 28; 57(16):7031-41). More recently, highly potent and selective Mer kinase inhibitors were disclosed in U.S. Pat. No. 10,125,118 and WO2018071343.

It is an object of the invention to provide reagents and methods of modulating receptor tyrosines kinases Mer and Axl. This and other objects of the invention are provided by one or more of the embodiments described below.

### DISCLOSURE

### Technical Problem

The present invention provides novel compounds capable of selectively inhibiting Mer and Axl kinases, which compounds are useful for the prevention and/or the treatment of cancer and other immune-related disease such as infection and sepsis. Highly potent and selective receptor tyrosine kinase Mer and Axl inhibitors based on an aminopyridine scaffold are described. Such compounds have the following formula (I).

The present disclosure is directed to compounds having a structure of formula (I):

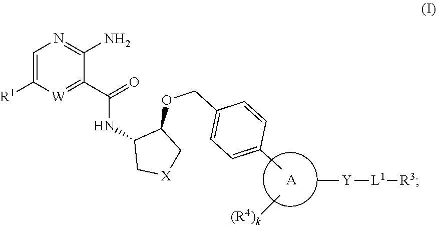

or a pharmaceutically acceptable salt thereof, wherein:
Ring A is selected from the group consisting of benzofuran, benzoimidazole, chromane, dihydroindene, dihydropyrrolopyridine, indazole, indole, indoline, isoindoline, pyrrolopyridine, tetrahydroisoquinoline, tetrahydronaphthalene, tetrahydronaphthyridine, and tetrahydroquinoline;
W is selected from the group consisting of N and C—R²;
X is selected from the group consisting of —CR⁶R⁷—, —O—, and —NR⁸—;
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)ₙ—, —N(R^{a})—, and —NHCO₂—(CH₂)ₚ—;
R^{a} is C₁-C₄ alkyl;
R¹ is selected from the group consisting of hydrogen, C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen or C₁-C₄ alkoxy;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR^{3c}, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁴ is selected from the group consisting of F, Cl, Br, I, ═O, C₁-C₄ alkyl, C₁-C₄ alkoxyalkyl, and C₁-C₄ hydroxyalkyl;
wherein two R⁴ substituents together may be combined with the atoms to which they are attached to form a cycloalkyl ring or a heterocycloalkyl ring;
R⁶ is selected from the group consisting of hydrogen, C₁-C₄ alkyl, and —OH;
R⁷ is selected from the group consisting of hydrogen, and C₁-C₄ alkyl;
R⁸ is C₁-C₄ alkyl;
k is 1, 2, 3, or 4;
m is 1, 2, or 3;
n is 0, 1, 2, or 3; and
p is 0, 1, 2, or 3.

In one aspect of the compounds of formula (I), W is C—R²; and the remaining variables are as defined for formula (I) above. In other aspects, W is C—R²; X is —CR⁶R⁷—; R¹ is selected from the group consisting of C₁-C₈alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; R², R⁶, and R⁷ are each independently hydrogen; and the remaining variables are as defined for formula (I) above. In another aspect, Ring A is selected from the group consisting of dihydroindene, and tetrahydronaphthalene; W is C—R²; X is —CR⁶R⁷—; R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; R², R⁶, and R⁷ are each independently hydrogen; and the remaining variables are as defined for formula (I) above. In yet another aspect of the compounds of formula (I), Ring A is indoline; W is C—R²; and the remaining variables are as defined for formula (I) above.

In one embodiment, the present disclosure is directed to compounds having a structure of formula (II):

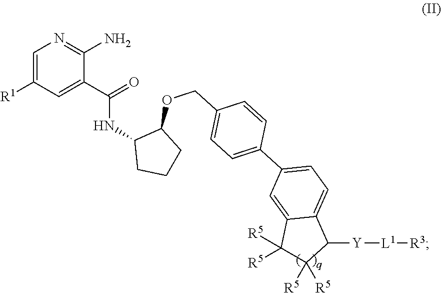

wherein,
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)ₙ—, —N(R^{a})—, and —NHCO₂—(CH₂)ₚ—;
R^{a} is C₁-C₄ alkyl;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR^{3c}, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein no more than three R⁵ are other than hydrogen;
wherein two R⁵ substituents together may be combined with the atoms to which they are attached to form a cycloalkyl ring or a heterocycloalkyl ring;
m is 1, 2, or 3;
n is 0, 1, 2, or 3;
p is 0, 1, 2, or 3; and
q is 1 or 2.

In one aspect of the compounds of formula (II), L¹ is a bond; R³ is selected from the group consisting of hydrogen, C₁-C₄ alkyl, C₁-C₄ hydroxyalkyl, —OH, —NR^{3a}R^{3b}; R⁵ is selected from the group consisting of F, and C₁-C₄ alkyl; and the remaining variables are as defined for formula (II) above.

In one embodiment, the present disclosure is directed to compounds having a structure of formula (III):

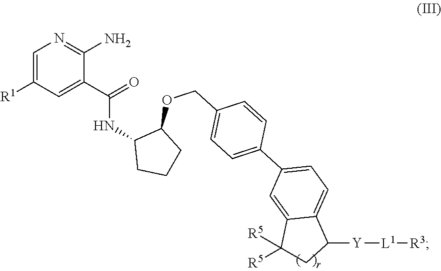

wherein
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)ₙ—, —N(R^{a})—, and —NHCO₂—(CH₂)ₚ—;
R^{a} is C₁-C₄ alkyl;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR^{3c}, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein no more than three R⁵ are other than hydrogen;
wherein two R⁵ substituents together may be combined with the atoms to which they are attached to form a cycloalkyl ring or a heterocycloalkyl ring;
m is 1, 2, or 3;
n is 0, 1, 2, or 3;
p is 0, 1, 2, or 3; and
r is 1 or 2.

In one aspect of the compounds of formula (III), Y is piperazinediyl; L¹ is a bond;
and the remaining variables are as defined for formula (III) above. In another aspect of the compounds of formula (III), Y is octahydropyrrolopyrazinediyl; L¹ is a bond; and the remaining variables are as defined for formula (III) above.

In another embodiment, the present disclosure is directed to compounds having a structure of formula (IV):

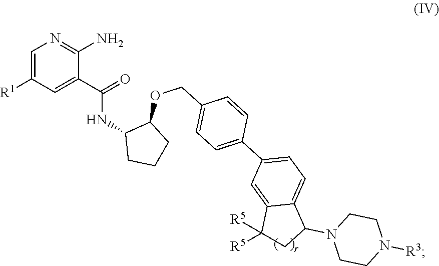

wherein
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, and C₁-C₄ alkyl, and C₁-C₄ hydroxyalkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein two R⁵ substituents together may be combined with the atoms to which they are attached to form a cycloalkyl ring or a heterocycloalkyl ring; and
r is 1 or 2.

In one aspect of the compounds of formula (IV), R³ is C₁-C₄ hydroxyalkyl; and
the remaining variables are as defined for formula (IV) above. In another aspect of the compounds of formula (IV), R¹ is selected from the group consisting of pyrazolyl, pyridinyl, and thiazolyl; wherein each R¹ pyrazolyl, pyridinyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; and the remaining variables are as defined for formula (IV) above.

In another aspect of the compounds of formula (IV), R¹ is selected from the group consisting of

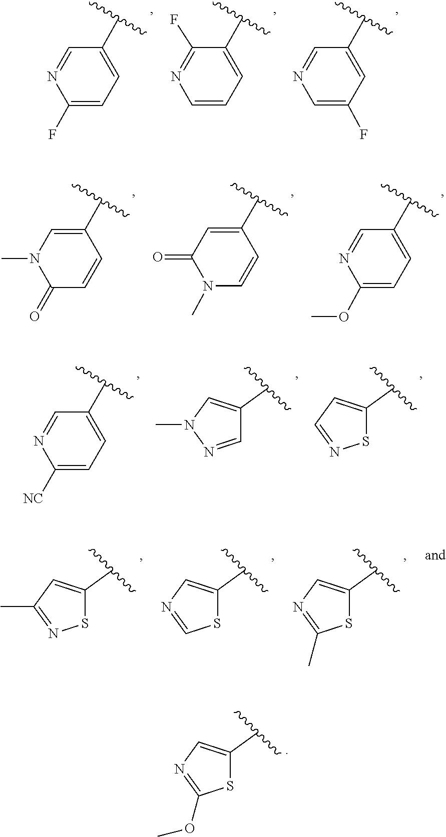

and the remaining variables are as defined for formula (IV) above.

In another embodiment, the present disclosure is directed to compounds having a structure of formula (V):

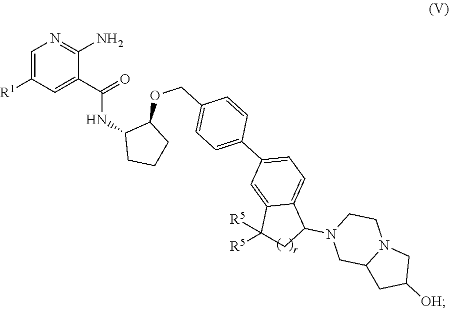

wherein
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of C₁-C₄ hydroxyalkyl, —OH, —NR^{3a}R^{3b}, —C(O)OH, C(O)OR^{3c}, and —C(O)NH₂;
R⁵ is selected from the group consisting of hydrogen, F, Cl, Br, I, ═O, C₁-C₄ alkyl, C₁-C₄ alkoxyalkyl, and C₁-C₄ hydroxyalkyl; and
r is 1 or 2.

In one aspect of the compounds of formula (V), Ring A is indole; and the remaining variables are as defined for formula (V) above. In another aspect of the compounds of formula (V), Ring A is indole; Y is piperidinediyl; L¹ is a bond; and the remaining variables are as defined for formula (V) above.

Another aspect of the present disclosure relates to pharmaceutical compositions comprising compounds of the present disclosure or pharmaceutically acceptable salts thereof, and a pharmaceutically acceptable excipient.

Moreover, the compounds of the present disclosure or pharmaceutically acceptable salts thereof, useful in the pharmaceutical compositions and treatment methods disclosed herein, are pharmaceutically acceptable as prepared and used.

In embodiments, the present disclosure relates to a method of treating a disease, disorder, or condition, the method comprising administering an effective amount of a compound of the present disclosure, or pharmaceutically acceptable salt thereof, or a pharmaceutical composition, to a subject in need thereof, wherein the disease, disorder, or condition is cancer or an immune-related disease

These and other objects of the present disclosure are described in the following paragraphs. These objects should not be deemed to narrow the scope of the present disclosure.

### Technical Solution

Disclosed herein are compounds of formula (I), or a pharmaceutically acceptable salt thereof,

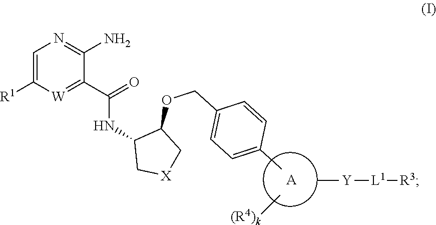

wherein R¹, R³, R⁴, L¹, k, X, Y, W, and ring A are as described above in the Summary.

In one embodiment, compounds of the present disclosure are represented by formula (II), or a pharmaceutically acceptable salt thereof,

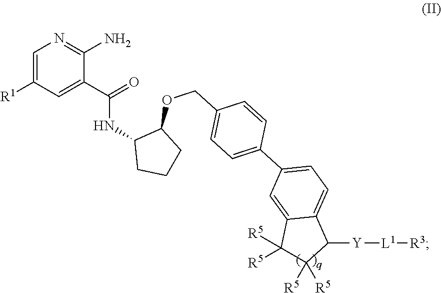

wherein R¹, R³, R⁵, L¹, Y and q are as described in the Summary.

In another embodiment, compounds of the present disclosure are represented by formula (III), or a pharmaceutically acceptable salt thereof,

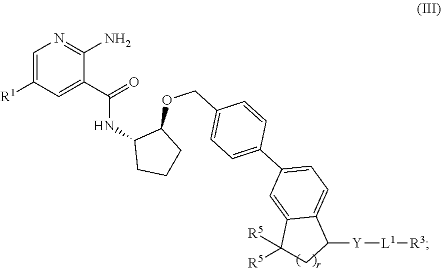

wherein R¹, R³, R⁵, L¹, Y, and q are as described in the Summary.

In yet another embodiment, compounds of the present disclosure are represented by formula (IV), or a pharmaceutically acceptable salt thereof,

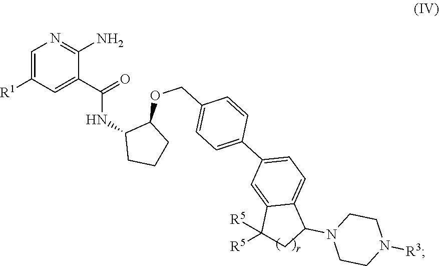

wherein R¹, R³, R⁵, and r are as described in the Summary.

In yet another embodiment, compounds of the present disclosure are represented by formula (V), or a pharmaceutically acceptable salt thereof,

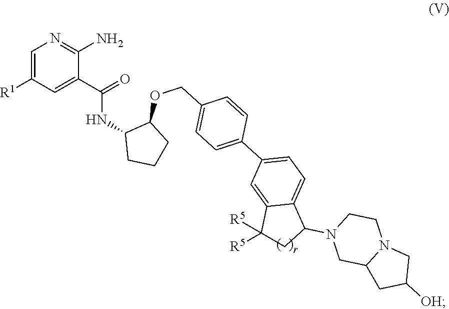

wherein R¹, R⁵, and r are as described in the Summary.

Further, compositions comprising any such compounds of formula (I, II, III, IV, or V) and methods for treating conditions and disorders using such compounds and compositions are also described.

Compounds included herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds, which can be isolated from a reaction mixture.

### DEFINITION Of Terms

As used in the specification and the appended claims, unless specified to the contrary, the following terms have the meaning indicated.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds. Reference to “a pharmaceutically acceptable carrier” means a single pharmaceutically acceptable carrier as well as one or more pharmaceutically acceptable carriers. —

The term “alkenyl,” as used herein, refers to a straight or branched hydrocarbon chain radical containing at least one carbon-carbon double bond. Representative examples of alkenyl include, but are not limited to, ethenyl, 2-propenyl, 3-butenyl, 4-pentenyl, 5-hexenyl, 2-heptenyl, 2-methyl-1-heptenyl, 3-decenyl, buta-1,3-dienyl, and the like.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, hexyloxy, and the like.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, methoxymethyl, and the like.

The term “alkyl,” as used herein, refers to a saturated, straight or branched hydrocarbon chain radical. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl, and the like.

With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The term “cycloalkyl,” as used herein, refers to a saturated hydrocarbon ring radical containing carbon ring atoms. The cycloalkyl may be a monocyclic, a bicyclic, a tricyclic, or a spirocyclic cycloalkyl. The monocyclic cycloalkyl is a carbocyclic ring system containing three to eight carbon atoms, zero heteroatoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring, or a bridged monocyclic ring system in which two non-adjacent carbon atoms of the monocyclic ring are linked by an alkylene bridge containing one, two, three, or four carbon atoms. Representative examples of bicyclic ring systems include, but are not limited to, bicyclo[3.1.1]heptanyl, bicyclo[2.2.1]heptanyl, bicyclo[2.2.2]octanyl, bicyclo[3.2.2]nonanyl, bicyclo[3.3.1]nonanyl, and bicyclo[4.2.1]nonanyl, and the like. Tricyclic cycloalkyls are exemplified by a bicyclic cycloalkyl fused to a monocyclic cycloalkyl, or a bicyclic cycloalkyl in which two non-adjacent carbon atoms of the ring systems are linked by an alkylene bridge of 1, 2, 3, or 4 carbon atoms. Representative examples of tricyclic ring systems include, but are not limited to, tricyclo[3.3.1.03,7]nonanyl (octahydro-2,5-methanopentalenyl or noradamantanyl), and tricyclo[3.3.1.13,7]decanyl (adamantane), and the like. Spirocyclic cycloalkyl is exemplified by a monocyclic or a bicyclic cycloalkyl, wherein two of the substituents on the same carbon atom of the ring, together with said carbon atom, form a 4-, 5-, or 6-membered monocyclic cycloalkyl. An example of a spirocyclic cycloalkyl is spiro[2.5]octanyl.

The term “haloalkyl,” as used herein, refers to an alkyl group, as defined herein, in which one or more hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, trifluoropropyl, and the like.

The term “halo” or “halogen,” as used herein, means Cl, Br, I, and F.

The term “heteroatom,” as used herein, means a nitrogen, oxygen, or sulfur atom.

The term “heterocycloalkyl,” refers to a non-aromatic saturated monocyclic or polycyclic heterocycloalkane radical having carbon atoms and 1 or more heteroatoms independently selected from S, N or O. A heterocycloalkyl may be a single (monocyclic) ring. Examples of single-ring heterocycloalkyls include, but are not limited to, oxetanyl, azetidinyl, thietanyl, tetrahydrofuranyl, dihydrofuranyl, tetrahydrothienyl, dihydrothienyl, imidazolidinyl, oxazolidinyl, imidazolinyl, isoxazolidinyl, pyrrolidinyl, 2-pyrrolinyl, 3-pyrrolinyl, tetrahydropyranyl, dihydropyranyl, dioxanyl, 1,3-dioxolanyl, 1,4-dithianyl, hexahydropyrimidine, morpholinyl, piperazinyl, piperidinyl, 2H-pyranyl, 4H-pyranyl, pyrazolidinyl, pyrazolinyl, 1,2,3,6-tetrahydropyridinyl, tetrahydrothiopyranyl, thiomorpholinyl, thioxanyl, trithianyl, azepanyl, 2,3,4,5-tetrahydro-1H-azepinyl, oxepanyl, 2,3,4,5-tetrahydro-1H-oxepinyl, thiepanyl, 3,4,5-tetrahydro-1H-thiepinyl, azocanyl, thiocanyl, oxocanyl, tetrahydro-2H-thiopyranyl 1,1-dioxide, 3,4,5,6-tetrahydro-2H-oxocinyl, and the like. A heterocycloalkyl may alternatively be polycyclic (contain more than one ring). Examples of polycyclic heterocycloalkyls include bridged, fused, and spirocyclic heterocycloalkyls in which at least one ring is a heterocycloalkyl and the others are heterocycloalkyl, or cycloalkyl rings.

The term “hydroxy” or “hydroxyl” means —OH.

The term “hydroxyalkyl,” as used herein, refers to a hydroxy group, as defined herein, appended to the parent molecular moiety through an alkyl group, as defined herein. The “hydroxyalkyl” group may include one or more hydroxyl groups. Representative examples of hydroxyalkyl include, but are not limited to, hydroxymethyl, 2-hydroxyethyl, 3-hydroxypropyl, 2-ethyl-4-hydroxyheptyl, 2,3-dihydroxypropyl, and the like.

In some instances, the number of carbon atoms in a moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms and “C₁-C₃ alkyl” means an alkyl substituent containing from 1 to 3 carbon atoms. Also, for example, “C₆-C₁₀ aryl” as used herein, means phenyl or a bicyclic aryl with 6 to 10 carbon atoms. In some instances, the number of ring atoms in a moiety is indicated by the prefix “x-y membered”, wherein x is the minimum and y is the maximum number of ring atoms in the substituent. Thus, for example, the term “5- to 6-membered heteroaryl” means a heteroaryl containing 5 to 6 ring atoms.

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The term “oxo,” as used herein, means a ═O group.

The phrase “pharmaceutical composition” refers to a composition suitable for administration in medical or veterinary use.

The phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts have been described in S. M. Berge et al. Journal of Pharmaceutical Science, vol. 66, 1977, pp. 1-16.

The terms “prevent,” “preventing,” and “prevention” refer to a method of preventing the onset of a disease and/or its attendant symptoms or barring a subject from acquiring a disease. As used herein, “prevent,” “preventing,” and “prevention” also include delaying the onset of a disease and/or its attendant symptoms and reducing a subject's risk of acquiring or developing a disease or disorder.

The term “subject,” as used herein, refers to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

If a moiety is described as “substituted,” a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

The term “tautomer,” as used herein, refers to an equilibrium mixture wherein a proton of a compound shifts from one atom to another. Examples of tautomers include, but are not limited to, keto-enol, phenol-keto, oxime-nitroso, nitro-aci, imine-enamine and the like. Tautomeric forms are intended to be encompassed by the scope of this invention, even though only one tautomeric form may be depicted.

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered alone or in conjunction with one or more therapeutic agents for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The terms “treat,” “treating,” and “treatment,” as used herein, refer to a method of alleviating or abrogating a disease and/or its attendant symptoms.

### Compounds

Compounds of the present disclosure can have the formula (I) as described above in the Summary and Detailed Description.

In one embodiment, compounds of the present disclosure are represented by formula (I), and pharmaceutically acceptable salt thereof, where W is C—R²; and the remaining variables are as defined for formula (I).

In one embodiment, compounds of the present disclosure are represented by formula (I), and pharmaceutically acceptable salt thereof, where W is C—R²; Ring A is indoline; and the remaining variables are as defined for formula (I).

In one embodiment, compounds of the present disclosure are represented by formula (I), and pharmaceutically acceptable salt thereof, where W is C—R²; X is —CR⁶R⁷—;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R², R⁶, and R⁷ are each independently hydrogen; and
the remaining variables are as defined for formula (I).

In one embodiment, compounds of the present disclosure are represented by formula (I), and pharmaceutically acceptable salt thereof, where W is C—R²; X is —CR⁶R⁷—;
Ring A is indole;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R², R⁶, and R⁷ are each independently hydrogen; and
the remaining variables are as defined for formula (I).

In one embodiment, compounds of the present disclosure are represented by formula (I), and pharmaceutically acceptable salt thereof, where W is C—R²; X is —CR⁶R⁷—; Y is piperidinediyl; L¹ is a bond;
Ring A is indole;
R¹ is selected from the group consisting of C₁-C₈alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R², R⁶, and R⁷ are each independently hydrogen; and
the remaining variables are as defined for formula (I).

In one embodiment, compounds of the present disclosure are represented by formula (I), and pharmaceutically acceptable salt thereof, where W is C—R²; X is —CR⁶R⁷—;
Ring A is selected from the group consisting of dihydroindene, and tetrahydronaphthalene;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R², R⁶, and R⁷ are each independently hydrogen; and
the remaining variables are as defined for formula (I).

In one embodiment, compounds of the present disclosure are represented by formula (I), and pharmaceutically acceptable salt thereof, where X is —O—; and the remaining variables are as defined for formula (I).

In one embodiment, compounds of the present disclosure are represented by formula (I), and pharmaceutically acceptable salt thereof, where W is N; X is —O—; and the remaining variables are as defined for formula (I).

In one embodiment, compounds of the present disclosure are represented by formula (I), and pharmaceutically acceptable salt thereof, where W is C—R²; X is —O—; and the remaining variables are as defined for formula (I).

In one embodiment, compounds of the present disclosure are represented by formula (I), and pharmaceutically acceptable salt thereof, where W is C—R²; X is —O—;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen; and the remaining variables are as defined for formula (I).

In one embodiment, compounds of the present disclosure are represented by formula (I), and pharmaceutically acceptable salt thereof, where W is C—R²; X is —O—;
R¹ is selected from the group consisting of pyrazolyl, pyridinyl, and thiazolyl; wherein each R¹ pyrazolyl, pyridinyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen; and the remaining variables are as defined for formula (I).

In one embodiment, compounds of the present disclosure are represented by formula (I), and pharmaceutically acceptable salt thereof, where X is —NR⁸—; and the remaining variables are as defined for formula (I).

In one embodiment, compounds of the present disclosure are represented by formula (I), and pharmaceutically acceptable salt thereof, where W is N; X is —NR⁸—; and the remaining variables are as defined for formula (I).

In one embodiment, compounds of the present disclosure are represented by formula (I), and pharmaceutically acceptable salt thereof, where W is C—R²; X is —NR⁸—; and the remaining variables are as defined for formula (I).

In one embodiment, compounds of the present disclosure are represented by formula (I), and pharmaceutically acceptable salt thereof, where W is C—R²; X is —NR⁸—;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen; and the remaining variables are as defined for formula (I).

In one embodiment, compounds of the present disclosure are represented by formula (I), and pharmaceutically acceptable salt thereof, where W is C—R²; X is —NR⁸—;
R¹ is selected from the group consisting of pyrazolyl, pyridinyl, and thiazolyl; wherein each R¹ pyrazolyl, pyridinyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen; and the remaining variables are as defined for formula (I).

In one embodiment, compounds of the present disclosure are represented by formula (I), and pharmaceutically acceptable salt thereof, where ring A is benzofuran; and the remaining variables are as defined for formula (I).

In one embodiment, compounds of the present disclosure are represented by formula (I), and pharmaceutically acceptable salt thereof, where W is C—R²; X is —CR⁶R⁷—; ring A is benzofuran; and the remaining variables are as defined for formula (I).

In one embodiment, compounds of the present disclosure are represented by formula (I), and pharmaceutically acceptable salt thereof, where ring A is benzoimidazole; and the remaining variables are as defined for formula (I).

In one embodiment, compounds of the present disclosure are represented by formula (I), and pharmaceutically acceptable salt thereof, where W is C—R²; X is —CR⁶R⁷—; ring A is benzoimidazole; and the remaining variables are as defined for formula (I).

In one embodiment, compounds of the present disclosure are represented by formula (I), and pharmaceutically acceptable salt thereof, where ring A is chromane; and the remaining variables are as defined for formula (I).

In one embodiment, compounds of the present disclosure are represented by formula (I), and pharmaceutically acceptable salt thereof, where W is C—R²; X is —CR⁶R⁷—; ring A is chromane; and the remaining variables are as defined for formula (I).

In one embodiment, compounds of the present disclosure are represented by formula (I), and pharmaceutically acceptable salt thereof, where ring A is dihydroindene; and the remaining variables are as defined for formula (I).

In one embodiment, compounds of the present disclosure are represented by formula (I), and pharmaceutically acceptable salt thereof, where W is C—R²; X is —CR⁶R⁷—; ring A is dihydroindene; and the remaining variables are as defined for formula (I).

In one embodiment, compounds of the present disclosure are represented by formula (I), and pharmaceutically acceptable salt thereof, where ring A is dihydropyrrolopyridine; and the remaining variables are as defined for formula (I).

In one embodiment, compounds of the present disclosure are represented by formula (I), and pharmaceutically acceptable salt thereof, where W is C—R²; X is —CR⁶R⁷—; ring A is dihydropyrrolopyridine; and the remaining variables are as defined for formula (I).

In one embodiment, compounds of the present disclosure are represented by formula (I), and pharmaceutically acceptable salt thereof, where ring A is indazole; and the remaining variables are as defined for formula (I).

In one embodiment, compounds of the present disclosure are represented by formula (I), and pharmaceutically acceptable salt thereof, where W is C—R²; X is —CR⁶R⁷—; ring A is indazole; and the remaining variables are as defined for formula (I).

In one embodiment, compounds of the present disclosure are represented by formula (I), and pharmaceutically acceptable salt thereof, where ring A is indole; and the remaining variables are as defined for formula (I).

In one embodiment, compounds of the present disclosure are represented by formula (I), and pharmaceutically acceptable salt thereof, where W is C—R²; X is —CR⁶R⁷—; ring A is indole; and the remaining variables are as defined for formula (I).

In one embodiment, compounds of the present disclosure are represented by formula (I), and pharmaceutically acceptable salt thereof, where ring A is indoline; and the remaining variables are as defined for formula (I).

In one embodiment, compounds of the present disclosure are represented by formula (I), and pharmaceutically acceptable salt thereof, where W is C—R²; X is —CR⁶R⁷—; ring A is indoline; and the remaining variables are as defined for formula (I).

In one embodiment, compounds of the present disclosure are represented by formula (I), and pharmaceutically acceptable salt thereof, where ring Ais isoindoline; and the remaining variables are as defined for formula (I).

In one embodiment, compounds of the present disclosure are represented by formula (I), and pharmaceutically acceptable salt thereof, where W is C—R²; X is —CR⁶R⁷—; ring A is isoindoline; and the remaining variables are as defined for formula (I).

In one embodiment, compounds of the present disclosure are represented by formula (I), and pharmaceutically acceptable salt thereof, where ring A is pyrrolopyridine; and the remaining variables are as defined for formula (I).

In one embodiment, compounds of the present disclosure are represented by formula (I), and pharmaceutically acceptable salt thereof, where W is C—R²; X is —CR⁶R⁷—; ring A is pyrrolopyridine; and the remaining variables are as defined for formula (I).

In one embodiment, compounds of the present disclosure are represented by formula (I), and pharmaceutically acceptable salt thereof, where ring A is tetrahydroisoquinoline; and the remaining variables are as defined for formula (I).

In one embodiment, compounds of the present disclosure are represented by formula (I), and pharmaceutically acceptable salt thereof, where W is C—R²; X is —CR⁶R⁷—; ring A is tetrahydroisoquinoline; and the remaining variables are as defined for formula (I).

In one embodiment, compounds of the present disclosure are represented by formula (I), and pharmaceutically acceptable salt thereof, where ring A is tetrahydronaphthalene; and the remaining variables are as defined for formula (I).

In one embodiment, compounds of the present disclosure are represented by formula (I), and pharmaceutically acceptable salt thereof, where W is C—R²; X is —CR⁶R⁷—; ring A is tetrahydronaphthalene; and the remaining variables are as defined for formula (I).

In one embodiment, compounds of the present disclosure are represented by formula (I), and pharmaceutically acceptable salt thereof, where ring A is tetrahydronaphthyridine; and the remaining variables are as defined for formula (I).

In one embodiment, compounds of the present disclosure are represented by formula (I), and pharmaceutically acceptable salt thereof, where W is C—R²; X is —CR⁶R⁷—; ring A is tetrahydronaphthyridine; and the remaining variables are as defined for formula (I).

In one embodiment, compounds of the present disclosure are represented by formula (I), and pharmaceutically acceptable salt thereof, where ring A is tetrahydroquinoline; and the remaining variables are as defined for formula (I).

In one embodiment, compounds of the present disclosure are represented by formula (I), and pharmaceutically acceptable salt thereof, where W is C—R²; X is —CR⁶R⁷—; ring A is tetrahydroquinoline; and the remaining variables are as defined for formula (I).

In one embodiment, compounds of the present disclosure are represented by formula (II), and pharmaceutically acceptable salt thereof, where R¹ is selected from the group consisting of pyrazolyl, and pyridinyl; wherein each R¹ pyrazolyl, and pyridinyl is optionally substituted with one or more substituents independently selected from the group consisting of C₁-C₄ alkyl, C₁-C₄ haloalkyl, C₁-C₄ alkoxy, CN, F, Cl, Br, and I; and the remaining variables are as defined for formula (II).

In one embodiment, compounds of the present disclosure are represented by formula (II), and pharmaceutically acceptable salt thereof, where L¹ is a bond; R³ is selected from the group consisting of hydrogen, C₁-C₄ alkyl, C₁-C₄ hydroxyalkyl, —OH, —NR^{3a}R^{3b}; R⁵ is selected from the group consisting of F, and C₁-C₄ alkyl; and the remaining variables are as defined for formula (II).

In one embodiment, compounds of the present disclosure are represented by formula (III), and pharmaceutically acceptable salt thereof, where Y is piperazinediyl; L¹ is a bond; and the remaining variables are as defined for formula (III).

In one embodiment, compounds of the present disclosure are represented by formula (III), and pharmaceutically acceptable salt thereof, where Y is octahydropyrrolopyrazinediyl; L¹ is a bond; and the remaining variables are as defined for formula (III).

In one embodiment, compounds of the present disclosure are represented by formula (III), and pharmaceutically acceptable salt thereof, where Y is a bond; L¹ is selected from the group consisting of —N(R^{a})—, and —NHCO₂—(CH₂)ₚ—; and the remaining variables are as defined for formula (III).

In one embodiment, compounds of the present disclosure are represented by formula (III), and pharmaceutically acceptable salt thereof, where Y is a bond; L¹ is selected from the group consisting of —N(R^{a})—, and —NHCO₂—(CH₂)ₚ—; R³ is selected from the group consisting of C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR^{3c}, and —C(O)NH₂; wherein each R³ azetidinyl and dioxolanyl is optionally substituted with one or more C₁-C₄ alkyl substituents; and the remaining variables are as defined for formula (III).

In one embodiment, compounds of the present disclosure are represented by formula (III), and pharmaceutically acceptable salt thereof, where Y is a bond; L¹ is selected from the group consisting of —N(R^{a})—, and —NHCO₂—(CH₂)ₚ—; R³ is selected from the group consisting of C₁-C₄ alkyl, C₁-C₄ hydroxyalkyl, azetidinyl, and —NR^{3a}R^{3b}; wherein each R³ azetidinyl and dioxolanyl is optionally substituted with one or more C₁-C₄ alkyl substituents; and the remaining variables are as defined for formula (III).

In one embodiment, compounds of the present disclosure are represented by formula (III), and pharmaceutically acceptable salt thereof, where Y is a bond; L¹ is selected from the group consisting of —N(R^{a})—, and —NHCO₂—(CH₂)ₚ—; R¹ is selected from the group consisting of pyrazolyl, pyridinyl, and thiazolyl; wherein each R¹ pyrazolyl, pyridinyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; and the remaining variables are as defined for formula (III).

In one embodiment, compounds of the present disclosure are represented by formula (III), and pharmaceutically acceptable salt thereof, where Y is a bond; L¹ is selected from the group consisting of —N(R^{a})—, and —NHCO₂—(CH₂)ₚ—; R¹ is selected from the group consisting of pyrazolyl, and pyridinyl, wherein each R¹ pyrazolyl, and pyridinyl, is optionally substituted with one or more substituents independently selected from the group consisting of C₁-C₄ alkyl, and F; and the remaining variables are as defined for formula (III).

In one embodiment, compounds of the present disclosure are represented by formula (IV), and pharmaceutically acceptable salt thereof, where r is 1; and the remaining variables are as defined for formula (IV).

In one embodiment, compounds of the present disclosure are represented by formula (IV), and pharmaceutically acceptable salt thereof, where r is 2; and the remaining variables are as defined for formula (IV).

In one embodiment, compounds of the present disclosure are represented by formula (IV), and pharmaceutically acceptable salt thereof, where R¹ is selected from the group consisting of pyrazolyl, and pyridinyl, wherein each R¹ pyrazolyl, and pyridinyl, is optionally substituted with one or more substituents independently selected from the group consisting of C₁-C₄ alkyl, C₁-C₄ haloalkyl, C₁-C₄ alkoxy, CN, F, Cl, Br, and I; and the remaining variables are as defined for formula (IV).

In one embodiment, compounds of the present disclosure are represented by formula (IV), and pharmaceutically acceptable salt thereof, where R⁵ is selected from the group consisting of hydrogen, F, and C₁-C₄ alkyl; and the remaining variables are as defined for formula (IV).

In one embodiment, compounds of the present disclosure are represented by formula (IV), and pharmaceutically acceptable salt thereof, where R¹ is selected from the group consisting of pyrazolyl, and pyridinyl, wherein each R¹ pyrazolyl, and pyridinyl, is optionally substituted with one or more substituents independently selected from the group consisting of C₁-C₄ alkyl, C₁-C₄ haloalkyl, C₁-C₄ alkoxy, CN, F, Cl, Br, and I; R is selected from the group consisting of C₁-C₄ alkyl, and C₁-C₄ hydroxyalkyl; R⁵ is selected from the group consisting of hydrogen, F, and C₁-C₄ alkyl; and the remaining variables are as defined for formula (IV).

In one embodiment, compounds of the present disclosure are represented by formula (IV), and pharmaceutically acceptable salt thereof, where R³ is selected from the group consisting of C₁-C₄ alkyl, and C₁-C₄ hydroxyalkyl; R⁵ is selected from the group consisting of hydrogen, F, and C₁-C₄ alkyl; and the remaining variables are as defined for formula (IV).

In one embodiment, compounds of the present disclosure are represented by formula (IV), and pharmaceutically acceptable salt thereof, where R³ is selected from the group consisting of C₁-C₄ alkyl, and C₁-C₄ hydroxyalkyl; and the remaining variables are as defined for formula (IV).

In one embodiment, compounds of the present disclosure are represented by formula (IV), and pharmaceutically acceptable salt thereof, where R³ is C₁-C₄ hydroxyalkyl; and the remaining variables are as defined for formula (IV).

In one embodiment, compounds of the present disclosure are represented by formula (IV), and pharmaceutically acceptable salt thereof, where R¹ is selected from the group consisting of pyrazolyl, pyridinyl, and thiazolyl; wherein each R¹ pyrazolyl, pyridinyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; R³ is C₁-C₄ hydroxyalkyl; and the remaining variables are as defined for formula (IV).

In one embodiment, compounds of the present disclosure are represented by formula (IV), and pharmaceutically acceptable salt thereof, where R³ is C₁-C₄ hydroxyalkyl;
R¹ is selected from the group consisting of

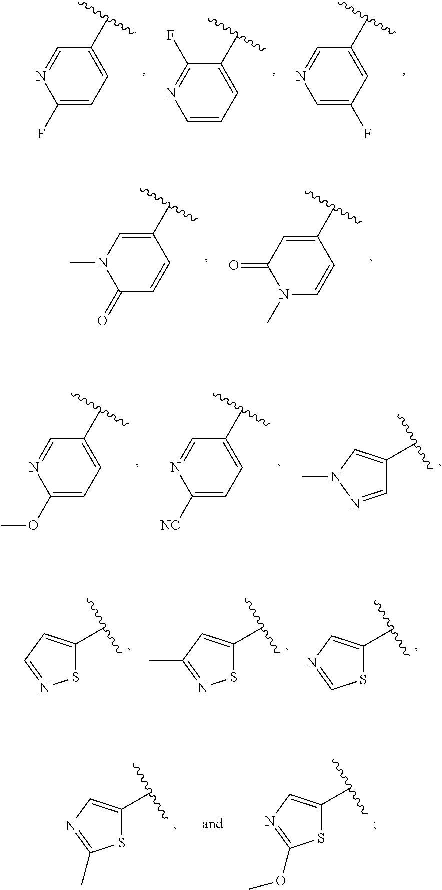

and the remaining variables are as defined for formula (IV).

In one embodiment, compounds of the present disclosure are represented by formula (V), and pharmaceutically acceptable salt thereof, where r is 1; and the remaining variables are as defined for formula (V).

In one embodiment, compounds of the present disclosure are represented by formula (V), and pharmaceutically acceptable salt thereof, where r is 2; and the remaining variables are as defined for formula (V).

In one embodiment, compounds of the present disclosure are represented by formula (V), and pharmaceutically acceptable salt thereof, where R⁵ is hydrogen; and the remaining variables are as defined for formula (V).

In one embodiment, compounds of the present disclosure are represented by formula (V), and pharmaceutically acceptable salt thereof, where R¹ is selected from the group consisting of pyrazolyl, and pyridinyl, wherein each R¹ pyrazolyl, and pyridinyl, is optionally substituted with one or more substituents independently selected from the group consisting of C₁-C₄ alkyl, C₁-C₄ haloalkyl, C₁-C₄ alkoxy, CN, F, Cl, Br, and I; R⁵ is hydrogen; and the remaining variables are as defined for formula (V).

In one embodiment, compounds of the present disclosure are selected from the group consisting of 2-amino-N-{(1S,2S)-2-[(4-{2-[3-(dimethylamino)propanoyl]-2,3-dihydro-1H-isoindol-5-yl}phenyl)methoxy]cyclopentyl}-5-(1-methyl-1H-pyrazol-4-yl)pyridine-3-carboxamide; 2-amino-N-{(1S,2S)-2-[(4-{1-[(dimethylamino)acetyl]-2,3-dihydro-1H-indol-5-yl}phenyl)methoxy]cyclopentyl}-5-(1-methyl-1H-pyrazol-4-yl)pyridine-3-carboxamide; 2-amino-N-[(1S,2S)-2-{[4-(1-methyl-2-oxo-2,3-dihydro-1H-indol-5-yl)phenyl]methoxy}cyclopentyl]-5-(1-methyl-1H-pyrazol-4-yl)pyridine-3-carboxamide; tert-butyl 4-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}piperidine-1-carboxylate; 2-amino-5-(1-methyl-1H-pyrazol-4-yl)-N-[(1S,2S)-2-({4-[1-(piperidin-4-yl)-1H-indol-5-yl]phenyl}methoxy)cyclopentyl]pyridine-3-carboxamide; 2-amino-N-[(1S,2S)-2-({4-[1-(1-methylpiperidin-4-yl)-1H-indol-5-yl]phenyl}methoxy)cyclopentyl]-5-(1-methyl-1H-pyrazol-4-yl)pyridine-3-carboxamide; 2-amino-N-{(1S,2S)-2-[(4-{1-[1-(2-hydroxyethyl)piperidin-4-yl]-1H-indol-5-yl}phenyl)methoxy]cyclopentyl}-5-(1-methyl-iH-pyrazol-4-yl)pyridine-3-carboxamide; 2-amino-N-{(1S,2S)-2-[(4-{1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{2-[2-(dimethylamino)acetamido]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{2-[(1-methylpiperidine-4-carbonyl)amino]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{2-[(1-methylazetidine-3-carbonyl)amino]-2,3-dihydro-1H-inden-5-yl}phenyl)methoxy]cyclopentyl}-5-(1-methyl-1H-pyrazol-4-yl)pyridine-3-carboxamide; 2-amino-N-[(1S,2S)-2-({4-[3,3-dimethyl-1-(1-methylpiperidin-4-yl)-2,3-dihydro-1H-indol-5-yl]phenyl}methoxy)cyclopentyl]-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide; 2-amino-N-{(3S,4R)-4-[(4-{1-[1-(2-hydroxyethyl)piperidin-4-yl]-1H-indol-5-yl}phenyl)methoxy]oxolan-3-yl}-5-(1-methyl-1H-pyrazol-4-yl)pyridine-3-carboxamide; 2-amino-N-[(1S,2S)-2-({4-[3-(1-methylpiperidin-4-yl)-1H-indol-6-yl]phenyl}methoxy)cyclopentyl]-5-(1-methyl-1H-pyrazol-4-yl)pyridine-3-carboxamide; 2-amino-N-[(3S,4R)-4-{[4-(1-methyl-1H-indol-5-yl)phenyl]methoxy}oxolan-3-yl]-5-(1-methyl-1H-pyrazol-4-yl)pyridine-3-carboxamide; 2-amino-5-(1-methyl-1H-pyrazol-4-yl)-N-[(1S,2S)-2-{[4-(1,3,3-trimethyl-2,3-dihydro-1H-indol-5-yl)phenyl]methoxy}cyclopentyl]pyridine-3-carboxamide; 2-amino-N-{(3S,4R)-4-[(4-{1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide; 6-amino-6′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide; 6-amino-6′-fluoro-N-{(3S,4R)-4-[(4-{1-[4-(2-hydroxyethyl)piperazin-1-yl]-2,3-dihydro-1H-inden-5-yl}phenyl)methoxy]oxolan-3-yl}[3,3′-bipyridine]-5-carboxamide; 2-amino-N-{(1S,2S)-2-[(4-{1-[1-(2-hydroxyethyl)piperidin-4-yl]-1H-indol-5-yl}phenyl)methoxy]cyclopentyl}-5-(trifluoromethyl)pyridine-3-carboxamide; 6-amino-6′-fluoro-N-{(1S,2S)-2-[(4-{1-[1-(2-hydroxyethyl)piperidin-4-yl]-1H-indol-5-yl}phenyl)methoxy]cyclopentyl}[3,3′-bipyridine]-5-carboxamide; 2-amino-N-{(1S,2S)-2-[(4-{1-[1-(2,3-dihydroxypropyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide; 2-amino-N-{(3S,4R)-4-[(4-{1-[1-(2-hydroxyethyl)piperidin-4-yl]-1H-indol-5-yl}phenyl)methoxy]oxolan-3-yl}-5-(trifluoromethyl)pyridine-3-carboxamide; 6-amino-6′-fluoro-N-{(3S,4R)-4-[(4-{1-[1-(2-hydroxyethyl)piperidin-4-yl]-1H-indol-5-yl}phenyl)methoxy]oxolan-3-yl}[3,3′-bipyridine]-5-carboxamide; 2-amino-N-{(1S,2S)-2-[(4-{3-[1-(2-hydroxyethyl)piperidin-4-yl]-1-methyl-1H-indol-6-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-amino-2-oxoethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide; 2-amino-N-{(1S,2S)-2-[(4-{1′-[1-(2-hydroxyethyl)piperidin-4-yl]-1′,2′-dihydrospiro[cyclopropane-1,3′-indol]-5′-yl}phenyl)methoxy]cyclopentyl}-5-(1-methyl-1H-pyrazol-4-yl)pyridine-3-carboxamide; 2-amino-N-[(1S,2S)-2-{[4-(1H-indol-7-yl)phenyl]methoxy}cyclopentyl]-5-(1-methyl-1H-pyrazol-4-yl)pyridine-3-carboxamide; 6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-2′-oxo-1′,2′-dihydro[3,4′-bipyridine]-5-carboxamide; 6-amino-6′-fluoro-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}[3,3′-bipyridine]-5-carboxamide; 2-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-5-(1-methyl-1H-pyrazol-4-yl)pyridine-3-carboxamide; 2-amino-N-{(1S,2S)-2-[(4-{(1R)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 6-amino-6′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide; 6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-6′-oxo-1′,6′-dihydro[3,3′-bipyridine]-5-carboxamide; 6-amino-6′-fluoro-N-{(3S,4S)-4-[(4-{1-[1-(2-hydroxyethyl)piperidin-4-yl]-3,3-dimethyl-2,3-dihydro-1H-indol-5-yl}phenyl)methoxy]-1-methylpyrrolidin-3-yl}[3,3′-bipyridine]-5-carboxamide; rac-2-amino-N-{(1S,2S,4S)-4-hydroxy-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3-methyl-3-propyl-2,3-dihydro-1H-indol-5-yl}phenyl)methoxy]cyclopentyl}-5-(1-methyl-1H-pyrazol-4-yl)pyridine-3-carboxamide; 2-amino-N-{(1S,2S)-2-[(4-{3-ethyl-1-[1-(2-hydroxyethyl)piperidin-4-yl]-3-methyl-2,3-dihydro-1H-indol-5-yl}phenyl)methoxy]cyclopentyl}-5-(1-methyl-1H-pyrazol-4-yl)pyridine-3-carboxamide; 3-amino-6-(6-fluoropyridin-3-yl)-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyrazine-2-carboxamide; 2-amino-5-(1,3-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide; 2-amino-N-{(1S,2S)-2-[(4-{1-[1-(2-hydroxyethyl)piperidin-4-yl]-1,2-dihydrospiro[indole-3,3′-oxolan]-5-yl}phenyl)methoxy]cyclopentyl}-5-(1-methyl-1H-pyrazol-4-yl)pyridine-3-carboxamide; 6-amino-5′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide; 2-amino-N-{(3S,4R)-4-[(4-{(1S)-1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide; 6-amino-6′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide; 2-amino-5-(1,5-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide; 6-amino-2′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide; 2-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(prop-1-en-2-yl)pyridine-3-carboxamide; 3-amino-6-(6-fluoropyridin-3-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyrazine-2-carboxamide; 2-amino-5-(1-methyl-1H-pyrazol-4-yl)-N-[(1S,2S)-2-({4-[2-(trifluoromethyl)-1H-indol-5-yl]phenyl}methoxy)cyclopentyl]pyridine-3-carboxamide; 2-amino-N-[(1S,2S)-2-{[4-(1,3-dimethyl-1H-indol-5-yl)phenyl]methoxy}cyclopentyl]-5-(1-methyl-iH-pyrazol-4-yl)pyridine-3-carboxamide; 2-amino-N-[(1S,2S)-2-{[4-(2,3-dimethyl-1H-indol-5-yl)phenyl]methoxy}cyclopentyl]-5-(1-methyl-1H-pyrazol-4-yl)pyridine-3-carboxamide; 2-amino-N-[(1S,2S)-2-{[4-(2-methyl-1H-indol-5-yl)phenyl]methoxy}cyclopentyl]-5-(1-methyl-1H-pyrazol-4-yl)pyridine-3-carboxamide; 2-amino-N-[(1S,2S)-2-({4-[1-(2-hydroxyethyl)-1H-indol-5-yl]phenyl}methoxy)cyclopentyl]-5-(1-methyl-1H-pyrazol-4-yl)pyridine-3-carboxamide; 3-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}propanoic acid; 2-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-5-(1-methyl-1H-pyrazol-4-yl)pyridine-3-carboxamide; 2-amino-5-(1-methyl-1H-pyrazol-4-yl)-N-[(1S,2S)-2-({4-[1-(propan-2-yl)-1H-indol-5-yl]phenyl}methoxy)cyclopentyl]pyridine-3-carboxamide; {5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}acetic acid; 6-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-6′-fluoro[3,3′-bipyridine]-5-carboxamide; 6-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-1′-methyl-2′-oxo-1′,2′-dihydro[3,4′-bipyridine]-5-carboxamide; 2-amino-N-[(1S,2S)-2-({4-[1-(difluoromethyl)-1H-indol-5-yl]phenyl}methoxy)cyclopentyl]-5-(1-methyl-1H-pyrazol-4-yl)pyridine-3-carboxamide; 2-amino-N-{(1S,2S)-2-[(4-{(1R)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{(1S)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 2-amino-N-[(1S,2S)-2-({4-[1-(2-aminoethyl)-1H-indol-5-yl]phenyl}methoxy)cyclopentyl]-5-(1-methyl-1H-pyrazol-4-yl)pyridine-3-carboxamide; 2-amino-N-[(1S,2S)-2-{[4-(1H-indol-5-yl)phenyl]methoxy}cyclopentyl]-5-(1-methyl-1H-pyrazol-4-yl)pyridine-3-carboxamide; 2-amino-N-{(1S,2S)-2-[(4-{3-[4-(2-hydroxyethyl)piperazin-1-yl]-2-oxo-2,3-dihydro-1H-indol-6-yl}phenyl)methoxy]cyclopentyl}-5-(1-methyl-1H-pyrazol-4-yl)pyridine-3-carboxamide; 2-amino-N-{(1S,2S)-2-[(4-{1-[2-(dimethylamino)ethyl]-1H-indol-5-yl}phenyl)methoxy]cyclopentyl}-5-(1-methyl-1H-pyrazol-4-yl)pyridine-3-carboxamide; 3-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-6-(1-methyl-iH-pyrazol-4-yl)pyrazine-2-carboxamide; 2-amino-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide; 6-amino-6′-fluoro-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}[3,3′-bipyridine]-5-carboxamide; 6-amino-6′-fluoro-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}[3,3′-bipyridine]-5-carboxamide; 2-amino-N-{(1S,2S)-2-[(4-{1-[1-(2-hydroxyethyl)piperidin-4-yl]-3,3-dimethyl-2-oxo-2,3-dihydro-1H-pyrrolo[2,3-b]pyridin-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3,3-dimethyl-2,3-dihydro-1H-pyrrolo[2,3-b]pyridin-5-yl}phenyl)methoxy]cyclopentyl}-5-(1-methyl-1H-pyrazol-4-yl)pyridine-3-carboxamide; 6-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-6′-fluoro[3,3′-bipyridine]-5-carboxamide; 2-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-5-(1-methyl-1H-pyrazol-4-yl)pyridine-3-carboxamide; 6-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide; 2-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 6-amino-2′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide; 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(1-methyl-iH-pyrazol-4-yl)pyridine-3-carboxamide; 6-amino-6′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 2-amino-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 6-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-6′-fluoro[3,3′-bipyridine]-5-carboxamide; 6-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide; 2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide; 6-amino-6′-fluoro-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}[3,3′-bipyridine]-5-carboxamide; 6-amino-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}-1′-methyl-6′-oxo-1′,6′-dihydro[3,3′-bipyridine]-5-carboxamide; 6-amino-2′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide; 6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide; 2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide; 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-6′-fluoro[3,3′-bipyridine]-5-carboxamide; 2-amino-N-{(1S,2S)-2-[(4-{3-[4-(2-hydroxyethyl)piperazin-1-yl]-1-benzofuran-6-yl}phenyl)methoxy]cyclopentyl}-5-(1-methyl-1H-pyrazol-4-yl)pyridine-3-carboxamide; 2-amino-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}-5-(1-methyl-iH-pyrazol-4-yl)pyridine-3-carboxamide; 6-amino-6′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 2-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide; 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide; 2-amino-N-{(1S,2S)-2-[(4-{(1R)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide; 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-2′-fluoro[3,3′-bipyridine]-5-carboxamide; 2-amino-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide; 6-amino-6′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 2-amino-N-{(1S,2S)-2-[(4-{1-[4-(2-hydroxyethyl)piperazin-1-yl]-2,2-dimethyl-2,3-dihydro-1H-inden-5-yl}phenyl)methoxy]cyclopentyl}-5-(1-methyl-1H-pyrazol-4-yl)pyridine-3-carboxamide; rac-6-amino-6′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide; 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-1′-methyl-2′-oxo-1′,2′-dihydro[3,4′-bipyridine]-5-carboxamide; 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide; 2-amino-N-[(1S,2S)-2-({4-[1-(dimethylamino)-2,2-dimethyl-2,3-dihydro-1H-inden-5-yl]phenyl}methoxy)cyclopentyl]-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3-methyl-1H-pyrrolo[2,3-b]pyridin-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-1H-indazol-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-1,2,3,4-tetrahydroquinolin-6-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-3,3-dimethyl-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide; 2-amino-N-{(1S,2S)-2-[(4-{1-[1-(2-hydroxyethyl)piperidin-4-yl]-4,4-dimethyl-1,2,3,4-tetrahydroquinolin-6-yl}phenyl)methoxy]cyclopentyl}-5-(1-methyl-1H-pyrazol-4-yl)pyridine-3-carboxamide; 6-amino-1′-methyl-N-[(1S,2S)-2-({4-[(1S)-1-(4-methylpiperazin-1-yl)-2,3-dihydro-1H-inden-5-yl]phenyl}methoxy)cyclopentyl]-2′-oxo-1′,2′-dihydro[3,4′-bipyridine]-5-carboxamide; 6-amino-2′-fluoro-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide; 6-amino-6′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide; 6-amino-6′-fluoro-N-{(1S,2S)-2-[(4-{(1R)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide; 6-amino-2′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 2-amino-N-[(1S,2S)-2-({4-[5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide; 2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(1-methyl-1H-pyrazol-4-yl)pyridine-3-carboxamide; 2-amino-5-(1-methyl-1H-pyrazol-4-yl)-N-[(1S,2S)-2-({4-[5-(piperazin-1-yl)-5,6,7,8-tetrahydronaphthalen-2-yl]phenyl}methoxy)cyclopentyl]pyridine-3-carboxamide; 6-amino-5′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide; 2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(trifluoromethyl)pyridine-3-carboxamide; 2-amino-N-{(1S,2S)-2-[(4-{1-[(3,4-dihydroxybutyl)(methyl)amino]-2,3-dihydro-1H-inden-5-yl}phenyl)methoxy]cyclopentyl}-5-(1-methyl-1H-pyrazol-4-yl)pyridine-3-carboxamide; 6-amino-N-{(1S,2S)-2-[(4-{1-[(3,4-dihydroxybutyl)(methyl)amino]-2,3-dihydro-H-inden-5-yl}phenyl)methoxy]cyclopentyl}-6′-fluoro[3,3′-bipyridine]-5-carboxamide; 6-amino-N-{(1S,2S)-2-[(4-{1-[(3,4-dihydroxybutyl)(methyl)amino]-2,3-dihydro-H-inden-5-yl}phenyl)methoxy]cyclopentyl}-2′-fluoro[3,3′-bipyridine]-5-carboxamide; 2-amino-N-{(1S,2S)-2-[(4-{(1S)-1-[4-(1,3-dihydroxypropan-2-yl)piperazin-1-yl]-2,3-dihydro-H-inden-5-yl}phenyl)methoxy]cyclopentyl}-5-(1-methyl-1H-pyrazol-4-yl)pyridine-3-carboxamide; 6-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-2′-fluoro[3,3′-bipyridine]-5-carboxamide; 2-amino-N-{(1S,2S)-2-[(4-{1-[4-(2-hydroxyethyl)piperazin-1-yl]-3,3-dimethyl-2,3-dihydro-H-inden-5-yl}phenyl)methoxy]cyclopentyl}-5-(trifluoromethyl)pyridine-3-carboxamide; 2-amino-N-{(1S,2S)-2-[(4-{(1S)-1-[4-(2-hydroxyethyl)piperazin-1-yl]-2,3-dihydro-H-inden-5-yl}phenyl)methoxy]cyclopentyl}-5-(trifluoromethyl)pyridine-3-carboxamide; 6-amino-5′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide; 2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(trifluoromethyl)pyridine-3-carboxamide; 6-amino-2′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide; 6-amino-2′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-4-methoxypyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxypyridine-3-carboxamide; 2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-4-methoxypyridine-3-carboxamide; 2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]pyridine-3-carboxamide; 6-amino-N-{(1S,2S)-2-[(4-{1-[4-(1,3-dihydroxypropan-2-yl)piperazin-1-yl]-3,3-dimethyl-2,3-dihydro-1H-inden-5-yl}phenyl)methoxy]cyclopentyl}-2′-fluoro[3,3′-bipyridine]-5-carboxamide; 2-amino-5-(1-methyl-1H-pyrazol-4-yl)-N-[(1S,2S)-2-({4-[(5S)-5-(piperazin-1-yl)-5,6,7,8-tetrahydronaphthalen-2-yl]phenyl}methoxy)cyclopentyl]pyridine-3-carboxamide; 2-amino-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide; 2-amino-N-[(1S,2S)-2-({4-[(1S)-1-(4-methylpiperazin-1-yl)-2,3-dihydro-1H-inden-5-yl]phenyl}methoxy)cyclopentyl]-5-(trifluoromethyl)pyridine-3-carboxamide; 2-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide; 2-amino-N-[(1S,2S)-2-({4-[(1R)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide; 2-amino-N-[(1S,2S)-2-({4-[8,8-dimethyl-5-(piperazin-1-yl)-5,6,7,8-tetrahydronaphthalen-2-yl]phenyl}methoxy)cyclopentyl]-5-(1-methyl-iH-pyrazol-4-yl)pyridine-3-carboxamide; 2-amino-N-[(1S,2S)-2-({4-[8,8-dimethyl-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide; 2-amino-N-{(1S,2S)-2-[(4-{5-[4-(2-hydroxyethyl)piperazin-1-yl]-8,8-dimethyl-5,6,7,8-tetrahydronaphthalen-2-yl}phenyl)methoxy]cyclopentyl}-5-(1-methyl-1H-pyrazol-4-yl)pyridine-3-carboxamide; 6-amino-2′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide; 6-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide; 6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6′-methoxy[3,3′-bipyridine]-5-carboxamide; 6-amino-6′-methoxy-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide; 6-amino-6′-cyano-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide; 6-amino-6′-cyano-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxy-5-(1-methyl-1H-pyrazol-4-yl)pyridine-3-carboxamide; 2-amino-N-{(1S,2S)-2-[(4-{1-[1-(2-hydroxyethyl)piperidin-4-yl]-1H-benzimidazol-5-yl}phenyl)methoxy]cyclopentyl}-5-(1-methyl-1H-pyrazol-4-yl)pyridine-3-carboxamide; 2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide; 2-amino-N-{(1S,2S)-2-[(4-{8-[1-(2-hydroxyethyl)piperidin-4-yl]-5,5-dimethyl-5,6,7,8-tetrahydro-1,8-naphthyridin-3-yl}phenyl)methoxy]cyclopentyl}-5-(1-methyl-1H-pyrazol-4-yl)pyridine-3-carboxamide; 6-amino-2′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide; 6-amino-2′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide; 2-amino-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}-5-(1-methyl-1H-pyrazol-4-yl)pyridine-3-carboxamide; 2-amino-N-{(1S,2S)-2-[(4-{8-[1-(2-hydroxyethyl)piperidin-4-yl]-5,6,7,8-tetrahydro-1,8-naphthyridin-3-yl}phenyl)methoxy]cyclopentyl}-5-(1-methyl-1H-pyrazol-4-yl)pyridine-3-carboxamide; 6-amino-2′-fluoro-N-[(1S,2S)-2-({4-[(5S)-5-(4-methylpiperazin-1-yl)-5,6,7,8-tetrahydronaphthalen-2-yl]phenyl}methoxy)cyclopentyl][3,3′-bipyridine]-5-carboxamide; 2-amino-N-[(1S,2S)-2-({4-[(5S)-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide; 6-amino-2′-fluoro-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide; 6-amino-N-[(1S,2S)-2-({4-[(1S)-1-{4-[(2S)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide; 6-amino-N-[(1S,2S)-2-({4-[(1S)-1-{4-[(2R)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide; 6-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-2′-fluoro[3,3′-bipyridine]-5-carboxamide; 2-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-5-(1-methyl-1H-pyrazol-4-yl)pyridine-3-carboxamide; 2-amino-N-[(1S,2S)-2-({4-[(5R)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide; 2-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-iH-pyrazol-4-yl)pyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-5-yl)pyridine-3-carboxamide; 6-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-2′-fluoro[3,3′-bipyridine]-5-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,2-thiazol-5-yl)pyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(3-methyl-1,2-thiazol-5-yl)pyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,3-thiazol-5-yl)pyridine-3-carboxamide; 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[2,3′-bipyridine]-5′-carboxamide; 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6-methoxy[2,3′-bipyridine]-5′-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methoxy-1,3-thiazol-4-yl)pyridine-3-carboxamide; 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-4-yl)pyridine-3-carboxamide; and 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(4-methyl-1,3-thiazol-2-yl)pyridine-3-carboxamide.

Compound names are assigned by using Name 2018 by Advanced Chemical Development (ACD)/ChemSketch 2018.1.1 naming algorithm.

Compounds of the present disclosure may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The present disclosure contemplates various stereoisomers and mixtures thereof and these are specifically included within the scope of this disclosure. Stereoisomers include enantiomers and diastereomers, and mixtures of enantiomers or diastereomers. Individual stereoisomers of compounds of the present disclosure may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the present disclosure may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the present disclosure may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the present disclosure may possess tautomeric forms, as well as geometric isomers, and that these also constitute an aspect of the present disclosure.

The present disclosure includes all pharmaceutically acceptable isotopically-labelled compounds of formula (I) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P and sulfur, such as ³⁵S. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the present disclosure encompasses any tautomeric, geometric, or stereoisomeric form, and mixtures thereof, and is not to be limited merely to any one tautomeric, geometric, or stereoisomeric form utilized within the formula drawings.

### General Synthesis

The compounds of the present disclosure can be better understood in connection with the following synthetic schemes and methods which illustrate a means by which the compounds can be prepared. The compounds of this disclosure can be prepared by a variety of synthetic procedures. Representative procedures are shown in, but are not limited to, Schemes 1-5. In Schemes 1-5, the variables R¹, X, and W are as described in the Summary.

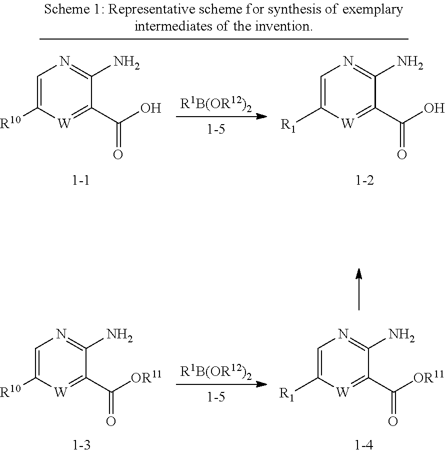

As shown in Scheme 1, compounds of formula (1-2) can be prepared from compounds of formula (1-1). Compounds of formula (1-1) where R¹⁰ is I, Br, or Cl can be coupled with boronic acids of formula (1-5) where R¹² is H (or the boronic ester equivalents where R¹² is alkyl), under Suzuki coupling conditions known to those skilled in the art and widely available in the literature to afford compounds of formula (1-2). The reaction typically requires the use of a base and a catalyst. Examples of bases include, but are not limited to, potassium carbonate, potassium tert-butoxide, sodium carbonate, cesium carbonate, and cesium fluoride. Examples of catalysts include, but are not limited to, [1,1′-bis(diphenylphosphino)ferrocene]dichloropalladium(II) tetrakis(triphenylphosphine)palladium(O), [1,1′-bis(diphenylphosphino)ferrocene]dichloropalladium(II) dichloromethane, bis(triphenylphosphine)palladium(II) dichloride, and tris(dibenzylideneacetone)dipalladium(O). The reaction may be conducted in a solvent such as, but not limited to, water, dioxane, 1,2-dimethoxyethane, N,N-dimethylformamide, toluene, ethanol, tetrahydrofuran and the like, or mixtures thereof. The reaction may be conducted at ambient or elevated temperatures, and optionally in a microwave oven. Alternatively, the ester of formula (1-3) where R¹⁰ is I, Br, or Cl; and R¹¹ is alkyl, may be coupled with boronic acids of formula (1-5) where R¹² is H (or the boronic ester equivalents where R¹² is alkyl), under Suzuki coupling conditions to afford compounds of formula (1-4). Compounds of formula (1-4) may be hydrolyzed to carboxylic acids of formula (1-2) using conditions known to one of skill in the art including under basic conditions such as, for example, lithium hydroxide.

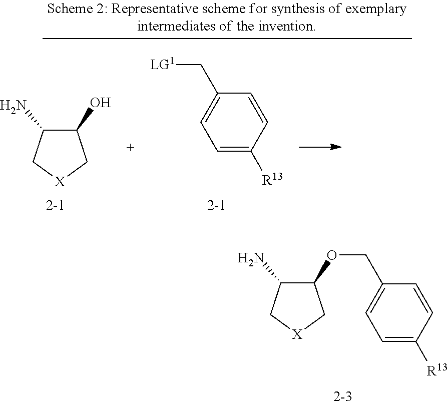

As shown in Scheme 2, compounds of formula (2-3) can be prepared from compounds of formula (2-1). Compounds of formula (2-1) can be treated with compounds of formula (2-2) wherein R¹³ is I, Br, or Cl; and LG¹ is a leaving group, e.g., halogen or sulfonate, under nucleophilic substitution reaction conditions to give compounds of formula (2-3). The nucleophilic substitution reaction is perfomed in the presence of a suitable base including, for example, potassium tert-butoxide in a suitable solvent including, for example, dimethyl sulfoxide.

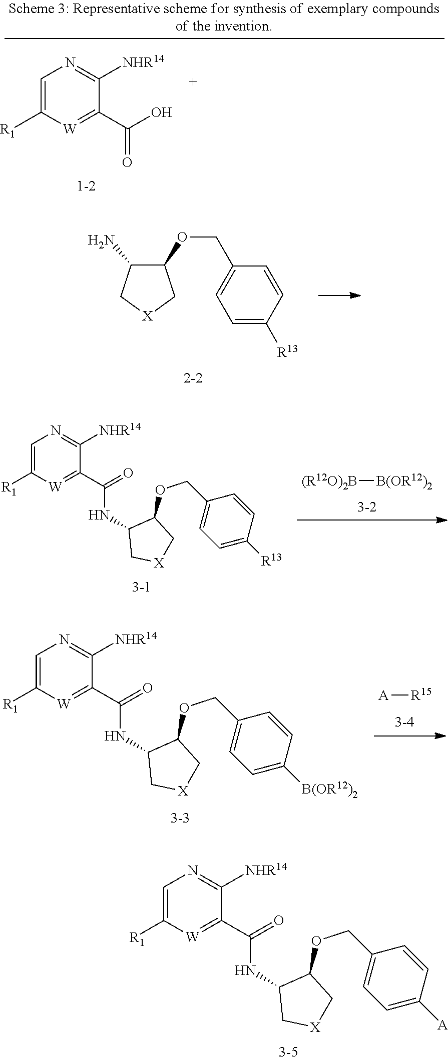

As shown in Scheme 3, compounds of formula (3-5) can be prepared from compounds of formula (1-2). Carboxylic acids of formula (1-2) where R¹⁴ is H, may be coupled with amines of formula (2-2) where R¹³ is I, Br, or Cl under amide bond forming conditions to provide compounds of formula (3-1). Examples of conditions known to generate amides from a mixture of a carboxylic acid and an amine include, but are not limited to, adding a coupling reagent such as, but not limited to, N-(3-dimethylaminopropyl)-N-ethylcarbodiimide or 1-(3-dimethylaminopropyl)-3-ethylcarbodiimide (EDC, EDAC or EDCI) or the corresponding hydrochloride salt, 1,3-dicyclohexylcarbodiimide (DCC), bis(2-oxo-3-oxazolidinyl)phosphinic chloride (BOPCl), N-[(dimethylamino)-1H-1,2,3-triazolo-[4,5-b]pyridin-1-ylmethylene]-N-methylmethanaminium hexafluorophosphate N-oxide or 2-(7-azabenzotriazol-1-yl)-N,N,N′,N′-tetramethyluronium hexafluorophosphate or 1-[bis(dimethylamino)methylene]-1H-1,2,3-triazolo[4,5-b]pyridinium 3-oxid hexafluorophosphate (HATU), O-(benzotriazol-1-yl)-N,N,N′,NV-tetramethyluronium tetrafluoroborate (TBTU), 2-(1H-benzo[d][1,2,3]triazol-1-yl)-1,1,3,3-tetramethylisouronium hexafluorophosphate(V) (HBTU), and 2,4,6-tripropyl-1,3,5,2,4,6-trioxatriphosphinane 2,4,6-trioxide (T3P®). The coupling reagents may be added as a solid, a solution, or as the reagent bound to a solid support resin.

Compounds of formula (3-3) may be prepared by treating compounds of formula (3-1) where R¹³ is I, Br, or Cl; with hypodiboric acids of formula (3-2) where R¹² is H (or the hypodiboric ester equivalents where R¹² is alkyl) in the presence of a palladium(II) catalyst, including, for example, chloro[(tri-tert-butylphosphine)-2-(2-aminobiphenyl)]palladium(II), and a suitable base including, for example, potassium acetate. Compounds of formula (3-5) may be prepared from compounds of formula (3-3). Compounds of formula (3-3) where R¹² is H (or the hypodiboric ester equivalents where R¹² is alkyl) can be coupled with compounds of formula (3-4) where R¹⁵ is is I, Br, or Cl under Suzuki coupling conditions as described above for Scheme 1.

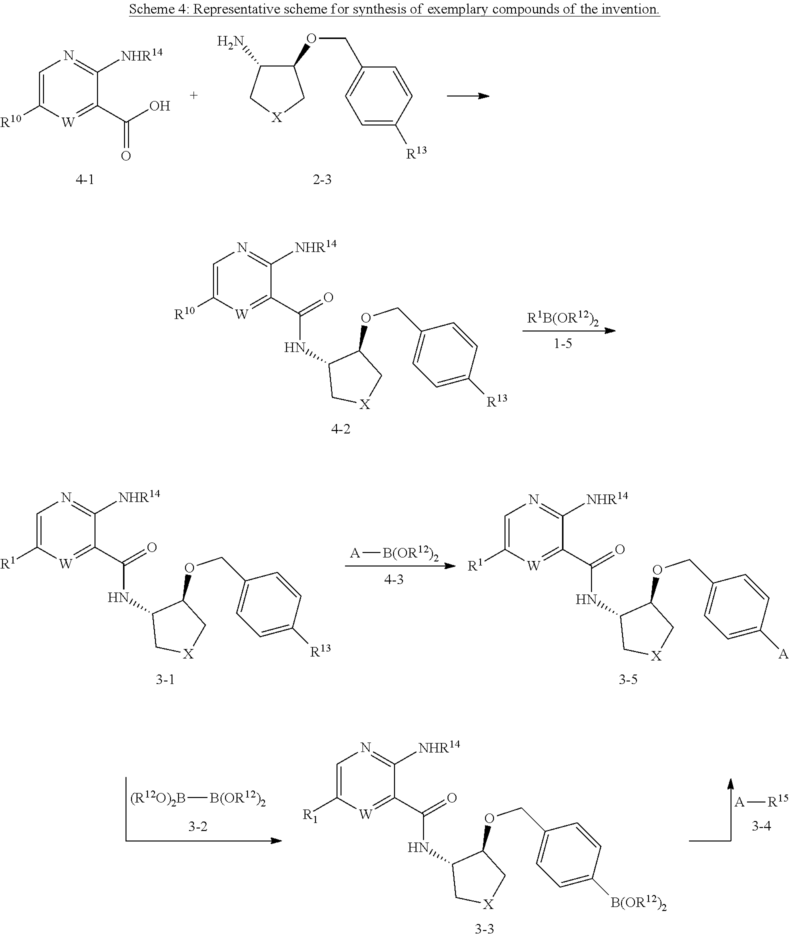

As shown in Scheme 4, compounds of formula (3-5) can be prepared from compounds of formula (4-1). Compounds of formula (4-1) where R¹⁰ is I can be coupled with compounds of formula (2-3) where R¹³ is Br, or Cl, or alternatively where R¹⁰ is Br and R¹³ is Cl; under amide bond forming conditions as described above in Scheme 3 to afford compounds of formula (4-2). Compounds of formula (4-2) can be coupled with boronic acids of formula (1-5) where R¹² is H (or the boronic ester equivalents where R¹² is alkyl), under Suzuki coupling conditions as described above in Scheme 1 to afford compounds of formula (3-1). Compounds of formula (3-5) can be prepared from compounds of formula (3-1) via compounds of formula (3-3) converted to compounds of formula (3-5) as described in Scheme 3. Alternatively, compounds of formula (3-1) can be coupled with compounds of formula (3-3) where R¹² is H (or the hypodiboric ester equivalents where R¹² is alkyl) under Suzuki coupling conditions as described above for Scheme 1 to afford compounds of formula (3-5).

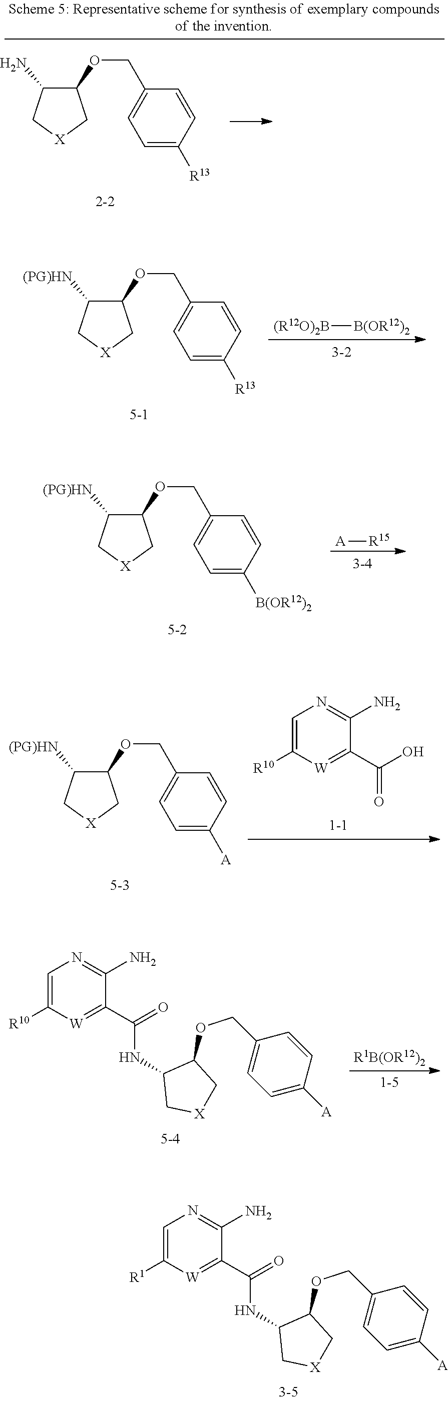

As shown in Scheme 5, compounds of formula (3-5) can be prepared from compounds of formula (2-2). Amines of formula (2-2) where R¹³ is I, Br, or Cl; can be protected with a suitable protecting group (PG) to provide compounds of formula (5-1). For example, amines of formula (2-2) can be treated with di-tert-butyl dicarbonate at ambient temperature in a solvent such as, but not limited to, tetrahydrofuran to provide compounds of formula (5-1) wherein PG is tert-butoxycarbonyl. Compounds of formula (5-2) may be prepared by treating compounds of formula (5-1) with hypodiboric acids of formula (3-2) where R¹² is H (or the hypodiboric ester equivalents where R¹² is alkyl) in the presence of a palladium(II) catalyst, including, for example, chloro[(tri-tert-butylphosphine)-2-(2-aminobiphenyl)]palladium(II), and a suitable base including, for example, potassium acetate. Compounds of formula (5-2) may be coupled with compounds of formula (3-4) where R¹⁵ is is I, Br, or Cl under Suzuki coupling conditions as described above for Scheme 1 to afford compounds of formula (5-3).

Compounds of formula (5-4) may be prepared from compounds of formula (5-3) by removing the protecting group (PG) under conditions known to one of skill in the art to expose the corresponding amine. For example, when PG is tert-butoxycarbonyl, treatment under acidic conditions such as trifluoroacetic acid in dichloromethane removes the protecting group to afford the corresponding amine. The resulting amines may coupled with carboxylic acids of formula (1-1) under amide bond forming conditions such as those described in Scheme 3 to afford compounds of formula (5-4). Coupling compounds of formula (5-4) with boronic acids of formula (1-5) where R¹² is H (or the boronic ester equivalents where R¹² is alkyl), under Suzuki coupling conditions as described in Scheme 1 affords compounds of formula (3-5).

The compounds and intermediates of the present disclosure may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

Many of the compounds of the present disclosure have at least one basic nitrogen whereby the compound can be treated with an acid to form a desired salt. For example, a compound may be reacted with an acid at or above room temperature to provide the desired salt, which is deposited, and collected by filtration after cooling.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the present disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the present disclosure can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the present disclosure is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the present disclosure is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as illustrated in the Examples section are illustrative and are not to be read as limiting the scope of the present disclosure as it is defined in the appended claims. All alternatives, modifications, and equivalents of the synthetic methods and specific examples are included within the scope of the claims.

### Pharmaceutical Compositions

This disclosure also provides for pharmaceutical compositions comprising a therapeutically effective amount of a compound disclosed herein, or a pharmaceutically acceptable salt thereof together with a pharmaceutically acceptable carrier.

The term “pharmaceutically acceptable carrier” as used herein, means a non-toxic, inert solid, semi-solid or liquid filler, diluent, encapsulating material or formulation auxiliary of any type.

The pharmaceutical composition may be a unit dosage form. In such form the preparation is subdivided into unit doses containing appropriate quantities of the active component.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc.

In one embodiment, the composition comprises a compound of any one of formulae (I, II, III, IV, or V), or a pharmaceutically acceptable salt thereof, and a pharmaceutically acceptable excipient.

In one embodiment, the composition comprises a compound of formulae (I), or a pharmaceutically acceptable salt thereof, and a pharmaceutically acceptable excipient.

In one embodiment, the composition comprises a compound of formulae (II), or a pharmaceutically acceptable salt thereof, and a pharmaceutically acceptable excipient.

In one embodiment, the composition comprises a compound of formulae (III), or a pharmaceutically acceptable salt thereof, and a pharmaceutically acceptable excipient.

In one embodiment, the composition comprises a compound of formulae (IV), or a pharmaceutically acceptable salt thereof, and a pharmaceutically acceptable excipient.

In one embodiment, the composition comprises a compound of formulae (V), or a pharmaceutically acceptable salt thereof, and a pharmaceutically acceptable excipient.

### Methods of Use

Novel compounds according to the present invention, a stereoisomer thereof, an enantiomer thereof, or a pharmaceutically acceptable salt thereof exhibit the effect of modulating Mer kinase.

Novel compounds according to the present invention, a stereoisomer thereof, an enantiomer thereof, or a pharmaceutically acceptable salt thereof exhibit the effect of modulating Axl kinase.

Novel compounds according to the present invention, a stereoisomer thereof, an enantiomer thereof, or a pharmaceutically acceptable salt can be used for the prevention or treatment of cancer or immune-related disease.

The compounds described herein, or pharmaceutically acceptable salts thereof, and pharmaceutical compositions comprising a compound described herein, or a pharmaceutically acceptable salt thereof, may be administered to a subject suffering from a disorder or condition associated with Mer kinase expression/over-expression or up-regulation and with activating genetic and epigenetic alterations. The term “administering” refers to the method of contacting a compound with a subject.

A “Mer kinase-mediated disorder or condition” is characterized by the participation of Mer kinase in the inception, manifestation of one or more symptoms or disease markers, maintenance, severity, or progression or resistance to therapeutic intervention of a disorder or condition. Accordingly, in embodiments, the present disclosure provides a method for treating cancer. The method comprises the step of administering to a subject in need thereof a therapeutically effective amount of a compound disclosed herein with or without a pharmaceutically acceptable carrier.

An “Axl kinase-mediated disorder or condition” is characterized by the participation of Axl kinase in the inception, manifestation of one or more symptoms or disease markers, maintenance, severity, or progression or resistance to therapeutic intervention of a disorder or condition. Accordingly, in embodiments, the present disclosure provides a method for treating cancer. The method comprises the step of administering to a subject in need thereof a therapeutically effective amount of a compound disclosed herein with or without a pharmaceutically acceptable carrier.

In embodiments, the present disclosure provides compounds of the disclosure, or pharmaceutical compositions comprising a compound of the disclosure, for use in medicine. In embodiments, the present disclosure provides compounds of the disclosure, or pharmaceutical compositions comprising a compound of the disclosure, for use in the treatment of diseases or disorders as described herein above.

In one embodiment, a method of treating a disease, disorder, or condition, is provided, wherein the method comprises administering an effective amount of a compound of formulae (I, II, III, IV, or V), or pharmaceutically acceptable salt thereof, or a pharmaceutical composition comprising a compound of formulae (I, II, III, IV, or V), or pharmaceutically acceptable salt thereof, and a pharmaceutically acceptable excipient, to a subject in need thereof, wherein the disease, disorder, or condition is cancer or an immune-related disease.

In one embodiment, a method of treating a disease, disorder, or condition, is provided, wherein the method comprises administering an effective amount of a compound of formulae (I, II, III, IV, or V), or pharmaceutically acceptable salt thereof, to a subject in need thereof, wherein the disease, disorder, or condition is cancer or an immune-related disease.e

In one embodiment, a method of treating a disease, disorder, or condition, is provided, wherein the method comprises administering an effective amount of a pharmaceutical composition comprising a compound of formulae (I, II, III, IV, or V), or pharmaceutically acceptable salt thereof, and a pharmaceutically acceptable excipient, to a subject in need thereof, wherein the disease, disorder, or condition is cancer or an immune-related disease.

One embodiment is directed to the use of a compound disclosed herein or a pharmaceutically acceptable salt thereof in the preparation of a medicament. The medicament optionally can comprise at least one additional therapeutic agent. In some embodiments the medicament is for use in the treatment of diseases and disorders as described herein above.

This disclosure is also directed to the use of a compound disclosed herein or a pharmaceutically acceptable salt thereof in the manufacture of a medicament for the treatment of the diseases and disorders as described herein above. The medicament optionally can comprise at least one additional therapeutic agent.

The compounds disclosed herein may be administered as the sole active agent or may be co-administered with other therapeutic agents, including other compounds that demonstrate the same or a similar therapeutic activity and that are determined to be safe and efficacious for such combined administration. The term “co-administered” means the administration of two or more different therapeutic agents or treatments (e.g., radiation treatment) that are administered to a subject in a single pharmaceutical composition or in separate pharmaceutical compositions. Thus co-administration involves administration at the same time of a single pharmaceutical composition comprising two or more different therapeutic agents or administration of two or more different compositions to the same subject at the same or different times.

The compounds of the present disclosure may be co-administered with a therapeutically effective amount of at least one additional therapeutic agent to treat cancer.

Further benefits of Applicants' disclosure will be apparent to one skilled in the art from reading this patent application.

The following Examples may be used for illustrative purposes and should not be deemed to narrow the scope of the present disclosure.

### Advantageous Effects

The compounds or a pharmaceutically acceptable salt thereof of the present disclosure exhibit the effects of modulating Mer kinase and Axl kinase.

### MODE FOR INVENTION

### EXAMPLES

### General

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

Common abbreviations well known to those skilled in the art which are used throughout include: DMSO for dimethyl sulfoxide; HATU for 1-[bis(dimethylamino)methylene]-1H-1,2,3-triazolo[4,5-b]pyridinium 3-oxid hexafluorophosphate or N,N,N′,N′-tetramethyl-3-oxo-1H-3λ⁵-[1,2,3]triazolo[4,5-b]pyridine-1-carboximidamidium hexafluoridophosphate(1-) or N,N,N′,N′-tetramethyl-1-oxo-3H-1λ⁵-[1,2,3]triazolo[4,5-b]pyridine-3-carboximidamidium hexafluoridophosphate(1-); and TFA for trifluoroacetic acid.

Other common abbreviations well known to those skilled in the art which are used throughout include: ATP for adenosine triphosphate; BSA for bovine serum albumin; EDTA for ethylenediaminetetraacetic acid; DMEM for Dulbecco's Modified Eagle's Medium; DTT for dithiothreitol; FBS for fetal bovine serum; GI5o for half-maximal growth inhibitory concentration; FRET for fluorescence energy transfer; GT for glutamate-tyrosine; HEPES for (4-(2-hydroxyethyl)-1-piperazineethanesulfonic acid); HTRF for homogeneous time-resolved fluorescence; IC₅₀ for half maximal inhibitory concentration; RPMI for Roswell Park Memorial Institute; SAR for structure-activity relationship; and TK for tyrosine kinase.

Yet other abbreviations well known to those skilled in the art which are used throughout include: APCI for atmospheric-pressure chemical ionization; atm for atmospheres of gas pressure; ESI for electrospray ionization; g for gram; h for hour or hours; HPLC for high performance liquid chromatography; LC/MS or LCMS for liquid chromatography—mass spectrometry; μL for microliter; μm for micrometer; mg for milligram; min for minute; mL for milliliter; mmol for millimoles; MS for mass spectrum; NMR for nuclear magnetic resonance; psi for pounds per square inch; rt for room temperature; SFC for supercritical fluid chromatography; and UPLC® or UHPLC for Ultra High Performance Liquid Chromatography.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on an Agilent 400 MHz NMR spectrometer, a Varian or Bruker 500 MHz spectrometer or a 501 MHz spectrometer. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), doublet of doublets of doublets (ddd), doublet of doublets of doublets of doublets (dddd), doublet of doublets of quartets (ddq), doublet of doublets of triplets (ddt), doublet of quartets (dq), doublet of triplets of doublets (dtd), heptet (hept), triplet (t), triplet of doublets of doublets (tdd), triplet of quartets (tq), quartet (q), quartet of doublets (qd), quartet of triplets (qt), quintuplet (quin), multiplet (m) and broad (br). Electrospray Ionization MS spectra were obtained on a Thermo Vanquish UPLC coupled to a Thermo MSQ Plus mass spectrometer using a Waters Xselect HSS T3 C18 2.5 μm column (2.1 mm×50 mm). A gradient of water containing 0.1% formic acid and 10 mM ammonium acetate (A) and acetonitrile with 0.1% formic acid (B) was used (0-2.3 min linear gradient 100-20% A). Microwave heating was performed with a Biotage® Initiator.

Flash chromatography purifications were performed on a Biotage® Isolera One Flash, Teledyne ISCO CombiFlash RF+ or an Analogix IntelliFlash 280 system. Reverse phase HPLC purifications were performed on a Waters Prep LC 2487, a Gilson PLC 2020 or a Gilson GX-281 system. Super critical fluid chromatography purifications were performed on a That SFC 80 system. The mobile phase comprised of supercritical CO₂ supplied by a Dewar of bone-dry non-certified CO2 pressurized to 350 psi with a methanol flow rate of 80 mL/min. The column was at ambient temperature and the backpressure regulator was set to maintain 100 bar. The sample was dissolved in methanol at a concentration of 50 mg/mL and was loaded into the modifier stream in 2 mL (100 mg) injections. The mobile phase was held isocratically at 20% co-solvent/CO₂.

### Intermediate 1

### (1S,2S)-2-[(4-bromophenyl)methoxy]cyclopentan-1-amine

To a solution of (1S,2S)-2-aminocyclopentanol hydrochloride (25 g, 182 mmol) in dimethyl sulfoxide (250 mL) was added potassium tert-butoxide (51 g, 454 mmol) in portions maintaining the temperature between 20-40° C. After stirring 1 hour, a solution of 1-bromo-4-(bromomethyl)benzene (49.9 g, 200 mmol) in dimethyl sulfoxide (150 mL) was added over 1.5 hours and the mixture was stirred at 25° C. for 1 hour. This reaction was repeated 19 times on the same scale. The reaction mixtures were combined, diluted with ethyl acetate and washed with water. The organic layer was dried over Na₂SO₄, filtered, and concentrated. The residue was treated with 4 M HCl in ethyl acetate at 25° C., and the precipitate was filtered and dried under vacuum to afford the title compound as a HCl salt (520 g). ¹H NMR (400 MHz, DMSO-d₆) δ 1.49-1.81 (m, 4H), 1.86-2.14 (m, 2H), 3.36-3.46 (m, 1H), 3.93-4.03 (m, 1H), 4.45-4.54 (m, 2H), 7.33 (d, J=8.44 Hz, 2H), 7.54 (d, J=8.44 Hz, 2H), 8.37 (br s, 3H).

### Intermediate 2

### 2-amino-5-(1-methyl-1H-pyrazol-4-yl)pyridine-3-carboxylic acid

To a solution of 2-amino-5-bromopyridine-3-carboxylic acid (80 g, 369 mmol) in dioxane (720 mL) and water (240 mL) was added 1-methyl-4-(4,4,5,5-tetramethyl-1,3,2-dioxaborolan-2-yl)-1H-pyrazole (100 g, 479 mmol), K₂CO₃ (153 g, 1106 mmol) and tetrakis(triphenylphosphine)palladium(0) (21.3 g, 18.43 mmol). The mixture was heated at 110° C. for 4 hours under nitrogen. After cooling to 20° C., the mixture was poured into water and extracted with ethyl acetate. The aqueous layer was acidified to a pH of about 4-5 with 10% HCl and stirred for 10 minutes. The precipitate was collected by filtration and dried under vacuum to afford the title compound (25 g). ¹H NMR (400 MHz, DMSO-d₆) δ 3.84 (s, 3H), 7.78 (s, 1H), 8.07 (s, 1H), 8.15 (d, J=2.45 Hz, 1H), 8.43 (d, J=2.45 Hz, 1H).

### Intermediate 3

### 2-amino-N-{(1S,2S)-2-[(4-bromophenyl)methoxy]cyclopentyl}-5-(1-methyl-1H-pyrazol-4-yl)pyridine-3-carboxamide

A mixture of Intermediate 2 (30 g, 137 mmol), (1-[bis(dimethylamino)methylene]-1H-1,2,3-triazolo[4,5-b]pyridinium 3-oxid hexafluorophosphate) (HATU, 62.7 g, 165 mmol) and N,N-diisopropylethylamine (36 mL, 206 mmol) in N,N-dimethylformamide (400 mL) was stirred at 15° C. until the solution became clear. A solution of Intermediate 1 (44.6 g, 165 mmol) in N,N-dimethylformamide (100 mL) was added, and the mixture was stirred at 15° C. for 2 hours. The reaction was repeated 2 times on the same scale and the mixtures were combined, diluted with ethyl acetate and washed with water. The organic layer was dried over Na₂SO₄, filtered, and concentrated. The residue was triturated with ethyl acetate, filtered and dried under vacuum to afford the title compound (78 g). ¹H NMR (400 MHz, DMSO-d₆) δ 1.52-1.77 (m, 4H), 1.86-2.08 (m, 2H), 3.83-3.92 (m, 4H), 4.22-4.33 (m, 1H), 4.49-4.60 (m, 1H), 4.49-4.60 (m, 2H), 6.96 (s, 2H), 7.28 (d, J=8.16 Hz, 2H), 7.50 (d, J=8.38 Hz, 2H), 7.78 (s, 1H), 7.95-8.03 (m, 2H), 8.27-8.37 (m, 2H).

### Intermediate 4

### 6-amino[3,3′-bipyridine]-5-carboxylic acid

To a solution of 2-amino-5-bromopyridine-3-carboxylic acid (15 g, 69.1 mmol) in dioxane (135 mL) and water (45 mL) was added pyridin-3-ylboronic acid (12.74 g, 104 mmol) and K₂CO₃ (28.7 g, 207 mmol) followed by tetrakis(triphenylphosphine)palladium(0) (3.99 g, 3.46 mmol) under nitrogen, and the mixture was stirred at 110° C. for 16 hours. This reaction was repeated one time on the same scale, and the reaction mixtures were combined, diluted with water, and extracted with ethyl acetate. The aqueous layer was acidified to a pH of about 5-6 with 3 M HCl. The precipitate was collected by filtration and dried under vacuum to afford the title compound (18 g). ¹H NMR (400 MHz, DMSO-d₆) δ 7.36-7.84 (m, 3H), 8.35 (br d, J=8.07 Hz, 1H), 8.43 (d, J=2.57 Hz, 1H), 8.59-8.70 (m, 2H), 9.01 (d, J=1.71 Hz, 1H).

### Intermediate 5

### 6-amino-5′-fluoro[3,3′-bipyridine]-5-carboxylic acid

To a solution of 2-amino-5-bromopyridine-3-carboxylic acid (8 g, 36.9 mmol) in ethanol (40 mL), tetrahydrofuran (40 mL) and water (40 mL) was added (5-fluoropyridin-3-yl)boronic acid (7.79 g, 55.3 mmol), Na₂CO₃ (7.81 g, 73.7 mmol) and (1,1′-bis(diphenylphosphino)ferrocenedichloro palladium(II) (2.70 g, 3.69 mmol). The mixture was stirred at 80° C. for 16 hours under nitrogen and then cooled to 20° C. This reaction was repeated 3 times on the same scale, and the reaction mixtures were combined, poured into water, and washed with ethyl acetate. The aqueous solution was adjusted to a pH of about 5-6 with 1 N HCl and stirred for 20 minutes. The precipitate was filtered, washed with water, and dried under vacuum to afford the title compound (16.7 g). ¹H NMR (400 MHz, D₂O) δ 7.47 (br d, J=10.03 Hz, 1H) 7.97 (d, J=2.32 Hz, 1H) 8.02 (d, J=2.45 Hz, 1H) 8.14 (d, J=2.45 Hz, 1H) 8.23 (s, 1H).

### Intermediate 6

### 6-amino-2′-fluoro[3,3′-bipyridine]-5-carboxylic acid

To a solution of 2-amino-5-bromopyridine-3-carboxylic acid (10 g, 46.1 mmol) in ethanol (50 mL), tetrahydrofuran (50 mL) and water (50 mL) was added (6-fluoropyridin-3-yl)boronic acid (9.74 g, 69.1 mmol), Na₂CO₃ (9.77 g, 92 mmol) and 1,1′-bis(diphenylphosphino)ferrocenedichloro palladium(II) (3.37 g, 4.61 mmol). The mixture was stirred at 90° C. for 16 hours under nitrogen, and then cooled to 20° C. This reaction was repeated 3 times on the same scale. The reaction mixtures were combined, poured into water and washed with ethyl acetate. The aqueous solution was adjusted to a pH of about 5-6 with 1 N HCl and stirred for 20 minutes. The precipitate was filtered, washed with water and dried under vacuum to afford the title compound (15 g). ¹H NMR (400 MHz, DMSO-d₆) δ 7.43 (ddd, J=7.18, 4.98, 1.77 Hz, 3H) 8.11-8.20 (m, 2H) 8.28 (s, 1H) 8.46 (s, 1H).

### Intermediate 7

### 6-amino-6′-fluoro[3,3′-bipyridine]-5-carboxylic acid

To a solution of 2-amino-5-bromopyridine-3-carboxylic acid (20 g, 92 mmol) in dioxane (180 mL) and water (60 mL) was added (6-fluoropyridin-3-yl)boronic acid (15.58 g, 111 mmol), tetrakis(triphenylphosphine)palladium(0) (2.130 g, 1.843 mmol) and K₂CO₃ (25.5 g, 184 mmol). The mixture was stirred at 110° C. for 4 hours under nitrogen and then cooled to 20° C. This reaction was repeated 9 times on the same scale. The reaction mixtures were combined, poured into water, and extracted with ethyl acetate. The aqueous layer was acidified to a pH of about 5-6 with 1 N HCl, stirred for 20 minutes, and filtered. The solid was washed with water and dried under vacuum to afford the title compound (102 g). ¹H NMR (400 MHz, DMSO-d₆) δ 7.23 (dd, J=8.56, 2.81 Hz, 1H) 7.40 (br s, 1H) 8.24 (td, J=8.19, 2.57 Hz, 1H) 8.31 (d, J=2.45 Hz, 1H) 8.49 (d, J=1.96 Hz, 1H) 8.56 (d, J=2.45 Hz, 1H).

### Intermediate 8

### 5-bromo-3,3-dimethyl-2,3-dihydro-1H-inden-1-one

### Intermediate 8A

### dimethyl [2-(4-bromophenyl)-2-oxoethyl]phosphonate

To trimethyl phosphite (500 mL, 4317 mmol) was added 2-bromo-1-(4-bromophenyl)ethanone (100 g, 360 mmol), and the mixture was stirred at 130° C. for 15 hours. This reaction was repeated 9 times on the same scale. The reaction mixtures were combined and concentrated to remove excess trimethyl phosphite. The residue was purified by flash chromatography on silica gel eluting with 8/1 to 2/1 petroleum ether/ethyl acetate to afford the title compound (900 g). ¹H NMR (400 MHz, CDCl₃) δ 1.44 (4H, d, J=17.64 Hz) 3.52-3.62 (2H, m) 3.68 (3H, s) 3.71 (3H, s) 3.72-3.76 (4H, m) 7.59 (2H, d, J=8.60 Hz) 7.80-7.85 (2H, m).

### Intermediate 8B

### 1-(4-bromophenyl)-3-methylbut-2-en-1-one

To a solution of Intermediate 8A (75 g, 147 mmol) in ethanol (750 mL) was added K₂CO₃ (60.8 g, 440 mmol) and acetone (108 mL, 1465 mmol), and the mixture was stirred at 80° C. for 15 hours. This reaction was repeated 11 times on the same scale and the reaction mixtures were combined and filtered. The filtrate was concentrated, added to water and extracted with ethyl acetate. The organic layer was dried over Na₂SO₄, filtered, and concentrated, and the residue purified by flash chromatography on silica gel eluting with 20/1 petroleum ether/ethyl acetate to afford the title compound (200 g). ¹H NMR (400 MHz, CDCl₃) δ 2.02 (3H, s) 2.21 (3H, s) 6.69 (1H, s) 7.57 (2H, d, J=8.38 Hz) 7.75-7.81 (2H, m).

### Intermediate 8C

### 5-bromo-3,3-dimethyl-2,3-dihydro-1H-inden-1-one

Intermediate 8B (40 g, 167 mmol) was added to polyphosphoric acid (230 mL, 167 mmol), and the mixture was stirred at 135° C. for 3 hours. This reaction was repeated 4 times on the same scale. The reaction mixtures were combined, poured into ice water with stirring and adjusted to pH 8 with saturated NaHCO₃. The mixture was extracted with ethyl acetate, washed with brine, dried over Na₂SO₄, filtered, and concentrated. Purification by flash chromatography on silica gel eluting with petroleum ether gave the title compound (73 g). ¹H NMR (400 MHz, CDCl₃) δ 1.42 (6H, s) 2.58 (2H, s) 7.26 (1H, s) 7.48-7.52 (1H, m) 7.53-7.58 (1H, m) 7.65 (1H, d, J=1.32 Hz).

### Intermediate 9

### 1-(5-bromo-3,3-dimethyl-2,3-dihydro-1H-inden-1-yl)piperazine Intermediate 9A tert-butyl 4-(5-bromo-3,3-dimethyl-2,3-dihydro-1H-inden-1-yl)piperazine-1-carboxylate

To a solution of Intermediate 8 (25 g, 105 mmol) and tert-butyl piperazine-1-carboxylate (38.9 g, 209 mmol) in dichloromethane (500 mL) was added a solution of TiCl₄ (52.3 mL, 52.3 mmol) in dichloromethane (52 mL) dropwise and the mixture was stirred at 25° C. for 17 hours. NaCNBH₄ (13.14 g, 209 mmol) was added, and the mixture was stirred for 8 hours. This reaction was repeated 4 times on the same scale. The reaction mixtures were combined, poured into ice water, and the organic layer was separated. The aqueous layer was extracted with dichloromethane. The combined organic layers were washed with brine, dried over Na₂SO₄, filtered, and concentrated. Purification by flash chromatography on silica gel eluting with 10/1 to 3/1 petroleum ether/ethyl acetate gave the title compound (140 g). ¹H NMR (400 MHz, CDCl₃) δ 1.21 (3H, s) 1.40-1.46 (12H, m) 1.89-2.35 (2H, m) 2.52-2.92 (2H, m) 3.00-3.52 (2H, m) 4.14 (4H, br s) 5.14 (1H, br t, J=8.16 Hz) 7.32 (1H, d, J=1.76 Hz) 7.43 (1H, dd, J=8.38, 1.76 Hz) 8.20 (1H, d, J=8.38 Hz).

### Intermediate 9B

### 1-(5-bromo-3,3-dimethyl-2,3-dihydro-1H-inden-1-yl)piperazine

To a solution of Intermediate 9A (35 g, 85 mmol) in ethyl acetate (350 mL) was added a solution of 4 M HCl in ethyl acetate (70 mL, 280 mmol) at 0° C. The mixture was stirred at 25° C. for 3 hours. This reaction was repeated 3 times on the same scale. The reaction mixtures were combined and concentrated to a volume of about 500 mL. The solid was collected by filtration, washed with methyl tert-butyl ether, and dried under vacuum to afford the title compound as a HCl salt (134 g). ¹H NMR (400 MHz, methanol-d₄) δ 1.26 (3H, s) 1.49 (3H, s) 2.18-2.50 (2H, m) 3.38-3.49 (2H, m) 3.57-3.80 (6H, m) 5.29 (1H, s) 7.49-7.56 (2H, m) 7.82 (1H, d, J=7.94 Hz).

### Intermediate 10

### 1-(5-bromo-3,3-dimethyl-2,3-dihydro-1H-inden-1-yl)-4-methylpiperazine

To a solution of Intermediate 9 (20 g, 64.7 mmol) in methanol (400 mL) was added formaldehyde (7.22 mL, 97 mmol) at 20° C. After stirring for 15 minutes, NaCNBH₄ (4.06 g, 64.7 mmol) was added in portions, and the mixture was stirred for 3 hours. This reaction was repeated 2 times on the same scale. The reaction mixtures were combined, quenched with water, stirred for 30 minutes and partially concentrated. The aqueous layer was extracted with ethyl acetate, dried over Na₂SO₄, filtered, and concentrated. Purification by flash chromatography on silica gel eluting with 2/1 petroleum ether/ethyl acetate to 100% ethyl acetate, followed by trituration with ethanol (200 mL) gave the title compound (50 g). ¹H NMR (400 MHz, DMSO-d₆) δ 1.11 (3H, s) 1.29 (3H, s) 1.83 (2H, dd, J=7.94, 4.41 Hz) 2.14 (3H, s) 2.25-2.46 (7H, m) 4.32 (1H, t, J=8.05 Hz) 7.11 (1H, d, J=7.94 Hz) 7.33 (1H, dd, J=8.05, 1.87 Hz) 7.37 (1H, d, J=1.76 Hz).

### Intermediate 11

### 1-[(1S)-5-bromo-3,3-dimethyl-2,3-dihydro-H-inden-1-yl]-4-methylpiperazine

Intermediate 10 was separated by super critical fluid chromatography (Column: Chiralpak AD-H; Mobile phase: A: CO₂ and B: methanol (0.1% diethylamine), 25% B in A) to afford Intermediate 11 (20.7 g) as the first eluting peak.

### Intermediate 12

### 1-[(1R)-5-bromo-3,3-dimethyl-2,3-dihydro-1H-inden-1-yl]-4-methylpiperazine

Intermediate 12 (21.5 g) was obtained in the super critical fluid chromatography purification described for Intermediate 11 as the second eluting peak. ¹H NMR (400 MHz, DMSO-d₆) δ 1.12 (3H, s) 1.29 (3H, s) 1.83 (2H, dd, J=8.05, 3.20 Hz) 2.16 (3H, s) 2.35 (7H, br d, J=5.95 Hz) 3.43 (2H, br s) 4.32 (1H, s) 7.11 (1H, d, J=7.94 Hz) 7.33 (1H, dd, J=7.94, 1.76 Hz) 7.38 (1H, d, J=1.76 Hz).

### Intermediate 13

### 2-[4-(5-bromo-3,3-dimethyl-2,3-dihydro-1H-inden-1-yl)piperazin-1-yl]ethan-1-ol

To a solution of Intermediate 9 (20 g, 64.7 mmol) in methanol (300 mL) was added K₂CO₃ (22.35 g, 162 mmol) and 2-bromoethanol (14.55 g, 116 mmol) at 25° C. for 20 hours. This reaction was repeated 2 times on the same scale. The reaction mixtures were combined, diluted with ethyl acetate, filtered, and concentrated. The residue was suspended in ethanol (200 mL) and stirred for 1 hour. The precipitate was collected by filtration and dried under vacuum to afford the title compound (53 g).

### Intermediate 14

### 2-{4-[(1S)-5-bromo-3,3-dimethyl-2,3-dihydro-1H-inden-1-yl]piperazin-1-yl}ethan-1-ol

Intermediate 13 (53 g, 150 mmol) was separated by super critical fluid chromatography (Column: Chiralpak AD-H; Mobile phase: A: CO₂ and B: methanol (0.1% diethylamine), 25% B inA) to afford Intermediate 14 (15.0 g) as the first eluting peak. ¹H NMR (400 MHz, DMSO-d₆) δ 1.12 (3H, s) 1.29 (3H, s) 1.84 (2H, t, J=7.50 Hz) 1.97 (1H, s) 2.31-2.47 (9H, m) 3.46 (2H, br t, J=5.40 Hz) 4.28-4.39 (2H, m) 7.12 (1H, d, J=7.94 Hz) 7.33 (1H, dd, J=8.16, 1.76 Hz) 7.38 (1H, d, J=1.76 Hz).

### Intermediate 15

### 2-{4-[(TR)-5-bromo-3,3-dimethyl-2,3-dihydro-1H-inden-1-yl]piperazin-1-yl}ethan-1-ol

Intermediate 15 (15.2 g) was obtained in the super critical fluid chromatography purification described in the preparation of Intermediate 14 as the second eluting peak. ¹H NMR (400 MHz, DMSO-d₆) δ 1.12 (3H, s) 1.29 (3H, s) 1.84 (2H, t, J=7.50 Hz) 1.97 (1H, s) 2.31-2.47 (9H, m) 3.46 (2H, br t, J=5.40 Hz) 4.28-4.39 (2H, m) 7.12 (1H, d, J=7.94 Hz) 7.33 (1H, dd, J=8.16, 1.76 Hz) 7.38 (1H, d, J=1.76 Hz).

### Intermediate 16

### 1-[(1S)-5-bromo-2,3-dihydro-1H-inden-1-yl]piperazine Intermediate 16A 1-[(1S)-5-bromo-2,3-dihydro-1H-inden-1-yl]-4-(4-methylbenzene-1-sulfonyl)piperazine

A mixture of (1S)-5-bromo-2,3-dihydro-1H-inden-1-amine (20 g, 94 mmol) and N,N-bis(2-chloroethyl)-4-methylbenzenesulfonamide (30.7 g, 104 mmol) in N,N-diisopropylethylamine (200 mL, 1145 mmol) were stirred at 145° C. for 12 hours. This reaction was repeated 1 time on the same scale. The reaction mixtures were combined and concentrated under reduced pressure. The residue was diluted with ethyl acetate, washed with water, dried over Na₂SO₄, filtered, and concentrated under reduced pressure. The residue was purified by flash chromatography on silica gel eluting with 10/1 petroleum ether/ethyl acetate to afford the title compound (40 g). ¹H NMR (400 MHz, CDCl₃) δ 1.92-1.98 (m, 2H) 2.37 (s, 3H) 2.49 (tdd, J=15.74, 15.74, 11.13, 4.71 Hz, 4H) 2.66-2.85 (m, 2H) 2.93 (br s, 4H) 4.16 (t, J=7.03 Hz, 1H) 7.00 (d, J=7.95 Hz, 1H) 7.14-7.22 (m, 1H) 7.23-7.31 (m, 3H) 7.56 (d, J=8.19 Hz, 2H).

### Intermediate 16B

### 1-[(1S)-5-bromo-2,3-dihydro-1H-inden-1-yl]piperazine

To a solution of Intermediate 16A (20 g, 45.9 mmol) in 33% HBr/acetic acid (200 mL, 698 mmol) was added 4-hydroxybenzoic acid (19.03 g, 138 mmol), and the mixture was stirred at 90° C. for I h, and then cooled to 25° C. This reaction was repeated 1 time on the same scale. The reaction mixtures were combined and concentrated under reduced pressure. The residue was poured into water, and the precipitate filtered and washed with water The filtrate was washed with ethyl acetate. The aqueous layer was adjusted to pH 10 with 2 M Na₂CO₃ and then extracted with ethyl acetate. The combined organic phases were dried over Na₂SO₄, filtered, and concentrated under reduced pressure. The residue was dissolved in 4 M HCl in ethyl acetate (500 mL) and concentrated under reduced pressure. The residue was washed with ethyl acetate and dried under vacuum to afford the title compound as a HCl salt (25.9 g). ¹H NMR (400 MHz, methanol-d₄) δ 2.54-2.68 (m, 2H) 3.01-3.14 (m, 1H) 3.20-3.29 (m, 1H) 3.37-3.52 (m, 2H) 3.55-3.73 (m, 6H) 5.01-5.13 (m, 1H) 7.55 (d, J=8.19 Hz, 1H) 7.63 (s, 1H) 7.69 (br d, J=8.19 Hz, 1H).

### Intermediate 17

### 1-[(1R)-6-bromo-1,2,3,4-tetrahydronaphthalen-1-yl]piperazine

### Intermediate 17A

### tert-butyl 4-(6-bromo-1,2,3,4-tetrahydronaphthalen-1-yl)piperazine-1-carboxylate

To a solution of 6-bromo-3,4-dihydronaphthalen-1 (2H)-one (20 g, 89 mmol) in anhydrous dichloromethane (200 mL) was added tert-butyl piperazine-1-carboxylate (49.6 g, 267 mmol) and triethylamine (37.2 mL, 267 mmol). A solution of TiCl₄ (6.86 mL, 62.2 mmol) in dichloromethane (44 mL) was added, and the mixture was stirred at 25° C. for 24 hours under nitrogen. NaCNBH₄ (22.34 g, 355 mmol) in methanol (60 mL) was added dropwise and the mixture was stirred at 25° C. for 8 hours. This reaction was repeated 2 times on the same scale. The reaction mixtures were combined, quenched with aqueous Na₂CO₃, and filtered. The mixture was extracted with dichloromethane, washed with brine, dried over Na₂SO₄, filtered, and concentrated under reduced pressure. The residue was purified by flash chromatography on silica gel eluting with 100/1 to 5/1 petroleum ether/ethyl acetate to afford the title compound (210 g). ¹H NMR (400 MHz, methanol-d₄) δ 1.45 (s, 9H) 1.65-1.68 (m, 2H) 1.87-2.02 (m, 2H) 2.09-2.13 (m, 2H) 2.44-2.51 (m, 4H) 2.61-2.69 (m, 5H) 2.95-2.98 (m, 3H) 3.38-3.41 (m, 4H) 3.78-3.80 (m, 1H) 7.20-7.25 (m, 2H) 7.48 (dd, J=8.55, 1.97 Hz, 1H) 7.53-7.59 (m, 1H) 7.58 (d, J=8.33 Hz, 1H) 7.83 (d, J=8.33 Hz, 1H).

### Intermediate 17B

### 1-(6-bromo-1,2,3,4-tetrahydronaphthalen-1-yl)piperazine

To a solution of Intermediate 17A (30 g, 45.5 mmol) in ethyl acetate (60 mL) was added HCl (16.60 g, 455 mmol) in ethyl acetate (180 mL), and the mixture was stirred at 25° C. for 4 hours under nitrogen. This reaction was repeated 2 times on the same scale and the reaction mixtures were combined and filtered. The solid was washed with ethyl acetate (300 mL) and diluted with water (800 mL). The mixture was adjusted to pH 10 by slow addition of saturated K₂CO₃ (100 mL) and extracted with ethyl acetate. The organic layer was washed with brine, filtered, and concentrated under reduced pressure to afford the title compound (80 g). ¹H NMR (400 MHz, methanol-d₄) δ 1.70-1.72 (m, 2H) 1.98-2.01 (m, 2H) 2.72-2.81 (m, 6H) 3.20-3.24 (m, 4H) 3.90-3.91 (m, 1H) 7.25 (s, 1H) 7.28 (dd, J=8.31, 1.96 Hz, 1H) 7.55 (d, J=8.31 Hz, 1H).

### Intermediate 17C

### 1-[(1R)-6-bromo-1,2,3,4-tetrahydronaphthalen-1-yl]piperazine

Intermediate 17B (80 g, 135 mmol) was purified by super critical fluid chromatography using a ChiralPak AD-H 250×50 mm i. d. column to afford the crude title compound as the first eluting peak. The crude product was dissolved in ethyl acetate (100 mL) and 4 N HCl in ethyl acetate (100 mL) was added. The mixture was stirred at 25° C. for 2 hours and filtered. The filter cake was triturated with 10/1 ethyl acetate/methanol (50 mL) and filtered. The filtrate was concentrated under reduced pressure and purified by super critical fluid chromatography a second time to afford 10.8 g of the title compound. The filter cake was dissolved in 1 N K₂CO₃, adjusted to pH 10, and extracted with ethyl acetate. The organic layer was washed with brⁱne, dried over Na₂SO₄, filtered, and concentrated to afford the title compound (17 g) for a combined total of 27.8 g of title compound. ¹H NMR (400 MHz, methanol-d₄) δ 1.69-1.71 (m, 2H) 1.95-1.98 (m, 2H) 2.48-2.51 (m, 2H) 2.52-2.62 (m, 2H) 2.64-2.87 (m, 6H) 3.73 (br dd, J=9.05, 4.77 Hz, 1H) 7.21 (s, 1H) 7.24 (dd, J=8.38, 2.02 Hz, 1H) 7.57 (d, J=8.44 Hz, 1H).

### Intermediate 18

### 1-[(1S)-6-bromo-1,2,3,4-tetrahydronaphthalen-1-yl]piperazine

The title compound (26 g) was obtained in the super critical fluid chromatography purification described for Intermediate 17C as the second eluting peak. ¹H NMR (400 MHz, methanol-d₄) δ 1.69-1.71 (m, 2H) 1.95-1.98 (m, 2H) 2.48-2.51 (m, 2H) 2.52-2.62 (m, 2H) 2.64-2.88 (m, 6H) 3.73 (br dd, J=9.05, 4.77 Hz, 1H) 7.21 (s, 1H) 7.24 (dd, J=8.38, 2.02 Hz, 1H) 7.57 (d, J=8.44 Hz, 1H).

### Intermediate 19

### 6-amino-2′-fluoro-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

### Intermediate 19A

### 6-amino-N-{(1S,2S)-2-[(4-bromophenyl)methoxy]cyclopentyl}-2′-fluoro[3,3′-bipyridine]-5-carboxamide

To a solution of Intermediate 6 (5.5 g, 23.59 mmol) in NN-dimethylformamide (100 mL) was added Intermediate 1 (6.57 g, 21.44 mmol), 1-hydroxybenzotriazole hydrate (4.93 g, 32.2 mmol) and 1-(3-dimethylaminopropyl)-3-ethylcarbodiimide hydrochloride (4.11 g, 21.44 mmol), and the mixture was stirred at 25° C. for 5 minutes. N,N-Diisopropylethylamine (9.36 mL, 53.6 mmol) was added, and the mixture was stirred at 25° C. for 18 hours. This reaction was repeated 5 times on the same scale. The reaction mixtures were combined, water was added, and the mixture was extracted with ethyl acetate. The organic layer was washed with brine, dried over Na₂SO₄, filtered, and concentrated. The residue was purified by flash chromatography on silica gel eluting with 10/1 to 1/1 petroleum ether/ethyl acetate to afford the title compound (35 g). ¹H NMR (400 MHz, DMSO-d₆) δ 1.54-1.71 (m, 4H) 1.88-1.94 (m, 1H) 2.00-2.05 (m, 1H) 3.84-3.88 (m, 1H) 4.25-4.28 (m, 1H) 4.51-4.56 (m, 2H) 7.27 (d, J=8.31 Hz, 2H) 7.31 (s, 2H) 7.47-7.50 (m, 3H) 8.09 (d, J=1.83 Hz, 1H) 8.11-8.18 (m, 1H) 8.19-8.20 (m, 1H) 8.34-8.38 (m, 2H).

### Intermediate 19B

### 6-amino-2′-fluoro-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

To a solution of Intermediate 19A (7 g, 14.42 mmol) in tetrahydrofuran (70 mL) was added potassium acetate (2.83 g, 28.8 mmol), and the mixture was stirred under nitrogen for 1 hour. Tris(dibenzylideneacetone) dipalladium(0) (0.132 g, 0.144 mmol) and dicyclohexyl(2′,4′,6′-triisopropyl-[1,1′-biphenyl]-2-yl)phosphine (0.275 g, 0.577 mmol) were added, and the mixture was stirred at 75° C. for 18 hours. This reaction was repeated 5 times on the same scale. The reaction mixtures were combined, diluted with water, and extracted with ethyl acetate. The organic layer was washed with brine, dried over Na₂SO₄, filtered, and concentrated. The residue was purified by flash chromatography on silica gel eluting with 10/1 to 1/1 petroleum ether/ethyl acetate to afford the title compound (22.23 g). ¹H NMR (400 MHz, methanol-d₄) δ 1.27-1.41 (m, 12H) 1.53-1.67 (m, 1H) 1.69-1.86 (m, 3H) 1.93-2.00 (m, 1H) 2.09-2.22 (m, 1H) 3.94 (dt, J=6.51, 4.33 Hz, 1H) 4.39 (td, J=7.24, 4.59 Hz, 1H) 4.60-4.69 (m, 2H) 7.34 (d, J=7.95 Hz, 2H) 7.40 (ddd, J=7.27, 5.07, 1.83 Hz, 1H) 7.67 (d, J=7.95 Hz, 2H) 8.02-8.11 (m, 2H) 8.12-8.21 (m, 1H) 8.32 (t, J=2.08 Hz, 1H). The corresponding boronic acid was also obtained (1.64 g). ¹H NMR (400 MHz, methanol-d₄) δ 1.30-1.36 (m, 1H) 1.53-1.65 (m, 1H) 1.69-1.85 (m, 3H) 1.94-2.06 (m, 1H) 2.10-2.23 (m, 1H) 3.36 (s, 1H) 3.95 (dt, J=6.57, 4.42 Hz, 1H) 4.34-4.44 (m, 1H) 4.58-4.69 (m, 2H) 7.29-7.37 (m, 2H) 7.40 (ddd, J=7.27, 5.07, 1.83 Hz, 1H) 7.52-7.70 (m, 2H) 8.01-8.10 (m, 2H) 8.12-8.22 (m, 1H) 8.33 (t, J=2.02 Hz, 1H).

### Intermediate 20

### 6-amino-6′-methoxy-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide Intermediate 20A 6-amino-6′-methoxy[3,3′-bipyridine]-5-carboxylic acid

To a solution of 2-amino-5-bromopyridine-3-carboxylic acid (6 g, 27.6 mmol) in dioxane (60 mL) and water (20 mL) was added (6-methoxypyridin-3-yl)boronic acid (8.46 g, 55.3 mmol) and K₂CO₃ (7.64 g, 55.3 mmol) followed by tetrakis(triphenylphosphine)palladium(0) (1.597 g, 1.382 mmol) under nitrogen. The mixture was stirred at 110° C. for 12 hours and then cooled to 25° C. This reaction was repeated 4 times on the same scale. The reaction mixtures were combined, diluted with water, and extracted with ethyl acetate. The aqueous layer was acidified to a pH of about 5-6 with 6 M HCl. The precipitate was collected and dried under vacuum to afford the title compound (26.5 g) which was used directly in the next step. ¹H NMR (400 MHz, DMSO-d₆) δ 3.87 (s, 3H), 6.75-6.92 (m, 1H), 6.87 (d, J=8.77 Hz, 1H), 7.13-7.85 (m, 2H), 7.95 (dd, J=8.77, 2.63 Hz, 1H), 8.23 (d, J=2.63 Hz, 1H), 8.40 (d, J=2.19 Hz, 1H), 8.48 (d, J=2.63 Hz, 1H).

### Intermediate 20B

### 6-amino-N-{(1S,2S)-2-[(4-bromophenyl)methoxy]cyclopentyl}-6′-methoxy[3,3′-bipyridine]-5-carboxamide

A mixture of Intermediate 20A (5.3 g, 21.61 mmol), (1-[bis(dimethylamino)methylene]-1H-1,2,3-triazolo[4,5-b]pyridinium 3-oxid hexafluorophosphate) (HATU, 9.86 g, 25.9 mmol) and triethylamine (4.52 mL, 32.4 mmol) in N,N-dimethylformamide (50 mL) was stirred at 20° C. until the solution become clear. A solution of Intermediate 1 (7.95 g, 25.9 mmol) in N,N-dimethylformamide (10 mL) was added, and the mixture was stirred at 20° C. for 12 hours. This reaction was repeated 4 times on the same scale. The reaction mixtures were combined, diluted with water, and extracted with ethyl acetate. The organic layer was washed brine, dried over Na₂SO₄, filtered, and concentrated to afford the title compound (30 g) which was used directly in next step.

### Intermediate 20C

### 6-amino-6′-methoxy-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

A mixture of Intermediate 20B (5 g, 10 mmol) and 4,4,4′,4′,5,5,5′,5′-octamethyl-2,2′-bi(1,3,2-dioxaborolane) (4.85 g, 19.1 mmol) in dioxane (100 mL) was treated with potassium acetate (2.96 g, 30.2 mmol) followed by 1,1′-bis(diphenylphosphino)ferrocenedichloro palladium(II) dichloromethane complex (0.588 g, 0.804 mmol). The mixture was heated under nitrogen at 100° C. for 12 hours and then cooled to ambient temperature. This reaction was repeated 14 times on the same scale. The reaction mixtures were combined, diluted with ethyl acetate, and washed with water. The organic layer was washed with brine, dried over Na₂SO₄, and concentrated. The residue was purified by flash chromatography on silica gel eluting with 50/1 petroleum ether/ethyl acetate to 100% ethyl acetate to afford the title compound (16.21 g). ¹H NMR (400 MHz, methanol-d₄) δ 1.20 (s, 1H), 1.31 (s, 12H), 1.54-1.63 (m, 1H), 1.72-1.83 (m, 3H), 1.96-2.04 (m, 1H), 2.11-2.20 (m, 1H), 3.94 (s, 4H), 4.36-4.45 (m, 1H), 4.65 (s, 2H), 6.88 (d, J=8.60 Hz, 1H), 7.34 (d, J=7.94 Hz, 2H), 7.66 (d, J=7.94 Hz, 2H), 7.89 (dd, J=8.60, 2.43 Hz, 1H), 8.03 (d, J=2.20 Hz, 1H), 8.24-8.40 (m, 2H). LCMS (ESI+): m/z 545.2 (M+H)+.

### Intermediate 21

### 5-bromo-3,3-dimethyl-2,3-dihydro-1H-indole

### Intermediate 21A

### 3,3-dimethyl-1,3-dihydro-2H-indol-2-one

To a solution of indolin-2-one (20 g, 150 mmol) and LiCl (22.29 g, 526 mmol) in tetrahydrofuran (400 mL) was added n-butyllithium (120 mL, 300 mmol) at −78° C., and the mixture was stirred at −78° C. for 20 minutes. Iodomethane (18.78 mL, 300 mmol) was added, and the mixture was stirred at 25° C. for 12 hours. This reaction was repeated 4 times on the same scale. The reaction mixtures were combined, quenched with saturated NH₄Cl and extracted with ethyl acetate. The organic phase was washed water and brine, dried over Na₂SO₄, filtered, and concentrated. Flash chromatography on silica gel eluting with 50:1 to 5:1 petroleum ether/ethyl acetate, followed by precipitation gave the title compound (62 g). ¹H NMR (400 MHz, DMSO-d₆) δ 1.23 (s, 6H), 6.84 (d, J=7.72 Hz, 1H), 6.95 (td, J=7.50, 0.88 Hz, 1H), 7.15 (td, J=7.66, 1.21 Hz, 1H), 7.26 (d, J=7.50 Hz, 1H), 10.30 (br s, 1H).

### Intermediate 21B

### 5-bromo-3,3-dimethyl-1,3-dihydro-2H-indol-2-one

To a solution of Intermediate 21A (10 g, 58.9 mmol) and sodium acetate (4.83 g, 58.9 mmol) in acetic acid (80 mL) at 25° C. was added dropwise Br₂ (3.04 mL, 58.9 mmol) in acetic acid (80 mL), and the mixture was stirred at 25° C. for 50 minutes. This reaction was repeated 5 times on the same scale. The reaction mixtures were combined, added to water and adjusted to a pH of about 7-8 with aqueous Na₂CO₃. The mixture was extracted with ethyl acetate, and the organic layers were dried over MgSO₄, filtered, and concentrated to afford the title compound (80 g). ¹H NMR (400 MHz, DMSO-d₆) δ 1.25 (s, 6H), 6.80 (d, J=8.16 Hz, 1H), 7.33 (dd, J=8.27, 2.09 Hz, 1H), 7.53 (d, J=1.98 Hz, 1H), 10.44 (s, 1H).

### Intermediate 21C

### 5-bromo-3,3-dimethyl-2,3-dihydro-1H-indole

A solution of Intermediate 21B (10 g, 39.6 mmol) in tetrahydrofuran (20 mL) was added dropwised to borane-tetrahydrofuran (396 mL, 396 mmol) at 0° C., and the mixture was stirred at 50° C. for 2 hours. The mixture was cooled to 0° C., diluted slowly with methanol (41.5 mL, 1026 mmol), followed by addition of HCl (11 mL, 132 mmol). The mixture was stirred at 25° C. for 1 hour and adjusted to a pH of about 8-9 with 10% NaOH (125 mL). This reaction was repeated 7 times on the same scale, and the reaction mixtures were combined and extracted with ethyl acetate. The organic layers were washed with brine, dried over MgSO₄, filtered, and concentrated. The residue was purified by flash chromatography on silica gel eluting with 1000:1 to 50:1 petroleum ether/ethyl acetate to afford the title compound (32 g). ¹H NMR (400 MHz, DMSO-d₆) δ 1.21 (s, 6H), 3.18 (d, J=1.76 Hz, 2H), 5.63 (br s, 1H), 6.42 (d, J=8.16 Hz, 1H), 7.02 (dd, J=8.16, 1.98 Hz, 1H), 7.10 (d, J=1.98 Hz, 1H. LCMS (ESI+) m/z 225.9, 227.9 (M+H)+.

### Intermediate 22

### 5-bromo-3,3-dimethyl-1-(piperidin-4-yl)-2,3-dihydro-1H-indole-hydrogen chloride (1/1)

### Intermediate 22A

### tert-butyl 4-(5-bromo-3,3-dimethyl-2,3-dihydro-1H-indol-1-yl)piperidine-1-carboxylate

To a solution of 5-bromo-3,3-dimethylindoline (18 g, 80 mmol) in methanol (200 mL) was added tert-butyl 4-oxopiperidine-1-carboxylate (31.7 g, 159 mmol) and acetic acid (10 mL, 175 mmol), and the mixture was stirred at 25° C. for 2 hours and then cooled to 0° C. NaCNBH₄ (12.51 g, 199 mmol) was added, and the mixture was stirred at 25° C. for 12 hours. This reaction was repeated 4 times on the same scale. The reaction mixtures were combined, poured into water and extracted with ethyl acetate. The organic layers were washed with brine, dried over Na₂SO₄, and concentrated. The residue was purified by flash chromatography on silica gel eluting with 10/1 petroleum ether/ethyl acetate to afford the title compound (92 g). ¹H NMR (400 MHz, methanol-d₄) δ 1.24 (s, 6H) 1.44-1.46 (m, 9H) 1.49 -1.56 (m, 2H) 1.74 (br d, J=12.35 Hz, 2H) 2.84 (br s, 2H) 3.11 (s, 2H) 3.56 (tt, J=11.74, 3.69 Hz, 1H) 4.17 (br d, J=13.23 Hz, 2H) 6.38 (d, J=8.38 Hz, 1H) 7.02 (d, J=1.98 Hz, 1H) 7.08 (dd, J=8.38, 1.98 Hz, 1H).

### Intermediate 22B

### 5-bromo-3,3-dimethyl-1-(piperidin-4-yl)-2,3-dihydro-1H-indole-hydrogen chloride (1/1)

To a solution of Intermediate 22A (20 g, 48.9 mmol) in methanol (100 mL) was added 4 M HCl in methanol (100 mL, 400 mmol), and the mixture was stirred at 25° C. for 4 hours. This reaction was repeated 3 times on the same scale. The reaction mixtures were combined, concentrated, and washed with ethyl acetate to afford the title compound as a HCl salt (70 g). ¹H NMR (400 MHz, methanol-d₄) δ 1.31 (s, 6H) 1.85-2.09 (m, 4H) 3.08-3.22 (m, 2H) 3.28 (s, 2H) 3.52 (br d, J=12.79 Hz, 2H) 3.83-3.98 (m, 1H) 6.64-6.71 (m, 1H) 7.19 (d, J=1.98 Hz, 1H) 7.22 (dd, J=8.27, 1.87 Hz, 1H).

### Intermediate 23

### 2-[4-(5-bromo-3,3-dimethyl-2,3-dihydro-1H-indol-1-yl)piperidin-1-yl]ethan-1-ol-hydrogen chloride (1/1)

To a solution of Intermediate 22 (15 g, 39.3 mmol) in methanol (150 mL) was added K₂CO₃ (27.1 g, 196 mmol) and 2-bromoethanol (12.26 g, 98 mmol), and the mixture was stirred at 60° C. for 16 hours and then cooled to 25° C. This reaction was repeated 3 times on the same scale. The reaction mixtures were combined, filtered, and the solid washed with methanol. The filtrate was concentrated under reduced pressure, poured into water and extracted with ethyl acetate. The organic layers were dried over Na₂SO₄, filtered, and concentrated. The residue was poured into 4 M HCl in ethyl acetate (200 mL), concentrated, washed with methanol and dried under vacuum to afford the title compound as a HCl salt (33 g). ¹H NMR (400 MHz, DMSO-d₆) δ 1.22 (s, 6H) 1.81 (br d, J=12.79 Hz, 2H) 1.92-2.15 (m, 2H) 3.04-3.36 (m, 6H) 3.58 (br d, J=11.91 Hz, 2H) 3.65-3.83 (m, 3H) 6.29-6.59 (m, 1H) 7.03-7.25 (m, 2H) 10.39 (br s, 1H).

### Intermediate 24

### [4—({[(1S,2S)-2-{[2-amino-5-(1-methyl-1H-pyrazol-4-yl)pyridine-3-carbonyl]amino}cyclopentyl]oxy}methyl)phenyl]boronic acid

A flask was dried for 1 hour in a 50° C. vacuum oven, cooled under nitrogen, and then charged with ethylene glycol (34 mL), methanol (136 mL) and sparged with nitrogen for 1 hour. A separate flask containing oven dried potassium acetate (13.37 g, 136 mmol) was dried in a 50° C. vacuum oven for 1 hour, and then cooled to ambient temperature under nitrogen. This flask was charged with Intermediate 3 (32.05 g, 68.1 mmol), hypodiboric acid (12.22 g, 136 mmol), chloro[(tri-tert-butylphosphine)-2-(2-aminobiphenyl)]palladium(II) (1.746 g, 3.41 mmol) and sparged with nitrogen for 1 hour. Both flasks were cooled to 0° C., and the contents of the first flask were transferred via cannula under nitrogen to the second flask. The mixture was stirred at 10-20° C. for 2 hours and quenched by cannulating into water with vigorous stirring. After stirring for 10 minutes, the solids were filtered and washed with water. The solids were dissolved in 10% methanol in dichloromethane (4 L), and the solution was washed with brine, dried over MgSO₄, and filtered. Concentration and filtration through a silica gel plug using 15% methanol/dichloromethane afforded the title compound (21.3 g). ¹H NMR (500 MHz, DMSO-d₆) δ 8.39 (d, J=7.7 Hz, 1H), 8.29 (d, J=2.2 Hz, 1H), 7.98 (d, J=2.3 Hz, 1H), 7.96 (s, 1H), 7.77 (d, J=0.8 Hz, 1H), 7.73-7.68 (m, 2H), 7.30-7.23 (m, 2H), 4.63-4.43 (m, 2H), 4.27 (qd, J=7.6, 3.8 Hz, 1H), 3.89 (dt, J=6.4, 4.0 Hz, 1H), 3.83 (s, 3H), 2.02 (qq, J=7.9, 4.9, 3.3 Hz, 1H), 1.94-1.83 (m, 1H), 1.75-1.59 (m, 2H), 1.55 (ddd, J=12.4, 8.0, 6.2 Hz, 1H). LCMS (APCI) m/z 435.38 (M+H)+.

### Intermediate 25

### [4—({[(1S,2S)-2-{[2-amino-5-(trifluoromethyl)pyridine-3-carbonyl]amino}cyclopentyl]oxy}methyl)phenyl]boronic acid

### Intermediate 25A

### 2-amino-N-{(1S,2S)-2-[(4-bromophenyl)methoxy]cyclopentyl}-5-(trifluoromethyl)pyridine-3-carboxamide

To a solution of 2-amino-5-(trifluoromethyl)pyridine-3-carboxylic acid (5.25 g, 25.5 mmol) and 1-[bis(dimethylamino)methylene]-1H-1,2,3-triazolo[4,5-b]pyridinium 3-oxid hexafluorophosphate (HATU, 10.65 g, 28.0 mmol) in N,N-dimethylformamide (42.5 mL) under nitrogen was added N,N-diisopropylethylamine (4.45 mL, 25.5 mmol), and the mixture was stirred for 1 hour. Intermediate 1 (7.89 g, 25.7 mmol) was added, followed by N,N-diisopropylethylamine (9.34 mL, 53.5 mmol), and the mixture was stirred for 1 hour. The mixture was partitioned between ethyl acetate and water, and the aqueous layer was extracted with ethyl acetate. The organic layers were dried over MgSO₄, filtered, and concentrated. The residue was triturated with dichloromethane and filtered to afford the title compound (8.52 g). ¹H NMR (400 MHz, DMSO-d₆) δ 8.52 (d, J=7.6 Hz, 1H), 8.39 (dt, J=2.3, 0.9 Hz, 1H), 8.16 (d, J=2.4 Hz, 1H), 7.72 (s, 2H), 7.54-7.44 (m, 2H), 7.35-7.22 (m, 2H), 4.52 (d, J=1.9 Hz, 2H), 4.25 (qd, J=7.5, 3.8 Hz, 1H), 3.92-3.82 (m, 1H), 2.07-1.96 (m, 1H), 1.95-1.85 (m, 1H), 1.78-1.47 (m, 4H). MS (DCI+) m/z 458.0 (M+H)+.

### Intermediate 25B

### [4-({[(1S,2S)-2-{[2-amino-5-(trifluoromethyl)pyridine-3-carbonyl]amino}cyclopentyl]oxy}methyl)phenyl]boronic acid

A mixture of the product of Intermediate 25A (8.52 g, 18.59 mmol) and potassium acetate (3.65 g, 37.2 mmol) was dried in a vacuum oven at 50° C. for 1 hour. The mixture was cooled under nitrogen and hypodiboric acid (3.33 g, 37.2 mmol) and chloro[(tri-tert-butylphosphine)-2-(2-aminobiphenyl)]palladium(II) (0.476 g, 0.930 mmol) were added under nitrogen. A mixture of 30% ethylene glycol/methanol (100 mL) which was sparged with nitrogen for 1 hour and then cooled to 0° C., was transferred via cannula. After 30 minutes, a thick slurry formed and 30% ethylene glycol/methanol (100 mL) was added. After 1 hour, the mixture was poured into water, and the precipitate filtered and washed with water. The precipitate was dissolved in 2% methanol/dichloromethane (400 mL), and ammonium pyrrolidine dithiocarbamate (1 g) was added. The mixture was stirred for 15 minutes, then dried over MgSO₄, filtered, and concentrated. The residue was purified by flash chromatography on silica gel eluting with 0-5% methanol/dichloromethane to afford the title compound (3.36 g). ¹H NMR (501 MHz, DMSO-d₆-D₂O) δ 8.36 (dd, J=2.4, 1.0 Hz, 1H), 8.14 (dd, J=2.5, 0.7 Hz, 1H), 7.75-7.67 (m, 2H), 7.28-7.21 (m, 2H), 4.57-4.45 (m, 2H), 4.24 (ddd, J=8.0, 6.4, 4.1 Hz, 1H), 3.86 (dt, J=6.3, 4.0 Hz, 1H), 2.00 (dtd, J=12.9, 7.9, 5.1 Hz, 1H), 1.93-1.83 (m, 1H), 1.75-1.58 (m, 3H), 1.52 (ddt, J=13.1, 8.5, 6.9 Hz, 1H). MS (ESI−) m/z 422.1 (M−H)⁻.

### Intermediate 26

### [4—({[(3R,4S)-4-{[2-amino-5-(trifluoromethyl)pyridine-3-carbonyl]amino}oxolan-3-yl]oxy}methyl)phenyl]boronic acid

### Intermediate 26A

### 2-amino-N-{(3S,4R)-4-[(4-bromophenyl)methoxy]oxolan-3-yl}-5-(trifluoromethyl)pyridine-3-carboxamide

To a mixture of 2-amino-5-(trifluoromethyl)pyridine-3-carboxylic acid (14.24 g, 44.5 mmol), 2-(3H-[1,2,3]triazolo[4,5-b]pyridin-3-yl)-1,1,3,3-tetramethylisouronium hexafluorophosphate (18.61 g, 48.9 mmol) in NN-dimethylformamide (37.1 mL) was added N-ethyl-N-isopropylpropan-2-amine (16.32 mL, 93 mmol), and the mixture was stirred for 1 hour under nitrogen. A solution of Intermediate 28 (12.35 g, 45.4 mmol) in N,N-dimethylformamide (37.1 mL) was added via cannula followed by N-ethyl-N-isopropylpropan-2-amine (7.77 mL, 44.5 mmol), and the mixture stirred for 2 hours. 2-(3H-[1,2,3]Triazolo[4,5-b]pyridin-3-yl)-1,1,3,3-tetramethylisouronium hexafluorophosphate (18 g, 47.3 mmol) and N-ethyl-N-isopropylpropan-2-amine (40 mL, 230 mmol) were added, and the mixture was stirred for 2 hours. The mixture was partitioned between ethyl acetate and water, and the aqueous layer was extracted with ethyl acetate. The organic layers were dried over MgSO₄, filtered, and concentrated, and the residue was purified by flash chromatography on silica gel eluting with 0-5% methanol/dichloromethane to afford the title compound (14.3 g). ¹H NMR (400 MHz, DMSO-d₆) δ 8.79 (d, J=6.7 Hz, 1H), 8.44-8.37 (m, 1H), 8.27 (d, J=2.4 Hz, 1H), 7.74 (s, 2H), 7.56-7.52 (m, 2H), 7.34-7.28 (m, 2H), 4.67 (d, J=12.4 Hz, 1H), 4.57 (d, J=12.4 Hz, 1H), 4.43 (dddd, J=6.5, 5.2, 3.3, 1.5 Hz, 1H), 4.09 (dt, J=4.7, 1.8 Hz, 1H), 4.01 (dd, J=9.4, 6.1 Hz, 1H), 3.94 (dd, J=10.0, 4.8 Hz, 1H), 3.73 (ddd, J=9.1, 6.4, 2.7 Hz, 2H). MS (ESI+) m/z 461.8 (M+H)+.

### Intermediate 26B

### [4-({[(3R,4S)-4-{[2-amino-5-(trifluoromethyl)pyridine-3-carbonyl]amino}oxolan-3-yl]oxy}methyl)phenyl]boronic acid

A mixture of the product of Step 26A (4.84 g, 10.52 mmol) and potassium acetate (2.167 g, 22.08 mmol) was dried in a vacuum oven at 50° C. for 30 minutes. The mixture was cooled under nitrogen and tetrahydroxydiboron (1.886 g, 21.03 mmol) and chloro[(tri-tert-butylphosphine)-2-(2-aminobiphenyl)]palladium(II) (0.269 g, 0.526 mmol) were added. A 30% methanol/ethylene glycol mixture (35.1 mL) sparged with nitrogen for 1 hour and then cooled to 0° C., was transferred via cannula. The mixture was stirred for 2 hours and poured into water. The mixture was extracted with 5% methanol/dichloromethane. The organic layers were dried over MgSO₄, filtered, and concentrated. The residue was purified by flash chromatography on silica gel eluting with 0-20% methanol/dichloromethane to afford the title compound (1.9 g). ¹H NMR (400 MHz, DMSO-d₆) δ 8.37 (dd, J=2.4, 1.0 Hz, 1H), 8.22 (d, J=2.4 Hz, 1H), 7.80-7.68 (m, 2H), 7.29 (d, J=7.8 Hz, 2H), 4.67 (d, J=12.4 Hz, 1H), 4.56 (d, J=12.4 Hz, 1H), 4.42 (ddd, J=5.6, 3.5, 1.5 Hz, 1H), 4.07 (dt, J=4.9, 1.9 Hz, 1H), 4.00 (dd, J=9.4, 6.2 Hz, 1H), 3.91 (dd, J=10.1, 4.8 Hz, 1H), 3.75-3.71 (m, 2H), 3.38 (s, 1H), 3.15 (s, 1H). MS (ESI+) m/z (M+H)⁺426.3.

### Intermediate 27

### [(2-nitrobenzene-1-sulfonyl)azanediyl]di(ethane-2,1-diyl) bis(2-nitrobenzene-1-sulfonate)

To a solution of 2,2′-azanediyldiethanol (10 g, 95 mmol) in dichloromethane (700 mL) was added 2-nitrobenzene-1-sulfonyl chloride (65.3 g, 295 mmol) and triethylenediamine (33.1 g, 295 mmol) portionwise at 0° C., and the mixture was stirred at 25° C. for 1 hour. The mixture was poured into water, and the organic phase was washed with 2 N HCl and brine, dried over Na₂SO₄, filtered, and concentrated. The residue was purified by flash chromatography on silica gel eluting with 5/1 petroleum ether/ethyl acetate to afford the title compound. ¹H NMR (400 MHz, CDCl₃) δ 3.84 (t, J=5.32 Hz, 4H), 4.42 (t, J=5.32 Hz, 4H), 7.59-7.68 (m, 1H), 7.70-7.76 (m, 2H), 7.77-7.88 (m, 6H), 8.05 (dd, J=5.81, 3.48 Hz, 1H), 8.12 (d, J=7.34 Hz, 2H).

### Intermediate 28

### (3S,4R)-4-[(4-bromophenyl)methoxy]oxolan-3-amine

To a solution of (3R,4S)-4-aminotetrahydrofuran-3-ol (7.5 g, 72.7 mmol) in N,N-dimethylformamide (145 mL) at 17° C. was added 1 M potassium tert-butoxide in tetrahydrofuran (100 mL, 100 mmol) via cannula and the mixture was stirred at 17° C. for 30 minutes. 1-Bromo-4-(bromomethyl)benzene (21.81 g, 87 mmol) was added in portions, whereupon a mild exotherm was observed and a precipitate formed. The cooling bath was removed, and the mixture was stirred at ambient temperature for 2 hours. The reaction was quenched with methanol (10 mL) and stirred for 30 minutes. The mixture was poured into water and extracted with ethyl acetate. The extracts were washed with brine, dried over Na₂SO₄, and concentrated. The residue was purified by flash chromatography on silica gel eluting with 0-10% methanol/dichloromethane to afford the title compound (14.7 g). MS (ESI+) m/z 272, 274 (M+H)⁺.

### Intermediate 29

### 2-amino-N-{(3S,4R)-4-[(4-bromophenyl)methoxy]oxolan-3-yl}-5-(1-methyl-1H-pyrazol-4-yl)pyridine-3-carboxamide

To a solution of Intermediate 28 (25.07 g, 92 mmol) in N,N-dimethylformamide (200 mL) was added Intermediate 2 (16.75 g, 77 mmol) and N-ethyl-N-isopropylpropan-2-amine (33.2 mL, 192 mmol), and the mixture was stirred until most of the solid had dissolved. 2-(3H-[1,2,3]Triazolo[4,5-b]pyridin-3-yl)-1,1,3,3-tetramethylisouronium hexafluorophosphate(V) (32.1 g, 84 mmol) was added, and the mixture was stirred at ambient temperature overnight. The mixture was poured into water, treated with saturated NaHCO₃ and extracted with ethyl acetate. The extracts were treated with mercapto-functionalized silica gel (PhosPhonics SPM32, 3 g) and stirred for 30 minutes, then dried over Na₂SO₄, filtered, and concentrated to about 500 mL. The mixture was allowed to stand overnight, during which time a white precipitate was formed. The slurry was further concentrated to a volume of 400 mL and heptane (100 mL) was added. The mixture was allowed to stand for 30 minutes, and the precipitate was collected by filtration and rinsed with 1:1 ethyl acetate/heptane to afford a first crop (17.7 g). Further concentration of the mother liquor yielded additional precipitate, which was precipitated from hot ethanol to afford a second crop of product (3.93 g). A third crop was also collected from the filtrate (862 mg) to afford a total of 22.21 g of the title compound. MS (ESI+) m/z 472, 474 (M+H)⁺.

### Intermediate 30

### [4-({[(3R,4S)-4-{[2-amino-5-(1-methyl-1H-pyrazol-4-yl)pyridine-3-carbonyl]amino}oxolan-3-yl]oxy}methyl)phenyl]boronic acid

A mixture of Intermediate 29 (1.0 g, 2.12 mmol), tetrahydroxydiboron (380 mg, 4.23 mmol), chloro(2-dicyclohexylphosphino-2′,4′,6′-triisopropyl-1,1′-biphenyl)[2-(2′-amino-1,1′-biphenyl)]palladium(II) (42 mg, 0.053 mmol), dicyclohexyl(2′,4′,6′-triisopropyl-[1-1′-biphenyl]-2-yl)phosphine (61 mg, 127 mmol) and potassium acetate (436 mg, 4.45 mmol) was degassed with nitrogen for 30 minutes. Nitrogen-sparged ethanol (10 mL) was added, and the mixture was heated at 80° C. for 1.5 hours. The mixture was cooled to ambient temperature, filtered, and rinsed with ethanol. The filtrate was concentrated, and the residue was dissolved in 0.1 N NaOH (100 mL). The aqueous mixture was washed with CH₂Cl₂, filtered, and the filtrate was adjusted to pH 7 by addition of 10% citric acid. The slurry was stirred for 2 hours and the precipitate was collected by filtration and rinsed with water to afford the title compound (590 mg). MS (ESI+) m/z 438 (M+H)⁺.

### Intermediate 31

### 6-amino-N-{(1S,2S)-2-[(4-bromophenyl)methoxy]cyclopentyl}-6′-fluoro[3,3′-bipyridine]-5-carboxamide

To a suspension of Intermediate 7 (100 g, 429 mmol) in N,N-dimethylformamide (1600 mL) was added N-ethyl-N-isopropylpropan-2-amine (150 mL, 858 mmol), followed by N¹-((ethylimino)methylene)-N³,N³-dimethylpropane-1,3-diamine hydrochloride (123 g, 643 mmol). 1H-Benzo[d][1,2,3]triazol-1-ol hydrate (8.21 g, 42.9 mmol) was added, the mixture was stirred for 10 minutes. Intermediate 1 (131 g, 429 mmol) was added, and the mixture was stirred at ambient temperature for 12 hours. The mixture was taken up in ethyl acetate and the organics washed successively with water, saturated NaHCO₃, water and brine. The organics were dried over MgSO₄ and concentrated. The crude material was suspended in methyl tert-butyl ether and the suspension was stirred vigorously for 2.5 hours, filtered and dried under vacuum to afford the title compound (92 g), which was used without further purification. ¹H NMR (501 MHz, DMSO-d₆) δ 8.54 (d, J=2.6 Hz, 1H), 8.45 (d, J=2.4 Hz, 1H), 8.35 (d, J=7.6 Hz, 1H), 8.25 (td, J=8.2, 2.7 Hz, 1H), 8.14 (d, J=2.4 Hz, 1H), 7.52-7.40 (m, 2H), 7.32-7.14 (m, 5H), 4.59-4.46 (m, 2H), 4.27 (qd, J=7.6, 3.8 Hz, 1H), 3.86 (dt, J=6.3, 3.9 Hz, 1H), 2.02 (dtd, J=12.9, 7.9, 5.0 Hz, 1H), 1.95-1.84 (m, 1H), 1.79-1.46 (m, 4H). MS (ESI+) m/z 485.2 (M+H)⁺.

### Intermediate 32

### 6-amino-6′-fluoro-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

To a mixture of Intermediate 31 (50 g, 103 mmol), 4,4,4′,4′,5,5,5′,5′-octamethyl-2,2′-bi(1,3,2-dioxaborolane) (39.2 g, 155 mmol) and potassium acetate (20.22 g, 206 mmol) was added 2-methyltetrahydrofuran (500 mL), and the solution was degassed with nitrogen for 2 hours. Tris(dibenzylideneacetone)dipalladium(0) (0.943 g, 1.030 mmol) and 2-dicyclohexylphosphino-2′,4′,6′-triisopropylbiphenyl (1.964 g, 4.12 mmol) were quickly added, and the mixture was heated at 75° C. overnight. The mixture was cooled, filtered, and rinsed with tetrahydrofuran. The filtrate was taken up in ethyl acetate. and the organics washed with water and brine, dried over MgSO₄ and concentrated. Flash chromatography on silica gel eluting with 45-90% ethyl acetate/heptane afforded the title compound (48 g). ¹H NMR (400 MHz, DMSO-d₆) δ 8.52 (d, J=2.6 Hz, 1H), 8.44 (d, J=2.3 Hz, 1H), 8.35 (d, J=7.6 Hz, 1H), 8.23 (td, J=8.2, 2.7 Hz, 1H), 8.14 (d, J=2.4 Hz, 1H), 7.61-7.53 (m, 2H), 7.33-7.16 (m, 5H), 4.66-4.48 (m, 2H), 4.37-4.20 (m, 1H), 3.89-3.82 (m, 1H), 2.09-1.95 (m, 1H), 1.89 (dtd, J=8.3, 6.6, 5.0 Hz, 1H), 1.76-1.45 (m, 4H), 1.23 (s, 12H). MS (ESI+) m/z 533.4 (M+H)⁺.

### Intermediate 33

### 2-amino-5-(1-methyl-1H-pyrazol-4-yl)-N-[(1S,2S)-2-{[4-(4,4,5,5-tetramethyl-1,3,2-dioxaborolan-2-yl)phenyl]methoxy}cyclopentyl]pyridine-3-carboxamide

A mixture of Intermediate 3 (0.86 g, 1.828 mmol) and 4,4,4′,4′,5,5,5′,5′-octamethyl-2,2′-bi(1,3,2-dioxaborolane) (0.929 g, 3.66 mmol) in degassed dioxane (10 mL) was treated with potassium acetate (0.538 g, 5.49 mmol) followed by [1,1′ bis(dipheniylphosphino)ferrocene]dichlioropalIadiurn(II) (0.119 g, 0.146 mmol), and the mixture was heated under nitrogen at 105° C. for 2.5 hours. After cooling to ambient temperature, the mixture was partitioned in ethyl acetate and water, and the solids were filtered off through diatomaceous earth. The organic layer was dried over Na₂SO₄, filtered, and concentrated. Purification by flash chromatography on silica gel eluting with 0-10% methanol/ethyl acetate provided the title compound (620 mg). MS (ESI+) m/z 518.5 (M+H)⁺.

### Intermediate 34

### 2-amino-N-[(1S,2S)-2-{[4-(4,4,5,5-tetramethyl-1,3,2-dioxaborolan-2-yl)phenyl]methoxy}cyclopentyl]-5-(trifluoromethyl)pyridine-3-carboxamide

A mixture of Intermediate 25A (200 mg, 0.436 mmol), 4,4,4′,4′,5,5,5′,5′-octamethyl-2,2′-bi(1,3,2-dioxaborolane) (222 mg, 0.873 mmol), tetrakis(triphenylphosphine)palladium(0) (25.2 mg, 0.022 mmol) and potassium acetate (128 mg, 1.309 mmol) was evacuated and back-filled with nitrogen. Dioxane (2.2 mL) was added, and the mixture was stirred under nitrogen at 100° C. for 16 hours. The mixture was directly purified by flash chromatography on silica gel eluting with 0-30% ethyl acetate/heptanes to afford the title compound (193 mg). ¹H NMR (400 MHz, CDCl₃) δ 8.38 (dd, J=2.3, 1.0 Hz, 1H), 7.86-7.70 (m, 2H), 7.64 (d, J=2.3 Hz, 1H), 7.34 (d, J=7.7 Hz, 2H), 6.77 (s, 2H), 5.88 (d, J=7.1 Hz, 1H), 4.75-4.57 (m, 2H), 4.35 (qd, J=7.0, 4.3 Hz, 1H), 3.84 (dt, J=6.5, 4.4 Hz, 1H), 2.28 (dtd, J=13.4, 7.8, 5.3 Hz, 1H), 2.05-1.82 (m, 2H), 1.82-1.67 (m, 2H), 1.50 (tt, J=13.6, 6.7 Hz, 1H), 1.34 (s, 12H). LCMS (ESI+) m/z 506.36 (M+H)⁺.

### Intermediate 35

### (3S,4S)-4-[(4-bromophenyl)methoxy]-1-methylpyrrolidin-3-amine Intermediate 35A tert-butyl (3S,4S)-3-(1,3-dioxo-1,3-dihydro-2H-isoindol-2-yl)-4-hydroxypyrrolidine-1-carboxylate

A mixture of (3S,4S)-tert-butyl 3-amino-4-hydroxypyrrolidine-1-carboxylate (5 g, 24.72 mmol), phthalic anhydride (3.66 g, 24.72 mmol), toluene (125 mL), and triethylamine (2.50 g, 24.72 mmol) were heated to reflux overnight with a Dean-Stark apparatus. The mixture was cooled to ambient temperature and diluted with ethyl acetate and 10% citric acid. A precipitate (4.48 g) was collected by filtration, and the ethyl acetate layer was washed with 1 N NaOH, water and brine, dried over Na₂SO₄, and concentrated to afford additional product (2.46 g). An additional crop (518 mg) was collected from the aqueous layer for a combined total of 7.46 g. MS (ESI+) m/z 233.1 (M-Boc)⁺, 387.2 (M+methanol+Na)⁺.

### Intermediate 35B

### 2-[(3S,4S)-4-hydroxypyrrolidin-3-yl]-1H-isoindole-1,3 (2H)-dione

To a solution of Intermediate 35A (6.61 g, 19.89 mmol) in dioxane (40 mL) and methanol (10 mL) was added 4 N HCl in dioxane (26 mL, 104 mmol), and the mixture was stirred for 90 minutes (gas evolution ceased). The mixture was diluted with methyl tert-butylether (50 mL) and allowed to stand at ambient temperature for 1.5 hours. The precipitate was collected and rinsed with methyl tert-butylether to afford the title compound as a HCl salt (5.11 g). MS (ESI+) m/z 233.1 (M+H)⁺.

### Intermediate 35C

### 2-[(3S,4S)-4-hydroxy-1-methylpyrrolidin-3-yl]-1H-isoindole-1,3 (2H)-dione

To a solution of Intermediate 35B (2 g, 7.44 mmol) in acetonitrile (37.2 mL) was added 37% aqueous formaldehyde (2.79 mL, 37.2 mmol), followed by portionwise addition of sodium triacetoxyhydroborate (5.52 g, 26.1 mmol), maintaining a temperature <25° C. during the addition. The reaction was stirred at ambient temperature for 3 hours and poured into saturated NaHCO₃ (150 mL). The mixture was stirred for 20 minutes and extracted with ethyl acetate, dried over Na₂SO₄, and concentrated. The residue was purified by flash chromatography on silica gel eluting with 0-15% methanol/dichloromethane to afford the title compound (1.37 g). MS (ESI+) m/z 247.3 (M+H)⁺.

### Intermediate 35D

### 2-{(3S,4S)-4-[(4-bromophenyl)methoxy]-1-methylpyrrolidin-3-yl}-1H-isoindole-1,3 (2H)-dione

A solution of Intermediate 35C (500 mg, 2.030 mmol) in tetrahydrofuran (10 mL) was cooled in an ice bath and 1 M potassium tert-butoxide in tetrahydrofuran (2.23 mL, 2.23 mmol) was added. The mixture was stirred at 4-7° C. for 20 minutes and 1-bromo-4-(bromomethyl)benzene (609 mg, 2.436 mmol) was added. The ice bath was removed, and the mixture was stirred for 30 minutes. The mixture was quenched with methanol, stirred for 1 hour and concentrated. Purification by flash chromatography on silica gel eluting with 0-7% methanol/CH₂Cl₂ gave the title compound (440 mg). MS (ESI+) m/z 415.2, 417.2 (M+H)⁺, Br pattern.

### Intermediate 35E

### (3S,4S)-4-[(4-bromophenyl)methoxy]-1-methylpyrrolidin-3-amine

To a solution of Intermediate 35D (3.4 g, 8.19 mmol) in ethanol (80 mL) was added hydrazine hydrate (0.798 mL, 16.37 mmol), and the mixture was heated to reflux for 3 hours, then cooled to ambient temperature and filtered through diatomaceous earth. The filtrate was concentrated and partitioned between 1 N NaOH and dichloromethane. The organic extracts were dried over Na₂SO₄, and concentrated to afford the title compound (2.16 g). MS (ESI+) m/z 285.2, 287.2 (M+H)⁺, Br pattern.

### Intermediate 36

### 2-amino-5-bromo-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyridine-3-carboxamide

### Intermediate 36A

### tert-butyl {(1S,2S)-2-[(4-bromophenyl)methoxy]cyclopentyl}carbamate

A mixture of Intermediate 1 (10 g, 32.6 mmol), tetrahydrofuran (100 mL), di-tert-butyl dicarbonate (32.6 mL, 32.6 mmol), triethylamine (11.36 mL, 82 mmol) and 4-dimethylaminopyridine (0.398 g, 3.26 mmol) was stirred at ambient temperature for 2 days. The mixture was diluted with ethyl acetate, filtered, concentrated and purified by flash chromatography on silica gel eluting with 0-15% ethyl acetate/heptanes to afford the title compound (4.84 g). ¹H NMR (400 MHz, DMSO-d₆) δ 7.58-7.42 (m, 2H), 7.32-7.19 (m, 2H), 6.88 (d, J=7.7 Hz, 1H), 4.67-4.33 (m, 2H), 3.74 (d, J=5.9 Hz, 1H), 3.68 (dt, J=6.1, 3.5 Hz, 1H), 1.93-1.83 (m, 1H), 1.79 (ddd, J=11.7, 7.9, 5.6 Hz, 1H), 1.68-1.48 (m, 3H), 1.45-1.32 (m, 10H). MS (DCI+) m/z 387.1 (M+NH₄)⁺.

### Intermediate 36B

### tert-butyl [(1S,2S)-2-{[4-(4,4,5,5-tetramethyl-1,3,2-dioxaborolan-2-yl)phenyl]methoxy}cyclopentyl]carbamate

A mixture of Intermediate 36A (3.84 g, 10.37 mmol), potassium acetate (2.036 g, 20.74 mmol), 4,4,4′,4′,5,5,5′,5′-octamethyl-2,2′-bi(1,3,2-dioxaborolane) (3.95 g, 15.56 mmol) in 2-methyltetrahydrofuran (50 mL) was sparged with nitrogen for 30 minutes. Tris(dibenzylideneacetone)dipalladium(0) (0.095 g, 0.104 mmol) and dicyclohexyl(2′,4′,6′-triisopropyl-[1,1′-biphenyl]-2-yl)phosphine (0.203 g, 0.425 mmol) were added, and the mixture was heated at 75° C. for 16 hours and then cooled to ambient temperature. The mixture was diluted with methyl tert-butylether, filtered, and the filtrate was washed with water and brine, dried over MgSO₄, filtered, and concentrated. The residue was purified by flash chromatography on silica gel eluting with 0-20% ethyl acetate/heptanes to afford the title compound (3.0 g). ¹H NMR (501 MHz, DMSO-d₆) δ 7.72-7.53 (m, 2H), 7.35-7.24 (m, 2H), 6.86 (d, J=7.8 Hz, 1H), 4.63-4.46 (m, 2H), 3.75 (s, 1H), 3.68 (dt, J=6.4, 3.6 Hz, 1H), 1.93-1.84 (m, 1H), 1.79 (ddd, J=11.9, 7.9, 6.0 Hz, 1H), 1.68-1.49 (m, 3H), 1.43-1.36 (m, 10H), 1.28 (s, 12H). MS (DCI+) m/z 435.3 (M+NH₄)⁺.

### Intermediate 36C

### tert-butyl {(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}carbamate

A mixture of Intermediate 36B (3 g, 7.19 mmol), Intermediate 23 (2.54 g, 7.19 mmol) and Cs₂CO₃ (4.68 g, 14.38 mmol) in dioxane (30 mL) and water (10 mL) was sparged with nitrogen for 1 hour. Tris(dibenzylideneacetone)dipalladium(0) (0.066 g, 0.072 mmol) and 4-(di-tert-butylphosphino)-N,N-dimethylaniline (0.042 g, 0.158 mmol) were added, and the mixture was heated at 75° C. for 6 hours and then cooled to ambient temperature. Ammonium pyrrolidinedithiocarbamate (200 mg) and water (10 mL) were added and after stirring for 15 minutes, the mixture was partitioned between ethyl acetate and water. The aqueous layer was washed with ethyl acetate and the organic layers were dried over MgSO₄, filtered, and concentrated. The residue was purified by flash chromatography on silica gel eluting with 0-5% methanol/dichloromethane to afford the title compound (2.7 g). ¹H NMR (500 MHz, DMSO-d₆) δ 7.52-7.47 (m, 2H), 7.32-7.24 (m, 4H), 6.89 (d, J=7.8 Hz, 1H), 6.50 (d, J=8.1 Hz, 1H), 4.52 (d, J=12.1 Hz, 1H), 4.47 (d, J=12.1 Hz, 1H), 4.37 (s, 1H), 3.78 (h, J=7.4, 6.7 Hz, 1H), 3.71 (dt, J=6.4, 3.5 Hz, 1H), 3.49 (t, J=6.4 Hz, 2H), 3.44-3.36 (m, 2H), 3.14 (s, 2H), 2.94 (dt, J=12.8, 3.5 Hz, 2H), 2.40 (t, J=6.3 Hz, 2H), 2.07 (td, J=11.5, 2.8 Hz, 2H), 1.89 (m, 1H), 1.80 (dtt, J=11.9, 7.3, 3.6 Hz, 1H), 1.71-1.50 (m, 7H), 1.40 (s, 9H), 1.26 (s, 6H). MS (DCI+) m/z 564.4 (M+H)⁺.

### Intermediate 36D

### 2-(4-{5-[4-({[(1S,2S)-2-aminocyclopentyl]oxy}methyl)phenyl]-3,3-dimethyl-2,3-dihydro-1H-indol-1-yl}piperidin-1-yl)ethan-1-ol

A solution of Intermediate 36C (0.211 g, 0.374 mmol) in tetrahydrofuran (3.8 mL) was cooled to 0° C. and 4 M HCl in dioxane (0.936 mL, 3.74 mmol) was added. The mixture was warmed to ambient temperature and stirred until complete. Concentration afforded the title compound as a HCl salt (0.23 g). ¹H NMR (501 MHz, DMSO-d₆) δ 10.34 (s, 1H), 8.29 (s, 3H), 7.58-7.51 (m, 2H), 7.39-7.30 (m, 4H), 6.63 (d, J=8.2 Hz, 1H), 4.52 (s, 2H), 3.98 (dt, J=6.3, 4.2 Hz, 1H), 3.79 (dd, J=6.3, 4.3 Hz, 2H), 3.72-3.63 (m, 1H), 3.55-3.36 (m, 2H), 3.23-3.03 (m, 6H), 2.18-1.93 (m, 4H), 1.86 (dt, J=14.9, 7.4 Hz, 2H), 1.78-1.47 (m, 5H), 1.29 (s, 6H). MS (DCI+) m/z 464.4 (M+H)⁺.

### Intermediate 36E

### 2-amino-5-bromo-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyridine-3-carboxamide

To Intermediate 36D (0.230 g, 0.401 mmol) was added 2-amino-5-bromopyridine-3-carboxylic acid (0.096 g, 0.442 mmol), 1-(3-dimethylaminopropyl)-3-ethylcarbodiimide hydrochloride (0.115 g, 0.602 mmol), 1-hydroxybenzotriazole hydrate (0.092 g, 0.602 mmol), N,N-dimethylformamide (4 mL) and N,N-diisopropylethylamine (0.351 mL, 2.007 mmol), and the mixture was stirred for 16 hours. The mixture was partitioned between ethyl acetate and water, and the aqueous layer was washed with ethyl acetate. The organic layers were dried over MgSO₄, filtered, and concentrated. The residue was purified by flash chromatography on silica gel eluting with 0-5% methanol/dichloromethane to afford the title compound (143 mg). ¹H NMR (501 MHz, DMSO-d₆) δ 8.41 (d, J=7.7 Hz, 1H), 8.14 (d, J=2.4 Hz, 1H), 8.06 (d, J=2.4 Hz, 1H), 7.56-7.41 (m, 2H), 7.32-7.24 (m, 4H), 7.20 (s, 2H), 6.50 (d, J=8.1 Hz, 1H), 4.58-4.50 (m, 2H), 4.40 (s, 1H), 4.26 (qd, J=7.6, 3.9 Hz, 1H), 3.87 (dt, J=6.2, 3.9 Hz, 1H), 3.50 (q, J=5.8 Hz, 3H), 3.14 (s, 2H), 2.96 (d, J=11.0 Hz, 2H), 2.42 (t, J=6.3 Hz, 2H), 2.11 (t, J=11.0 Hz, 2H), 2.04-1.96 (m, 1H), 1.91 (dt, J=12.3, 7.1 Hz, 1H), 1.75-1.45 (m, 8H), 1.26 (s, 6H). LCMS (APCI) 662.5 (M+H)⁺.

### Intermediate 37

### 6-amino-6′-fluoro-N-[(3S,4R)-4-{[4-(4,4,5,5-tetramethyl-1,3,2-dioxaborolan-2-yl)phenyl]methoxy}oxolan-3-yl][3,3′-bipyridine]-5-carboxamide

### Intermediate 37A

### 6-amino-N-{(3S,4R)-4-[(4-bromophenyl)methoxy]oxolan-3-yl}-6′-fluoro[3,3′-bipyridine]-5-carboxamide

To a mixture of Intermediate 7 (0.25 g, 1.1 mmol) and triethylamine (217 g, 2.2 mmol) in N,N-dimethylformamide (5 mL) was added 1-[bis(dimethylamino)methylene]-1H-1,2,3-triazolo[4,5-b]pyridinium 3-oxid hexafluorophosphate (HATU, 0.45 g, 1.2 mmol) followed by Intermediate 28 (0.29 mg, 0.11 mmol), and the mixture was stirred at room temperature for 1 hour. The mixture was quenched with water and partitioned between water and ethyl acetate. The organic phase was dried over MgSO₄, concentrated, and purified by flash chromatography on silica gel eluting with 10% methanol/ethyl acetate to afford the title compound (348 mg). ¹H NMR (400 MHz, CDCl₃) δ 8.29 (d, J=2.3 Hz, 1H), 8.16 (dt, J=1.9, 0.9 Hz, 1H), 7.82 (ddd, J=8.4, 7.5, 2.6 Hz, 1H), 7.67 (d, J=2.3 Hz, 1H), 7.51-7.43 (m, 2H), 7.33-7.22 (m, 2H), 6.97 (ddd, J=8.5, 3.0, 0.7 Hz, 1H), 6.76 (d, J=7.1 Hz, 1H), 6.61 (s, 2H), 4.83 (d, J=12.3 Hz, 1H), 4.67 (d, J=12.2 Hz, 1H), 4.62 (dd, J=7.1, 4.3 Hz, 1H), 4.18 (t, J=5.0 Hz, 1H), 4.13 (dd, J=8.2, 6.0 Hz, 1H), 4.08 (dd, J=9.9, 4.4 Hz, 1H), 3.94 (d, J=9.9 Hz, 1H), 3.76 (dd, J=9.9, 2.2 Hz, 1H). MS (DCI/NH₃) m/z 488 (M+H)⁺.

### Intermediate 37B

### 6-amino-6′-fluoro-N-[(3S,4R)-4-{[4-(4,4,5,5-tetramethyl-1,3,2-dioxaborolan-2-yl)phenyl]methoxy}oxolan-3-yl][3,3′-bipyridine]-5-carboxamide

A mixture of Intermediate 37A (5 g, 10 mmol), 4,4,4′,4′,5,5,5′,5′-octamethyl-2,2′-bi(1,3,2-dioxaborolane) (5.2 g, 20 mmol), tetrakis(triphenylphosphine)palladium(0) (590 mg, 0.5 mmol) and potassium acetate (2 g, 21 mmol) was degassed with argon, and dioxane (40 mL) was added. The mixture was stirred under argon at 110° C. overnight. The mixture was quenched with water and extracted with ethyl acetate. The organic phase was dried over MgSO₄, concentrated and purified by flash chromatography on silica gel eluting with 10% methanol/ethyl acetate to afford the title compound (5.2 g). ¹H NMR (501 MHz, CDCl₃) δ 8.28 (d, J=2.3 Hz, 1H), 8.13 (tt, J=3.4, 0.9 Hz, 1H), 7.85-7.76 (m, 1H), 7.68 (dd, J=5.6, 2.4 Hz, 1H), 7.63-7.56 (m, 2H), 7.52-7.42 (m, 2H), 6.96 (tdd, J=8.4, 3.0, 0.7 Hz, 1H), 6.87 (dd, J=21.9, 7.1 Hz, 1H), 6.66 (d, J=7.6 Hz, 2H), 4.92 (dd, J=13.6, 12.2 Hz, 1H), 4.76 (t, J=12.1 Hz, 1H), 4.73-4.64 (m, 1H), 4.25-4.08 (m, 3H), 4.01-3.93 (m, 1H), 3.85-3.76 (m, 1H), 1.36 (s, 9H). MS (DCI/NH₃) m/z 535 (M+H)⁺.

### Example 1

### 2-amino-N-{(1S,2S)-2-[(4-{2-[3-(dimethylamino)propanoyl]-2,3-dihydro-H-isoindol-5-yl}phenyl)methoxy]cyclopentyl}-5-(1-methyl-1H-pyrazol-4-yl)pyridine-3-carboxamide

### Step 1A

### tert-butyl 5-[4-({[(1S,2S)-2-{[2-amino-5-(1-methyl-1H-pyrazol-4-yl)pyridine-3-carbonyl]amino}cyclopentyl]oxy}methyl)phenyl]-1,3-dihydro-2H-isoindole-2-carboxylate

A mixture of Intermediate 3 (0.15 g, 0.319 mmol), tert-butyl 5-(4,4,5,5-tetramethyl-1,3,2-dioxaborolan-2-yl)isoindoline-2-carboxylate (0.127 g, 0.367 mmol), 1,1′-bis(diphenylphosphino)ferrocene]palladium(II) dichloride (0.021 g, 0.026 mmol) and K₂CO₃ (0.11 g, 0.797 mmol) in degassed dioxane (1.6 mL) and degassed water (0.5 mL) was heated under nitrogen at 100° C. for 3 hours. The reaction was allowed to cool to ambient temperature and was partitioned in ethyl acetate and water. The organic layer was dried over Na₂SO₄, and concentrated. The residue was purified by flash chromatography on silica gel eluting with 0-10% methanol/ethyl acetate to afford the title compound (143 mg). MS (ESI+) m/z 609.2 (M+H)⁺.

### Step 1B

### 2-amino-N-[(1S,2S)-2-{[4-(2,3-dihydro-1H-isoindol-5-yl)phenyl]methoxy}cyclopentyl]-5-(1-methyl-1H-pyrazol-4-yl)pyridine-3-carboxamide

A solution of the product of Step TA (0.14 g, 0.230 mmol) and trifluoroacetic acid (0.354 mL, 4.60 mmol) in dichloromethane (2.5 mL) was stirred at ambient temperature for 3 hours. The mixture was concentrated, and the residue was dissolved in dichloromethane (60 mL) and washed with saturated NaHCO₃. The organic layer was dried over Na₂SO₄, and concentrated to afford the title compound (108 mg). MS (ESI+) m/z 509.2 (M+H)⁺.

### Step 1C

### 2-amino-N-{(1S,2S)-2-[(4-{2-[3-(dimethylamino)propanoyl]-2,3-dihydro-1H-isoindol-5-yl}phenyl)methoxy]cyclopentyl}-5-(1-methyl-1H-pyrazol-4-yl)pyridine-3-carboxamide

To a mixture of 3-(dimethylamino)propanoic acid (6.08 mg, 0.052 mmol) and triethylamine (0.024 mL, 0.173 mmol) in N,N-dimethylformamide (0.3 mL) was added 2-(3H-[1,2,3]triazolo[4,5-b]pyridin-3-yl)-1,1,3,3-tetramethylisouronium hexafluorophosphate(V) (0.019 g, 0.050 mmol) followed by the product of Step 1B (0.022 g, 0.043 mmol). The mixture was stirred at ambient temperature for 4 hours and was directly purified by reverse phase HPLC eluting with 10-70% acetonitrile in water (0.1% trifluoroacetic acid) to afford the title compound (10 mg) as a trifluoroacetic acid salt. ¹H NMR (400 MHz, methanol-d₄) δ 8.47 (d, J=2.1 Hz, 1H), 8.21 (d, J=2.1 Hz, 1H), 7.97 (s, 1H), 7.85 (s, 1H), 7.63-7.51 (m, 4H), 7.47-7.40 (m, 3H), 4.71-4.63 (m, 3H), 4.49-4.37 (m, 1H), 4.07-3.96 (m, 1H), 3.90 (s, 3H), 3.09-3.01 (m, 11H), 2.27-2.12 (m, 1H), 2.12-1.98 (m, 1H), 1.92-1.73 (m, 3H), 1.73-1.58 (m, 1H). MS (ESI+) m/z 607.4 (M+H)⁺.

### Example 2

### 2-amino-N-{(1S,2S)-2-[(4-{1-[(dimethylamino)acetyl]-2,3-dihydro-1H-indol-5-yl}phenyl)methoxy]cyclopentyl}-5-(1-methyl-1H-pyrazol-4-yl)pyridine-3-carboxamide

### Step 2A

### 1-(5-bromo-2,3-dihydro-1H-indol-1-yl)-2-(dimethylamino)ethan-1-one

To a mixture of 5-bromoindoline (0.50 g, 2.52 mmol), 1H-benzo[d][1,2,3]triazol-1-ol hydrate (0.387 g, 2.52 mmol), N,N-dimethylformamide (9.5 mL), N-methylmorpholine (1.11 mL, 10.10 mmol) and 2-(dimethylamino)acetic acid (0.299 g, 2.90 mmol) was added 1-(3-dimethylaminopropyl)-3-ethylcarbodiimide hydrochloride (0.678 g, 3.53 mmol), and the mixture was stirred at ambient temperature for 18 hours. The mixture was diluted with water and extracted with 1:1 ether/ethyl acetate. The organic layer was washed with water and brine, dried over Na₂SO₄, and concentrated. The residue was purified by flash chromatography on silica gel eluting with 0-5% methanol/dichloromethane to afford the title compound (580 mg). MS (DCI+) m/z 283.0 (M+H)⁺.

### Step 2B

### 2-amino-N-{(1S,2S)-2-[(4-{1-[(dimethylamino)acetyl]-2,3-dihydro-1H-indol-5-yl}phenyl)methoxy]cyclopentyl}-5-(1-methyl-1H-pyrazol-4-yl)pyridine-3-carboxamide

A mixture of Intermediate 33 (0.03 g, 0.058 mmol), the product of Step 2A (0.019 g, 0.067 mmol), dichloro[1,1′-bis(diphenylphosphino)ferrocene]palladium(II) dichloromethane adduct (3.79 mg, 4.64 μmol) and K₂CO₃ (0.022 g, 0.162 mmol) in degassed water (0.2 mL) and degassed dioxane (0.6 mL) was heated at 95° C. for 1.5 hours. The mixture was cooled to ambient temperature and directly purified by reverse phase HPLC eluting with 10-70% acetonitrile in water (0.1% trifluoroacetic acid) to afford the title compound (29 mg) as a trifluoroacetic acid salt. ¹H NMR (400 MHz, methanol-d₄) δ 8.48 (d, J=2.1 Hz, 1H), 8.21-8.11 (m, 2H), 7.96 (s, 1H), 7.84 (d, J=0.8 Hz, 1H), 7.56-7.50 (m, 2H), 7.49-7.45 (m, 1H), 7.44-7.35 (m, 3H), 4.70-4.59 (m, 2H), 4.47-4.37 (m, 1H), 4.33 (s, 2H), 4.16-4.08 (m, 2H), 4.05-3.96 (m, 1H), 3.90 (s, 3H), 3.34-3.26 (m, 2H), 3.04 (s, 6H), 2.26-2.12 (m, 1H), 2.12-2.00 (m, 1H), 1.93-1.73 (m, 3H), 1.71-1.57 (m, 1H). MS (ESI+) m/z 594.3 (M+H)⁺.

### Example 3

### 2-amino-N-[(1S,2S)-2-{[4-(1-methyl-2-oxo-2,3-dihydro-1H-indol-5-yl)phenyl]methoxy}cyclopentyl]-5-(1-methyl-1H-pyrazol-4-yl)pyridine-3-carboxamide

The title compound was prepared as described in Step 2B substituting 5-bromo-1-methylindolin-2-one for the product of Step 2A. ¹H NMR (500 MHz, methanol-d₄) δ 8.47 (d, J=2.2 Hz, 1H), 8.18 (d, J=2.1 Hz, 1H), 7.95 (s, 1H), 7.84 (s, 1H), 7.54-7.44 (m, 4H), 7.43-7.37 (m, 2H), 7.03-6.96 (m, 1H), 4.71-4.60 (m, 2H), 4.45-4.37 (m, 1H), 3.99 (dq, J=6.5, 4.7 Hz, 1H), 3.89 (s, 3H), 3.58 (s, 2H), 3.24 (s, 3H), 2.24-2.12 (m, 1H), 2.11-2.00 (m, 1H), 1.89-1.73 (m, 3H), 1.69-1.58 (m, 1H). MS (ESI+) m/z 537.26 (M+H)⁺.

### Example 4tert-butyl 4-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}piperidine-1-carboxylate

### Step 4A

### tert-butyl 4-(5-bromo-2,3-dihydro-1H-indol-1-yl)piperidine-1-carboxylate

A solution of 5-bromoindoline (0.5 g, 2.52 mmol) in acetic acid (6 mL) was treated with tert-butyl 4-oxopiperidine-1-carboxylate (0.578 g, 2.90 mmol) followed by sodium triacetoxyhydroborate (0.963 g, 4.54 mmol) portionwise over 5 minutes. The mixture was stirred at ambient temperature for 1 hour, poured into ethyl acetate and water, cooled in an ice bath, and brought to about pH 10 with 6 N NaOH. The organic phase was washed with brine, dried over Na₂SO₄, and concentrated. The residue was purified by flash chromatography on silica gel eluting with 0-30% ethyl acetate/hexanes to afford the title compound (720 mg). MS (ESI+) m/z 380.9 (M+H)⁺.

### Step 4B

### tert-butyl 4-(5-bromo-1H-indol-1-yl)piperidine-1-carboxylate

A 0° C. solution of the product of Step 4A (0.67 g, 1.757 mmol) in tetrahydrofuran (8 mL) was treated with a solution of 2,3-dichloro-5,6-dicyano-1,4-benzoquinone (0.439 g, 1.933 mmol) in tetrahydrofuran (4 mL) added dropwise over 5 minutes. The mixture was stirred at 0° C. for 1 hour and was stored at 10° C. for 16 hours. After stirring at ambient temperature for 1 hour, the mixture was partitioned in ethyl acetate and water. The aqueous layer was brought to pH 10 with 6 N NaOH and the bilayer was shaken vigorously. The aqueous layer was extracted with ethyl acetate and the combined organic layers were washed with brine, dried over Na₂SO₄, and concentrated. The residue was purified by flash chromatography on silica gel eluting with 0-26% ethyl acetate/hexanes to afford the title compound (530 mg). ¹H NMR (400 MHz, methanol-d₄) δ 7.67 (d, J=1.9 Hz, 1H), 7.41 (d, J=8.8 Hz, 1H), 7.36 (d, J=3.3 Hz, 1H), 7.23 (dd, J=8.8, 1.9 Hz, 1H), 6.43 (dd, J=3.3, 0.8 Hz, 1H), 4.51 (tt, J=11.8, 4.0 Hz, 1H), 4.34-4.20 (m, 2H), 3.11-2.87 (m, 2H), 2.11-1.98 (m, 2H), 1.89 (qd, J=12.5, 4.4 Hz, 2H), 1.49 (s, 9H). MS (ESI+) m/z 379.1 (M+H)⁺.

### Step 4C

### tert-butyl 4-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}piperidine-1-carboxylate

A mixture of Intermediate 33 (0.04 g, 0.077 mmol), the product of Step 4B (0.033 g, 0.088 mmol), bis(di-tert-butyl(4-dimethylaminophenyl)phosphine)dichloropalladium(II) (3.28 mg, 4.64 μmol) and K₂CO₃ (0.032 g, 0.232 mmol) in degassed water (0.2 mL) and degassed dioxane (0.6 mL) was heated at 90° C. for 1 hour. The mixture was cooled to ambient temperature and partitioned in ethyl acetate and water. The organic layer was washed with brine, dried over Na₂SO₄, and concentrated. The residue was purified by flash chromatography on silica gel eluting with 0-8% methanol/ethyl acetate to afford the title compound (36 mg). ¹H NMR (400 MHz, methanol-d₄) δ 8.35-8.16 (m, 1H), 7.94 (d, J=2.2 Hz, 1H), 7.78-7.63 (m, 3H), 7.59-7.42 (m, 3H), 7.42-7.28 (m, 4H), 6.49 (d, J=3.2 Hz, 1H), 4.65 (q, J=12.4 Hz, 2H), 4.60-4.55 (m, 1H), 4.48-4.36 (m, 1H), 4.35-4.22 (m, 2H), 3.97 (q, J=5.6 Hz, 1H), 3.75 (s, 3H), 3.15-2.90 (m, 2H), 2.24-1.69 (m, 9H), 1.68-1.53 (m, 1H), 1.50 (s, 9H). MS (ESI+) m/z 690.2 (M+H)⁺.

### Example 5

### 2-amino-5-(1-methyl-1H-pyrazol-4-yl)-N-[(1S,2S)-2-({4-[1-(piperidin-4-yl)-1H-indol-5-yl]phenyl}methoxy)cyclopentyl]pyridine-3-carboxamide

A 0° C. solution of the product of Example 4 (0.024 g, 0.035 mmol) in methanol (0.696 mL) was treated with 4 M HCl in dioxane (0.078 mL, 0.313 mmol) dropwise over 3 minutes. The mixture was stirred at ambient temperature for 20 hours and was partitioned in ethyl acetate and saturated Na₂CO₃. The aqueous layer was extracted with ethyl acetate and the organic layers were washed with brine, dried over Na₂SO₄, filtered, and concentrated to afford the title compound (19 mg). ¹H NMR (500 MHz, methanol-d₄) δ 8.23 (d, J=2.3 Hz, 1H), 7.94 (d, J=2.3 Hz, 1H), 7.75-7.70 (m, 2H), 7.67 (d, J=1.7 Hz, 1H), 7.57-7.51 (m, 2H), 7.49-7.44 (m, 1H), 7.41-7.29 (m, 4H), 6.49 (dd, J=3.3, 0.7 Hz, 1H), 4.65 (q, J=12.4 Hz, 2H), 4.51-4.39 (m, 2H), 4.00-3.94 (m, 1H), 3.74 (s, 3H), 3.26-3.17 (m, 2H), 2.85 (td, J=12.6, 2.7 Hz, 2H), 2.21-2.10 (m, 1H), 2.10-1.89 (m, 5H), 1.87-1.69 (m, 3H), 1.65-1.52 (m, 1H). MS (ESI+) m/z 590.2 (M+H)⁺.

### Example 6

### 2-amino-N-[(1S,2S)-2-({4-[1-(1-methylpiperidin-4-yl)-1H-indol-5-yl]phenyl}methoxy)cyclopentyl]-5-(1-methyl-1H-pyrazol-4-yl)pyridine-3-carboxamide Step 6A 5-bromo-1-(piperidin-4-yl)-1H-indole

The title compound was prepared as described in Example 5 substituting the product of Step 4B for the product of Example 4. MS (ESI+) m/z 279.0 (M+H)⁺.

### Step 6B

### 5-bromo-1-(1-methylpiperidin-4-yl)-1H-indole

A solution of the product of Step 6A (0.0385 g, 0.138 mmol) in methanol (0.8 mL) was treated with acetic acid (0.016 mL, 0.276 mmol) and formaldehyde (37 wt % in water, 0.016 mL, 0.221 mmol), and the mixture was stirred at ambient temperature for 15 minutes. Sodium triacetoxyhydroborate (0.088 g, 0.414 mmol) was added, and the mixture was stirred at ambient temperature for 16 hours. The mixture was concentrated, dissolved in water and adjusted to pH 10 with 6 N NaOH. The suspension was extracted with dichloromethane, dried over Na₂SO₄, and concentrated to afford the title compound. MS (ESI+) m/z 295.2 (M+H)⁺.

### Step 6C

### 2-amino-N-[(1S,2S)-2-({4-[1-(1-methylpiperidin-4-yl)-1H-indol-5-yl]phenyl}methoxy)cyclopentyl]-5-(1-methyl-1H-pyrazol-4-yl)pyridine-3-carboxamide

The title compound was prepared as described in Step 4C substituting the product of Step 6B for the product of Step 4B. ¹H NMR (501 MHz, methanol-d₄) δ 8.23 (d, J=2.2 Hz, 1H), 7.94 (d, J=2.3 Hz, 1H), 7.75-7.71 (m, 2H), 7.67 (d, J=1.8 Hz, 1H), 7.57-7.51 (m, 2H), 7.46 (d, J=8.6 Hz, 1H), 7.40-7.29 (m, 4H), 6.49 (d, J=3.2 Hz, 1H), 4.65 (q, J=12.3 Hz, 2H), 4.45-4.33 (m, 2H), 4.01-3.93 (m, 1H), 3.75 (s, 3H), 3.11-3.02 (m, 2H), 2.43-2.31 (m, 5H), 2.19-2.01 (m, 6H), 1.87-1.70 (m, 3H), 1.63-1.53 (m, 1H). MS (ESI+) m/z 604.2 (M+H)⁺.

### Example 7

### 2-amino-N-{(1S,2S)-2-[(4-{1-[1-(2-hydroxyethyl)piperidin-4-yl]-1H-indol-5-yl}phenyl)methoxy]cyclopentyl}-5-(1-methyl-1H-pyrazol-4-yl)pyridine-3-carboxamide Step 7A 2-[4-(5-bromo-1H-indol-1-yl)piperidin-1-yl]ethan-1-ol

A solution of the product of Step 6A (0.06 g, 0.215 mmol) in methanol (1.6 mL) was treated with K₂CO₃ (0.089 g, 0.645 mmol) followed by 2-bromoethanol (0.036 mL, 0.429 mmol), and the mixture was stirred at ambient temperature for 30 hours and at 60° C. for 24 hours. The mixture was filtered through diatomaceous earth with dichloromethane and the filtrate concentrated. The residue was purified by flash chromatography on silica gel eluting with 0-10% methanol/dichloromethane to afford the title compound (22 mg). MS (ESI+) m/z 323.2 (M+H)⁺.

### Step 7B

### 2-amino-N-{(1S,2S)-2-[(4-{1-[1-(2-hydroxyethyl)piperidin-4-yl]-1H-indol-5-yl}phenyl)methoxy]cyclopentyl}-5-(1-methyl-1H-pyrazol-4-yl)pyridine-3-carboxamide

The title compound was prepared as described in Step 4C substituting the product of Step 7A for the product of Step 4B. ¹H NMR (501 MHz, methanol-d₄) δ 8.23 (d, J=2.3 Hz, 1H), 7.94 (d, J=2.3 Hz, 1H), 7.76-7.70 (m, 2H), 7.67 (d, J=1.7 Hz, 1H), 7.57-7.50 (m, 2H), 7.46 (d, J=8.6 Hz, 1H), 7.42-7.29 (m, 4H), 6.49 (d, J=3.2 Hz, 1H), 4.65 (q, J=12.3 Hz, 2H), 4.48-4.30 (m, 2H), 4.01-3.93 (m, 1H), 3.79-3.71 (m, 5H), 3.16 (d, J=12.4 Hz, 2H), 2.63 (t, J=6.0 Hz, 2H), 2.38 (td, J=12.0, 2.6 Hz, 2H), 2.22-2.02 (m, 6H), 1.86-1.70 (m, 3H), 1.64-1.53 (m, 1H). MS (APCI+) m/z 634.4 (M+H)⁺.

### Example 8

### 2-amino-N-{(1S,2S)-2-[(4-{1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 8A

### tert-butyl 4-(5-bromo-2,3-dihydro-1H-inden-1-yl)piperazine-1-carboxylate

A solution of 5-bromo-2,3-dihydro-1H-inden-1-one (0.5 g, 2.369 mmol) in tetrahydrofuran (10 mL) was treated with tert-butyl piperazine-1-carboxylate (0.794 g, 4.26 mmol) followed by titanium(IV) isopropoxide (2.082 mL, 7.11 mmol), and the mixture was stirred at 65° C. for 4 hours. The mixture was cooled to ambient temperature and sodium cyanoborohydride (0.298 g, 4.74 mmol) was added. The mixture was heated at 65° C. for 15 hours and then cooled to ambient temperature. The mixture was diluted with ethyl acetate and washed with saturated NaHCO₃. The bilayer was filtered through diatomaceous earth with ethyl acetate and the organic layer was washed with brine, dried over Na₂SO₄, filtered, and concentrated. The residue was purified by flash chromatography on silica gel eluting with 0-5% methanol/dichloromethane to afford the title compound (360 mg). MS (ESI+) m/z 381.1 (M+H)⁺.

### Step 8B

### 1-(5-bromo-2,3-dihydro-1H-inden-1-yl)piperazine

The title compound was prepared as described in Example 5 substituting the product of Step 8A for the product of Example 4. MS (APCI+) m/z 283.1 (M+H)⁺.

### Step 8C

### 2-[4-(5-bromo-2,3-dihydro-1H-inden-1-yl)piperazin-1-yl]ethan-1-ol

A solution of the product of Step 8B (0.75 g, 2.67 mmol) in methanol (12 mL) was treated with K₂CO₃ (0.922 g, 6.67 mmol) followed by 2-bromoethanol (0.34 mL, 4.80 mmol), and the mixture was stirred at ambient temperature for 24 hours. The mixture was diluted with dichloromethane and filtered through diatomaceous earth. The filtrate was washed with water and brine, dried over Na₂SO₄, concentrated and purified by flash chromatography on silica gel eluting with 0-10% methanol/dichloromethane to afford the title compound (508 mg). MS (ESI+) m/z 325.2 (M+H)⁺.

### Step 8D

### 2-amino-N-{(1S,2S)-2-[(4-{1-[4-(2-hydroxyethyl)piperazin-1-yl]-2,3-dihydro-H-inden-5-yl}phenyl)methoxy]cyclopentyl}-5-(1-methyl-1H-pyrazol-4-yl)pyridine-3-carboxamide

A mixture of Intermediate 33 (0.025 g, 0.048 mmol), the product of Step 8C (0.017 g, 0.053 mmol), bis(di-tert-butyl(4-dimethylaminophenyl)phosphine)dichloropalladium(II) (2.053 mg, 2.90 μmol) and K₂CO₃ (0.020 g, 0.145 mmol) in degassed water (0.15 mL) and degassed dioxane (0.5 mL) was heated at 80° C. for 90 minutes. The mixture was cooled to ambient temperature and directly purified by reverse phase HPLC eluting with 10-70% acetonitrile in water (0.1% trifluoroacetic acid) to afford the title compound (27 mg) as a trifluoroacetic acid salt. ¹H NMR (500 MHz, methanol-d₄) δ 8.51 (dd, J=2.6, 1.3 Hz, 1H), 8.20 (dd, J=2.2, 1.1 Hz, 1H), 7.98 (dd, J=5.3, 0.8 Hz, 1H), 7.88-7.83 (m, 1H), 7.58-7.52 (m, 2H), 7.52-7.39 (m, 5H), 4.74-4.62 (m, 3H), 4.46-4.38 (m, 1H), 4.06-3.99 (m, 1H), 3.91 (d, J=3.0 Hz, 3H), 3.87-3.82 (m, 2H), 3.46-3.34 (m, 3H), 3.24-2.90 (m, 8H), 2.42-2.26 (m, 2H), 2.24-2.14 (m, 1H), 2.11-2.01 (m, 1H), 1.89-1.75 (m, 3H), 1.69-1.59 (m, 1H). MS (ESI+) m/z 636.3 (M+H)⁺.

### Example 9

### 2-amino-N-{(1S,2S)-2-[(4-{2-[2-(dimethylamino)acetamido]-2,3-dihydro-1H-inden-5-yl}phenyl)methoxy]cyclopentyl}-5-(1-methyl-1H-pyrazol-4-yl)pyridine-3-carboxamide

### Step 9A

### 2-(dimethylamino)-N-(5-iodo-2,3-dihydro-1H-inden-2-yl)acetamide

A mixture of 2-(dimethylamino)acetic acid (0.048 g, 0.463 mmol) and triethylamine (0.19 mL, 1.35 mmol) in N,N-dimethylformamide (1.3 mL) was treated with 1-[bis(dimethylamino)methylene]-1H-1,2,3-tri azolo[4,5-b]pyridinium 3-oxid hexafluorophosphate (HATU, 0.169 g, 0.444 mmol), and the mixture was stirred for 5 minutes. A solution of 5-iodo-2,3-dihydro-1H-inden-2-amine (0.1 g, 0.386 mmol) in N,N-dimethylformamide (0.2 mL) was added, and the mixture was stirred at ambient temperature for 16 hours. The mixture was partitioned in ethyl acetate and water, and the organic layer was washed with saturated NaHCO₃ and brine, dried over Na₂SO₄, and concentrated. The residue was purified by flash chromatography on silica gel eluting with 0-10% methanol/dichloromethane to afford the title compound (94 mg). MS (ESI+) m/z 345.0 (M+H)⁺.

### Step 9B

### 2-amino-N-{(1S,2S)-2-[(4-{2-[2-(dimethylamino)acetamido]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 8D substituting the product of Step 9A for the product of Step 8C. ¹H NMR (400 MHz, methanol-d₄) δ 8.50 (t, J=2.0 Hz, 1H), 8.18 (d, J=2.1 Hz, 1H), 7.99-7.94 (m, 1H), 7.85 (dd, J=5.5, 0.8 Hz, 1H), 7.56-7.48 (m, 2H), 7.44-7.37 (m, 3H), 7.37-7.31 (m, 1H), 7.26 (d, J=7.9 Hz, 1H), 4.73-4.58 (m, 3H), 4.46-4.36 (m, 1H), 4.05-3.97 (m, 1H), 3.93-3.87 (m, 5H), 3.39-3.33 (m, 1H), 2.96-2.90 (m, 8H), 2.88 (d, J=5.2 Hz, 1H), 2.24-1.99 (m, 2H), 1.89-1.72 (m, 3H), 1.69-1.57 (m, 1H). MS (ESI+) m/z 608.3 (M+H)⁺.

### Example 10

### 2-amino-N-{(1S,2S)-2-[(4-{2-[(1-methylpiperidine-4-carbonyl)amino]-2,3-dihydro-1H-inden-5-yl}phenyl)methoxy]cyclopentyl}-5-(1-methyl-1H-pyrazol-4-yl)pyridine-3-carboxamide

### Step 10A

### N-(5-iodo-2,3-dihydro-1H-inden-2-yl)-1-methylpiperidine-4-carboxamide

The title compound was prepared as described in Step 9A substituting 1-methylpiperidine-4-carboxylic acid for 2-(dimethylamino)acetic acid. MS (ESI+) m/z 385.1 (M+H)⁺.

### Step 10B

### 2-amino-N-{(1S,2S)-2-[(4-{2-[(1-methylpiperidine-4-carbonyl)amino]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 8D substituting the product of Step 10A for the product of Step 8C. ¹H NMR (400 MHz, methanol-d₄) δ 8.31-8.17 (m, 1H), 7.96-7.90 (m, 1H), 7.80 (dd, J=6.1, 0.8 Hz, 1H), 7.73 (dd, J=7.6, 0.9 Hz, 1H), 7.56-7.44 (m, 2H), 7.43-7.27 (m, 4H), 7.22 (d, J=7.9 Hz, 1H), 4.72-4.51 (m, 4H), 4.46-4.34 (m, 1H), 4.04-3.92 (m, 1H), 3.84 (d, J=8.7 Hz, 3H), 3.27-3.20 (m, 1H), 2.96-2.75 (m, 4H), 2.25 (s, 3H), 2.21-1.92 (m, 5H), 1.89-1.69 (m, 7H), 1.66-1.51 (m, 1H). MS (ESI+) m/z 648.3 (M+H)⁺.

### Example 11

### 2-amino-N-{(1S,2S)-2-[(4-{2-[(1-methylazetidine-3-carbonyl)amino]-2,3-dihydro-1H-inden-5-yl}phenyl)methoxy]cyclopentyl}-5-(1-methyl-1H-pyrazol-4-yl)pyridine-3-carboxamide

### Step 11A

### N-(5-iodo-2,3-dihydro-1H-inden-2-yl)-1-methylazetidine-3-carboxamide

The title compound was prepared as described in Step 9A substituting 1-methylazetidine-3-carboxylic acid for 2-(dimethylamino)acetic acid. MS (ESI+) m/z 385.1 (M+H)⁺.

### Step 11B

### 2-amino-N-{(1S,2S)-2-[(4-{2-[(1-methylazetidine-3-carbonyl)amino]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 8D substituting the product of Step 11A for the product of Step 8C. ¹H NMR (400 MHz, methanol-d₄) δ 8.29-8.20 (m, 1H), 7.98-7.91 (m, 1H), 7.80 (d, J=8.5 Hz, 1H), 7.77-7.68 (m, 1H), 7.48 (d, J=8.2 Hz, 2H), 7.43-7.32 (m, 3H), 7.32-7.26 (m, 1H), 7.26-7.19 (m, 1H), 4.73-4.58 (m, 3H), 4.46-4.36 (m, 1H), 4.01-3.91 (m, 1H), 3.84 (d, J=12.3 Hz, 3H), 3.61-3.50 (m, 2H), 3.37-3.32 (m, 2H), 3.29-3.19 (m, 3H), 2.83 (dd, J=15.8, 5.5 Hz, 2H), 2.33 (s, 3H), 2.22-2.10 (m, 1H), 2.09-2.01 (m, 1H), 1.87-1.69 (m, 3H), 1.66-1.53 (m, 1H). MS (ESI+) m/z 620.3 (M+H)⁺.

### Example 12 2-amino-N-[(1S,2S)-2-({4-[3,3-dimethyl-1-(1-methylpiperidin-4-yl)-2,3-dihydro-1H-indol-5-yl]phenyl}methoxy)cyclopentyl]-5-(1-methyl-1H-pyrazol-4-yl)pyridine-3-carboxamide

### Step 12A

### tert-butyl 4-(5-bromo-3,3-dimethylindolin-1-yl)piperidine-1-carboxylate

A solution of 5-bromo-3,3-dimethylindoline (0.455 g, 2.012 mmol) in acetic acid (5.7 mL) was treated with tert-butyl 4-oxopiperidine-1-carboxylate (0.441 g, 2.213 mmol) followed by sodium triacetoxyhydroborate (0.682 g, 3.22 mmol) over 5 minutes. After stirring at ambient temperature for 2 hours, the mixture was poured into ethyl acetate and water, cooled in an ice bath and adjusted to pH 10 by dropwise addition of 6 N NaOH. The organic phase was washed with brine, dried over Na₂SO₄, and concentrated. The residue was purified by flash chromatography on silica gel eluting with 0-25% ethyl acetate/heptane to afford the title compound (670 mg). MS (ESI+) m/z 408.9 (M+H)⁺.

### Step 12B

### 5-bromo-3,3-dimethyl-1-(piperidin-4-yl)-2,3-dihydro-1H-indole

The title compound was prepared as described in Example 5 substituting the product of Step 12A for the product of Example 4. MS (ESI+) m/z 309.2 (M+H)⁺.

### Step 12C

### 5-bromo-3,3-dimethyl-1-(1-methylpiperidin-4-yl)-2,3-dihydro-1H-indole

The title compound was prepared as described in Step 6B substituting the product of Step 12B for the product of Step 6A. MS (ESI+) m/z 323.3 (M+H)⁺.

### Step 12D

### 2-amino-N-[(1S,2S)-2-({4-[3,3-dimethyl-1-(1-methylpiperidin-4-yl)-2,3-dihydro-1H-indol-5-yl]phenyl}methoxy)cyclopentyl]-5-(1-methyl-1H-pyrazol-4-yl)pyridine-3-carboxamide

A mixture of Intermediate 24 (0.030 g, 0.069 mmol), the product of Step 12C (0.023 g, 0.072 mmol), bis(di-tert-butyl(4-dimethylaminophenyl)phosphine)dichloropalladium(II) (2.93 mg, 4.14 μmol) and K₂CO₃ (0.029 g, 0.207 mmol) in degassed water (0.2 mL) and degassed dioxane (0.6 mL) was heated at 75° C. for 1 hour. The mixture was cooled to ambient temperature and partitioned in water and ethyl acetate. The aqueous layer was extracted with ethyl acetate and the organic layers were washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (15 mg). ¹H NMR (501 MHz, methanol-d₄) δ 8.25 (d, J=2.3 Hz, 1H), 7.96 (d, J=2.3 Hz, 1H), 7.79 (s, 1H), 7.74 (s, 1H), 7.47-7.40 (m, 2H), 7.37-7.30 (m, 2H), 7.24-7.10 (m, 2H), 6.48 (d, J=8.3 Hz, 1H), 4.67-4.62 (m, 2H), 4.46-4.36 (m, 1H), 4.01-3.92 (m, 1H), 3.83 (s, 3H), 3.53-3.40 (m, 1H), 3.18 (s, 2H), 3.03-2.92 (m, 2H), 2.31 (s, 3H), 2.25-2.10 (m, 3H), 2.09-1.97 (m, 1H), 1.84-1.68 (m, 7H), 1.65-1.53 (m, 1H), 1.28 (d, J=6.5 Hz, 6H). MS (APCI+) m/z 634.41 (M+H)⁺.

### Example 13

### 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

### Step 13A

### 2-[4-(5-bromo-3,3-dimethyl-2,3-dihydro-1H-indol-1-yl)piperidin-1-yl]ethan-1-ol

A solution of the product of Step 12B (0.77 g, 2.490 mmol) in methanol (15 mL) was treated with K₂CO₃ (1.032 g, 7.47 mmol) followed by 2-bromoethanol (0.529 mL, 7.47 mmol), and the mixture was stirred at ambient temperature for 18 hours. The mixture was diluted with ethyl acetate, filtered, and concentrated. The residue was purified by flash chromatography on silica gel eluting with 0-100% (89:10:1 dichloromethane/methanol/NH₄OH) in dichloromethane to afford the title compound (543 mg). MS (ESI+) m/z 353.2 (M+H)⁺.

### Step 13B

### 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

The title compound was prepared as described in Step 12D substituting the product of Step 13A for the product of Step 12C. ¹H NMR (501 MHz, methanol-d₄) δ 8.25 (d, J=2.3 Hz, 1H), 7.96 (d, J=2.3 Hz, 1H), 7.79 (s, 1H), 7.74 (s, 1H), 7.44 (d, J=8.1 Hz, 2H), 7.33 (d, J=8.1 Hz, 2H), 7.20 (dd, J=8.2, 1.9 Hz, 1H), 7.16 (d, J=1.9 Hz, 1H), 6.48 (d, J=8.2 Hz, 1H), 4.69-4.58 (m, 2H), 4.48-4.37 (m, 1H), 4.01-3.92 (m, 1H), 3.83 (s, 3H), 3.71 (t, J=6.1 Hz, 2H), 3.48 (p, J=8.7, 7.5 Hz, 1H), 3.19 (s, 2H), 3.11 (d, J=11.7 Hz, 2H), 2.59 (t, J=6.1 Hz, 2H), 2.32-2.09 (m, 3H), 2.08-1.97 (m, 1H), 1.88-1.68 (m, 7H), 1.66-1.53 (m, 1H), 1.28 (d, J=5.3 Hz, 6H). MS (ESI+) m/z 664.0 (M+H)⁺.

### Example 142-amino-N-{(3S,4R)-4-[(4-{1-[1-(2-hydroxyethyl)piperidin-4-yl]-1H-indol-5-yl}phenyl)methoxy]oxolan-3-yl}-5-(1-methyl-1H-pyrazol-4-yl)pyridine-3-carboxamide

A mixture of Intermediate 30 (0.037 g, 0.085 mmol), the product of Step 7A (0.029 g, 0.089 mmol), bis(di-tert-butyl(4-dimethylaminophenyl)phosphinedichloropalladium(II) (3.59 mg, 5.08 μmol) and K₂CO₃ (0.029 g, 0.212 mmol) in degassed water (0.2 mL) and of degassed dioxane (0.6 mL) was heated at 75° C. for 1 hour. The mixture was cooled to ambient temperature and partitioned in water and ethyl acetate. The aqueous layer was extracted with ethyl acetate and the organic layers were washed with brine, dried over Na₂SO₄, and concentrated. The residue was purified by flash chromatography on silica gel eluting with 0-10% methanol/dichloromethane to provide the title compound (16 mg). ¹H NMR (400 MHz, DMSO-d₆) δ 8.88 (d, J=6.8 Hz, 1H), 8.47-8.35 (m, 2H), 8.10 (s, 1H), 7.99-7.81 (m, 2H), 7.78-7.60 (m, 3H), 7.60-7.38 (m, 3H), 6.57 (d, J=3.2 Hz, 1H), 4.84-4.61 (m, 3H), 4.56-4.46 (m, 1H), 4.22-4.14 (m, 1H), 4.12-3.91 (m, 3H), 3.87 (s, 3H), 3.83-3.69 (m, 6H), 3.27-3.18 (m, 3H), 2.44-2.10 (m, 4H). MS (APCI+) m/z 636.32 (M+H)⁺.

### Example 15 2-amino-N-[(1S,2S)-2-({4-[3-(1-methylpiperidin-4-yl)-1H-indol-6-yl]phenyl}methoxy)cyclopentyl]-5-(1-methyl-1H-pyrazol-4-yl)pyridine-3-carboxamide

### Step 15A

### 6-chloro-3-(1-methylpiperidin-4-yl)-1H-indole

A solution of 6-chloro-3-(piperidin-4-yl)-1H-indole (0.10 g, 0.426 mmol) in methanol (2 mL) was treated with acetic acid (0.049 mL, 0.852 mmol) and formaldehyde (37 wt % in water, 0.063 mL, 0.852 mmol). After 10 minutes, sodium triacetoxyhydroborate (0.271 g, 1.278 mmol) was added, and the mixture was stirred at ambient temperature 5 hours. The mixture was concentrated, and the residue was dissolved in water and adjusted to pH 9 with 3 N NaOH. The suspension was extracted with ethyl acetate and the organic layer was washed with brine, dried over Na₂SO₄, filtered, and concentrated to afford the title compound (97 mg). MS (ESI+) m/z 249.1 (M+H)⁺.

### Step 15B

### 2-amino-N-[(1S,2S)-2-({4-[3-(1-methylpiperidin-4-yl)-1H-indol-6-yl]phenyl}methoxy)cyclopentyl]-5-(1-methyl-1H-pyrazol-4-yl)pyridine-3-carboxamide

A mixture of Intermediate 24 (0.034 g, 0.078 mmol), the product of Step 15A (0.019 g, 0.078 mmol), bis(di-tert-butyl(4-dimethylaminophenyl)phosphine)dichloropalladium(II) (3.32 mg, 4.69 μmol) and K₂CO₃ (0.027 g, 0.195 mmol) in degassed water (0.2 mL) and degassed dioxane (0.6 mL) was heated in a microwave reactor at 130° C. for 30 minutes. The mixture was cooled to ambient temperature and partitioned in water and ethyl acetate. The aqueous layer was extracted with ethyl acetate and the organic layers were washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (22 mg). ¹H NMR (501 MHz, methanol-d₄) δ 8.22 (d, J=2.3 Hz, 1H), 7.93 (d, J=2.2 Hz, 1H), 7.76-7.68 (m, 2H), 7.59 (d, J=8.3 Hz, 1H), 7.56-7.50 (m, 2H), 7.47 (d, J=1.7 Hz, 1H), 7.42-7.34 (m, 2H), 7.19 (dd, J=8.3, 1.6 Hz, 1H), 7.05 (d, J=0.7 Hz, 1H), 4.65 (q, J=12.4 Hz, 2H), 4.42 (td, J=7.7, 4.8 Hz, 1H), 3.97 (dt, J=6.7, 4.7 Hz, 1H), 3.73 (s, 3H), 3.05-2.96 (m, 2H), 2.90-2.79 (m, 1H), 2.25 (td, J=12.0, 2.5 Hz, 2H), 2.19-2.10 (m, 1H), 2.10-1.98 (m, 3H), 1.94-1.69 (m, 5H), 1.64-1.53 (m, 1H). MS (ESI+) m/z 604.1 (M+H)⁺.

### Example 16

### 2-amino-N-[(3S,4R)-4-{[4-(1-methyl-1H-indol-5-yl)phenyl]methoxy}oxolan-3-yl]-5-(1-methyl-1H-pyrazol-4-yl)pyridine-3-carboxamide

The title compound was prepared as described in Example 14 substituting 5-bromo-1-methyl-1H-indole for the product of Step 7A. ¹H NMR (501 MHz, DMSO-d₆) δ 8.58 (d, J=6.8 Hz, 1H), 8.34 (d, J=2.3 Hz, 1H), 8.09 (d, J=2.3 Hz, 1H), 8.01 (d, J=0.8 Hz, 1H), 7.84-7.76 (m, 2H), 7.70-7.62 (m, 2H), 7.55-7.48 (m, 1H), 7.47-7.40 (m, 3H), 7.35 (d, J=3.0 Hz, 1H), 7.01 (s, 2H), 6.47 (dd, J=3.1, 0.8 Hz, 1H), 4.75 (d, J=12.0 Hz, 1H), 4.64 (d, J=12.0 Hz, 1H), 4.56-4.46 (m, 1H), 4.18-4.12 (m, 1H), 4.06 (dd, J=9.4, 6.2 Hz, 1H), 3.98 (dd, J=10.0, 4.8 Hz, 1H), 3.85 (s, 3H), 3.81 (s, 3H), 3.79-3.71 (m, 2H). MS (ESI+) m/z 523.2 (M+H)⁺.

### Example 17

### 2-amino-5-(1-methyl-1H-pyrazol-4-yl)-N-[(1S,2S)-2-{[4-(1,3,3-trimethyl-2,3-dihydro-1H-indol-5-yl)phenyl]methoxy}cyclopentyl]pyridine-3-carboxamide

### Step 17A

### 5-bromo-1,3,3-trimethyl-2,3-dihydro-1H-indole

A suspension of 5-bromo-3,3-dimethylindoline (0.13 g, 0.575 mmol) in N,N-dimethylformamide (3.4 mL) at 0° C. was treated with 60% sodium hydride (0.057 g, 1.437 mmol), and the mixture was stirred at ambient temperature for 2 hours and then cooled to 0° C. Iodomethane (0.144 mL, 2.3 mmol) was added, and the mixture was stirred at ambient temperature 16 hours. The mixture was quenched with water and extracted with ethyl acetate. The organic layer was washed with brine, dried over Na₂SO₄, and concentrated. The residue was purified by flash chromatography on silica gel eluting with 0-15% ethyl acetate/heptane to afford the title compound (25 mg). MS (ESI+) m/z 240.2 (M+H)⁺.

### Step 17B

### 2-amino-5-(1-methyl-1H-pyrazol-4-yl)-N-[(1S,2S)-2-{[4-(1,3,3-trimethyl-2,3-dihydro-1H-indol-5-yl)phenyl]methoxy}cyclopentyl]pyridine-3-carboxamide

The title compound was prepared as described in Step 12D substituting the product of Step 17A for the product of Step 12C. ¹H NMR (501 MHz, DMSO-d₆) δ 8.41-8.28 (m, 2H), 8.06-7.95 (m, 2H), 7.78 (d, J=0.8 Hz, 1H), 7.57-7.45 (m, 2H), 7.36-7.23 (m, 4H), 6.96 (s, 2H), 6.54 (d, J=8.0 Hz, 1H), 4.63-4.51 (m, 2H), 4.36-4.25 (m, 1H), 3.95-3.89 (m, 1H), 3.83 (s, 3H), 3.07 (s, 2H), 2.73 (s, 3H), 2.12-2.00 (m, 1H), 2.00-1.87 (m, 1H), 1.80-1.62 (m, 3H), 1.63-1.50 (m, 1H), 1.27 (d, J=2.0 Hz, 6H). MS (ESI+) m/z 551.2 (M+H)⁺.

### Example 18

### 2-amino-N-{(3S,4R)-4-[(4-{1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide

The title compound was prepared as described in Example 14 substituting the product of Step 8C for the product of Step 7A. ¹H NMR (501 MHz, DMSO-d₆) δ 8.25 (d, J=2.3 Hz, 1H), 8.04 (d, J=2.3 Hz, 1H), 7.90-7.83 (m, 1H), 7.76 (d, J=0.9 Hz, 1H), 7.60-7.52 (m, 2H), 7.49-7.36 (m, 5H), 4.79-4.68 (m, 2H), 4.63-4.56 (m, 1H), 4.33 (dd, J=7.7, 5.3 Hz, 1H), 4.23-4.17 (m, 1H), 4.13 (dd, J=9.5, 5.9 Hz, 1H), 4.07 (dd, J=10.1, 5.2 Hz, 1H), 3.89 (s, 3H), 3.86-3.77 (m, 2H), 3.67 (t, J=6.0 Hz, 2H), 3.00 (dt, J=15.2, 7.2 Hz, 1H), 2.93-2.80 (m, 1H), 2.75-2.47 (m, 10H), 2.26-2.08 (m, 2H). MS (ESI+) m/z 638.2.2 (M+H)⁺.

### Example 19

### 6-amino-6′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide

A mixture of Intermediate 32 (0.027 g, 0.051 mmol), the product of Step 8C (0.017 g, 0.053 mmol), bis(di-tert-butyl(4-dimethylaminophenyl)phosphine)dichloropalladium(II) (2.154 mg, 3.04 μmol) and K₂CO₃ (0.018 g, 0.127 mmol) in degassed water (0.2 mL) and degassed dioxane (0.6 mL) was heated at 75° C. for 1 hour. The mixture was cooled to ambient temperature and partitioned in water and ethyl acetate. The organic layer was washed with brine, dried over Na₂SO₄, and concentrated. The residue was purified by flash chromatography on silica gel eluting with 0-10% methanol/dichloromethane followed by a gradient of from 0-100% (89:10:1 dichloromethane/methanol/NH₄OH) in dichloromethane to afford the title compound (21 mg). ¹H NMR (501 MHz, DMSO-d₆) δ 8.42-8.26 (m, 2H), 8.11-8.02 (m, 1H), 7.98 (t, J=2.3 Hz, 1H), 7.47 (d, J=7.9 Hz, 2H), 7.42-7.35 (m, 3H), 7.35-7.24 (m, 2H), 7.08-6.97 (m, 1H), 4.73-4.57 (m, 2H), 4.46-4.37 (m, 1H), 4.33 (dd, J=7.6, 5.4 Hz, 1H), 4.01-3.91 (m, 1H), 3.67 (t, J=6.0 Hz, 2H), 3.05-2.92 (m, 1H), 2.91-2.77 (m, 1H), 2.77-2.41 (m, 10H), 2.25-2.01 (m, 4H), 1.88-1.69 (m, 3H), 1.65-1.50 (m, 1H). MS (ESI+) m/z 651.4 (M+H)⁺.

### Example 20

### 6-amino-6′-fluoro-N-{(3S,4R)-4-[(4-{1-[4-(2-hydroxyethyl)piperazin-1-yl]-2,3-dihydro-1H-inden-5-yl}phenyl)methoxy]oxolan-3-yl}[3,3′-bipyridine]-5-carboxamide

The title compound was prepared as described in Example 19 substituting Intermediate 37 (0.035 g, 0.065 mmol) for Intermediate 32. ¹H NMR (400 MHz, methanol-d₄) δ 8.45-8.37 (m, 1H), 8.33 (d, J=2.3 Hz, 1H), 8.18-8.08 (m, 2H), 7.60-7.51 (m, 2H), 7.49-7.35 (m, 5H), 7.09 (dd, J=8.5, 2.7 Hz, 1H), 4.80-4.69 (m, 2H), 4.64-4.57 (m, 1H), 4.34 (dd, J=7.6, 5.3 Hz, 1H), 4.22-4.17 (m, 1H), 4.13 (dd, J=9.5, 6.0 Hz, 1H), 4.07 (dd, J=10.0, 5.2 Hz, 1H), 3.86-3.76 (m, 2H), 3.67 (t, J=6.0 Hz, 2H), 3.07-2.94 (m, 1H), 2.92-2.81 (m, 1H), 2.75-2.48 (m, 10H), 2.26-2.10 (m, 2H). MS (ESI+) m/z 653.4 (M+H)⁺.

### Example 21

### 2-amino-N-{(1S,2S)-2-[(4-{1-[1-(2-hydroxyethyl)piperidin-4-yl]-1H-indol-5-yl}phenyl)methoxy]cyclopentyl}-5-(trifluoromethyl)pyridine-3-carboxamide

The title compound was prepared as described in Step 4C substituting Intermediate 25 for Intermediate 33 and substituting the product of Step 7A for the product of Step 4B. ¹H NMR (500 MHz, DMSO-d₆) δ 8.58 (d, J=7.7 Hz, 1H), 8.41 (dd, J=2.4, 1.1 Hz, 1H), 8.22 (d, J=2.4 Hz, 1H), 7.84-7.73 (m, 3H), 7.66-7.57 (m, 3H), 7.53 (d, J=3.2 Hz, 1H), 7.44-7.32 (m, 3H), 6.51 (d, J=3.3 Hz, 1H), 4.66-4.54 (m, 2H), 4.44 (t, J=5.3 Hz, 1H), 4.41-4.25 (m, 2H), 4.11 (q, J=5.3 Hz, 1H), 3.92 (dt, J=6.3, 3.8 Hz, 1H), 3.60-3.48 (m, 2H), 3.18 (d, J=4.7 Hz, 2H), 3.03 (dt, J=12.2, 3.2 Hz, 2H), 2.25 (td, J=11.8, 2.5 Hz, 2H), 2.08-1.90 (m, 5H), 1.80-1.52 (m, 4H). MS (ESI+) m/z 622 (M+H)⁺.

### Example 22

### 6-amino-6′-fluoro-N-{(1S,2S)-2-[(4-{1-[1-(2-hydroxyethyl)piperidin-4-yl]-1H-indol-5-yl}phenyl)methoxy]cyclopentyl}[3,3′-bipyridine]-5-carboxamide

The title compound was prepared as described in Step 4C substituting Intermediate 32 for Intermediate 33 and substituting the product of Step 7A for the product of Step 4B. ¹H NMR (500 MHz, DMSO-d₆) δ 8.57 (d, J=2.7 Hz, 1H), 8.48 (d, J=2.4 Hz, 1H), 8.43 (d, J=7.8 Hz, 1H), 8.27 (td, J=8.2, 2.7 Hz, 1H), 8.19 (d, J=2.4 Hz, 1H), 7.75 (d, J=1.8 Hz, 1H), 7.61-7.55 (m, 3H), 7.52 (d, J=3.2 Hz, 1H), 7.41-7.34 (m, 3H), 7.29 (s, 2H), 7.22 (dd, J=8.6, 2.8 Hz, 1H), 6.50 (d, J=3.2 Hz, 1H), 4.60 (d, J=1.9 Hz, 2H), 4.46 (d, J=5.4 Hz, 1H), 4.35 (ddt, J=10.3, 7.7, 5.0 Hz, 2H), 3.93 (dt, J=6.3, 4.0 Hz, 1H), 3.55 (td, J=6.3, 3.1 Hz, 2H), 3.04 (dt, J=12.7, 3.4 Hz, 2H), 2.48 (t, J=6.3 Hz, 2H), 2.26 (td, J=11.8, 2.5 Hz, 2H), 2.14-1.86 (m, 6H), 1.83-1.50 (m, 4H). MS (ESI+) m/z 649 (M+H)⁺.

### Example 23

### 2-amino-N-{(1S,2S)-2-[(4-{1-[1-(2,3-dihydroxypropyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

### Step 23A

### 5-bromo-1-{1-[(2,2-dimethyl-1,3-dioxolan-4-yl)methyl]piperidin-4-yl}-3,3-dimethyl-2,3-dihydro-1H-indole

A solution of the product of Step 12B (0.075 g, 0.243 mmol) and (4R)-2,2-dimethyl-1,3-dioxolane-4-carbaldehyde (0.032 g, 0.243 mmol) in dichloromethane (2.5 mL) was stirred at ambient temperature 1 hour. Sodium triacetoxyhydroborate (0.129 g, 0.606 mmol) was added in several portions, and the mixture was stirred at ambient temperature for 2 days. The mixture was diluted with dichloromethane, washed with saturated Na₂CO₃, dried over Na₂SO₄, and concentrated. The residue was purified by flash chromatography on silica gel eluting with 0-4% methanol/dichloromethane to afford the title compound (66 mg). MS (ESI+) m/z 425.2 (M+H)⁺.

### Step 23B

### 2-amino-N-[(1S,2S)-2-{[4-(1-{1-[(2,2-dimethyl-1,3-dioxolan-4-yl)methyl]piperidin-4-yl}-3,3-dimethyl-2,3-dihydro-1H-indol-5-yl)phenyl]methoxy}cyclopentyl]-5-(1-methyl-1H-pyrazol-4-yl)pyridine-3-carboxamide

The title compound was prepared as described in Step 12D substituting the product of Step 23A for the product of Step 12C. MS (APCI+) m/z 734.48 (M+H)⁺.

### Step 23C

### 2-amino-N-{(1S,2S)-2-[(4-{1-[1-(2,3-dihydroxypropyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

A solution of the product of Step 23B (0.034 g, 0.046 mmol) and trifluoroacetic acid (0.071 mL, 0.927 mmol) in dichloromethane (0.4 mL) was stirred at ambient temperature for 16 hours. The mixture was partitioned in saturated NaHCO₃ and ethyl acetate and the organic layer was washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (13 mg). ¹H NMR (501 MHz, methanol-d₄) δ 8.25 (d, J=2.3 Hz, 1H), 7.95 (d, J=2.3 Hz, 1H), 7.82-7.72 (m, 2H), 7.47-7.41 (m, 2H), 7.36-7.31 (m, 2H), 7.20 (dd, J=8.1, 1.9 Hz, 1H), 7.16 (d, J=1.9 Hz, 1H), 6.48 (d, J=8.2 Hz, 1H), 4.67-4.59 (m, 2H), 4.41 (td, J=7.8, 4.8 Hz, 1H), 3.96 (dt, J=6.7, 4.7 Hz, 1H), 3.86-3.78 (m, 4H), 3.57-3.41 (m, 3H), 3.19 (s, 2H), 3.17-3.05 (m, 2H), 2.56-2.43 (m, 2H), 2.33-2.11 (m, 3H), 2.08-1.99 (m, 1H), 1.85-1.70 (m, 7H), 1.64-1.54 (m, 1H), 1.29 (s, 3H), 1.28 (s, 3H). MS (ESI+) m/z 694.3 (M+H)⁺.

### Example 242-amino-N-{(3S,4R)-4-[(4-{1-[1-(2-hydroxyethyl)piperidin-4-yl]-1H-indol-5-yl}phenyl)methoxy]oxolan-3-yl}-5-(trifluoromethyl)pyridine-3-carboxamide

The title compound was prepared as described in Step 4C substituting Intermediate 26 for Intermediate 33. and substituting the product of Step 7A for the product of Step 4B. ¹H NMR (400 MHz, DMSO-d₆) δ 8.79 (d, J=6.7 Hz, 1H), 8.37 (dd, J=2.4, 1.0 Hz, 1H), 8.25 (d, J=2.5 Hz, 1H), 7.84-7.69 (m, 3H), 7.65-7.52 (m, 3H), 7.49 (d, J=3.2 Hz, 1H), 7.42-7.31 (m, 3H), 6.47 (d, J=3.2 Hz, 1H), 4.73-4.52 (m, 2H), 4.45 (td, J=6.6, 3.8 Hz, 1H), 4.40-4.24 (m, 2H), 4.10 (dt, J=4.9, 1.8 Hz, 1H), 3.97 (ddd, J=25.6, 9.7, 5.4 Hz, 2H), 3.81-3.62 (m, 2H), 3.51 (q, J=6.0 Hz, 2H), 3.09-2.90 (m, 2H), 2.43 (d, J=6.4 Hz, 2H), 2.21 (td, J=11.6, 2.6 Hz, 2H), 2.05-1.80 (m, 4H). MS (ESI+) m/z 624 (M+H)⁺.

### Example 25

### 6-amino-6′-fluoro-N-{(3S,4R)-4-[(4-{1-[1-(2-hydroxyethyl)piperidin-4-yl]-1H-indol-5-yl}phenyl)methoxy]oxolan-3-yl}[3,3′-bipyridine]-5-carboxamide

The title compound was prepared as described in Step 4C, substituting Intermediate 37 for Intermediate 33 and substituting the product of Step 7A for the product of 4B. ¹H NMR (400 MHz, DMSO-d₆) δ 8.64 (d, J=6.9 Hz, 1H), 8.54 (d, J=2.6 Hz, 1H), 8.45 (d, J=2.3 Hz, 1H), 8.25 (tq, J=4.4, 2.6 Hz, 2H), 7.76 (d, J=1.8 Hz, 1H), 7.58 (dd, J=16.5, 8.4 Hz, 3H), 7.48 (d, J=3.3 Hz, 1H), 7.43-7.34 (m, 3H), 7.27 (s, 2H), 7.22 (dd, J=8.6, 2.9 Hz, 1H), 6.47 (d, J=3.2 Hz, 1H), 4.78-4.55 (m, 2H), 4.54-4.28 (m, 4H), 4.11 (dt, J=4.3, 1.9 Hz, 1H), 3.99 (ddd, J=29.9, 9.7, 5.4 Hz, 2H), 3.73 (ddd, J=9.5, 7.5, 2.7 Hz, 2H), 3.52 (q, J=5.6 Hz, 3H), 3.02 (d, J=11.3 Hz, 2H), 2.25 (d, J=11.8 Hz, 2H), 2.12-1.78 (m, 4H). MS (ESI+) m/z 651 (M+H)⁺.

### Example 26

### 2-amino-N-{(1S,2S)-2-[(4-{3-[1-(2-hydroxyethyl)piperidin-4-yl]-1-methyl-1H-indol-6-yl}phenyl)methoxy]cyclopentyl}-5-(1-methyl-1H-pyrazol-4-yl)pyridine-3-carboxamide

### Step 26A

### tert-butyl 4-(6-chloro-1H-indol-3-yl)piperidine-1-carboxylate

A solution of 6-chloro-3-(piperidin-4-yl)-1H-indole (0.15 g, 0.639 mmol) and di-tert-butyl dicarbonate (0.139 g, 0.639 mmol) in tetrahydrofuran (2.5 mL) was stirred at ambient temperature 16 hours. The mixture was concentrated, and the residue was purified by flash chromatography on silica gel eluting with 0-30% ethyl acetate/heptane to afford the title compound (182 mg). MS (ESI+) m/z 333.0 (M−H)⁺.

### Step 26B

### tert-butyl 4-(6-chloro-1-methyl-1H-indol-3-yl)piperidine-1-carboxylate

A suspension of the product of Step 26A (0.179 g, 0.535 mmol) N,N-dimethylformrnamide (3.3 mL) at 0° C. was treated with 60% sodium hydride (0.053 g, 1.336 mmol) in one portion. The mixture was stirred at ambient temperature for 1 hour and then cooled to 0° C. Iodomethane (0.134 mL, 2.138 mmol) was added, and the mixture was stirred at ambient temperature 16 hours. The mixture was quenched with water and extracted with ethyl acetate. The organic layer was washed with brine, dried over Na₂SO₄, and concentrated to afford the title compound (210 mg). MS (ESI+) m/z 349.2 (M+H)⁺.

### Step 26C

### 6-chloro-1-methyl-3-(piperidin-4-yl)-1H-indole

The title compound was prepared as described in Example 5 substituting the product of Step 26B for the product of Example 4. MS (ESI+) m/z 249.2 (M+H)⁺.

### Step 26D

### 2-[4-(6-chloro-1-methyl-1H-indol-3-yl)piperidin-1-yl]ethan-1-ol

The title compound was prepared as described in Step 13A substituting the product of Step 26C for the product of Step 12B. MS (ESI+) m/z 293.3 (M+H)⁺.

### Step 26E

### 2-amino-N-{(1S,2S)-2-[(4-{3-[1-(2-hydroxyethyl)piperidin-4-yl]-1-methyl-1H-indol-6-yl}phenyl)methoxy]cyclopentyl}-5-(1-methyl-1H-pyrazol-4-yl)pyridine-3-carboxamide

A mixture of Intermediate 24 (0.029 g, 0.067 mmol), the product of Step 26D (0.018 g, 0.060 mmol), bis(di-tert-butyl(4-dimethylaminophenyl)phosphine)dichloropalladium(II) (2.83 mg, 4.00 μmol) and K₂CO₃ (0.023 g, 0.167 mmol) in degassed water (0.2 mL) and degassed dioxane (0.6 mL) was heated in a microwave reactor at 110° C. for 30 minutes. The mixture was cooled to ambient temperature and partitioned in water and ethyl acetate. The aqueous layer was extracted with additional ethyl acetate and the combined organic layers were washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (22 mg). ¹H NMR (501 MHz, methanol-d₄) δ 8.21 (d, J=2.2 Hz, 1H), 7.92 (d, J=2.3 Hz, 1H), 7.72-7.68 (m, 2H), 7.61-7.56 (m, 3H), 7.44-7.35 (m, 3H), 7.20 (dd, J=8.3, 1.5 Hz, 1H), 6.96 (s, 1H), 4.70-4.59 (m, 2H), 4.42 (td, J=7.7, 4.8 Hz, 1H), 4.00-3.93 (m, 1H), 3.77-3.70 (m, 8H), 3.14-3.06 (m, 2H), 2.85 (tt, J=11.9, 3.8 Hz, 1H), 2.62 (t, J=6.2 Hz, 2H), 2.30 (td, J=12.0, 2.5 Hz, 2H), 2.19-2.10 (m, 1H), 2.08-1.99 (m, 3H), 1.91-1.69 (m, 5H), 1.62-1.53 (m, 1H). MS (ESI+) m/z 648.8 (M+H)⁺.

### Example 27

### 2-amino-N-{(1S,2S)-2-[(4-{1-[1-(2-amino-2-oxoethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

### Step 27A

### 2-(4-(5-bromo-3,3-dimethylindolin-1-yl)piperidin-1-yl)acetamide

The product of Step 12B (0.103 g, 0.270 mmol) and triethylamine (0.188 mL, 1.348 mmol) in N,N-dimethylformamide (1.684 mL) was treated with 2-bromoacetamide (0.043 g, 0.310 mmol), and the mixture was heated at 75° C. for 3 hours. The mixture was cooled to ambient temperature and partitioned in water and ethyl acetate. The organic layer was washed with brine, dried over Na₂SO₄, and concentrated to afford the title compound (86 mg). MS (ESI+) m/z 366.2 (M+H)⁺.

### Step 27B

### 2-amino-N-{(1S,2S)-2-[(4-{1-[1-(2-amino-2-oxoethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

A mixture of Intermediate 24 (0.035 g, 0.080 mmol), the product of Step 27A (0.028 g, 0.076 mmol), bis(di-tert-butyl(4-dimethylaminophenyl)phosphine)dichloropalladium(II) (3.42 mg, 4.82 μmol) and K₂CO₃ (0.028 g, 0.201 mmol) in degassed water (0.2 mL) and degassed dioxane (0.6 mL) was heated at 70° C. for 1 hour. The mixture was cooled to ambient temperature and partitioned in water and ethyl acetate. The aqueous layer was extracted with additional ethyl acetate and the combined organic layers were washed with brine, dried over Na₂SO₄, and concentrated. The residue was purified by reverse phase HPLC on a Phenomenex® Luna® C₈ (2) column eluting with 10-70% acetonitrile in 0.1% trifluoroacetic acid in water to afford the title compound (38 mg) as a trifluoroacetic acid salt. ¹H NMR (400 MHz, methanol-d₄) δ 8.48 (d, J=2.1 Hz, 1H), 8.19 (d, J=2.1 Hz, 1H), 7.95 (s, 1H), 7.85 (s, 1H), 7.50-7.43 (m, 2H), 7.40-7.32 (m, 2H), 7.28-7.20 (m, 2H), 6.58 (d, J=8.2 Hz, 1H), 4.70-4.58 (m, 2H), 4.46-4.36 (m, 1H), 4.06-3.95 (m, 3H), 3.92-3.79 (m, 4H), 3.79-3.64 (m, 2H), 3.29-3.16 (m, 4H), 2.24-2.12 (m, 1H), 2.11-1.97 (m, 5H), 1.90-1.72 (m, 3H), 1.69-1.57 (m, 1H), 1.31 (d, J=1.3 Hz, 6H). MS (ESI+) m/z 677.3 (M+H)⁺.

### Example 28

### 2-amino-N-{(1S,2S)-2-[(4-{1′-[1-(2-hydroxyethyl)piperidin-4-yl]-1′,2′-dihydrospiro[cyclopropane-1,3′-indol]-5′-yl}phenyl)methoxy]cyclopentyl}-5-(1-methyl-1H-pyrazol-4-yl)pyridine-3-carboxamide

### Step 28A

### tert-butyl 4-(5′-bromospiro[cyclopropane-1,3′-indol]-1′(2′H)-yl)piperidine-1-carboxylate

The title compound was prepared as described in Step 12A substituting 5′-bromospiro[cyclopropane-1,3′-indoline] for 5-bromo-3,3-dimethylindoline. MS (ESI+) m/z 407.0 (M+H)⁺.

### Step 28B

### 5′-bromo-1′-(piperidin-4-yl)-1′,2′-dihydrospiro[cyclopropane-1,3′-indole]

The title compound was prepared as described in Example 5 substituting the product of Step 28A for the product of Example 4. MS (ESI+) m/z 307.1 (M+H)⁺.

### Step 28C

### 2-[4-(5′-bromospiro[cyclopropane-1,3′-indol]-1′(2′H)-yl)piperidin-1-yl]ethan-1-ol

The title compound was prepared as described in Step 13A substituting the product of Step 28B for the product of Step 12B. MS (ESI+) m/z 351.2 (M+H)⁺.

### Step 28D

### 2-amino-N-{(1S,2S)-2-[(4-{1′-[1-(2-hydroxyethyl)piperidin-4-yl]-1′,2′-dihydrospiro[cyclopropane-1,3′-indol]-5′-yl}phenyl)methoxy]cyclopentyl}-5-(1-methyl-1H-pyrazol-4-yl)pyridine-3-carboxamide

The title compound was prepared as described in Step 12D substituting the product of Step 28C for the product of Step 12C. ¹H NMR (400 MHz, methanol-d₄) δ 8.24 (d, J=2.3 Hz, 1H), 7.93 (d, J=2.3 Hz, 1H), 7.76 (s, 1H), 7.73 (s, 1H), 7.39 (d, J=8.3 Hz, 2H), 7.31 (d, J=8.3 Hz, 2H), 7.16 (dd, J=8.1, 1.9 Hz, 1H), 6.71 (d, J=1.9 Hz, 1H), 6.47 (d, J=8.2 Hz, 1H), 4.61 (q, J=12.3 Hz, 2H), 4.40 (td, J=7.6, 4.8 Hz, 1H), 3.94 (dt, J=6.7, 4.7 Hz, 1H), 3.83 (s, 3H), 3.70 (t, J=6.1 Hz, 2H), 3.56-3.48 (m, 1H), 3.46 (s, 2H), 3.13-3.06 (m, 2H), 2.57 (t, J=6.1 Hz, 2H), 2.23 (td, J=11.6, 3.5 Hz, 2H), 2.18-2.09 (m, 1H), 2.07-1.98 (m, 1H), 1.83-1.71 (m, 7H), 1.63-1.53 (m, 1H), 1.00-0.92 (m, 4H). MS (APCI+) m/z 662.37 (M+H)⁺.

### Example 29

### 2-amino-N-[(1S,2S)-2-{[4-(1H-indol-7-yl)phenyl]methoxy}cyclopentyl]-5-(1-methyl-1H-pyrazol-4-yl)pyridine-3-carboxamide

The title compound was prepared as described in Step 4C substituting 7-bromo-1H-indole for the product of Step 4B. ¹H NMR (400 MHz, DMSO-d₆) δ 10.90 (s, 1H), 8.34 (d, J=7.6 Hz, 1H), 8.29 (t, J=2.3 Hz, 1H), 8.05-7.87 (m, 2H), 7.76 (d, J=0.9 Hz, 1H), 7.60-7.40 (m, 5H), 7.28-7.18 (m, 1H), 7.10-7.01 (m, 2H), 6.94 (s, 2H), 6.47 (dd, J=3.1, 1.8 Hz, 1H), 4.70-4.56 (m, 2H), 4.31 (dt, J=11.5, 7.7 Hz, 1H), 3.95 (dt, J=6.0, 3.5 Hz, 1H), 3.80 (s, 3H), 2.09-1.91 (m, 2H), 1.80-1.49 (m, 4H). MS (ESI+) m/z 507 (M+H)⁺.

### Example 30

### 6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-2′-oxo-1′,2′-dihydro[3,4′-bipyridine]-5-carboxamide

### Step 30A

### 6-amino-1′-methyl-2′-oxo-1′,2′-dihydro[3,4′-bipyridine]-5-carboxylic acid

The title compound was prepared as described in Intermediate 2 substituting (1-methyl-2-oxo-1,2-dihydropyridin-4-yl)boronic acid for 1-methyl-4-(4,4,5,5-tetramethyl-1,3,2-dioxaborolan-2-yl)-1H-pyrazole. MS (APCI) m/z 246 (M+H)⁺.

### Step 30B

### 6-amino-N-{(1S,2S)-2-[(4-bromophenyl)methoxy]cyclopentyl}-1′-methyl-2′-oxo-1′,2′-dihydro[3,4′-bipyridine]-5-carboxamide

The title compound was prepared as described in Intermediate 3 substituting the product of Step 30A for Intermediate 2. MS (APCI) m/z 499 (M+H)⁺.

### Step 30C

### 6-amino-1′-methyl-2′-oxo-N-[(1S,2S)-2-{[4-(4,4,5,5-tetramethyl-1,3,2-dioxaborolan-2-yl)phenyl]methoxy}cyclopentyl]-1′,2′-dihydro[3,4′-bipyridine]-5-carboxamide

The title compound was prepared as described in Intermediate 19B substituting the product of Step 30B for Intermediate 19A. MS (ESI+) m/z 545 (M+H)⁺.

### Step 30D

### 6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-2′-oxo-1′,2′-dihydro[3,4′-bipyridine]-5-carboxamide

The title compound was prepared as described in Step 12D substituting the product of Step 30C for Intermediate 24 and substituting Intermediate 13 for the product of Step 12C. ¹H NMR (400 MHz, DMSO-d₆) δ 8.54-8.40 (m, 2H), 8.18 (d, J=2.4 Hz, 1H), 7.66 (d, J=7.2 Hz, 1H), 7.43 (dd, J=7.3, 5.3 Hz, 4H), 7.27 (d, J=8.1 Hz, 2H), 7.24-7.14 (m, 2H), 6.78 (d, J=2.1 Hz, 1H), 6.60 (dd, J=7.2, 2.1 Hz, 1H), 6.49-6.41 (m, 1H), 4.64-4.44 (m, 2H), 4.38-4.21 (m, 2H), 3.89 (dt, J=6.3, 4.0 Hz, 1H), 3.45 (q, J=6.0 Hz, 2H), 3.39 (s, 3H), 3.10 (s, 2H), 2.91 (d, J=11.0 Hz, 2H), 2.37 (t, J=6.3 Hz, 2H), 2.13-1.83 (m, 4H), 1.79-1.48 (m, 7H), 1.22 (d, J=2.5 Hz, 6H). MS (ESI+) m/z 691 (M+H)⁺.

### Example 31

### 6-amino-6′-fluoro-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}[3,3′-bipyridine]-5-carboxamide

The title compound was prepared as described in Example 19 substituting the product of Step 13A for the product of Step 8C. ¹H NMR (501 MHz, methanol-d₄) δ 8.35 (d, J=2.7 Hz, 1H), 8.31 (d, J=2.4 Hz, 1H), 8.03 (ddd, J=8.5, 7.6, 2.7 Hz, 1H), 7.97 (d, J=2.3 Hz, 1H), 7.43-7.38 (m, 2H), 7.35-7.30 (m, 2H), 7.18-7.12 (m, 2H), 6.98 (dd, J=8.5, 2.6 Hz, 1H), 6.46 (d, J=8.1 Hz, 1H), 4.68-4.57 (m, 2H), 4.41 (td, J=7.7, 5.0 Hz, 1H), 3.98-3.92 (m, 1H), 3.71 (t, J=6.1 Hz, 2H), 3.52-3.43 (m, 1H), 3.19 (s, 2H), 3.14-3.06 (m, 2H), 2.58 (t, J=6.1 Hz, 2H), 2.29-2.20 (m, 2H), 2.19-2.10 (m, 1H), 2.08-1.99 (m, 1H), 1.84-1.72 (m, 7H), 1.61-1.52 (m, 1H), 1.28 (d, J=8.5 Hz, 6H). MS (ESI+) m/z 679.2 (M+H)⁺.

### Example 32

### 2-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-5-(1-methyl-1H-pyrazol-4-yl)pyridine-3-carboxamide

The title compound was prepared as described in Example 14 substituting the product of Step 13A for the product of Step 7A. ¹H NMR (501 MHz, methanol-d₄) δ 8.28-8.24 (m, 1H), 8.04 (d, J=2.3 Hz, 1H), 7.85 (s, 1H), 7.77 (s, 1H), 7.48 (d, J=8.3 Hz, 2H), 7.37 (d, J=8.1 Hz, 2H), 7.25 (dd, J=8.1, 2.0 Hz, 1H), 7.20 (d, J=1.9 Hz, 1H), 6.49 (d, J=8.3 Hz, 1H), 4.79-4.63 (m, 3H), 4.22-4.16 (m, 1H), 4.16-4.02 (m, 2H), 3.88 (s, 3H), 3.84-3.76 (m, 2H), 3.70 (t, J=6.1 Hz, 2H), 3.53-3.41 (m, 1H), 3.19 (s, 2H), 3.13-3.05 (m, 2H), 2.57 (t, J=6.1 Hz, 2H), 2.29-2.16 (m, 2H), 1.83-1.73 (m, 4H), 1.29 (d, J=2.3 Hz, 6H). MS (ESI+) m/z 666.3 (M+H)⁺.

### Example 33

### 2-amino-N-{(1S,2S)-2-[(4-{(1R)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 33A

### 2-{4-[(1R)-5-bromo-2,3-dihydro-1H-inden-1-yl]piperazin-1-yl}ethan-1-ol

Chiral preparative super critical fluid chromatography was performed on racemic 1-(5-bromo-2,3-dihydro-1H-inden-1-yl) piperazine (Step 8C, 500 mg) using a ChiralPak AD-H column. The preparative super critical fluid chromatography afforded the title compound (205 mg) as the first eluting compound. ¹H NMR (400 MHz, methanol-d₄) δ 7.38 (d, J=1.8 Hz, 1H), 7.36-7.30 (m, 1H), 7.27 (d, J=8.1 Hz, 1H), 4.56 (brs, 1H), 4.27 (dd, J=7.8, 5.4 Hz, 1H), 3.66 (t, J=6.1 Hz, 2H), 3.04-2.89 (m, 1H), 2.89-2.77 (m, 1H), 2.70-2.45 (m, 10H), 2.23-2.06 (m, 2H). MS (APCI+) m/z 325.0 (M+H)⁺.

### Step 33B

### 2-amino-N-{(1S,2S)-2-[(4-{(1R)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 12D substituting the product of Step 33A for the product of Step 12C. ¹H NMR (400 MHz, methanol-d₄) δ 8.23 (d, J=2.3 Hz, 1H), 7.95 (d, J=2.3 Hz, 1H), 7.80 (s, 1H), 7.73 (s, 1H), 7.54-7.47 (m, 2H), 7.43-7.31 (m, 5H), 4.71-4.60 (m, 2H), 4.41 (td, J=7.5, 4.6 Hz, 1H), 4.32 (dd, J=7.6, 5.3 Hz, 1H), 4.00-3.92 (m, 1H), 3.85 (s, 3H), 3.67 (t, J=6.0 Hz, 2H), 3.04-2.93 (m, 1H), 2.89-2.78 (m, 1H), 2.73-2.46 (m, 10H), 2.25-2.09 (m, 3H), 2.09-1.97 (m, 1H), 1.88-1.69 (m, 3H), 1.66-1.53 (m, 1H). MS (ESI+) m/z 636.3 (M+H)⁺.

### Example 34

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 34A

### 2-{4-[(1S)-5-bromo-2,3-dihydro-1H-inden-1-yl]piperazin-1-yl}ethan-1-ol

Chiral preparative super critical fluid chromatography was performed on racemic 1-(5-bromo-2,3-dihydro-1H-inden-1-yl) piperazine (Step 8C, 500 mg) as described in Step 33A to afford the title compound (203 mg) as the second eluting compound. ¹H NMR (501 MHz, methanol-d₄) δ 7.38 (d, J=1.7 Hz, 1H), 7.32 (dd, J=8.1, 1.8 Hz, 1H), 7.28 (d, J=8.1 Hz, 1H), 4.56 (brs, 1H), 4.27 (dd, J=7.9, 5.4 Hz, 1H), 3.66 (t, J=6.1 Hz, 2H), 3.02-2.89 (m, 1H), 2.89-2.76 (m, 1H), 2.70-2.45 (m, 10H), 2.24-2.05 (m, 2H). MS (APCI+) m/z 325.0 (M+H)⁺.

### Step 34B

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 12D substituting the product of Step 34A for the product of Step 12C. ¹H NMR (400 MHz, methanol-d₄) δ 8.23 (d, J=2.3 Hz, 1H), 7.95 (d, J=2.3 Hz, 1H), 7.81 (s, 1H), 7.74 (s, 1H), 7.54-7.47 (m, 2H), 7.44-7.31 (m, 5H), 4.71-4.61 (m, 2H), 4.41 (td, J=7.5, 4.6 Hz, 1H), 4.33 (dd, J=7.7, 5.3 Hz, 1H), 4.00-3.93 (m, 1H), 3.86 (s, 3H), 3.67 (t, J=6.1 Hz, 2H), 3.03-2.92 (m, 1H), 2.91-2.79 (m, 1H), 2.72-2.48 (m, 10H), 2.25-2.09 (m, 3H), 2.08-1.96 (m, 1H), 1.87-1.70 (m, 3H), 1.65-1.53 (m, 1H). MS (ESI+) m/z 636.2 (M+H)⁺.

### Example 35

### 6-amino-6′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide

A mixture of Intermediate 37 (0.045 g, 0.084 mmol), the product of Step 13A (0.031 g, 0.088 mmol), bis(di-tert-butyl(4-dimethylaminophenyl)phosphine)dichloropalladium (II) (3.58 mg, 5.05 μmol) and K₂CO₃ (0.029 g, 0.211 mmol) in degassed water (0.25 mL) and degassed dioxane (0.7 mL) was heated at 70° C. for 1 hour. The mixture was cooled to ambient temperature and partitioned in water and ethyl acetate. The aqueous layer was extracted with additional ethyl acetate and the combined organic layers were washed with brine, dried over Na₂SO₄, and concentrated. The residue was purified by flash chromatography on silica gel eluting with 0-8% (89:10:1 dichloromethane/methanol/NH₄OH) in dichloromethane to afford the title compound (25 mg). ¹H NMR (400 MHz, DMSO-d₆) δ 8.66 (d, J=6.9 Hz, 1H), 8.57 (d, J=2.6 Hz, 1H), 8.49 (d, J=2.4 Hz, 1H), 8.32-8.24 (m, 2H), 7.55-7.49 (m, 2H), 7.39-7.33 (m, 2H), 7.32-7.22 (m, 5H), 6.53-6.47 (m, 1H), 4.71 (d, J=12.1 Hz, 1H), 4.61 (d, J=12.1 Hz, 1H), 4.53-4.47 (m, 1H), 4.36 (t, J=5.4 Hz, 1H), 4.15-4.10 (m, 1H), 4.08-4.01 (m, 1H), 3.97 (dd, J=9.9, 4.9 Hz, 1H), 3.80-3.70 (m, 2H), 3.49 (q, J=6.2 Hz, 2H), 3.44-3.37 (m, 1H), 3.15 (s, 2H), 2.99-2.91 (m, 2H), 2.40 (t, J=6.3 Hz, 2H), 2.14-2.03 (m, 2H), 1.71-1.54 (m, 4H), 1.26 (d, J=1.2 Hz, 6H). MS ((ESI+) m/z 681.5 (M+H)⁺.

### Example 36

### 6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-6′-oxo-1′,6′-dihydro[3,3′-bipyridine]-5-carboxamide

A mixture of Intermediate 36 (65 mg, 0.098 mmol), 1-methyl-5-(4,4,5,5-tetramethyl-1,3,2-dioxaborolan-2-yl)pyridin-2 (1H)-one (34.6 mg, 0.147 mmol), 1,1′-bis(dipheniylphosphino)ferrocene]dichlioropaliadiurm(II) (6.41 mg, 7.85 μmol) and cesium carbonate (96 mg, 0.294 mmol) in dioxane (1.6 mL) and water (0.4 mL) were degassed and heated in a microwave at 120° C. for 20 minutes. Water was added, and the mixture was extracted with ethyl acetate, washed with brine, dried over MgSO₄ and purified by flash chromatography on silica gel eluting with 0-12% methanol/dichloromethane containing 1% NH₄OH to afford the title compound (41 mg). ¹H NMR (400 MHz, DMSO-d₆) δ 8.31 (d, J=7.8 Hz, 1H), 8.27 (d, J=2.4 Hz, 1H), 7.95 (dd, J=15.3, 2.6 Hz, 2H), 7.74 (dd, J=9.3, 2.7 Hz, 1H), 7.53-7.36 (m, 2H), 7.27 (d, J=8.2 Hz, 2H), 7.20 (dp, J=4.7, 2.2 Hz, 2H), 7.04 (s, 2H), 6.44 (dd, J=9.2, 5.9 Hz, 2H), 4.60-4.46 (m, 2H), 4.38-4.19 (m, 2H), 4.07 (q, J=5.2 Hz, 1H), 3.87 (dt, J=6.4, 3.8 Hz, 1H), 3.45 (m, 5H), 3.19-3.04 (m, 3H), 2.91 (d, J=11.3 Hz, 2H), 2.36 (t, J=6.3 Hz, 2H), 2.10-1.80 (m, 4H), 1.61 (m, 7H), 1.22 (d, J=2.8 Hz, 6H). MS (ESI+) m/z 691 (M+H)⁺.

### Example 37

### 6-amino-6′-fluoro-N-{(3S,4S)-4-[(4-{1-[1-(2-hydroxyethyl)piperidin-4-yl]-3,3-dimethyl-2,3-dihydro-1H-indol-5-yl}phenyl)methoxy]-1-methylpyrrolidin-3-yl}[3,3′-bipyridine]-5-carboxamide

### Step 37A

### 6-amino-N-{(3S,4S)-4-[(4-bromophenyl)methoxy]-1-methylpyrrolidin-3-yl}-6′-fluoro[3,3′-bipyridine]-5-carboxamide

The title compound was prepared as described in Intermediate 31 substituting Intermediate 35 for Intermediate 1. MS (ESI+) m/z 500 (M+H)⁺.

### Step 37B

### 6-amino-6′-fluoro-N-[(3S,4S)-1-methyl-4-{[4-(4,4,5,5-tetramethyl-1,3,2-dioxaborolan-2-yl)phenyl]methoxy}pyrrolidin-3-yl][3,3′-bipyridine]-5-carboxamide

The title compound was prepared as described in Intermediate 19B, substituting the product of Step 37A for Intermediate 19A. MS (ESI+) m/z 548 (M+H)⁺.

### Step 37C

### 6-amino-6′-fluoro-N-{(3S,4S)-4-[(4-{1-[1-(2-hydroxyethyl)piperidin-4-yl]-3,3-dimethyl-2,3-dihydro-1H-indol-5-yl}phenyl)methoxy]-1-methylpyrrolidin-3-yl}[3,3′-bipyridine]-5-carboxamide

The title compound was prepared as described in Step 12D substituting the product of Step 37B for Intermediate 24 and substituting Intermediate 13 for the product of Step 12C. ¹H NMR (400 MHz, DMSO-d₆) δ 8.67 (d, J=8.0 Hz, 1H), 8.54 (d, J=2.6 Hz, 1H), 8.44 (d, J=2.3 Hz, 1H), 8.31-8.12 (m, 2H), 7.51-7.36 (m, 2H), 7.30-7.22 (m, 3H), 7.23-7.11 (m, 3H), 6.48-6.41 (m, 1H), 4.65-4.45 (m, 2H), 4.43-4.21 (m, 2H), 4.15-3.92 (m, 2H), 3.46 (q, J=5.7 Hz, 2H), 3.12 (d, J=15.6 Hz, 3H), 2.96-2.73 (m, 4H), 2.38 (q, J=7.4, 6.3 Hz, 3H), 2.22 (s, 3H), 2.05 (td, J 11.4, 3.1 Hz, 2H), 1.59 (qd, J=11.8, 10.2, 4.5 Hz, 4H), 1.22 (d, J=3.5 Hz, 6H). MS (ESI+) m/z 694 (M+H)⁺.

### Example 38

### rel-2-amino-N-{(1S,2S,4S)-4-hydroxy-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

### Step 38A

### tert-butyl[(cyclopent-3-en-1-yl)oxy]dimethylsilane

To a mixture of cyclopent-3-enol (5.0 g, 59.4 mmol) and 1H-imidazole (8.09 g, 119 mmol) in tetrahydrofuran (200 mL) at 0° C. was added tert-butylchlorodimethylsilane (10.75 g, 71.3 mmol). The mixture was stirred at 0° C. for 1 hour and at ambient temperature overnight. Water was added, and the mixture was extracted with ethyl acetate, washed with brine, dried over MgSO₄, filtered, and concentrated. Purification by flash chromatography on silica gel eluting with 0-10% ethyl acetate/heptane gave the title compound (8.63 g). ¹H NMR (400 MHz, DMSO-d₆) δ 5.57 (s, 1H), 4.42 (tt, J=6.7, 3.3 Hz, 1H), 2.50-2.43 (m, 2H), 2.11-2.00 (m, 2H), 0.77 (s, 10H), -0.05 (s, 6H).

### Step 38B

### rel-tert-butyl(dimethyl){[(1,3r, 5)-6-oxabicyclo[3.1.0]hexan-3-yl]oxy}silane

To the product of Step 38A (2.04 g, 10.28 mmol) that was evacuated and back filled with nitrogen was added dichloromethane (10 mL), and the mixture was cooled to 0° C. A solution of 3-chloroperoxybenzoic acid (2.484 g, 14.40 mmol) in dichloromethane (25 mL) was added, and the mixture was stirred at ambient temperature for 2.5 hours. 10% Sodium bisulfite solution (30 mL) and dichloromethane (30 mL) were added, and the mixture was stirred for 20 minutes and poured into saturated NaHCO₃. The organic phase was separated, the aqueous phase was extracted with dichloromethane. The combined organic phases were washed with water, dried over MgSO₄, filtered, and concentrated. Purification by flash chromatography on silica gel eluting with 100% heptane then 0-10% ethyl acetate/heptane gave the title compound (452 mg) along with a diastereomeric isomer (1.02 g). MS (DCI+) m/z 215.1 (M+H)⁺.

### Step 38C

### rel-(1S,2S,4S)-2-azido-4-{[tert-butyl(dimethyl)silyl]oxy}cyclopentan-1-ol

A mixture of the product of Step 38B (500 mg, 2.332 mmol), NH₄Cl (387 mg, 7.23 mmol) and sodium azide (455 mg, 7.00 mmol) in ethanol (20 mL) and water (4 mL) was heated at 75° C. for 23 hours. Water was added, and the mixture was extracted with diethyl ether, washed with brine, dried over MgSO₄, filtered, and concentrated. Purification by flash chromatography on silica gel eluting with 0-30% ethyl acetate in heptane gave the title compound. MS (ESI+) m/z 258.1 (M+H)⁺.

### Step 38D

### rel-(1S,2S,4S)-2-amino-4-{[tert-butyl(dimethyl)silyl]oxy}cyclopentan-1-ol

A solution of the product of Step 38C (325 mg, 1.263 mmol) in tetrahydrofuran (4 mL) was added to Ra-Ni 2800, water slurry (165 mg, 1.263 mmol) in a 20 mL Barnstead Hast C reactor. The reactor was purged with argon, and the mixture was stirred under 54 psi of H₂ at 24° C. for 3.5 hours. The mixture was filtered, and the filtrate was concentrated and purified by flash chromatography on silica gel eluting with 0-12% methanol in dichloromethane with 1% NH₄OH to afford the title compound (207 mg). MS (DCI+) m/z 232.1 (M+H)⁺.

### Step 38E

### rel-(1S,2S,4S)-2-[(4-bromophenyl)methoxy]-4-{[tert-butyl(dimethyl)silyl]oxy}cyclopentan-1-amine

To a solution of the product of Step 38D (2.78 g, 12.01 mmol) in tetrahydrofuran (40 mL) at 0° C. was added 1 M potassium tert-butoxide in tetrahydrofuran (18.02 mL, 18.02 mmol), and the mixture was stirred at 0° C. for 20 minutes. A solution of 1-bromo-4-(bromomethyl)benzene (3.15 g, 12.61 mmol) in tetrahydrofuran (10 mL) was added, and the mixture was stirred at ambient temperature for 3 hours. The mixture was diluted with ethyl acetate and water, and the aqueous phase was extracted with ethyl acetate. The combined organic phases were washed with water and brine, dried over MgSO₄, filtered, and concentrated. Purification by flash chromatography on silica gel eluting with 0-12% methanol/dichloromethane gave the title compound (4.02 g). MS (ESI+) m/z 402.2 (M+H)⁺.

### Step 38F

### rel-2-amino-N-[(1S,2S,4S)-2-[(4-bromophenyl)methoxy]-4-{[tert-butyl(dimethyl)silyl]oxy}cyclopentyl]-5-(1-methyl-1H-pyrazol-4-yl)pyridine-3-carboxamide

A mixture of Intermediate 2 (0.807 g, 3.70 mmol), triethylamine (1.288 mL, 9.24 mmol) and 1-[bis(dimethylamino)methylene]-1H-1,2,3-triazolo[4,5-b]pyridinium 3-oxid hexafluorophosphate (HATU, 1.827 g, 4.8 mmol) in N,N-dimethylformamide (12 mL) was stirred for 15 minutes and a solution of the product of Step 38E (1.48 g, 3.70 mmol) in N,N-dimethylformamide (5 mL) was added. After stirring at ambient temperature for 16 hours, water was added, and the mixture was extracted with ethyl acetate, washed with water and brine, dried over MgSO₄, filtered, and concentrated. Purification by flash chromatography on silica gel eluting with 0-10% methanol/dichloromethane gave the title compound (1.38 g). MS (APCI+) m/z 600.3 (M+H)⁺.

### Step 38G

### rel-2-amino-N-{(1S,2S,4S)-2-[(4-bromophenyl)methoxy]-4-hydroxycyclopentyl}-5-(1-methyl-1H-pyrazol-4-yl)pyridine-3-carboxamide

To a solution of the product of Step 38F (1.38 g, 2.298 mmol) in tetrahydrofuran (10 mL) was added 1 M tetrabutylammonium fluoride in tetrahydrofuran (9.19 mL, 9.19 mmol), and the mixture was stirred at ambient temperature overnight. Water was added, and the mixture was extracted with ethyl acetate, washed with brine, dried over MgSO₄, filtered, and concentrated. Purification by flash chromatography on silica gel eluting with (0-12% methanol in dichloromethane) gave the title compound (1.04 g). MS (ESI+) m/z 486.3 (M+H)⁺.

### Step 38H

### rel-2-amino-N-[(1S,2S,4S)-4-hydroxy-2-{[4-(4,4,5,5-tetramethyl-1,3,2-dioxaborolan-2-yl)phenyl]methoxy}cyclopentyl]-5-(1-methyl-1H-pyrazol-4-yl)pyridine-3-carboxamide

A mixture of tetrakis(triphenylphosphine)palladium (314 mg, 0.271 mmol), the product of Step 38G (220 mg, 0.452 mmol), 4,4,4′,4′,5,5,5′,5′-octamethyl-2,2′-bi(1,3,2-dioxaborolane) (230 mg, 0.905 mmol) and potassium acetate (133 mg, 1.357 mmol) was purged with nitrogen for 10 minutes and dioxane (10 mL) was added. The mixture was purged with nitrogen for 5 minutes and heated at 100° C. for 16 hours. The mixture was cooled, diluted with ethyl acetate, filtered, and concentrated. Purification by flash chromatography on silica gel eluting with 0-12% methanol/dichloromethane gave the title compound (94 mg). MS (ESI+) m/z 534.4 (M+H)⁺.

### Step 38I

### rel-2-amino-N-{(1S,2S,4S)-4-hydroxy-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide

A mixture of the product of Step 38H (59 mg, 0.111 mmol), 1,1-bis(diphenylphosphino) ferroceineldichloropalladiurn(II)-CH₂Cl₂ adduct (9.03 mg, 0.011 mmol), Intermediate 23 (39.1 mg, 0.111 mmol) and cesium carbonate (108 mg, 0.332 mmol) was purged with nitrogen for 5 minutes. Dioxane (2 mL) and water (0.5 mL) were added, and the mixture was purged with nitrogen for 5 minutes and stirred in a Biotage Initiator microwave at 120° C. for 30 minutes. The mixture was diluted with ethyl acetate, washed with brine, dried over MgSO₄, filtered, and concentrated. Purification by flash chromatography on silica gel eluting with 0-14% methanol in dichloromethane with 1% NH₄OH gave the title compound (11 mg). ¹H NMR (400 MHz, DMSO-d₆) δ 8.38 (d, J=8.2 Hz, 1H), 8.33 (d, J=2.3 Hz, 1H), 8.00 (d, J=2.3 Hz, 1H), 7.97 (s, 1H), 7.78 (s, 1H), 7.49-7.42 (m, 2H), 7.29 (d, J=8.2 Hz, 2H), 7.26-7.19 (m, 2H), 6.98 (s, 2H), 6.48 (d, J=8.9 Hz, 1H), 4.70 (d, J=4.1 Hz, 1H), 4.59-4.47 (m, 3H), 4.35 (t, J=5.4 Hz, 1H), 4.19 (q, J=5.2 Hz, 1H), 3.93 (dt, J=7.9, 6.1 Hz, 1H), 3.82 (s, 3H), 3.49 (dt, J=9.6, 4.3 Hz, 2H), 3.39 (s, 1H), 3.14 (s, 2H), 2.99-2.90 (m, 2H), 2.40 (t, J=6.3 Hz, 2H), 2.37-2.29 (m, 1H), 2.08 (td, J=11.4, 3.1 Hz, 2H), 1.91 (ddd, J=12.8, 8.2, 4.1 Hz, 1H), 1.75 (ddd, J=13.5, 8.2, 6.1 Hz, 1H), 1.69-1.51 (m, 5H), 1.25 (d, J=2.5 Hz, 6H). MS (ESI+) m/z 680.1 (M+H)⁺.

### Example 39

### 2-amino-N-{(1S,2S)-2-[(4-{1-[1-(2-hydroxyethyl)piperidin-4-yl]-3-methyl-3-propyl-2,3-dihydro-1H-indol-5-yl}phenyl)methoxy]cyclopentyl}-5-(1-methyl-1H-pyrazol-4-yl)pyridine-3-carboxamide

### Step 39A

### 5-bromo-3-methyl-3-propyl-2,3-dihydro-1H-indole

A mixture of (4-bromophenyl)hydrazine hydrochloride (2 g, 8.95 mmol), acetic acid (15 mL) and 2-methylvaleraldehyde (0.896 g, 8.95 mmol) was heated at 60° C. for 3 hours under nitrogen. The mixture was diluted with 1,2-dichloroethane (15 mL), cooled to 0° C. Sodium triacetoxyborohydride (2.84 g, 13.42 mmol) was added, and the mixture was stirred for 20 minutes. The mixture was concentrated, and the residue dissolved in ethyl acetate and washed with saturated Na₂CO₃. The organic layer was dried over MgSO₄, filtered, and concentrated, and the residue was purified by flash chromatography on silica gel eluting with 0-50% ethyl acetate/heptanes to afford the title compound (740 mg). ¹H NMR (400 MHz, DMSO-d₆) δ 7.04 (d, J=2.1 Hz, 1H), 7.01 (dd, J=8.2, 2.1 Hz, 1H), 6.40 (d, J=8.1 Hz, 1H), 5.59 (d, J=1.9 Hz, 1H), 3.29 (dd, J=9.1, 1.8 Hz, 1H), 3.12 (dd, J=9.1, 2.1 Hz, 1H), 1.48 (dddd, J=29.6, 13.2, 11.7, 4.7 Hz, 2H), 1.36-1.21 (m, 1H), 1.19 (s, 3H), 1.17-1.00 (m, 1H), 0.84 (t, J=7.3 Hz, 3H). LCMS (APCI) m/z 254.83 (M+H)⁺.

### Step 39B

### tert-butyl 4-(5-bromo-3-methyl-3-propyl-2,3-dihydro-1H-indol-1-yl)piperidine-1-carboxylate

A mixture of the product of Step 39A (740 mg, 2.91 mmol), tert-butyl 4-oxopiperidine-1-carboxylate (638 mg, 3.20 mmol), acetic acid (8.3 mL) and sodium triacetoxyborohydride (1.23 g, 5.82 mmol) was stirred at ambient temperature overnight. The mixture was diluted with dichloromethane and washed with a saturated Na₂CO₃. The organic layer was dried over MgSO₄, filtered, and concentrated, and the residue was purified by flash chromatography on silica gel eluting with 0-10% ethyl acetate/heptanes to afford the title compound (1.03 g). ¹H NMR (400 MHz, DMSO-d₆) δ 7.09 (dd, J=8.3, 2.1 Hz, 1H), 7.04 (d, J=2.1 Hz, 1H), 6.42 (d, J=8.4 Hz, 1H), 4.13-3.95 (m, 2H), 3.55 (tt, J=11.7, 3.7 Hz, 1H), 3.21 (d, J=9.0 Hz, 1H), 3.02 (d, J=9.0 Hz, 1H), 2.79 (s, 2H), 1.63 (dd, J=10.8, 6.7 Hz, 2H), 1.57-1.42 (m, 4H), 1.40 (s, 9H), 1.27 (dp, J=17.1, 6.0, 5.5 Hz, 1H), 1.18 (s, 3H), 1.14-1.01 (m, 1H), 0.83 (t, J=7.2 Hz, 3H). LCMS (APCI) m/z 437.24 (M+H)⁺.

### Step 39C

### 5-bromo-3-methyl-1-(piperidin-4-yl)-3-propyl-2,3-dihydro-1H-indole

A mixture of the product of Step 39B (1.03 g, 2.3 mmol), tetrahydrofuran (10 mL) and 4 M HCl in dioxane (5.89 mL, 23.55 mmol) was stirred at ambient temperature overnight. After concentration, the residue was dissolved in ethyl acetate and washed with saturated NaHCO₃. The organic layer was dried over MgSO₄, filtered, and concentrated to afford the title compound (631 mg) which was used without further purification. ¹H NMR (501 MHz, DMSO-d₆) δ 7.08 (dd, J=8.3, 2.1 Hz, 1H), 7.03 (d, J=2.1 Hz, 1H), 6.38 (d, J=8.3 Hz, 1H), 3.41 (tt, J=11.9, 4.0 Hz, 1H), 3.22 (d, J=9.0 Hz, 1H), 3.04 (d, J=9.0 Hz, 1H), 3.00 (ddd, J=12.1, 4.1, 2.2 Hz, 2H), 2.59-2.52 (m, 2H), 1.65-1.36 (m, 7H), 1.34-1.22 (m, 1H), 1.19 (s, 3H), 1.08 (tdd, J=12.3, 7.0, 4.6 Hz, 1H), 0.83 (t, J=7.3 Hz, 3H). LCMS (APCI) m/z 336.97 (M+H)⁺.

### Step 39D

### 2-[4-(5-bromo-3-methyl-3-propyl-2,3-dihydro-1H-indol-1-yl)piperidin-1-yl]ethan-1-ol

A mixture of the product of Step 39C (631 mg, 1.87 mmol), methanol (10 mL), 2-bromoethanol (701 mg, 5.6 mmol) and K₂CO₃ (517 mg, 3.74 mmol) was stirred at ambient temperature for 48 hours. The mixture was diluted with ethyl acetate and the solids were filtered. Purification by flash chromatography on silica gel eluting with 0-10% methanol/dichloromethane afforded the title compound (474 mg). ¹H NMR (400 MHz, DMSO-d₆) δ 7.08 (dd, J=8.4, 2.1 Hz, 1H), 7.03 (d, J=2.1 Hz, 1H), 6.36 (d, J=8.4 Hz, 1H), 4.40 (dt, J=34.5, 5.4 Hz, 2H), 3.48 (q, J=5.7 Hz, 2H), 3.39 (t, J=2.1 Hz, 2H), 3.23 (d, J=9.0 Hz, 1H), 3.04 (d, J=9.0 Hz, 1H), 2.93 (d, J=11.2 Hz, 2H), 2.39 (t, J=6.4 Hz, 2H), 2.05 (td, J=11.1, 5.5 Hz, 2H), 1.67-1.37 (m, 5H), 1.35-1.21 (m, 1H), 1.19 (s, 3H), 1.07 (dt, J=12.4, 7.3 Hz, 1H), 0.83 (t, J=7.2 Hz, 3H). LCMS (APCI) m/z 381.08 (M+H)⁺.

### Step 39E

### 2-amino-N-{(1S,2S)-2-[(4-{1-[1-(2-hydroxyethyl)piperidin-4-yl]-3-methyl-3-propyl-2,3-dihydro-1H-indol-5-yl}phenyl)methoxy]cyclopentyl}-5-(1-methyl-1H-pyrazol-4-yl)pyridine-3-carboxamide

A mixture of the product of Step 39D (474 mg, 1.24 mmol), Intermediate 33 (643 mg, 1.24 mmol), cesium carbonate (1.21 g, 3.73 mmol) and 3/1 dioxane/water (8.29 mL) was sparged with argon for 20 minutes and tris(dibenzylideneacetone)dipalladium (23 mg, 0.025 mmol) and 4-(di-tert-butylphosphino)-N,N-dimethylaniline (16 mg, 0.062 mmol) were added. The reaction vial was heated at 70° C. for 16 hours, cooled to ambient temperature and diluted with ethyl acetate. The organic layer was dried over MgSO₄, filtered, and concentrated. The residue was purified using by flash chromatography on silica gel eluting with 0-10% methanol/dichloromethane to afford the title compound (306 mg), as a mixture of diastereomers. ¹H NMR (400 MHz, DMSO-d₆) δ 8.34 (s, 1H), 8.32 (d, J=2.3 Hz, 1H), 8.00 (d, J=2.3 Hz, 1H), 7.98 (s, 1H), 7.78 (d, J=0.8 Hz, 1H), 7.47 (d, J=8.0 Hz, 2H), 7.31 (d, J=8.0 Hz, 2H), 7.25 (dt, J=8.2, 1.4 Hz, 1H), 7.19 (d, J=1.7 Hz, 1H), 6.96 (s, 2H), 6.46 (d, J=8.3 Hz, 1H), 4.64-4.49 (m, 2H), 4.35 (t, J=5.3 Hz, 1H), 3.96-3.88 (m, 1H), 3.83 (s, 3H), 3.49 (q, J=6.0 Hz, 2H), 3.26 (d, J=8.9 Hz, 1H), 3.08 (d, J=5.3 Hz, 2H), 2.94 (d, J=11.2 Hz, 2H), 2.40 (t, J=6.4 Hz, 2H), 2.06 (d, J=9.9 Hz, 3H), 1.93 (dt, J=12.0, 7.0 Hz, 1H), 1.81-1.40 (m, 11H), 1.38-1.18 (m, 4H), 1.11 (s, 3H), 0.83 (td, J=7.2, 2.9 Hz, 3H). LCMS (APCI) m/z 692.62 (M+H)⁺.

### Example 40

### 2-amino-N-{(1S,2S)-2-[(4-{3-ethyl-1-[i-(2-hydroxyethyl)piperidin-4-yl]-3-methyl-2,3-dihydro-1H-indol-5-yl}phenyl)methoxy]cyclopentyl}-5-(1-methyl-1H-pyrazol-4-yl)pyridine-3-carboxamide

### Step 40A

### tert-butyl 4-(5-bromo-3-ethyl-3-methyl-2,3-dihydro-1H-indol-1-yl)piperidine-1-carboxylate

A mixture of 4-bromophenylhydrazine hydrochloride (14.27 g, 63.9 mmol), acetic acid (200 mL) and 2-methylbutyraldehyde (5 g, 58.1 mmol) was heated at 60° C. for 3 hours and allowed to cool to ambient temperature overnight. The mixture was diluted with dichloromethane (100 mL), cooled to 0° C. and sodium triacetoxyborohydride (36.9 g, 174 mmol) was added. The cooling bath was removed, and the reaction stirred until complete. tert-Butyl 4-oxopiperidine-1-carboxylate (13.6 g, 68.3 mmol) was added, and the mixture was stirred overnight. The mixture was poured into saturated NaHCO₃ and the aqueous layer was extracted with dichloromethane. The organic layers were dried over MgSO₄, filtered, and concentrated, and the residue was purified by flash chromatography on silica gel eluting with 0-50% ethyl acetate/heptanes to afford the title compound (2.06 g). ¹H NMR (400 MHz, DMSO-d₆) δ 7.10 (dd, J=8.3, 2.1 Hz, 1H), 7.04 (d, J=2.1 Hz, 1H), 6.42 (d, J=8.4 Hz, 1H), 4.04 (dd, J=9.5, 5.1 Hz, 2H), 3.56 (tt, J=11.6, 3.7 Hz, 1H), 3.01 (d, J=9.1 Hz, 1H), 2.80 (s, 2H), 1.63 (td, J=7.8, 6.1, 3.0 Hz, 2H), 1.59-1.47 (m, 2H), 1.45 (dd, J=7.8, 4.5 Hz, 1H), 1.42-1.37 (m, 11H), 1.17 (s, 3H), 0.76 (t, J=7.4 Hz, 3H). MS (DCI+) m/z 423.1 (M+H)⁺.

### Step 40B

### 5-bromo-3-ethyl-3-methyl-1-(piperidin-4-yl)-2,3-dihydro-1H-indole

A mixture of the product of Step 40A (2.06 g, 4.87 mmol), dichloromethane (10 mL) and trifluoroacetic acid (7.5 mL, 97 mmol) was stirred at ambient temperature overnight. The mixture was quenched with saturated NaHCO₃ and extracted with dichloromethane. The organic layers were dried over MgSO₄, filtered, and concentrated to provide the title compound (1.5 g) which was used without further purification. ¹H NMR (400 MHz, DMSO-d₆) δ 7.13 (dd, J=8.3, 2.1 Hz, 1H), 7.08 (d, J=2.1 Hz, 1H), 6.47 (d, J=8.4 Hz, 1H), 3.68 (tt, J=11.3, 4.3 Hz, 1H), 3.40-3.30 (m, 3H), 3.20 (d, J=8.8 Hz, 1H), 3.03 (d, J=8.9 Hz, 1H), 2.95 (td, J=12.6, 3.5 Hz, 2H), 1.86-1.65 (m, 4H), 1.64-1.47 (m, 2H), 1.20 (s, 3H), 0.76 (t, J=7.4 Hz, 3H). MS (APCI+) m/z 322.97 (M+H)⁺.

### Step 40C

### 2-[4-(5-bromo-3-ethyl-3-methyl-2,3-dihydro-1H-indol-1-yl)piperidin-1-yl]ethan-1-ol

A mixture of Step 40B (1.57 g, 4.87 mmol), methanol (49 mL), 2-bromoethanol (1.83 g, 14.6 mmol), K₂CO₃ (2.02 g, 14.6 mmol) and glass beads (2 g) was stirred overnight. The mixture was diluted with ethyl acetate, filtered through diatomaceous earth and concentrated. The residue was dissolved in ethyl acetate, washed with water and brine, dried over MgSO₄ and concentrated. The residue was purified by flash chromatography on silica gel eluting with 0-10% methanol/dichloromethane to afford the title compound (718 mg). ¹H NMR (501 MHz, DMSO-d₆) δ 7.10 (dd, J=8.4, 2.1 Hz, 1H), 7.04 (d, J=2.1 Hz, 1H), 6.39 (d, J=8.4 Hz, 1H), 4.57 (s, 1H), 3.54 (t, J=6.1 Hz, 2H), 3.22 (d, J=9.0 Hz, 1H), 3.12-3.06 (m, 2H), 3.04 (d, J=9.0 Hz, 1H), 2.57 (s, 2H), 2.29 (s, 2H), 1.66 (p, J=4.4 Hz, 4H), 1.61-1.45 (m, 2H), 1.18 (s, 3H), 0.76 (t, J=7.4 Hz, 3H). MS (APCI+) m/z 367.02 (M+H)⁺.

### Step 40D

### 2-amino-N-{(1S,2S)-2-[(4-{3-ethyl-1-[1-(2-hydroxyethyl)piperidin-4-yl]-3-methyl-2,3-dihydro-1H-indol-5-yl}phenyl)methoxy]cyclopentyl}-5-(1-methyl-1H-pyrazol-4-yl)pyridine-3-carboxamide

A mixture of Intermediate 33 (1.01 g, 1.96 mmol), the product of Step 40C (0.718 g, 1.96 mmol), cesium carbonate (1.91 g, 5.86 mmol), dioxane (10 mL) and water (3 mL) was sparged with argon for 20 minutes. 4-(Di-tert-butylphosphino)-N,N-dimethylaniline (26 mg, 0.098 mmol) and tris(dibenzylideneacetone)dipalladium (0.036 g, 0.039 mmol) were added, and the reaction vial was heated at 70° C. for 16 hours. The mixture was cooled to ambient temperature, ammonium pyrrolidinedithiocarbamate (70 mg) was added, and the mixture was stirred for 15 minutes. The mixture was partitioned between ethyl acetate and water, and the aqueous layer was washed with ethyl acetate. The organic layers were concentrated, and the residue was purified by flash chromatography on silica gel eluting with 0-10% methanol/dichloromethane to afford the title compound (505 mg) as a diastereomeric mixture. ¹H NMR (400 MHz, DMSO-d₆) δ 8.36-8.29 (m, 2H), 8.00 (d, J=2.3 Hz, 1H), 7.98 (s, 1H), 7.78 (d, J=0.7 Hz, 1H), 7.47 (d, J=8.0 Hz, 2H), 7.30 (d, J=7.9 Hz, 2H), 7.25 (dd, J=8.4, 1.9 Hz, 1H), 7.18 (d, J=1.9 Hz, 1H), 6.95 (s, 2H), 6.47 (d, J=8.3 Hz, 1H), 4.65-4.46 (m, 2H), 4.36 (t, J=5.4 Hz, 1H), 4.34-4.23 (m, 1H), 3.94-3.88 (m, 1H), 3.83 (d, J=1.3 Hz, 3H), 3.49 (q, J=6.0 Hz, 2H), 3.25 (d, J=8.9 Hz, 1H), 3.13-3.01 (m, 1H), 2.94 (d, J=11.0 Hz, 2H), 2.39 (t, J=6.3 Hz, 2H), 2.06 (td, J=13.0, 9.0 Hz, 3H), 1.92 (dt, J=11.9, 7.0 Hz, 1H), 1.83-1.44 (m, TOH), 1.22 (d, J=2.5 Hz, 3H), 1.10 (s, 1H), 0.79 (td, J=7.5, 2.2 Hz, 3H). MS (DCI+) m/z 678.4 (M+H)⁺.

### Example 41

### 3-amino-6-(6-fluoropyridin-3-yl)-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyrazine-2-carboxamide

### Step 41A

### methyl 3-amino-6-(6-fluoropyridin-3-yl)pyrazine-2-carboxylate

A mixture of methyl 3-amino-6-bromopyrazine-2-carboxylate (503 mg, 2.2 mmol), 2-fluoropyridine-5-boronic acid pinacol ester (537 mg, 2.4 mmol), [1,1F bis(diphenylphosphino) ferrocene]dichloropalladium (b complex with dichloromethane (93 mg, 0.11 mmol) and potassium phosphate (1.41 g, 6.6 mmol) in dioxane (10 mL) in a microwave vial evacuated and backfilled with nitrogen, was heated at 75° C. for 18 hours. After cooling to ambient temperature, the mixture was filtered through diatomaceous earth, concentrated and purified by flash chromatography on silica gel eluting with 0-100% ethyl acetate/hexanes to afford the title compound (367 mg). ¹H NMR (400 MHz, DMSO-d₆) δ 8.96 (s, 1H), 8.85-8.79 (m, 1H), 8.56-8.46 (m, 1H), 7.55 (s, 2H), 7.30 (dd, J=8.6, 2.8 Hz, 1H), 3.91 (s, 3H). MS (ESI+) m/z 255.1 (M+H)⁺.

### Step 41B

### 3-amino-6-(6-fluoropyridin-3-yl)pyrazine-2-carboxylic acid

To a solution of the product of Step 41A (367 mg, 1.5 mmol) in tetrahydrofuran (13 mL) was added LiOH (95 mg, 2.3 mmol) in water (2 mL), and the mixture was stirred at ambient temperature for 18 hours. 1 M HCl (2.3 mL, 2.3 mmol) was added, and the precipitate was collected, washed with water and dried under vacuum to afford the title compound (347 mg). ¹H NMR (400 MHz, DMSO-d₆) δ 8.97 (s, 1H), 8.94 (d, J=2.4 Hz, 1H), 8.66 (td, J=8.3, 2.6 Hz, 1H), 7.61 (s, 2H), 7.30 (dd, J=8.6, 2.8 Hz, 1H). MS (ESI+) m/z 253.2 (M+H)⁺.

### Step 41C

### 3-amino-N-{(1S,2S)-2-[(4-bromophenyl)methoxy]cyclopentyl}-6-(6-fluoropyridin-3-yl)pyrazine-2-carboxamide

To a solution of the product of Step 41B (253.7 mg, 1.083 mmol), Intermediate 1 (381.8 mg, 1.413 mmol) and N,N-diisopropylethylamine (0.5 mL, 2.86 mmol) in N,N-dimethylformamide (6 mL) was added 1-[bis(dimethylamino)methylene]-1H-1,2,3-triazolo[4,5-b]pyridinium 3-oxid hexafluorophosphate (HATU, 470.3 mg, 1.237 mmol), and the mixture was stirred for 72 hours. The mixture was then concentrated and purified by flash chromatography on silica gel eluting with 0-40% ethyl acetate/hexanes to afford the title compound (385 mg). ¹H NMR (501 MHz, DMSO-d₆) δ 9.05 (dd, J=2.6, 0.8 Hz, 1H), 8.90 (s, 1H), 8.74-8.66 (m, 2H), 7.46-7.40 (m, 2H), 7.33-7.22 (m, 3H), 4.52 (s, 2H), 4.30 (td, J=8.4, 5.2 Hz, 1H), 4.03 (dt, J=6.7, 4.8 Hz, 1H), 2.06-1.94 (m, 2H), 1.68 (dddd, J=20.3, 18.1, 10.0, 3.7 Hz, 4H). MS (ESI+) m/z 488.2 (M+H)⁺.

### Step 41D

### 3-amino-6-(6-fluoropyridin-3-yl)-N-[(1S,2S)-2-{[4-(4,4,5,5-tetramethyl-1,3,2-dioxaborolan-2-yl)phenyl]methoxy}cyclopentyl]pyrazine-2-carboxamide

A mixture of the product of Step 41C (385.4 mg, 0.792 mmol), 4,4,4′,4′,5,5,5′,5′-octamethyl-2,2′-bi(1,3,2-dioxaborolane) (411.6 mg, 1.621 mmol), tetrakis(triphenylphosphine)palladium(0) (46.9 mg, 0.041 mmol), potassium acetate (247.8 mg, 2.52 mmol) and dioxane (4 mL) in a microwave vial was evacuated and backfilled with nitrogen, was heated at 110° C. for 18 hours. The mixture was concentrated and purified by flash chromatography on silica gel eluting with 0-50% ethyl acetate/hexanes to afford the title compound (254 mg). ¹H NMR (501 MHz, DMSO-d₆) δ 9.03 (d, J=2.6 Hz, 1H), 8.90 (s, 1H), 8.77-8.62 (m, 2H), 7.58-7.52 (m, 2H), 7.33-7.24 (m, 3H), 4.57 (s, 2H), 4.31 (dd, J=8.4, 5.3 Hz, 1H), 4.03 (dt, J=6.7, 4.8 Hz, 1H), 2.08-1.91 (m, 2H), 1.82-1.52 (m, 4H), 1.26 (s, 12H). MS (ESI+) m/z 534.1 (M+H)⁺.

### Step 41E

### 3-amino-6-(6-fluoropyridin-3-yl)-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyrazine-2-carboxamide

A mixture of the product of Step 41D (128 mg, 0.24 mmol), Intermediate 23 (113 mg, 0.32 mmol), bis(di-tert-butyl(4-dimethylaminophenyl)phosphine)dichloropalladium(II) (8 mg, 0.011 mmol) and potassium phosphate (104 mg, 0.49 mmol) were combined in 3:1 dioxane/water (2 mL) was evacuated and backfilled with nitrogen. The mixture was heated at 100° C. for 18 hours. After cooling to ambient temperature, the mixture was filtered through diatomaceous earth and concentrated. The residue was purified by reverse phase HPLC using a gradient elution of 5/95 to 90/10 acetonitrile/0.1% trifluoroacetic acid in water to provide the title compound as a trifluoroacetic acid salt. The salt was dissolved in methanol and loaded onto an Agilent Bond Elut SCX column. The free base was eluted from the column with 1 M NH₃ in methanol and concentrated. The residue was dissolved in methanol and filtered through a 0.22 μM PTFE syringe filter and concentrated to afford the title compound (31 mg). ¹H NMR (400 MHz, methanol-d₄) δ 8.72 (dd, J=2.6, 0.9 Hz, 1H), 8.63 (s, 1H), 8.42 (ddd, J=8.6, 7.6, 2.6 Hz, 1H), 7.39-7.31 (m, 2H), 7.28 (d, J=8.3 Hz, 2H), 7.15-7.06 (m, 2H), 6.99 (ddd, J=8.6, 2.6, 0.7 Hz, 1H), 6.43 (d, J=8.0 Hz, 1H), 4.63 (d, J=12.3 Hz, 1H), 4.55 (d, J=12.2 Hz, 1H), 4.36 (td, J=8.1, 5.5 Hz, 1H), 4.08-3.96 (m, 1H), 3.70 (t, J=6.1 Hz, 2H), 3.18 (s, 2H), 3.15-3.04 (m, 2H), 2.58 (t, J=6.1 Hz, 2H), 2.31-1.99 (m, 4H), 1.87-1.52 (m, 9H), 1.27 (s, 6H). MS (ESI+) m/z 680.3 (M+H)⁺.

### Example 42

### 2-amino-5-(1,3-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide

### Step 42A

### 2-amino-5-(1,3-dimethyl-1H-pyrazol-4-yl)pyridine-3-carboxylic acid

The title compound was prepared as described in Intermediate 2 substituting 1,3-dimethyl-4-(4,4,5,5-tetramethyl-1,3,2-dioxaborolan-2-yl)-1H-pyrazole for 1-methyl-4-(4,4,5,5-tetramethyl-1,3,2-dioxaborolan-2-yl)-1H-pyrazole. MS (ESI+) m/z 233 (M+H)⁺.

### Step 42B

### 2-amino-N-{(3S,4R)-4-[(4-bromophenyl)methoxy]oxolan-3-yl}-5-(1,3-dimethyl-1H-pyrazol-4-yl)pyridine-3-carboxamide

The title compound was prepared as described in Intermediate 29 substituting the product of Step 42A for Intermediate 2. MS (ESI+) m/z 486 (M+H)⁺.

### Step 42C

### 2-amino-5-(1,3-dimethyl-1H-pyrazol-4-yl)-N-[(3S,4R)-4-{[4-(4,4,5,5-tetramethyl-1,3,2-dioxaborolan-2-yl)phenyl]methoxy}oxolan-3-yl]pyridine-3-carboxamide

The title compound was prepared as described in Intermediate 19B substituting the product of Step 42B for Intermediate 19A. MS (ESI+) m/z 534 (M+H)⁺.

### Step 42D

### 2-amino-5-(1,3-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide

The title compound was prepared as described in Step 12D substituting the product of Step 42C for Intermediate 24 and substituting Intermediate 13 for the product of Step 12C. ¹H NMR (400 MHz, DMSO-d₆) δ 8.53 (d, J=6.8 Hz, 1H), 8.11 (d, J=2.3 Hz, 1H), 7.90 (d, J=2.3 Hz, 1H), 7.72 (s, 1H), 7.59-7.42 (m, 2H), 7.31 (d, J=8.0 Hz, 2H), 7.25 (d, J=7.2 Hz, 2H), 6.95 (s, 2H), 6.52-6.44 (m, 1H), 4.75-4.51 (m, 2H), 4.50-4.23 (m, 2H), 4.07 (dt, J=4.4, 1.9 Hz, 1H), 4.00 (dd, J=9.3, 6.2 Hz, 1H), 3.89 (dd, J=10.0, 4.8 Hz, 1H), 3.79-3.58 (m, 4H), 3.46 (q, J=5.7 Hz, 2H), 3.36 (s, 2H), 3.09 (d, J=18.0 Hz, 2H), 2.92 (d, J=11.1 Hz, 2H), 2.37 (t, J=6.3 Hz, 2H), 2.20 (s, 3H), 2.06 (td, J=11.3, 3.1 Hz, 2H), 1.59 (dtt, J=19.7, 11.7, 5.7 Hz, 4H), 1.23 (s, 6H). MS (ESI+) m/z 680 (M+H)⁺.

### Example 43

### 2-amino-N-{(1S,2S)-2-[(4-{1-[1-(2-hydroxyethyl)piperidin-4-yl]-1,2-dihydrospiro[indole-3,3′-oxolan]-5-yl}phenyl)methoxy]cyclopentyl}-5-(1-methyl-1H-pyrazol-4-yl)pyridine-3-carboxamide

### Step 43A

### tert-butyl 4-(5-bromospiro[indole-3,3′-oxolan]-1 (2H)-yl)piperidine-1-carboxylate

The title compound was prepared as described in Step 12A substituting 5′-bromo-4,5-dihydro-2H-spiro[furan-3,3′-indoline] for 5-bromo-3,3-dimethylindoline. MS (ESI+) m/z 437.1 (M+H)⁺.

### Step 43B

### 5-bromo-1-(piperidin-4-yl)-1,2-dihydrospiro[indole-3,3′-oxolane]

The title compound was prepared as described in Example 5 substituting the product of Step 43A for the product of Example 4. MS (ESI+) m/z 337.3 (M+H)⁺.

### Step 43C

### 2-[4-(5-bromospiro[indole-3,3′-oxolan]-1 (2H)-yl)piperidin-1-yl]ethan-1-ol

The title compound was prepared as described in Step 13A substituting the product of Step 43B for the product of Step 12B. MS (ESI+) m/z 337.3 (M+H)⁺.

### Step 43D

### 2-amino-N-{(1S,2S)-2-[(4-{1-[1-(2-hydroxyethyl)piperidin-4-yl]-1,2-dihydrospiro[indole-3,3′-oxolan]-5-yl}phenyl)methoxy]cyclopentyl}-5-(1-methyl-1H-pyrazol-4-yl)pyridine-3-carboxamide

A mixture of Intermediate 33 (30 mg, 0.057 mmol), the product of Step 43C (23 mg, 0.06 mmol), bis(di-tert-butyl(4-dimethylaminophenyl)phosphine)dichloropalladium(II) (2 mg, 2.84 μmol) and K₂CO₃ (24 mg, 0.170 mmol) in degassed water (0.2 mL) and degassed dioxane (0.6 mL) was heated at 70° C. for 1 hour. The mixture was cooled to ambient temperature and partitioned in water and ethyl acetate. The organic layer was washed with brine, dried over Na₂SO₄, and concentrated. The concentrate was purified by flash chromatography on silica gel eluting with 0-15% methanol/dichloromethane to afford the title compound (19 mg). ¹H NMR (400 MHz, DMSO-d₆) δ 8.40-8.30 (m, 2H), 8.04-7.96 (m, 2H), 7.78 (d, J=0.8 Hz, 1H), 7.54-7.45 (m, 2H), 7.39-7.26 (m, 4H), 6.96 (s, 2H), 6.55 (d, J=8.3 Hz, 1H), 4.62-4.52 (m, 2H), 4.51-4.36 (m, 1H), 4.36-4.25 (m, 1H), 4.03-3.85 (m, 3H), 3.83 (s, 3H), 3.75 (d, J=8.3 Hz, 1H), 3.63 (dd, J=8.4, 4.6 Hz, 1H), 3.56-3.43 (m, 3H), 2.99 (d, J=10.9 Hz, 2H), 2.46 (brs, 2H), 2.28-1.85 (m, 6H), 1.80-1.52 (m, 8H). MS (ESI+) m/z 692.0 (M+H)⁺.

### Example 44

### 6-amino-5′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide

### Step 44A

### 6-amino-N-{(3S,4R)-4-[(4-bromophenyl)methoxy]oxolan-3-yl}-5′-fluoro[3,3′-bipyridine]-5-carboxamide

The title compound was prepared as described in Intermediate 29 substituting Intermediate 5 for Intermediate 2. MS (ESI+) m/z 487 (M+H)⁺.

### Step 44B

### 6-amino-5′-fluoro-N-[(3S,4R)-4-{[4-(4,4,5,5-tetramethyl-1,3,2-dioxaborolan-2-yl)phenyl]methoxy}oxolan-3-yl][3,3′-bipyridine]-5-carboxamide

The title compound was prepared as described in Intermediate 19B substituting the product of Step 44A for Intermediate 19A. MS (ESI+) m/z 535 (M+H)⁺.

### Step 44C

### 6-amino-5′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide

The title compound was prepared as described in Step 4C substituting the product of Step 44B for Intermediate 33 and substituting Intermediate 13 for the product of Step 4B. ¹H NMR (400 MHz, DMSO-d₆) δ 8.82 (t, J=1.8 Hz, 1H), 8.67 (d, J=6.9 Hz, 1H), 8.54 (d, J=2.3 Hz, 1H), 8.46 (d, J=2.6 Hz, 1H), 8.31 (d, J=2.4 Hz, 1H), 8.05 (dt, J=10.6, 2.4 Hz, 1H), 7.59-7.42 (m, 2H), 7.43-7.28 (m, 4H), 7.28-7.21 (m, 2H), 6.52-6.36 (m, 1H), 4.77-4.53 (m, 2H), 4.53-4.44 (m, 1H), 4.38 (s, 1H), 4.10 (dt, J=4.4, 1.9 Hz, 1H), 3.98 (ddd, J=27.7, 9.6, 5.4 Hz, 2H), 3.73 (dt, J=9.9, 3.0 Hz, 2H), 3.47 (t, J=6.3 Hz, 2H), 3.10 (s, 2H), 3.05 (m, 1H), 2.94 (d, J=11.0 Hz, 2H), 2.40 (t, J=6.3 Hz, 2H), 2.09 (dt, J=11.6, 5.8 Hz, 2H), 1.60 (dp, J=11.7, 4.5, 3.8 Hz, 4H), 1.23 (s, 6H). MS (ESI+) m/z 681 (M+H)⁺.

### Example 45

### 2-amino-N-{(3S,4R)-4-[(4-{(1S)-1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide

The title compound was prepared as described in Example 14 substituting the product of Step 34A for the product of Step 7A. ¹H NMR (501 MHz, methanol-d₄) δ 8.25 (d, J=2.3 Hz, 1H), 8.04 (d, J=2.3 Hz, 1H), 7.86 (d, J=0.7 Hz, 1H), 7.76 (d, J=0.8 Hz, 1H), 7.58-7.52 (m, 2H), 7.46-7.38 (m, 5H), 4.79-4.68 (m, 2H), 4.62-4.57 (m, 1H), 4.34 (dd, J=7.7, 5.2 Hz, 1H), 4.22-4.17 (m, 1H), 4.16-4.03 (m, 2H), 3.89 (s, 3H), 3.85-3.78 (m, 2H), 3.67 (t, J=6.0 Hz, 2H), 3.04-2.96 (m, 1H), 2.91-2.83 (m, 1H), 2.74-2.50 (m, 10H), 2.25-2.11 (m, 2H). MS (ESI+) m/z 638.40 (M+H)⁺.

### Example 46

### 6-amino-6′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide

The title compound was prepared as described in Example 19 substituting the product of Step 34A for the product of Step 8C. ¹H NMR (400 MHz, DMSO-d₆) δ 8.55 (d, J=2.6 Hz, 1H), 8.47 (d, J=2.3 Hz, 1H), 8.39 (d, J=7.8 Hz, 1H), 8.25 (td, J=8.2, 2.7 Hz, 1H), 8.16 (d, J=2.4 Hz, 1H), 7.59-7.52 (m, 2H), 7.45-7.35 (m, 4H), 7.33-7.20 (m, 4H), 4.60 (s, 2H), 4.38-4.23 (m, 3H), 3.96-3.89 (m, 1H), 3.47 (q, J=6.1 Hz, 2H), 2.96-2.74 (m, 2H), 2.55-2.46 (m, 2H), 2.45-2.32 (m, 7H), 2.12-1.90 (m, 4H), 1.79-1.52 (m, 4H). MS (ESI+) m/z 651.3 (M+H)⁺.

### Example 47

### 2-amino-5-(1,5-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide

### Step 47A

### 2-amino-5-(1,5-dimethyl-1H-pyrazol-4-yl)pyridine-3-carboxylic acid

The title compound was prepared as described in Intermediate 2 substituting 1,5-dimethyl-4-(4,4,5,5-tetramethyl-1,3,2-dioxaborolan-2-yl)-1H-pyrazole for 1-methyl-4-(4,4,5,5-tetramethyl-1,3,2-dioxaborolan-2-yl)-1H-pyrazole. MS (ESI+) m/z 233 (M+H)⁺.

### Step 47B

### 2-amino-N-{(3S,4R)-4-[(4-bromophenyl)methoxy]oxolan-3-yl}-5-(1,5-dimethyl-1H-pyrazol-4-yl)pyridine-3-carboxamide

The title compound was prepared as described in Intermediate 29 substituting the product of Step 47A for Intermediate 2. MS (ESI+) m/z 486 (M+H)⁺.

### Step 47C

### 2-amino-5-(1,5-dimethyl-1H-pyrazol-4-yl)-N-[(3S,4R)-4-{[4-(4,4,5,5-tetramethyl-1,3,2-dioxaborolan-2-yl)phenyl]methoxy}oxolan-3-yl]pyridine-3-carboxamide

The title compound was prepared as described in Intermediate 19B substituting the product of Step 47B for Intermediate 19A. MS (ESI+) m/z 534 (M+H)⁺.

### Step 47D

### 2-amino-5-(1,5-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide

The title compound was prepared as described in Step 4C substituting the product of Step 47C for Intermediate 33 and substituting Intermediate 13 for the product of Step 4B. ¹H NMR (400 MHz, DMSO-d₆) δ 8.54 (d, J=6.8 Hz, 1H), 8.07 (d, J=2.2 Hz, 1H), 7.90 (d, J=2.3 Hz, 1H), 7.48 (d, J=7.3 Hz, 2H), 7.34-7.28 (m, 2H), 7.28-7.18 (m, 2H), 7.18-7.05 (m, 1H), 6.97 (s, 2H), 6.49-6.44 (m, 1H), 4.70-4.51 (m, 2H), 4.49-4.39 (m, 1H), 4.33 (s, 1H), 4.08 (dt, J=4.3, 1.9 Hz, 1H), 4.00 (dd, J=9.3, 6.2 Hz, 1H), 3.89 (dd, J=10.0, 4.8 Hz, 1H), 3.76-3.63 (m, 4H), 3.50-3.42 (m, 3H), 3.11 (s, 2H), 2.99-2.86 (m, 2H), 2.37 (t, J=6.3 Hz, 2H), 2.27 (s, 3H), 2.05 (td, J=11.4, 3.1 Hz, 2H), 1.60 (qd, J=11.8, 3.6 Hz, 5H), 1.23 (d, J=4.3 Hz, 6H). MS (ESI+) m/z 680 (M+H)⁺.

### Example 49

### 6-amino-2′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide

### Step 49A

### 6-amino-N-{(3S,4R)-4-[(4-bromophenyl)methoxy]oxolan-3-yl}-2′-fluoro[3,3′-bipyridine]-5-carboxamide

The title compound was prepared as described in Intermediate 19A substituting Intermediate 28 for Intermediate 1. MS (ESI+) m/z 487 (M+H)⁺.

### Step 49B

### 6-amino-2′-fluoro-N-[(3S,4R)-4-{[4-(4,4,5,5-tetramethyl-1,3,2-dioxaborolan-2-yl)phenyl]methoxy}oxolan-3-yl][3,3′-bipyridine]-5-carboxamide

The title compound was prepared as described in Intermediate 19B, substituting the product of Step 49A for Intermediate 19A. MS (ESI+) m/z 535 (M+H)⁺.

### Step 49C

### 6-amino-2′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide

The title compound was prepared as described in Step 4C substituting the product of Step 49B for Intermediate 33 and substituting Intermediate 13 for the product of Step 4B. ¹H NMR (501 MHz, DMSO-d₆) δ 8.62 (d, J=6.9 Hz, 1H), 8.35 (t, J=2.3 Hz, 1H), 8.25-8.03 (m, 3H), 7.59-7.46 (m, 2H), 7.43 (ddd, J=7.0, 4.8, 1.9 Hz, 1H), 7.37-7.16 (m, 5H), 6.53-6.42 (m, 1H), 4.72-4.53 (m, 2H), 4.52-4.41 (m, 1H), 4.35 (d, J=5.4 Hz, 1H), 4.09 (dt, J=4.2, 1.9 Hz, 1H), 4.01 (dd, J=9.3, 6.1 Hz, 1H), 3.92 (dd, J=10.0, 4.9 Hz, 1H), 3.71 (ddd, J=13.0, 9.6, 2.7 Hz, 2H), 3.47 (dt, J=9.8, 4.8 Hz, 2H), 3.38 (s, 1H), 3.13 (s, 2H), 3.00-2.83 (m, 2H), 2.38 (t, J=6.4 Hz, 2H), 2.07 (td, J=11.5, 2.8 Hz, 2H), 1.60 (dtd, J=27.7, 12.1, 3.7 Hz, 4H), 1.25 (s, 6H). MS (ESI+) m/z 681 (M+H)⁺.

### Example 50

### 2-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 50A

### 5-bromo-2-fluoro-2,3-dihydro-1H-inden-1-one

To a solution of 5-bromo-1-indanone (200 mg, 0.948 mmol) in acetonitrile (4.7 mL) was added Na₂SO₄ (269 mg, 1.895 mmol) and 1-chloromethyl-4-fluoro-1,4-diazoniabicyclo[2.2.2]octane bis(tetrafluoroborate) (839 mg, 2.369 mmol), and the mixture was stirred at 80° C. for 16 hours. The mixture was diluted with ethyl acetate and water and extracted with dichloromethane. The organic layer was washed with water, dried over MgSO₄ and concentrated, and the residue purified by flash chromatography on silica gel eluting with 0-10% ethyl acetate/heptanes to afford the title compound (202 mg). ¹H NMR (400 MHz, CDCl₃) δ 7.66 (d, J=7.9 Hz, 2H), 7.58 (dt, J=7.4, 1.3 Hz, 1H), 5.25 (ddd, J=50.8, 7.7, 4.3 Hz, 1H), 3.68-3.52 (m, 1H), 3.22 (dddd, J=22.9, 17.2, 4.3, 1.2 Hz, 1H).

### Step 50B

### 5-bromo-2,2-difluoro-2,3-dihydro-1H-inden-1-one

To a solution of the product of Step 50A (202.2 mg, 0.883 mmol) in dichloromethane (5.9 mL) was added triethylamine (0.37 mL, 2.65 mmol) and tert-butyldimethylsilyl trifluoromethanesulfonate (350 mg, 1.32 mmol), and the mixture was stirred at 0° C. for 5 hours. The mixture was diluted with ethyl acetate and water and extracted with dichloromethane. The organic layer was washed with water, dried over MgSO₄, filtered, and concentrated. The crude product was dissolved in acetonitrile (5.9 mL) and 1-chloromethyl-4-fluoro-1,4-diazoniabicyclo[2.2.2]octane bis(tetrafluoroborate) (344 mg, 0.97 mmol) was added, and the mixture was stirred at ambient temperature overnight. The mixture was concentrated, diluted with dichloromethane, filtered, and concentrated. The residue was purified by flash chromatography on silica gel eluting with 0-10% ethyl acetate/heptanes to afford the title compound (154 mg). ¹H NMR (400 MHz, CDCl₃) δ 7.73 (d, J=8.2 Hz, 1H), 7.70-7.60 (m, 2H), 3.55 (td, J=12.5, 1.1 Hz, 2H).

### Step 50C

### tert-butyl 4-(5-bromo-2,2-difluoro-2,3-dihydro-1H-inden-1-yl)piperazine-1-carboxylate

A mixture of the product of Step 50B (150 mg, 0.607 mmol), triethylamine (0.25 mL, 1.82 mmol), 1-boc-piperazine (226 mg, 1.214 mmol) and titanium(IV) chloride (0.3 mL, 0.304 mmol) in dichloromethane (4.6 mL) was stirred at ambient temperature for 16 hours. A solution of sodium cyanoborohydride (153 mg, 2.429 mmol) in methanol (1.5 mL) was added, and the mixture was stirred at ambient temperature for 16 hours. The mixture was quenched with saturated NaHCO₃ and ethyl acetate. The insoluble material was filtered off and the organic layer was concentrated. The residue was purified by flash chromatography on silica gel eluting with 0-50% ethyl acetate/heptanes to afford the title compound (218 mg). LCMS (APCI+) m/z 417.20 (M+H)⁺.

### Step 50D

### 1-(5-bromo-2,2-difluoro-2,3-dihydro-1H-inden-1-yl)piperazine

To a solution of the product of Step 50C (0.26 g, 0.622 mmol) in dichloromethane (3.11 mL) at 0° C. was added 4 M HCl in dioxane (1.56 mL, 6.22 mmol), and the mixture was stirred at ambient temperature for 18 hours. The mixture was concentrated to afford the crude title compound which was used without further purification.

### Step 50E

### 2-[4-(5-bromo-2,2-difluoro-2,3-dihydro-1H-inden-1-yl)piperazin-1-yl]ethan-1-ol

To a solution of the product of Step 50D (243 mg, 0.62 mmol) in acetonitrile (3.1 mL) was added 2-bromoethanol (0.11 mL, 1.56 mmol), followed by K₂CO₃ (430 mg, 3.11 mmol), and the mixture was stirred at 70° C. for 18 hours. The solution was directly purified by flash chromatography on silica gel eluting with 0-10% methanol/dichloromethane to provide the title compound (18 mg). LCMS (APCI+) m/z 361.05 (M+H)⁺.

### Step 50F

### 2-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

A mixture of Intermediate 33 (25 mg, 0.048 mmol), the product of Step 50E (17.5 mg, 0.048 mmol), bis(di-tert-butyl(4-dimethylaminophenyl)phosphine)dichloropalladium(II) (1.71 mg, 2.416 μmol) and K₂CO₃ (20 mg, 0.145 mmol) in dioxane (0.24 mL) and water (0.08 mL) was sparged with nitrogen and heated at 100° C. for 2 hours. The mixture was cooled to ambient temperature and directly purified by flash chromatography on silica gel eluting with 0-10% methanol/dichloromethane with 3% NH₄OH. The product was further purified by reverse phase HPLC (C18 column) eluting with a gradient of 10-75% acetonitrile/water with 0.1% trifluoroacetic acid to afford the title compound (22 mg). ¹H NMR (400 MHz, methanol-d₄) δ 8.48 (dd, J=2.2, 1.4 Hz, 1H), 8.24 (d, J=2.1 Hz, 1H), 7.99 (d, J=4.4 Hz, 1H), 7.91-7.83 (m, 1H), 7.63-7.40 (m, 7H), 4.77-4.57 (m, 3H), 4.51-4.41 (m, 1H), 4.05 (dt, J=6.5, 4.3 Hz, 1H), 3.97-3.87 (m, 5H), 3.65 (d, J=12.0 Hz, 1H), 3.57 (d, J=8.5 Hz, 1H), 3.46 (dd, J=15.9, 13.2 Hz, 2H), 3.34-2.99 (m, 7H), 2.21 (dt, J=14.2, 6.9 Hz, 1H), 2.08 (ddd, J=12.4, 7.8, 5.7 Hz, 1H), 1.95-1.76 (m, 3H), 1.76-1.60 (m, 1H). MS (ESI+) m/z 672.2 (M+H)⁺.

### Example 51

### 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(prop-1-en-2-yl)pyridine-3-carboxamide

### Step 51A

### 2-amino-5-(prop-1-en-2-yl)pyridine-3-carboxylic acid

The title compound was prepared as described in Intermediate 2 substituting 4,4,5,5-tetramethyl-2-(prop-1-en-2-yl)-1,3,2-dioxaborolane for 1-methyl-4-(4,4,5,5-tetramethyl-1,3,2-dioxaborolan-2-yl)-1H-pyrazole. MS (ESI+) m/z 179 (M+H)⁺.

### Step 51B

### 2-amino-N-{(1S,2S)-2-[(4-bromophenyl)methoxy]cyclopentyl}-5-(prop-1-en-2-yl)pyridine-3-carboxamide

The title compound was prepared as described in Intermediate 3, substituting the product of Step 51A for Intermediate 2. MS (ESI+) m/z 430 (M+H)⁺.

### Step 51C

### 2-amino-5-(prop-1-en-2-yl)-N-[(1S,2S)-2-{[4-(4,4,5,5-tetramethyl-1,3,2-dioxaborolan-2-yl)phenyl]methoxy}cyclopentyl]pyridine-3-carboxamide

The title compound was prepared as described in Intermediate 19B substituting the product of Step 51B for Intermediate 19A. MS (ESI+) m/z 478 (M+H)⁺.

### Step 51D

### 2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(prop-1-en-2-yl)pyridine-3-carboxamide

The title compound was prepared as described in Step 4C substituting the product of Step 51C for Intermediate 33 and substituting Intermediate 13 for the product of Step 4B. ¹H NMR (400 MHz, DMSO-d₆) δ 8.38 (d, J=7.8 Hz, 1H), 8.20 (d, J=2.3 Hz, 1H), 7.92 (d, J=2.4 Hz, 1H), 7.44 (d, J=8.2 Hz, 2H), 7.36-7.15 (m, 4H), 7.04 (s, 2H), 6.46 (d, J=8.2 Hz, 1H), 5.32 (s, 1H), 4.91 (t, J=1.6 Hz, 1H), 4.62-4.41 (m, 2H), 4.43-4.12 (m, 2H), 3.87 (dt, J=6.3, 3.9 Hz, 1H), 3.52-3.43 (m, 2H), 3.11 (s, 2H), 2.97-2.72 (m, 2H), 2.38 (td, J=6.3, 3.7 Hz, 2H), 2.16-1.79 (m, 7H), 1.75-1.34 (m, 9H), 1.20 (d, J=24.4 Hz, 6H). MS (ESI+) m/z 624 (M+H)⁺.

### Example 52

### 3-amino-6-(6-fluoropyridin-3-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyrazine-2-carboxamide

### Step 52A

### 3-amino-N-{(3S,4R)-4-[(4-bromophenyl)methoxy]oxolan-3-yl}-6-(6-fluoropyridin-3-yl)pyrazine-2-carboxamide

The title compound was prepared as described in Step 41C using Intermediate 28 in place of Intermediate 1. MS (ESI+) m/z 487.9 (M+H)⁺.

### Step 52B

### 3-amino-6-(6-fluoropyridin-3-yl)-N-[(3S,4R)-4-{[4-(4,4,5,5-tetramethyl-1,3,2-dioxaborolan-2-yl)phenyl]methoxy}oxolan-3-yl]pyrazine-2-carboxamide

The title compound was prepared as described in Step 41D using the product of Step 52A in place of the product of Step 41C. MS (ESI+) m/z 536.4 (M+H)⁺.

### Step 52C

### 3-amino-6-(6-fluoropyridin-3-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyrazine-2-carboxamide

The title compound was prepared as described in Step 41E using the product of Step 52B in place of the product of Step 41D. ¹H NMR (400 MHz, methanol-d₄) δ 8.69 (d, J=2.5 Hz, 1H), 8.59 (s, 1H), 8.41 (td, J=8.1, 2.6 Hz, 1H), 7.39-7.31 (m, 2H), 7.26 (d, J=8.2 Hz, 2H), 7.15-7.05 (m, 2H), 6.99 (dd, J=8.6, 2.6 Hz, 1H), 6.39 (d, J=8.2 Hz, 1H), 4.65 (d, J=12.1 Hz, 1H), 4.57 (d, J=12.0 Hz, 1H), 4.50 (td, J=4.1, 2.1 Hz, 1H), 4.16 (dt, J=5.3, 2.6 Hz, 1H), 4.02 (ddd, J=13.7, 9.7, 5.8 Hz, 2H), 3.71 (ddd, J=9.3, 6.0, 3.5 Hz, 2H), 3.61 (t, J=6.1 Hz, 2H), 3.10 (s, 2H), 3.02 (d, J=11.5 Hz, 2H), 2.50 (t, J=6.1 Hz, 2H), 2.17 (s, 2H), 1.84 (s, 1H), 1.69 (td, J=10.8, 9.5, 3.3 Hz, 4H), 1.20 (s, 6H). MS (ESI+) m/z 682.2 (M+H)⁺.

### Example 53

### 2-amino-5-(1-methyl-1H-pyrazol-4-yl)-N-[(1S,2S)-2-({4-[2-(trifluoromethyl)-1H-indol-5-yl]phenyl}methoxy)cyclopentyl]pyridine-3-carboxamide

The title compound was prepared as described in Step 4C substituting 5-bromo-2-(trifluoromethyl)-1H-indole for the product of Step 4B. ¹H NMR (400 MHz, DMSO-d₆) δ 12.26 (s, 1H), 8.37-8.23 (m, 2H), 7.97 (dd, J=7.4, 1.6 Hz, 2H), 7.86 (t, J=1.2 Hz, 1H), 7.75 (d, J=0.8 Hz, 1H), 7.61-7.44 (m, 4H), 7.41-7.29 (m, 2H), 6.98 (d, J=32.4 Hz, 3H), 4.58 (d, J=2.9 Hz, 2H), 4.29 (dt, J=11.4, 7.3 Hz, 1H), 3.90 (q, J=3.2, 2.5 Hz, 1H), 3.79 (s, 3H), 2.10-1.85 (m, 2H), 1.79-1.46 (m, 4H). MS (ESI+) m/z 575 (M+H)⁺.

### Example 54

### 2-amino-N-[(1S,2S)-2-{[4-(1,3-dimethyl-1H-indol-5-yl)phenyl]methoxy}cyclopentyl]-5-(1-methyl-1H-pyrazol-4-yl)pyridine-3-carboxamide

The title compound was prepared as described in Step 4C, substituting 5-bromo-1,3-dimethyl-1H-indole for the product of Step 4B. ¹H NMR (400 MHz, DMSO-d₆) δ 8.42-8.14 (m, 2H), 7.96 (dd, J=10.8, 1.5 Hz, 2H), 7.75 (d, J=0.8 Hz, 1H), 7.67 (dd, J=1.6, 0.8 Hz, 1H), 7.64-7.52 (m, 2H), 7.47-7.24 (m, 4H), 7.06 (d, J=1.1 Hz, 1H), 6.94 (s, 2H), 4.66-4.48 (m, 2H), 4.29 (dt, J=11.5, 7.4 Hz, 1H), 3.90 (dt, J=6.2, 3.7 Hz, 1H), 3.79 (s, 3H), 3.70 (s, 3H), 2.24 (d, J=1.1 Hz, 3H), 2.09-1.84 (m, 2H), 1.77-1.45 (m, 4H). MS (ESI+) m/z 535 (M+H)⁺.

### Example 55

### 2-amino-N-[(1S,2S)-2-{[4-(2,3-dimethyl-1H-indol-5-yl)phenyl]methoxy}cyclopentyl]-5-(1-methyl-1H-pyrazol-4-yl)pyridine-3-carboxamide

The title compound was prepared as described in Step 4C substituting 5-bromo-2,3-dimethyl-1H-indole for the product of Step 4B. ¹H NMR (400 MHz, DMSO-d₆) δ 10.65 (s, 1H), 8.37-8.18 (m, 2H), 7.96 (dd, J=11.3, 1.5 Hz, 2H), 7.75 (d, J=0.8 Hz, 1H), 7.63-7.47 (m, 2H), 7.38-7.30 (m, 2H), 7.28-7.14 (m, 2H), 6.93 (s, 2H), 5.72 (s, 1H), 4.65-4.48 (m, 2H), 4.29 (p, J=7.3 Hz, 1H), 3.90 (dt, J=5.9, 3.7 Hz, 1H), 3.79 (s, 3H), 2.28 (s, 3H), 2.22-2.07 (m, 3H), 2.09-1.82 (m, 2H), 1.84-1.43 (m, 4H). MS (ESI+) m/z 535 (M+H)⁺.

### Example 56

### 2-amino-N-[(1S,2S)-2-{[4-(2-methyl-1H-indol-5-yl)phenyl]methoxy}cyclopentyl]-5-(1-methyl-1H-pyrazol-4-yl)pyridine-3-carboxamide

The title compound was prepared as described in Step 4C substituting 5-bromo-2-methyl-1H-indole for the product of Step 4B. ¹H NMR (400 MHz, DMSO-d₆) δ 10.90 (d, J=2.0 Hz, 1H), 8.36-8.21 (m, 2H), 8.03-7.89 (m, 2H), 7.76 (d, J=0.8 Hz, 1H), 7.65-7.47 (m, 3H), 7.37-7.17 (m, 4H), 6.94 (s, 2H), 6.12 (dt, J=1.9, 1.0 Hz, 1H), 4.65-4.48 (m, 2H), 4.29 (qd, J=7.6, 3.8 Hz, 1H), 3.90 (dt, J=6.2, 3.8 Hz, 1H), 3.80 (s, 3H), 2.35 (d, J=1.0 Hz, 3H), 2.05-1.86 (m, 2H), 1.73-1.50 (m, 4H). MS (ESI+) m/z 521 (M+H)⁺.

### Example 57

### 2-amino-N-[(1S,2S)-2-({4-[1-(2-hydroxyethyl)-1H-indol-5-yl]phenyl}methoxy)cyclopentyl]-5-(1-methyl-1H-pyrazol-4-yl)pyridine-3-carboxamide

The title compound was prepared as described in Step 4C substituting 2-(5-bromo-1H-indol-1-yl)ethanol for the product of Step 4B. ¹H NMR (400 MHz, DMSO-d₆) δ 8.35-8.24 (m, 2H), 8.01-7.89 (m, 2H), 7.74 (dd, J=12.6, 1.3 Hz, 2H), 7.62-7.53 (m, 2H), 7.49 (d, J=8.6 Hz, 1H), 7.39-7.27 (m, 4H), 6.94 (s, 2H), 6.43 (dd, J=3.1, 0.8 Hz, 1H), 4.85 (t, J=5.3 Hz, 1H), 4.66-4.48 (m, 2H), 4.29 (qd, J=7.5, 3.8 Hz, 1H), 4.20 (t, J=5.6 Hz, 2H), 3.90 (dt, J=6.0, 3.7 Hz, 1H), 3.79 (s, 3H), 3.70 (q, J=5.5 Hz, 2H), 2.13-1.85 (m, 2H), 1.81-1.51 (m, 4H). MS (ESI+) m/z 551 (M+H)⁺.

### Example 58

### 3-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}propanoic acid

The title compound was prepared as described in Step 4C substituting 3-(5-bromo-1H-indol-1-yl)propanoic acid for the product of Step 4B. ¹H NMR (501 MHz, DMSO-d₆) δ 8.36-8.26 (m, 2H), 8.00 (d, J=2.3 Hz, 1H), 7.97 (s, 1H), 7.77 (d, J=0.8 Hz, 1H), 7.74 (d, J=1.7 Hz, 1H), 7.62-7.55 (m, 2H), 7.53 (d, J=8.6 Hz, 1H), 7.40-7.26 (m, 4H), 6.96 (s, 2H), 6.45 (dd, J=3.2, 0.8 Hz, 1H), 4.69-4.51 (m, 2H), 4.40 (t, J=6.8 Hz, 2H), 4.31 (qd, J=7.6, 3.9 Hz, 1H), 3.92 (dt, J=6.3, 3.8 Hz, 1H), 3.81 (s, 3H), 2.74 (t, J=6.8 Hz, 2H), 2.08-1.87 (m, 2H), 1.78-1.52 (m, 4H). MS (ESI+) m/z 579 (M+H)⁺.

### Example 59

### 2-amino-N-{(1S,2S)-2-[(4-{1-[4-(2,3-dihydroxypropyl)piperazin-1-yl]-2,2-difluoro-2,3-dihydro-1H-inden-5-yl}phenyl)methoxy]cyclopentyl}-5-(1-methyl-1H-pyrazol-4-yl)pyridine-3-carboxamide

### Step 59A

### 1-(5-bromo-2,2-difluoro-2,3-dihydro-1H-inden-1-yl)-4-[(2,2-dimethyl-1,3-dioxolan-4-yl)methyl]piperazine

To a solution of the product of Step 50D (388 mg, 1.097 mmol) in 1,2-dichloroethane (7.3 mL) and methanol (3.7 mL) was added (4R)-2,2-dimethyl-1,3-dioxolane-4-carboxaldehyde (571 mg, 2.19 mmol), and the mixture was stirred for at ambient temperature for 15 minutes. Sodium triacetoxyborohydride (698 mg, 3.29 mmol) was added, and the mixture was stirred at ambient temperature for 16 hours. Additional (4R)-2,2-dimethyl-1,3-dioxolane-4-carboxaldehyde (571 mg, 2.19 mmol) was added, followed by sodium triacetoxyborohydride (698 mg, 3.29 mmol), and the mixture was stirred for 16 hours. The reaction was quenched with water and extracted with dichloromethane. The organic extract was concentrated and purified by flash chromatography on silica gel eluting with 0-10% methanol/dichloromethane to afford the title compound as a racemic mixture (471 mg). LCMS (APCI+) m/z 431.00 (M+H)⁺.

### Step 59B

### 3-[4-(5-bromo-2,2-difluoro-2,3-dihydro-1H-inden-1-yl)piperazin-1-yl]propane-1,2-diol

To a solution of the product of Step 59A (0.473 g, 1.097 mmol) in tetrahydrofuran (7.31 mL) was added 2 M HCl (10.97 mL, 21.94 mmol), and the mixture was stirred at ambient temperature for 18 hours. The mixture was concentrated to afford the title compound which was used without further purification. LCMS (ESI+) m/z 391.00 (M+H)⁺.

### Step 59C

### 2-amino-N-{(1S,2S)-2-[(4-{1-[4-(2,3-dihydroxypropyl)piperazin-1-yl]-2,2-difluoro-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 59B in place of the product of Step 50E. ¹H NMR (501 MHz, methanol-d₄) δ 8.50 (dd, J=2.2, 1.2 Hz, 1H), 8.21 (d, J=2.1 Hz, 1H), 7.99 (d, J=5.1 Hz, 1H), 7.86 (d, J=2.2 Hz, 1H), 7.60-7.52 (m, 3H), 7.50 (d, J=1.6 Hz, 1H), 7.48-7.38 (m, 3H), 4.75-4.65 (m, 2H), 4.62 (t, J=11.8 Hz, 1H), 4.44 (td, J=7.3, 4.4 Hz, 1H), 4.12-4.00 (m, 2H), 3.93 (d, J=2.2 Hz, 3H), 3.73-3.62 (m, 1H), 3.63-3.51 (m, 3H), 3.44 (dd, J=15.8, 12.9 Hz, 3H), 3.32-2.97 (m, 7H), 2.27-2.14 (m, 1H), 2.14-2.02 (m, 1H), 1.83 (dddd, J=22.7, 12.7, 6.7, 2.9 Hz, 3H), 1.67 (dq, J=13.2, 7.5 Hz, 1H). MS (ESI+) m/z 702.5 (M+H)⁺.

### Example 60

### 2-amino-5-(1-methyl-1H-pyrazol-4-yl)-N-[(1S,2S)-2-({4-[1-(propan-2-yl)-1H-indol-5-yl]phenyl}methoxy)cyclopentyl]pyridine-3-carboxamide

The title compound was prepared as described in Step 4C substituting 5-bromo-1-isopropyl-1H-indole for the product of Step 4B. ¹H NMR (400 MHz, DMSO-d₆) δ 8.36-8.25 (m, 2H), 8.02-7.87 (m, 2H), 7.74 (dd, J=11.6, 1.3 Hz, 2H), 7.59-7.42 (m, 4H), 7.38-7.30 (m, 3H), 6.94 (s, 2H), 6.46 (d, J=3.2 Hz, 1H), 4.73 (hept, J=6.4 Hz, 1H), 4.61-4.51 (m, 2H), 4.32-4.25 (m, 1H), 3.98-3.89 (m, 1H), 3.79 (s, 3H), 2.05-1.88 (m, 2H), 1.74-1.52 (m, 4H), 1.44 (d, J=6.6 Hz, 6H). MS (ESI+) m/z 549 (M+H)⁺.

### Example 61

### {5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}acetic acid

The title compound was prepared as described in Step 4C, substituting 2-(5-bromo-1H-indol-1-yl)acetic acid for the product of Step 4B. ¹H NMR (400 MHz, DMSO-d₆) δ 8.44-8.23 (m, 2H), 8.05-7.88 (m, 2H), 7.84-7.68 (m, 2H), 7.64-7.47 (m, 2H), 7.46-7.16 (m, 5H), 6.94 (s, 2H), 6.44 (d, J=3.2 Hz, 1H), 4.93 (s, 2H), 4.74-4.44 (m, 2H), 4.30 (pd, J=7.1, 2.9 Hz, 1H), 3.90 (dt, J=6.2, 3.8 Hz, 1H), 3.79 (s, 3H), 2.13-1.87 (m, 2H), 1.78-1.37 (m, 4H). MS (ESI+) m/z 565 (M+H)⁺.

### Example 62

### 6-amino-N-{(1S,2S)-2-[(4-{1-[4-(2,3-dihydroxypropyl)piperazin-1-yl]-2,2-difluoro-2,3-dihydro-1H-inden-5-yl}phenyl)methoxy]cyclopentyl}-6′-fluoro[3,3′-bipyridine]-5-carboxamide

The title compound was prepared as described in Step 50F using the product of Step 59B in place of the product of Step 50E and Intermediate 32 in place of Intermediate 33. ¹H NMR (501 MHz, methanol-d₄) δ 8.46 (dd, J=8.1, 2.7 Hz, 1H), 8.40 (dt, J=4.0, 2.1 Hz, 1H), 8.37 (d, J=2.3 Hz, 1H), 8.18 (dddd, J=8.7, 7.4, 2.6, 1.4 Hz, 1H), 7.57-7.51 (m, 3H), 7.51-7.39 (m, 4H), 7.16 (dt, J=8.5, 2.2 Hz, 1H), 4.77-4.59 (m, 3H), 4.46 (td, J=7.4, 4.6 Hz, 1H), 4.08 (ddt, J=14.2, 11.0, 4.8 Hz, 1H), 4.02 (dt, J=6.7, 4.6 Hz, 1H), 3.74-3.53 (m, 4H), 3.52-3.36 (m, 4H), 3.33-3.00 (m, 6H), 2.21 (dq, J=13.5, 7.2 Hz, 1H), 2.09 (dq, J=12.6, 7.1 Hz, 1H), 1.94-1.76 (m, 3H), 1.65 (dq, J=14.8, 7.5 Hz, 1H). MS (ESI+) m/z 717.5 (M+H)⁺.

### Example 63

### 6-amino-N-{(1S,2S)-2-[(4-{1-[4-(2,3-dihydroxypropyl)piperazin-1-yl]-2,2-difluoro-2,3-dihydro-1H-inden-5-yl}phenyl)methoxy]cyclopentyl}-1′-methyl-2′-oxo-1′,2′-dihydro[3,4′-bipyridine]-5-carboxamide

The title compound was prepared as described in Step 50F using the product of Step 59B in place of the product of Step 50E and the product of Step 30C in place of Intermediate 33. ¹H NMR (400 MHz, methanol-d₄) δ 8.47-8.37 (m, 2H), 7.73 (dd, J=7.1, 5.9 Hz, 1H), 7.60-7.38 (m, 7H), 6.82 (d, J=2.0 Hz, 1H), 6.70 (td, J=6.9, 2.1 Hz, 1H), 4.76-4.60 (m, 3H), 4.45 (td, J=7.4, 4.6 Hz, 1H), 4.13-3.98 (m, 2H), 3.73-3.53 (m, 7H), 3.52-3.37 (m, 3H), 3.30 (t, J=2.7 Hz, 1H), 3.28-2.99 (m, 5H), 2.28-2.14 (m, 1H), 2.14-2.03 (m, 1H), 1.95-1.75 (m, 3H), 1.66 (dq, J=14.7, 7.5 Hz, 1H). MS (ESI+) m/z 729.4 (M+H)⁺.

### Example 64

### 2-amino-N-[(1S,2S)-2-({4-[1-(difluoromethyl)-1H-indol-5-yl]phenyl}methoxy)cyclopentyl]-5-(1-methyl-1H-pyrazol-4-yl)pyridine-3-carboxamide

The title compound was prepared as described in Step 4C, substituting 5-bromo-1-(difluoromethyl)-1H-indole for the product of Step 4B. ¹H NMR (501 MHz, DMSO-d₆) δ 8.38-8.26 (m, 2H), 8.02-7.95 (m, 2H), 7.85 (d, J=1.7 Hz, 1H), 7.78-7.70 (m, 2H), 7.66-7.58 (m, 3H), 7.53 (dd, J=8.5, 1.8 Hz, 1H), 7.42-7.35 (m, 2H), 6.96 (s, 2H), 6.72 (dd, J=3.5, 0.8 Hz, 1H), 4.66-4.55 (m, 2H), 4.31 (qd, J=7.6, 4.0 Hz, 1H), 3.81 (s, 3H), 3.15 (d, J=5.1 Hz, 1H), 2.10-1.90 (m, 2H), 1.78-1.52 (m, 4H). MS (ESI+) m/z 557 (M+H)⁺.

### Example 65

### 2-amino-N-{(1S,2S)-2-[(4-{(1R)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 65A

### 2-{4-[(1R)-5-bromo-2,2-difluoro-2,3-dihydro-1H-inden-1-yl]piperazin-1-yl}ethan-1-ol

The product of Step 50E was subjected to chiral super critical fluid chromatography separation on an YMC Amylose-C column (20×250 mm ID) to afford the title compound (31 mg) as the first eluting peak. LCMS (APCI+) m/z 361.04 (M+H)⁺.

### Step 65B

### 2-amino-N-{(1S,2S)-2-[(4-{(1R)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 65A in place of the product of Step 50E. ¹H NMR (501 MHz, methanol-d₄) δ 8.51 (d, J=2.1 Hz, 1H), 8.21 (d, J=2.1 Hz, 1H), 8.00 (s, 1H), 7.86 (d, J=0.8 Hz, 1H), 7.60-7.52 (m, 3H), 7.50 (d, J=1.7 Hz, 1H), 7.44 (dd, J=15.0, 8.1 Hz, 3H), 4.75-4.65 (m, 2H), 4.62 (dd, J=12.9, 10.7 Hz, 1H), 4.44 (ddd, J=8.2, 6.8, 4.4 Hz, 1H), 4.04 (dt, J=6.5, 4.4 Hz, 1H), 3.96-3.86 (m, 5H), 3.64 (d, J=12.0 Hz, 1H), 3.56 (d, J=8.8 Hz, 1H), 3.41 (ddd, J=26.7, 15.8, 8.7 Hz, 3H), 3.31 (td, J=4.7, 1.3 Hz, 3H), 3.29-3.18 (m, 1H), 3.09 (dd, J=22.4, 10.8 Hz, 3H), 2.26-2.15 (m, 1H), 2.07 (ddd, J=15.0, 7.7, 5.8 Hz, 1H), 1.92-1.75 (m, 3H), 1.72-1.61 (m, 1H). MS (ESI+) m/z 672.3 (M+H)⁺.

### Example 66

### 2-amino-N-{(1S,2S)-2-[(4-{(1S)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 66A

### 2-{4-[(1S)-5-bromo-2,2-difluoro-2,3-dihydro-1H-inden-1-yl]piperazin-1-yl}ethan-1-ol

The title compound (30 mg) was obtained from the super critical fluid chromatography purification described in Step 65A as the second eluting peak. LCMS (APCI+) m/z 361.04 (M+H)⁺.

### Step 66B

### 2-amino-N-{(1S,2S)-2-[(4-{(1S)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 66A in place of the product of Step 50E. ¹H NMR (400 MHz, methanol-d₄) δ 8.50 (d, J=2.1 Hz, 1H), 8.21 (d, J=2.1 Hz, 1H), 7.99 (s, 1H), 7.86 (d, J=0.9 Hz, 1H), 7.56 (td, J=8.0, 1.8 Hz, 3H), 7.49 (d, J=1.6 Hz, 1H), 7.44 (dd, J=13.0, 8.1 Hz, 3H), 4.75-4.57 (m, 3H), 4.49-4.40 (m, 1H), 4.04 (dt, J=6.4, 4.3 Hz, 1H), 3.97-3.87 (m, 5H), 3.64 (d, J=12.0 Hz, 1H), 3.56 (d, J=8.2 Hz, 1H), 3.44 (dd, J=16.0, 13.2 Hz, 4H), 3.33-3.18 (m, 5H), 3.18-2.99 (m, 3H), 2.28-2.14 (m, 1H), 2.07 (ddd, J=14.8, 7.6, 5.5 Hz, 1H), 1.94-1.76 (m, 3H), 1.67 (dq, J=14.8, 7.5 Hz, 1H). MS (ESI+) m/z 672.4 (M+H)⁺.

### Example 67

### 2-amino-N-[(1S,2S)-2-({4-[1-(2-aminoethyl)-1H-indol-5-yl]phenyl}methoxy)cyclopentyl]-5-(1-methyl-1H-pyrazol-4-yl)pyridine-3-carboxamide

The title compound was prepared as described in Step 4C, substituting 2-(5-chloro-1H-indol-1-yl)ethanamine for the product of Step 4B. ¹H NMR (501 MHz, DMSO-d₆) δ 8.44-8.17 (m, 2H), 8.11-7.91 (m, 2H), 7.85-7.71 (m, 2H), 7.63-7.47 (m, 3H), 7.42-7.28 (m, 4H), 6.96 (s, 2H), 6.45 (dd, J=3.2, 0.8 Hz, 1H), 4.71-4.50 (m, 2H), 4.31 (qd, J=7.6, 3.9 Hz, 1H), 4.14 (t, J=6.5 Hz, 2H), 3.92 (dt, J=6.4, 3.8 Hz, 1H), 3.81 (s, 3H), 2.88 (t, J=6.5 Hz, 2H), 2.09-1.88 (m, 2H), 1.77-1.53 (m, 4H). MS (ESI+) m/z 550 (M+H)⁺.

### Example 68

### 2-amino-N-[(1S,2S)-2-{[4-(1H-indol-5-yl)phenyl]methoxy}cyclopentyl]-5-(1-methyl-1H-pyrazol-4-yl)pyridine-3-carboxamide

The title compound was described in Step 4C substituting 5-bromo-1H-indole for the product of Step 4B. ¹H NMR (400 MHz, DMSO-d₆) δ 11.08 (s, 1H), 8.40-8.19 (m, 2H), 8.05-7.88 (m, 2H), 7.82-7.69 (m, 2H), 7.61-7.50 (m, 2H), 7.47-7.23 (m, 5H), 6.94 (s, 2H), 6.44 (ddd, J=3.0, 2.0, 0.9 Hz, 1H), 4.67-4.50 (m, 2H), 4.29 (qd, J=7.7, 4.0 Hz, 1H), 3.91 (q, J=3.3, 2.5 Hz, 1H), 3.79 (s, 3H), 2.07-1.84 (m, 2H), 1.77-1.50 (m, 4H). MS (ESI+) m/z 507 (M+H)⁺.

### Example 69

### 2-amino-N-{(1S,2S)-2-[(4-{3-[4-(2-hydroxyethyl)piperazin-1-yl]-2-oxo-2,3-dihydro-1H-indol-6-yl}phenyl)methoxy]cyclopentyl}-5-(1-methyl-1H-pyrazol-4-yl)pyridine-3-carboxamide

### Step 69A

### 6-bromo-3-[4-(2-hydroxyethyl)piperazin-1-yl]-1,3-dihydro-2H-indol-2-one

To a mixture of 6-bromoisatin (500 mg, 2.21 mmol) and N-(2-hydroxyethyl)piperazine (374 mg, 2.88 mmol) in anhydrous dioxane (8 mL) was added titanium(IV) isopropoxide (1.31 mL). The mixture was heated at 80° C. for 2 hours and then cooled to ambient temperature. NaCNBH₃ (278 mg, 4.42 mmol) was added, and the mixture was heated at 60° C. overnight. After cooling, the mixture was stirred with ethyl acetate and aqueous NaOH. The solid was filtered through diatomaceous earth and the organic phase was washed with brine and concentrated. The residue was purified by flash chromatography on silica gel using 0-20% (3% NH₄OH in methanol)/dichloromethane to provide the title compound (92 mg). MS (ESI+) m/z 340, 342 (M+H)⁺.

### Step 69B

### 2-amino-N-{(1S,2S)-2-[(4-{3-[4-(2-hydroxyethyl)piperazin-1-yl]-2-oxo-2,3-dihydro-1H-indol-6-yl}phenyl)methoxy]cyclopentyl}-5-(1-methyl-1H-pyrazol-4-yl)pyridine-3-carboxamide

A mixture of Intermediate 33 (129 mg, 0.25 mmol), the product of Step 69A (85 mg, 0.25 mmol), bis(di-tert-butyl(4-dimethylaminophenyl) phosphine)dichloropalladium(II) (9 mg, 0.012 mmol), and potassium phosphate tribasic (318 mg, 1.50 mmol) was purged with nitrogen. Dioxane (6 mL) and water (1.87 mL) were added, and the mixture was purged with nitrogen and heated at 95° C. for 3 hours. After cooling, the mixture was partitioned between ethyl acetate and brine. The organic phase was concentrated, and the residue was purified by flash chromatography on silica gel using 0-20% (3% NH₄OH in methanol)/dichloromethane to provide the title compound (24 mg). ¹H NMR (400 MHz, DMSO-d₆) δ 10.55 (s, 1H), 9.41 (s, 1H), 8.52 (d, J=7.6 Hz, 1H), 8.34 (d, J=2.2 Hz, 1H), 8.18 (d, J=2.2 Hz, 1H), 8.05 (s, 1H), 7.83 (s, 1H), 7.56 (d, J=8.1 Hz, 2H), 7.41 (d, J=8.1 Hz, 2H), 7.32 (d, J=7.8 Hz, 1H), 7.25 (dt, J=7.6, 2.1 Hz, 1H), 7.01 (s, 1H), 4.67-4.56 (m, 2H), 4.54 (s, 1H), 4.31 (qd, J=7.2, 3.6 Hz, 1H), 3.95 (dt, J=6.4, 3.7 Hz, 1H), 3.85 (s, 3H), 3.72 (t, J=5.3 Hz, 2H), 3.52 (s, 7H), 2.42-1.53 (m, 6H). MS (ESI+) m/z 651 (M+H)⁺.

### Example 70

### 2-amino-N-{(1S,2S)-2-[(4-{1-[2-(dimethylamino)ethyl]-1H-indol-5-yl}phenyl)methoxy]cyclopentyl}-5-(1-methyl-1H-pyrazol-4-yl)pyridine-3-carboxamide

### Step 70A

### 2-(5-chloro-1H-indol-1-yl)-N,N-dimethylethan-1-amine

A mixture of 2-(5-chloro-1H-indol-1-yl)ethanamine (50 mg, 0.257 mmol) and formaldehyde (77 mg, 1.284 mmol) in dichloromethane (1 mL) was stirred at ambient temperature for 30 minutes and treated with sodium triacetoxyhydroborate (109 mg, 0.514 mmol). After stirring at ambient temperature overnight, water was added, and the mixture was extracted with ethyl acetate and purified by flash chromatography on silica gel eluting with 0-10% methanol/dichloromethane to afford the title compound (40 mg). MS (APCI) m/z 223 (M+H)⁺.

### Step 70B

### 2-amino-N-{(1S,2S)-2-[(4-{1-[2-(dimethylamino)ethyl]-1H-indol-5-yl}phenyl)methoxy]cyclopentyl}-5-(1-methyl-1H-pyrazol-4-yl)pyridine-3-carboxamide

The title compound was prepared as described in Step 4C substituting the product of Step 70A for the product of Step 4B. ¹H NMR (400 MHz, DMSO-d₆) δ 8.42-8.26 (m, 2H), 8.07-7.89 (m, 2H), 7.86-7.68 (m, 2H), 7.59-7.45 (m, 3H), 7.43-7.24 (m, 4H), 6.95 (s, 2H), 6.43 (d, J=3.1 Hz, 1H), 4.64-4.50 (m, 2H), 4.35-4.16 (m, 3H), 3.93 (ddt, J=13.9, 6.2, 3.3 Hz, 1H), 3.78 (d, J=4.5 Hz, 3H), 2.61 (t, J=6.6 Hz, 2H), 2.17 (s, 6H), 2.03-1.53 (m, 6H). MS (ESI+) m/z 578 (M+H)⁺.

### Example 71

### 3-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-6-(1-methyl-1H-pyrazol-4-yl)pyrazine-2-carboxamide

### Step 71A

### methyl 3-amino-6-(1-methyl-1H-pyrazol-4-yl)pyrazine-2-carboxylate

The title compound was prepared as described in Step 41A using 1-methyl-4-(4,4,5,5-tetramethyl-1,3,2-dioxaborolan-2-yl)-1H-pyrazole in place of 2-fluoropyridine-5-boronic acid pinacol ester. ¹H NMR (501 MHz, DMSO-d₆) δ 8.63 (s, 1H), 8.17 (d, J=0.7 Hz, 1H), 7.91 (d, J=0.8 Hz, 1H), 7.25 (s, 2H), 3.88 (d, J=2.4 Hz, 6H). MS (ESI+) m/z 234.2 (M+H)⁺.

### Step 71B

### 3-amino-6-(1-methyl-1H-pyrazol-4-yl)pyrazine-2-carboxylic acid

The title compound was prepared as described in Step 41B using the product of Step 71A in place of the product of Step 41A. ¹H NMR (501 MHz, DMSO-d₆) δ 12.75 (s, 1H), 8.63 (s, 1H), 8.28 (d, J=0.7 Hz, 1H), 8.02 (d, J=0.9 Hz, 1H), 7.30 (s, 2H), 3.88 (s, 3H). MS (ESI+) m/z 220.2 (M+H)⁺.

### Step 71C

### 3-amino-N-{(3S,4R)-4-[(4-bromophenyl)methoxy]oxolan-3-yl}-6-(1-methyl-1H-pyrazol-4-yl)pyrazine-2-carboxamide

To a solution of the product of Step 71B (318 mg, 1.4 mmol), Intermediate 28 (521 mg, 1.9 mmol), and N,N-diisopropylethylamine (0.8 mL, 4.6 mmol) in N,N-dimethylformamide (4 mL) was added 1-[bis(dimethylamino)methylene]-1H-1,2,3-triazolo[4,5-b]pyridinium 3-oxid hexafluorophosphate (HATU, 609 mg, 1.6 mmol). The mixture was stirred for 24 hours, concentrated and purified by flash chromatography on silica gel eluting with 0-60% 3:1 ethyl acetate/ethanol in hexanes to afford the title compound (720 mg). ¹H NMR (400 MHz, DMSO-d₆) δ 8.67-8.56 (m, 2H), 8.32 (s, 1H), 8.13 (s, 1H), 7.57-7.48 (m, 2H), 7.43 (s, 2H), 7.36-7.28 (m, 2H), 4.67 (d, J=12.3 Hz, 1H), 4.56 (d, J=12.3 Hz, 1H), 4.47 (qd, J=6.9, 2.0 Hz, 1H), 4.23 (dt, J=4.8, 2.3 Hz, 1H), 4.03 (ddd, J=18.1, 9.6, 5.9 Hz, 2H), 3.89 (s, 3H), 3.81-3.73 (m, 2H). MS (ESI+) m/z 475.3 (M+H)⁺.

### Step 71D

### 3-amino-6-(1-methyl-1H-pyrazol-4-yl)-N-[(3S,4R)-4-{[4-(4,4,5,5-tetramethyl-1,3,2-dioxaborolan-2-yl)phenyl]methoxy}oxolan-3-yl]pyrazine-2-carboxamide

The title compound was prepared as described in Step 41D using the product of Step 71C in place of the product of Step 41C. ¹H NMR (400 MHz, DMSO-d₆) δ 8.67-8.56 (m, 2H), 8.32 (s, 1H), 8.12 (s, 1H), 7.68-7.61 (m, 2H), 7.43 (s, 2H), 7.36 (d, J=7.8 Hz, 2H), 4.72 (d, J=12.5 Hz, 1H), 4.60 (d, J=12.5 Hz, 1H), 4.48 (tdd, J=7.0, 4.7, 2.0 Hz, 1H), 4.23 (dt, J=4.8, 2.2 Hz, 1H), 4.04 (ddd, J=20.4, 9.6, 5.9 Hz, 2H), 3.89 (s, 3H), 3.82-3.72 (m, 2H), 1.28 (s, 12H). MS (ESI+) m/z 521.3 (M+H)⁺.

### Step 71E

### 3-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-6-(1-methyl-1H-pyrazol-4-yl)pyrazine-2-carboxamide

The title compound was prepared as described in Step 41E using the product of Step 71D in place of Intermediate 32. ¹H NMR (501 MHz, methanol-d₄) δ 8.44 (s, 1H), 8.13 (s, 1H), 8.00 (d, J=0.7 Hz, 1H), 7.49-7.42 (m, 2H), 7.42-7.31 (m, 2H), 7.29-7.17 (m, 2H), 6.49 (d, J=8.2 Hz, 1H), 4.76 (d, J=11.9 Hz, 1H), 4.65 (d, J=11.9 Hz, 1H), 4.58 (ddd, J=6.1, 4.0, 2.0 Hz, 1H), 4.24 (dt, J=5.1, 2.4 Hz, 1H), 3.89 (s, 3H), 3.86-3.78 (m, 2H), 3.71 (t, J=6.1 Hz, 2H), 3.19 (s, 2H), 3.13 (d, J=11.9 Hz, 2H), 2.62 (t, J=6.0 Hz, 2H), 2.29 (s, 2H), 1.79 (td, J=10.8, 9.8, 3.4 Hz, 4H), 1.30 (s, 6H). MS (ESI+) m/z 667.2 (M+H)⁺.

### Example 72

### 2-amino-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}-5-(1-methyl-1H-pyrazol-4-yl)pyridine-3-carboxamide

### Step 72A

### 6′-bromo-2′,3′-dihydro-1′H-spiro[cyclopropane-1,4′-isoquinoline]

To a solution of tert-butyl 6′-bromo-1′H-spiro[cyclopropane-1,4′-isoquinoline]-2′(3′H)-carboxylate (1 g, 2.96 mmol) in dichloromethane (10 mL) was added 2 M HCl in ether (10 mL, 20 mmol). After stirring at ambient temperature for 18 hours, the mixture was concentrated to afford a HCl salt. The salt was dissolved in methanol and eluted through an Agilent Bond Elut SCX column with 1 M NH₃ in methanol to afford the free base of the title compound (644 mg). ¹H NMR (400 MHz, methanol-d₄) δ 7.19 (dd, J=8.2, 2.0 Hz, 1H), 6.94 (dd, J=8.3, 1.0 Hz, 1H), 6.86 (d, J=1.9 Hz, 1H), 3.99 (s, 2H), 2.85 (s, 2H), 1.06-0.88 (m, 4H). MS (ESI+) m/z 238.1 (M+H)⁺.

### Step 72B

### tert-butyl 4-(6′-bromo-1′H-spiro[cyclopropane-1,4′-isoquinolin]-2′(3′H)-yl)piperidine-1-carboxylate

The product of Step 72A (560 mg, 2.35 mmol), tert-butyl 4-oxopiperidine-1-carboxylate (516 mg, 2.59 mmol) and sodium triacetoxyhydroborate (996.5 mg, 4.70 mmol) were stirred in AcOH for 18 hours. The mixture was concentrated and partitioned between water and ethyl acetate. The organic phase was washed with saturated NaHCO₃ and brine, dried over MgSO₄, filtered, and concentrated. The residue was purified by flash chromatography on silica gel eluting with 60% (3:1 ethyl acetate/ethanol) in hexanes followed by 10% 1 M NH₃ in dichloromethane to afford the title compound (442 mg). MS (ESI+) m/z 421.0 (M+H)⁺.

### Step 72C

### 6′-bromo-2′-(piperidin-4-yl)-2′,3′-dihydro-1′H-spiro[cyclopropane-1,4′-isoquinoline]

The title compound was prepared as a HCl salt as described in Step 72A using the product of Step 72B in place of tert-butyl 6′-bromo-1′H-spiro[cyclopropane-1,4′-isoquinoline]-2′(3′H)-carboxylate. MS (ESI+) m/z 321.0 (M+H)⁺.

### Step 72D

### 2-[4-(6′-bromo-1′H-spiro[cyclopropane-1,4′-isoquinolin]-2′(3′H)-yl)piperidin-1-yl]ethan-1-ol

The product of Step 72C (292 mg, 0.74 mmol), 2-bromoethanol (296.6 mg, 2.37 mmol) and K₂CO₃ (312 mg, 2.26 mmol) were stirred in N,N-dimethylformamide (3 mL) for 24 hours. The mixture was filtered through diatomaceous earth, concentrated and purified by flash chromatography on silica gel eluting with 0-10% methanol/dichloromethane to afford the title compound (51 mg). ¹H NMR (501 MHz, methanol-d₄) δ 7.41 (dd, J=8.3, 1.9 Hz, 1H), 7.15 (d, J=8.3 Hz, 1H), 7.07 (d, J=2.0 Hz, 1H), 4.60 (s, 2H), 3.93-3.87 (m, 2H), 3.85 (d, J=12.5 Hz, 2H), 3.72 (tt, J=12.2, 4.0 Hz, 1H), 3.48 (s, 2H), 3.37-3.27 (m, 1H), 3.19 (d, J=14.9 Hz, 3H), 2.48 (d, J=13.3 Hz, 2H), 2.21 (dd, J=13.6, 10.0 Hz, 2H), 1.28 (s, 2H), 1.23 (d, J=4.0 Hz, 2H). MS (ESI+) m/z 365.2 (M+H)⁺.

### Step 72E

### 2-amino-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}-5-(1-methyl-1H-pyrazol-4-yl)pyridine-3-carboxamide

The product of Step 72D (29 mg, 0.049 mmol), Intermediate 33 (46 mg, 0.089 mmol), bis(di-tert-butyl(4-dimethylaminophenyl)phosphine)dichloropalladium(II) (5.9 mg, 8.33 μmol) and 2 M aqueous Na₂CO₃ (0.1 mL, 0.200 mmol) in N,N-dimethylformamide (1 mL) was evacuated and backfilled with nitrogen. The mixture was heated at 120° C. for 20 minutes in a Biotage Mircowave, cooled to ambient temperature and filtered through diatomaceous earth. Purification by reverse phase HPLC eluting with 5/95 to 50/50 acetonitrile/0.1% trifluoroacetic acid in water provided the title compound as a trifluoroacetic acid salt. The salt was dissolved in methanol, eluted with 1 M NH₃ in methanol through an Agilent Bond Elut SCX column and concentrated. The free base was dissolved in methanol, filtered, and concentrated to afford the title compound (12 mg). ¹H NMR (501 MHz, methanol-d₄) δ 8.24 (d, J=2.3 Hz, 1H), 7.95 (d, J=2.2 Hz, 1H), 7.80 (s, 1H), 7.74 (d, J=0.8 Hz, 1H), 7.49-7.42 (m, 2H), 7.41-7.35 (m, 2H), 7.23 (dd, J=7.9, 1.8 Hz, 1H), 7.09 (d, J=8.0 Hz, 1H), 6.83 (d, J=1.8 Hz, 1H), 4.70-4.59 (m, 2H), 4.41 (td, J=7.6, 4.6 Hz, 1H), 3.94 (d, J=18.6 Hz, 3H), 3.86 (s, 3H), 3.70 (t, J=6.0 Hz, 2H), 3.13 (d, J=11.6 Hz, 2H), 2.72 (s, 2H), 2.60 (t, J=6.0 Hz, 2H), 2.52 (tt, J=11.3, 3.7 Hz, 1H), 2.27-2.08 (m, 3H), 2.08-1.96 (m, 3H), 1.86-1.54 (m, 6H), 1.10-1.01 (m, 2H), 0.99-0.87 (m, 2H). MS (ESI+) m/z 676.6 (M+H)⁺.

### Example 73

### 2-amino-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide

### Step 73A

### tert-butyl 4-(5-bromo-3,3-dimethyl-2-oxo-2,3-dihydro-1H-pyrrolo[3,2-b]pyridin-1-yl)piperidine-1-carboxylate

5-Bromo-3,3-dimethyl-1H-pyrrolo[3,2-b]pyridin-2 (3H)-one (994 mg, 4.12 mmol), 4-(toluene-4-sulfonyloxy)-piperidine-1-carboxylic acid tert-butyl ester (1.77 g, 4.98 mmol) and sodium hydride (202.5 mg, 5.06 mmol) were stirred in N,N-dimethylformamide (4 mL) at 50° C. for 18 hours. The mixture was cooled to ambient temperature, partitioned between ethyl acetate and water, and the organic phase was washed with water and brine. After drying over MgSO₄, the mixture was filtered, concentrated and purified by flash chromatography on silica gel eluting with 0-50% ethyl acetate/hexanes to afford the title compound (425 mg). MS (ESI+) m/z 426.0 (M+H)⁺.

### Step 73B

### 5-bromo-3,3-dimethyl-1-(piperidin-4-yl)-1,3-dihydro-2H-pyrrolo[3,2-b]pyridin-2-one

The title compound was prepared as described in Step 72A using the product of Step73A in place of tert-butyl 6′-bromo-1′H-spiro[cyclopropane-1,4′-isoquinoline]-2′(3′H)-carboxylate. ¹H NMR (400 MHz, methanol-d₄) δ 7.47 (s, 2H), 4.58-4.16 (m, 1H), 3.56 (ddt, J=13.1, 4.2, 1.9 Hz, 3H), 3.17 (td, J=13.3, 3.0 Hz, 3H), 2.81-2.51 (m, 3H), 2.02 (ddq, J=15.5, 5.2, 2.8, 2.3 Hz, 2H), 1.36 (s, 6H). MS (ESI+) m/z 324.3 (M+H)⁺.

### Step 73C

### 5-bromo-1-[1-(2-hydroxyethyl)piperidin-4-yl]-3,3-dimethyl-1,3-dihydro-2H-pyrrolo[3,2-b]pyridin-2-one

The title compound was prepared as described in Step 72D using the product of Step 73B in place of the product of Step 72C. ¹H NMR (400 MHz, methanol-d₄) δ 7.63 (d, J=8.4 Hz, 1H), 7.44 (d, J=8.4 Hz, 1H), 4.27 (tt, J=12.5, 4.4 Hz, 1H), 3.71 (t, J=5.9 Hz, 2H), 3.13 (dp, J=11.4, 2.1 Hz, 2H), 2.59 (t, J=5.9 Hz, 2H), 2.43 (qd, J=12.6, 3.9 Hz, 2H), 2.24 (td, J=12.2, 2.4 Hz, 2H), 1.69 (ddd, J=12.1, 4.6, 2.1 Hz, 2H), 1.35 (s, 6H). MS (ESI+) m/z 370.2 (M+H)⁺.

### Step 73D

### 2-amino-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide

The title compound was prepared as described in Step 72E using the product of Step 73C in place of the product of Step 72D. ¹H NMR (501 MHz, methanol-d₄) δ 8.14 (d, J=2.3 Hz, 1H), 7.86 (d, J=2.3 Hz, 1H), 7.79 (dd, J=8.3, 2.2 Hz, 2H), 7.73 (d, J=2.3 Hz, 1H), 7.63 (d, J=2.2 Hz, 1H), 7.58 (dd, J=8.5, 2.3 Hz, 1H), 7.49 (dd, J=8.3, 2.2 Hz, 1H), 7.33 (dd, J=8.3, 2.2 Hz, 2H), 4.58 (t, J=2.9 Hz, 2H), 4.32 (dt, J=11.5, 6.1 Hz, 1H), 4.23 (s, 1H), 3.88 (ddt, J=8.9, 6.5, 3.0 Hz, 1H), 3.76 (d, J=2.2 Hz, 3H), 3.65 (td, J=5.9, 2.1 Hz, 2H), 3.11 (d, J=11.7 Hz, 2H), 2.57 (t, J=6.1 Hz, 2H), 2.49-2.37 (m, 2H), 2.25 (t, J=12.1 Hz, 2H), 2.06 (dt, J=14.1, 7.0 Hz, 1H), 1.97-1.88 (m, 1H), 1.71 (dd, J=12.2, 6.0 Hz, 2H), 1.66 (t, J=13.7 Hz, 3H), 1.51 (dt, J=13.3, 7.5 Hz, 1H), 1.28 (dd, J=5.4, 2.2 Hz, 6H). MS (ESI+) m/z 679.2 (M+H)⁺.

### Example 74

### 6-amino-6′-fluoro-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}[3,3′-bipyridine]-5-carboxamide

The title compound was prepared as described in Step 72E using the product of Step 73C in place of the product of Step 72D and Intermediate 32 in place of Intermediate 33. ¹H NMR (400 MHz, methanol-d₄) δ 8.33 (dd, J=9.6, 2.5 Hz, 2H), 8.06 (ddd, J=8.4, 7.5, 2.6 Hz, 1H), 8.00 (d, J=2.4 Hz, 1H), 7.88-7.81 (m, 2H), 7.66 (d, J=8.4 Hz, 1H), 7.54 (d, J=8.4 Hz, 1H), 7.42 (d, J=8.0 Hz, 2H), 7.00 (dd, J=8.6, 2.6 Hz, 1H), 4.70 (d, J=12.7 Hz, 1H), 4.64 (d, J=12.7 Hz, 1H), 4.47-4.26 (m, 2H), 3.96 (dt, J=6.4, 4.6 Hz, 1H), 3.74 (t, J=5.9 Hz, 2H), 3.18 (d, J=11.5 Hz, 2H), 2.63 (t, J=6.0 Hz, 2H), 2.51 (qd, J=12.4, 3.7 Hz, 2H), 2.30 (t, J=11.6 Hz, 2H), 2.16 (tt, J=13.7, 6.2 Hz, 1H), 2.04 (dt, J=12.5, 7.2 Hz, 1H), 1.89-1.69 (m, 5H), 1.58 (dq, J=12.9, 7.6 Hz, 1H), 1.38 (d, J=7.9 Hz, 6H). MS (ESI+) m/z 694.3 (M+H)⁺.

### Example 75

### 6-amino-6′-fluoro-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}[3,3′-bipyridine]-5-carboxamide

The title compound was prepared as described in Step 72E using Intermediate 32 in place of Intermediate 33. ¹H NMR (400 MHz, methanol-d₄) δ 8.35 (d, J=2.6 Hz, 1H), 8.31 (d, J=2.3 Hz, 1H), 8.04 (ddd, J=8.5, 7.5, 2.6 Hz, 1H), 7.98 (d, J=2.4 Hz, 1H), 7.46-7.33 (m, 4H), 7.19 (dd, J=7.9, 1.8 Hz, 1H), 7.11-6.99 (m, 2H), 6.79 (d, J=1.8 Hz, 1H), 4.68 (d, J=12.5 Hz, 1H), 4.60 (d, J=12.6 Hz, 1H), 4.41 (td, J=7.6, 4.8 Hz, 1H), 3.93 (d, J=11.9 Hz, 3H), 3.68 (t, J=6.1 Hz, 2H), 3.08 (d, J=11.4 Hz, 2H), 2.72 (s, 2H), 2.60-2.43 (m, 2H), 2.24-1.47 (m, 13H), 1.04 (t, J=3.1 Hz, 2H), 0.94 (t, J=3.2 Hz, 2H). MS (ESI+) m/z 691.3 (M+H)⁺.

### Example 76

### 2-amino-N-{(1S,2S)-2-[(4-{1-[1-(2-hydroxyethyl)piperidin-4-yl]-3,3-dimethyl-2-oxo-2,3-dihydro-1H-pyrrolo[2,3-b]pyridin-5-yl}phenyl)methoxy]cyclopentyl}-5-(1-methyl-H-pyrazol-4-yl)pyridine-3-carboxamide

### Step 76A

### tert-butyl 4-(5-bromo-3,3-dimethyl-2-oxo-2,3-dihydro-1H-pyrrolo[2,3-b]pyridin-1-yl)piperidine-1-carboxylate

The title compound was prepared as described in Step 73A using 5-bromo-3,3-dimethyl-1H-pyrrolo[2,3-b]pyridin-2 (3H)-one in place of 5-bromo-3,3-dimethyl-1H-pyrrolo[3,2-b]pyridin-2 (3H)-one. ¹H NMR (501 MHz, methanol-d₄) δ 8.22 (d, J=2.2 Hz, 1H), 7.83 (d, J=2.2 Hz, 1H), 4.47 (tt, J=12.3, 4.1 Hz, 1H), 4.22 (dd, J=13.4, 3.7 Hz, 2H), 2.86 (s, 2H), 2.52 (dd, J=12.6, 4.5 Hz, 2H), 1.68-1.61 (m, 2H), 1.49 (s, 9H), 1.35 (s, 6H).

### Step 76B

### 5-bromo-3,3-dimethyl-1-(piperidin-4-yl)-1,3-dihydro-2H-pyrrolo[2,3-b]pyridin-2-one

The title compound was prepared as described in Step 72A using the product of Step 76A in place of tert-butyl 6′-bromo-1′H-spiro[cyclopropane-1,4′-isoquinoline]-2′(3′H)-carboxylate. ¹H NMR (400 MHz, methanol-d₄) δ 8.24 (d, J=2.2 Hz, 1H), 7.88 (d, J=2.1 Hz, 1H), 4.63 (tt, J=12.2, 4.0 Hz, 1H), 3.59-3.49 (m, 2H), 3.22-3.10 (m, 2H), 2.96-2.80 (m, 2H), 2.00-1.91 (m, 2H), 1.38 (s, 6H). MS (ESI+) m/z 324.2 (M+H)⁺.

### Step 76C

### 5-bromo-1-[1-(2-hydroxyethyl)piperidin-4-yl]-3,3-dimethyl-1,3-dihydro-2H-pyrrolo[2,3-b]pyridin-2-one

The title compound was prepared as described in Step 72D using the product of Step 76B in place of the product of Step 72C. MS (ESI+) m/z 370.0 (M+H)⁺.

### Step 76D

### 2-amino-N-{(1S,2S)-2-[(4-{1-[1-(2-hydroxyethyl)piperidin-4-yl]-3,3-dimethyl-2-oxo-2,3-dihydro-1H-pyrrolo[2,3-b]pyridin-5-yl}phenyl)methoxy]cyclopentyl}-5-(1-methyl-1H-pyrazol-4-yl)pyridine-3-carboxamide

The title compound was prepared as described in Step 72E using the product of Step 76C in place of the product of Step 72D. ¹H NMR (400 MHz, methanol-d₄) δ 8.16 (dd, J=17.4, 2.2 Hz, 2H), 7.85 (d, J=2.3 Hz, 1H), 7.77-7.68 (m, 2H), 7.63 (d, J=0.8 Hz, 1H), 7.47-7.39 (m, 2H), 7.34 (d, J=8.2 Hz, 2H), 4.64-4.51 (m, 2H), 4.28 (dtt, J=20.6, 8.3, 4.4 Hz, 2H), 3.87 (dt, J=6.5, 4.4 Hz, 1H), 3.78 (s, 3H), 3.62 (t, J=6.0 Hz, 2H), 3.04 (d, J=12.0 Hz, 2H), 2.67 (dqd, J=38.7, 12.5, 3.5 Hz, 3H), 2.50 (t, J=6.1 Hz, 2H), 2.22-1.87 (m, 3H), 1.77-1.44 (m, 6H), 1.28 (d, J=2.8 Hz, 6H). MS (ESI+) m/z 679.4 (M+H)⁺.

### Example 77

### 2-amino-N-{(1S,2S)-2-[(4-{1-[1-(2-hydroxyethyl)piperidin-4-yl]-3,3-dimethyl-2,3-dihydro-1H-pyrrolo[2,3-b]pyridin-5-yl}phenyl)methoxy]cyclopentyl}-5-(1-methyl-1H-pyrazol-4-yl)pyridine-3-carboxamide

### Step 77A

### tert-butyl 4-(5-bromo-3,3-dimethyl-2,3-dihydro-1H-pyrrolo[2,3-b]pyridin-1-yl)piperidine-1-carboxylate

A mixture of 5-bromo-3,3-dimethyl-2,3-dihydro-1H-pyrrolo[2,3-b]pyridine (80.7 mg, 0.355 mmol), tert-butyl 4-oxopiperidine-1-carboxylate (146 mg, 0.732 mmol), titanium(IV) chloride (0.2 mL, 1.82 mmol) and triethylamine (0.15 mL, 1.076 mmol) was stirred in dichloromethane (6 mL) for 18 hours. Sodium cyanotrihydroborate (0.075 mL, 1.43 mmol) in methanol (1 mL) was added, and the mixture was heated at 40° C. for 18 hours. After quenching with saturated NaHCO₃ the mixture was filtered through diatomaceous earth and concentrated. Purification by flash chromatography on silica gel eluting with 0-40% ethyl acetate in hexanes gave the title compound (58 mg). ¹H NMR (501 MHz, methanol-d₄) δ 7.76 (d, J=2.2 Hz, 1H), 7.30 (d, J=2.1 Hz, 1H), 4.22-4.15 (m, 2H), 3.99 (tt, J=11.9, 3.9 Hz, 1H), 3.29 (s, 2H), 2.86 (s, 2H), 1.77-1.70 (m, 2H), 1.61 (qd, J=12.4, 4.4 Hz, 2H), 1.46 (s, 9H), 1.28 (s, 6H). MS (ESI+) m/z 409.9 (M+H)⁺.

### Step 77B

### 5-bromo-3,3-dimethyl-1-(piperidin-4-yl)-2,3-dihydro-1H-pyrrolo[2,3-b]pyridine

The title compound was prepared as described in Step 72A using the product of Step 77A in place of tert-butyl 6′-bromo-1′H-spiro[cyclopropane-1,4′-isoquinoline]-2′(3′H)-carboxylate. MS (ESI+) m/z 310.0 (M+H)⁺.

### Step 77C

### 2-[4-(5-bromo-3,3-dimethyl-2,3-dihydro-1H-pyrrolo[2,3-b]pyridin-1-yl)piperidin-1-yl]ethan-1-ol

The title compound was prepared as described in Step 72D using the product of Step 77B in place of the product of Step 72C. MS (ESI+) m/z 356.1 (M+H)⁺.

### Step 77D

### 2-amino-N-{(1S,2S)-2-[(4-{1-[1-(2-hydroxyethyl)piperidin-4-yl]-3,3-dimethyl-2,3-dihydro-1H-pyrrolo[2,3-b]pyridin-5-yl}phenyl)methoxy]cyclopentyl}-5-(1-methyl-1H-pyrazol-4-yl)pyridine-3-carboxamide

The title compound was prepared as described in Step 72E using the product of Step 77C in place of the product of Step 72D. ¹H NMR (400 MHz, methanol-d₄) δ 8.24 (d, J=2.3 Hz, 1H), 7.93 (dd, J=19.5, 2.2 Hz, 2H), 7.82 (d, J=0.8 Hz, 1H), 7.74 (d, J=0.8 Hz, 1H), 7.47-7.34 (m, 5H), 4.70-4.54 (m, 2H), 4.41 (td, J=7.4, 4.5 Hz, 1H), 4.04-3.91 (m, 3H), 3.87 (s, 3H), 3.73 (t, J=5.9 Hz, 2H), 3.34 (s, 2H), 3.19 (d, J=11.8 Hz, 2H), 2.69 (t, J=5.9 Hz, 2H), 2.40 (t, J=11.8 Hz, 2H), 2.22-2.09 (m, 1H), 2.02 (ddd, J=14.8, 7.6, 5.7 Hz, 1H), 1.96-1.68 (m, 6H), 1.67-1.53 (m, 1H), 1.32 (d, J=3.7 Hz, 6H). MS (ESI+) m/z 665.3 (M+H)⁺.

### Example 78

### 6-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-6′-fluoro[3,3′-bipyridine]-5-carboxamide

### Step 78A

### 1-(5-bromo-2,3-dihydro-1H-inden-1-yl)-4-[(2,2-dimethyl-1,3-dioxolan-4-yl)methyl]piperazine

The title compound was prepared as described in Step 23A substituting the product of Step 8B for the product of Step 12B. MS (ESI+) m/z 397.17 (M+H)⁺.

### Step 78B

### 6-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-6′-fluoro[3,3′-bipyridine]-5-carboxamide

The title compound was prepared as described in Example 19 substituting the product of Step 78A for the product of Step 8C. ¹H NMR (400 MHz, DMSO-d₆) δ 8.59-8.52 (m, 1H), 8.47 (d, J=2.4 Hz, 1H), 8.39 (d, J=7.8 Hz, 1H), 8.25 (td, J=8.2, 2.7 Hz, 1H), 8.20-8.14 (m, 1H), 7.61-7.52 (m, 2H), 7.47-7.34 (m, 4H), 7.34-7.20 (m, 4H), 4.66-4.55 (m, 2H), 4.39-4.23 (m, 2H), 4.22-4.11 (m, 1H), 3.98 (dd, J=8.1, 6.2 Hz, 1H), 3.95-3.87 (m, 1H), 3.55-3.45 (m, 1H), 2.97-2.86 (m, 1H), 2.86-2.72 (m, 1H), 2.52-2.44 (m, 4H), 2.43-2.29 (m, 6H), 2.12-1.90 (m, 4H), 1.78-1.51 (m, 4H), 1.30 (s, 3H), 1.27-1.21 (m, 3H). MS (ESI+) m/z 721.3 (M+H)⁺.

### Example 79

### 2-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-5-(1-methyl-1H-pyrazol-4-yl)pyridine-3-carboxamide

The title compound was prepared as described in Step 43D substituting the product of Step 78A for the product of Step 43C. ¹H NMR (400 MHz, DMSO-d₆) δ 8.37-8.29 (m, 2H), 8.02-7.95 (m, 2H), 7.78 (d, J=0.8 Hz, 1H), 7.56 (d, J=7.9 Hz, 2H), 7.47-7.35 (m, 4H), 7.30 (d, J=7.8 Hz, 1H), 6.96 (s, 2H), 4.66-4.55 (m, 2H), 4.37-4.24 (m, 2H), 4.22-4.12 (m, 1H), 3.98 (dd, J=8.1, 6.3 Hz, 1H), 3.96-3.89 (m, 1H), 3.83 (s, 3H), 3.54-3.46 (m, 1H), 2.98-2.86 (m, 1H), 2.85-2.74 (m, 1H), 2.53-2.44 (m, 4H), 2.44-2.31 (m, 6H), 2.12-1.89 (m, 4H), 1.80-1.53 (m, 4H), 1.29 (s, 3H), 1.27-1.22 (m, 3H). MS (ESI+) m/z 706.3 (M+H)⁺.

### Example 80

### 6-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide

The title compound was prepared as described in Step 23C substituting the product of Example 78 for the product of Step 23B. ¹H NMR (400 MHz, DMSO-d₆, 120° C.) δ 8.50-8.44 (m, 1H), 8.38 (d, J=2.4 Hz, 1H), 8.15 (td, J=8.1, 2.6 Hz, 1H), 8.09 (d, J=2.4 Hz, 1H), 8.07-7.97 (m, 1H), 7.56-7.49 (m, 2H), 7.43-7.34 (m, 4H), 7.31 (d, J=7.8 Hz, 1H), 7.12 (dd, J=8.5, 2.9 Hz, 1H), 6.85 (brs, 2H), 4.66-4.56 (m, 2H), 4.37-4.24 (m, 2H), 4.06-3.98 (m, 1H), 3.69-3.60 (m, 1H), 3.38 (h, J=5.2 Hz, 2H), 2.99-2.42 (m, 11H), 2.41-2.33 (m, 1H), 2.15-2.02 (m, 3H), 2.02-1.91 (m, 1H), 1.79-1.56 (m, 4H). MS (ESI+) m/z 681.2 (M+H)⁺.

### Example 81

### 2-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 23C substituting the product of Example 79 for the product of Step 23B. ¹H NMR (400 MHz, DMSO-d₆, 120° C.) δ 8.24 (d, J=2.3 Hz, 1H), 7.93 (dd, J=8.5, 4.7 Hz, 2H), 7.84 (s, 1H), 7.67 (s, 1H), 7.58-7.50 (m, 2H), 7.45-7.34 (m, 4H), 7.31 (d, J=7.8 Hz, 1H), 6.54 (brs, 2H), 4.67-4.55 (m, 2H), 4.35-4.22 (m, 2H), 4.05-3.98 (m, 1H), 3.83 (s, 3H), 3.66-3.58 (m, 1H), 3.36 (qd, J=10.8, 5.3 Hz, 2H), 2.99-2.36 (m, 11H), 2.36-2.26 (m, 1H), 2.13-1.92 (m, 4H), 1.79-1.58 (m, 4H). MS (ESI+) m/z 666.1 (M+H)⁺.

### Example 82

### 6-amino-2′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide

The title compound was prepared as described in Step 8D substituting Intermediate 19 for Intermediate 33. ¹H NMR (400 MHz, DMSO-d₆) δ 8.35 (d, J=7.7 Hz, 1H), 8.32 (t, J=2.3 Hz, 1H), 8.18-8.05 (m, 3H), 7.57-7.49 (m, 2H), 7.45-7.25 (m, 8H), 4.56 (d, J=1.9 Hz, 2H), 4.34-4.18 (m, 4H), 4.06 (s, 1H), 3.91-3.81 (m, 1H), 3.42 (d, J=6.1 Hz, 3H), 2.79 (dtdd, J=30.2, 23.2, 15.7, 6.8 Hz, 4H), 2.37 (s, 4H), 2.05-1.84 (m, 5H), 1.71-1.49 (m, 4H). MS (ESI+) m/z 651 (M+H)⁺.

### Example 83

### 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide

### Step 83A

### tert-butyl 4-(5-bromo-3,3-dimethyl-2,3-dihydro-1H-inden-1-yl)piperazine-1-carboxylate

A mixture of 5-bromo-3,3-dimethyl-2,3-dihydro-1H-inden-1-one (1.4 g, 5.86 mmol), triethylamine (2.45 mL, 17.57 mmol) and tert-butyl piperazine-1-carboxylate (2.18 g, 11.71 mmol) in dichloromethane (40 mL) was treated with 1 M titanium(IV) chloride in dichloromethane (2.93 mL, 2.93 mmol), and the solution was stirred at ambient temperature for 15 hours. Sodium cyanoborohydride (1.29 g, 20.49 mmol) was added, followed by methanol (15 mL), and the mixture was stirred at ambient temperature for 2 days. The mixture was partitioned in saturated NaHCO₃ and dichloromethane. The aqueous layer was extracted with additional dichloromethane and the combined organic layers were washed with brine, dried over Na₂SO₄, and concentrated. The residue was purified by flash chromatography on silica gel eluting with 0-25% ethyl acetate/heptane to afford the title compound (1.69 g). MS (ESI+) m/z 409.1 (M+H)⁺.

### Step 83B

### 1-(5-bromo-3,3-dimethyl-2,3-dihydro-1H-inden-1-yl)piperazine

The title compound was prepared as described in Example 5 substituting the product of Step 83A for the product of Example 4. MS (APCI+) m/z 309.2 (M+H)⁺.

### Step 83C

### 2-[4-(5-bromo-3,3-dimethyl-2,3-dihydro-1H-inden-1-yl)piperazin-1-yl]ethan-1-ol

The title compound was prepared as described in Step 13A substituting the product of Step 83B for the product of Step 12B. MS (ESI+) m/z 353.2 (M+H)⁺.

### Step 83D

### 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 43D substituting the product of Step 83C for the product of Step 43C. ¹H NMR (400 MHz, DMSO-d₆, 120° C.) δ 8.24 (d, J=2.3 Hz, 1H), 7.99-7.89 (m, 2H), 7.84 (s, 1H), 7.67 (s, 1H), 7.59-7.52 (m, 2H), 7.42-7.34 (m, 4H), 7.30-7.24 (m, 1H), 6.54 (brs, 2H), 4.67-4.55 (m, 2H), 4.38-4.26 (m, 2H), 4.05-3.97 (m, 1H), 3.82 (s, 3H), 3.77-3.69 (m, 1H), 3.50 (q, J=5.8 Hz, 2H), 2.57-2.39 (m, 10H), 2.13-1.88 (m, 4H), 1.80-1.57 (m, 4H), 1.37 (d, J=0.8 Hz, 3H), 1.20 (d, J=1.0 Hz, 3H). MS (ESI+) m/z 664.2 (M+H)⁺.

### Example 84

### 6-amino-6′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Example 19 substituting the product of Step 83C for the product of Step 8C. ¹H NMR (400 MHz, DMSO-d₆, 120° C.) δ 8.49-8.44 (m, 1H), 8.38 (d, J=2.4 Hz, 1H), 8.20-8.13 (m, 1H), 8.10 (d, J=2.4 Hz, 1H), 8.08-7.95 (m, 1H), 7.58-7.49 (m, 2H), 7.41-7.34 (m, 4H), 7.26 (d, J=7.7 Hz, 1H), 7.11 (dd, J=8.5, 3.0 Hz, 1H), 6.85 (brs, 2H), 4.67-4.56 (m, 2H), 4.38-4.27 (m, 2H), 4.06-3.98 (m, 1H), 3.72 (brs, 1H), 3.56-3.46 (m, 2H), 2.58-2.38 (m, 10H), 2.14-2.02 (m, 1H), 2.02-1.86 (m, 3H), 1.80-1.57 (m, 4H), 1.37 (d, J=1.2 Hz, 3H), 1.20 (d, J=1.3 Hz, 3H). MS (ESI+) m/z 679.2 (M+H)⁺.

### Example 85

### 2-amino-N-{(1S,2S)-2-[(4-{(1R*,2S*)-2-fluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 85A

### tert-butyl 4-[(1R*,2S*)-5-bromo-2-fluoro-2,3-dihydro-1H-inden-1-yl]piperazine-1-carboxylate

The title compound was prepared as described in Step 50C using the product of Step 50A in place of the product of Step 50B. The cis-diastereomer was isolated (411 mg) along with the minor trans-isomer (98 mg). LCMS (APCI+) m/z 399.07 (M+H)⁺.

### Step 85B

### 1-[(1R*,2S*)-5-bromo-2-fluoro-2,3-dihydro-1H-inden-1-yl]piperazine

The title compound was prepared as described in Step 50D using the product of Step 85A in place of the product of Step 50C. The crude product was used without further purification. LCMS (APCI+) m/z 298.92 (M+H)⁺.

### Step 85C

### 2-{4-[(1R*,2S*)-5-bromo-2-fluoro-2,3-dihydro-1H-inden-1-yl]piperazin-1-yl}ethan-1-ol

The title compound was prepared as described in Step 50E using the product of Step 85B in place of the product of Step 50D. LCMS (ESI+) m/z 343.02 (M+H)⁺.

### Step 85D

### 2-amino-N-{(1S,2S)-2-[(4-{(1R*,2S*)-2-fluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 85C in place of the product of Step 50E. ¹H NMR (400 MHz, methanol-d₄) δ 8.51 (dd, J=4.6, 2.1 Hz, 1H), 8.20 (dd, J=5.8, 2.1 Hz, 1H), 7.98 (dd, J=11.8, 0.8 Hz, 1H), 7.87 (dd, J=4.7, 0.8 Hz, 1H), 7.58 (dq, J=8.4, 2.0 Hz, 2H), 7.54-7.47 (m, 2H), 7.45 (d, J=7.9 Hz, 2H), 7.40 (ddt, J=6.9, 2.2, 1.2 Hz, 1H), 5.71 (dtd, J=54.1, 4.1, 2.5 Hz, 1H), 4.77-4.62 (m, 2H), 4.55-4.40 (m, 2H), 4.04 (dtd, J=7.0, 4.5, 2.8 Hz, 1H), 3.93 (t, J=5.4 Hz, 5H), 3.76-3.37 (m, 3H), 3.30-3.01 (m, 6H), 2.26-2.15 (m, 1H), 2.15-2.03 (m, 1H), 1.93-1.75 (m, 3H), 1.75-1.58 (m, 1H). MS (ESI+) m/z 654.4 (M+H)⁺.

### Example 86

### 6-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-6′-fluoro[3,3′-bipyridine]-5-carboxamide

The title compound was prepared as described in Step 50F using Intermediate 32 in place of Intermediate 33. ¹H NMR (400 MHz, methanol-d₄) δ 8.49 (ddd, J=8.5, 4.6, 2.4 Hz, 2H), 8.37 (d, J=2.2 Hz, 1H), 8.20 (tdd, J=7.3, 3.2, 1.0 Hz, 1H), 7.60-7.37 (m, 7H), 7.17 (dt, J=8.5, 2.0 Hz, 1H), 4.77-4.58 (m, 3H), 4.46 (td, J=7.3, 4.6 Hz, 1H), 4.03 (dt, J=6.5, 4.5 Hz, 1H), 3.98-3.87 (m, 2H), 3.65 (d, J=12.0 Hz, 1H), 3.57 (d, J=8.4 Hz, 1H), 3.52-3.35 (m, 4H), 3.33-3.18 (m, 3H), 3.18-2.94 (m, 3H), 2.29-2.15 (m, 1H), 2.08 (ddd, J=14.6, 7.4, 5.1 Hz, 1H), 1.84 (ttd, J=16.7, 8.0, 4.7 Hz, 3H), 1.66 (dq, J=14.9, 7.5 Hz, 1H). MS (ESI+) m/z 687.4 (M+H)⁺.

### Example 87

### amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide

The title compound was prepared as described in Step 50F using product of Step 30C in place of Intermediate 33. ¹H NMR (501 MHz, methanol-d₄) δ 8.43 (d, J=2.3 Hz, 1H), 8.36 (dt, J=7.5, 2.1 Hz, 1H), 7.72 (t, J=7.2 Hz, 1H), 7.59-7.51 (m, 3H), 7.49 (d, J=7.8 Hz, 1H), 7.46 (d, J=8.2 Hz, 2H), 7.42 (d, J=7.9 Hz, 1H), 6.81 (d, J=1.5 Hz, 1H), 6.70 (ddd, J=8.9, 7.0, 2.1 Hz, 1H), 4.80-4.59 (m, 3H), 4.45 (q, J=6.5 Hz, 1H), 4.04 (q, J=5.5 Hz, 1H), 3.97-3.88 (m, 2H), 3.70-3.51 (m, 5H), 3.51-3.35 (m, 4H), 3.34-3.18 (m, 4H), 3.11 (dq, J=20.0, 11.4, 10.1 Hz, 3H), 2.20 (dt, J=13.7, 6.8 Hz, 1H), 2.14-2.03 (m, 1H), 1.92-1.76 (m, 3H), 1.66 (dq, J=14.6, 7.4 Hz, 1H). MS (ESI+) m/z 699.2 (M+H)⁺.

### Example 88

### 2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide

### Step 88A

### tert-butyl 4-(6-bromo-1,2,3,4-tetrahydronaphthalen-1-yl)piperazine-1-carboxylate

To a solution of 6-bromo-3,4-dihydronaphthalen-1 (2H)-one (509 g, 2.26 mmol) and tert-butyl piperazine-1-carboxylate (552 mg, 2.97 mmol) in dioxane (8 mL) was added tetraisopropoxytitanium (1.4 mL, 4.7 mmol), and the mixture was evacuated and backfilled with nitrogen. The mixture was heated at 80° C. for 18 hours and then cooled to ambient temperature. Sodium cyanotrihydroborate (283 mg, 4.5 mmol) was added, and the mixture was evacuated and backfilled with nitrogen. The mixture was heated at 80° C. for 36 hours, cooled to ambient temperature and concentrated. The residue was partitioned between ethyl acetate and water and filtered through diatomaceous earth. The organic phase was washed with brine, dried over MgSO₄ filtered, and concentrated. Purification by flash chromatography on silica gel eluting with 0-20% ethyl acetate/hexanes gave the title compound (205 mg). MS (ESI+) m/z 395.2 (M+H)⁺.

### Step 88B

### 1-(6-bromo-1,2,3,4-tetrahydronaphthalen-1-yl)piperazine

The title compound was prepared as described in Step 72A using Step 88A in place of tert-butyl 6′-bromo-1′H-spiro[cyclopropane-1,4′-isoquinoline]-2′(3′H)-carboxylate. MS (ESI+) m/z 297.3 (M+H)⁺.

### Step 88C

### 2-[4-(6-bromo-1,2,3,4-tetrahydronaphthalen-1-yl)piperazin-1-yl]ethan-1-ol

The title compound was prepared as described in Step 72D using the product of Step 88B in place of the product of Step 72C. ¹H NMR (400 MHz, DMSO-d₆) δ 7.52 (dd, J=8.4, 0.9 Hz, 1H), 7.30 (dd, J=8.3, 2.2 Hz, 1H), 7.25 (d, J=2.1 Hz, 1H), 4.33 (t, J=5.3 Hz, 1H), 4.09 (q, J=5.3 Hz, 1H), 3.70 (q, J=5.1 Hz, 1H), 3.47 (td, J=6.3, 5.3 Hz, 2H), 3.17 (d, J=5.2 Hz, 1H), 2.67 (d, J=3.9 Hz, 1H), 2.43 (s, 6H), 2.43-2.31 (m, 3H), 1.94-1.82 (m, 2H), 1.61 (dd, J=6.5, 3.8 Hz, 1H), 1.62-1.53 (m, 1H). MS (ESI+) m/z 341.2 (M+H)⁺.

### Step 88D

### 2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide

To a mixture of Intermediate 33 (384.5 mg, 0.883 mmol), and the product of Step 88C in dioxane (10 mL) was added potassium phosphate (382 mg, 1.80 mmol) in water (2 mL), and the mixture was stirred for 1 hour while sparing with nitrogen. 4-(Di-tert-butylphosphino)-N,N-dimethylaniline (9.9 mg, 0.037 mmol) and tris(dibenzylideneacetone)dipalladium(0) (11.7 mg, 0.013 mmol) were added, and the mixture was heated at 75° C. for 4 hours. After cooling to ambient temperature, 3-mercaptopropyl-functionalized silica gel was added, and the mixture was filtered through diatomaceous earth and concentrated. Purification by reverse phase HPLC eluting with 5/95 to 70/30 acetonitrile/0.1% trifluoroacetic acid in water gave the title compound as a trifluoroacetic acid salt. The salt was dissolved in methanol, filtered through a Silicycle Carbonate column and concentrated to afford the free base of the title compound (390 mg). ¹H NMR (400 MHz, DMSO-d₆) δ 8.36 (d, J=7.7 Hz, 1H), 8.32 (d, J=2.2 Hz, 1H), 8.02 (d, J=2.3 Hz, 1H), 7.99 (d, J=2.1 Hz, 1H), 7.79 (s, 1H), 7.62 (d, J=8.1 Hz, 1H), 7.56 (d, J=7.9 Hz, 2H), 7.42-7.33 (m, 3H), 7.29 (d, J=2.0 Hz, 1H), 6.98 (s, 2H), 4.69-4.51 (m, 2H), 4.32 (qd, J=7.5, 3.9 Hz, 1H), 3.93 (dt, J=6.3, 3.7 Hz, 1H), 3.84 (s, 3H), 3.77 (q, J=5.1 Hz, 1H), 3.48 (t, J=6.3 Hz, 2H), 2.74 (q, J=4.8 Hz, 2H), 2.65-2.28 (m, 12H), 2.18-1.83 (m, 4H), 1.83-1.47 (m, 4H). MS (ESI+) m/z 650.2 (M+H)⁺.

### Example 89

### 6-amino-6′-fluoro-N-{(1S,2S)-2-[(4-{(1R*,2S*)-2-fluoro-1-[4-(2-hydroxyethyl)piperazin-1-yl]-2,3-dihydro-1H-inden-5-yl}phenyl)methoxy]cyclopentyl}[3,3′-bipyridine]-5-carboxamide

The title compound was prepared as described in Step 50F using the product of Step 85C in place of the product of Step 50E and Intermediate 32 in place of the product of Intermediate 33. ¹H NMR (501 MHz, methanol-d₄) δ 8.51-8.39 (m, 2H), 8.32 (t, J=2.2 Hz, 1H), 8.21-8.12 (m, 1H), 7.53 (dt, J=8.5, 2.0 Hz, 2H), 7.49-7.41 (m, 4H), 7.39 (d, J=8.0 Hz, 1H), 7.14 (dt, J=8.7, 2.7 Hz, 1H), 5.70 (dtd, J=54.1, 4.2, 2.2 Hz, 1H), 4.77-4.58 (m, 2H), 4.55-4.38 (m, 2H), 4.02 (dt, J=6.6, 4.7 Hz, 1H), 3.92 (dd, J=6.0, 4.4 Hz, 2H), 3.36 (s, 3H), 3.31-2.90 (m, 6H), 2.19 (dtd, J=13.2, 7.8, 5.3 Hz, 1H), 2.08 (dq, J=12.8, 7.0 Hz, 1H), 1.82 (dddd, J=21.3, 13.2, 11.0, 5.5 Hz, 3H), 1.64 (dq, J=13.1, 7.6 Hz, 1H). MS (ESI+) m/z 669.3 (M+H)⁺.

### Example 90

### 6-amino-N-{(1S,2S)-2-[(4-{(1R*,2S*)-2-fluoro-1-[4-(2-hydroxyethyl)piperazin-1-yl]-2,3-dihydro-1H-inden-5-yl}phenyl)methoxy]cyclopentyl}-1′-methyl-6′-oxo-1′,6′-dihydro[3,3′-bipyridine]-5-carboxamide

The title compound was prepared as described in Step 50F using the product of Step 85C in place of the product of Step 50E and the product of Step 30C in place of the Intermediate 33. ¹H NMR (400 MHz, methanol-d₄) δ 8.45-8.33 (m, 2H), 7.71 (dd, J=11.6, 7.1 Hz, 1H), 7.60-7.52 (m, 2H), 7.46 (dd, J=10.9, 7.6 Hz, 4H), 7.39 (dt, J=8.5, 1.4 Hz, 1H), 6.80 (dd, J=4.4, 2.1 Hz, 1H), 6.68 (ddd, J=16.0, 7.1, 2.1 Hz, 1H), 5.82-5.59 (m, 1H), 4.76-4.60 (m, 2H), 4.56-4.38 (m, 2H), 4.09-3.99 (m, 1H), 3.98-3.89 (m, 2H), 3.78-3.36 (m, 7H), 3.33-2.95 (m, 7H), 2.19 (ddt, J=13.0, 8.2, 3.8 Hz, 1H), 2.09 (dtd, J=14.5, 6.6, 2.2 Hz, 1H), 1.95-1.75 (m, 3H), 1.65 (dqd, J=12.8, 7.6, 3.0 Hz, 1H). MS (ESI+) m/z 681.5 (M+H)⁺.

### Example 91

### amino-2′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide

The title compound was prepared as described in Step 8D substituting Intermediate 19 for Intermediate 33 and substituting the product of Step 34A for the product of Step 8C. ¹H NMR (400 MHz, DMSO-d₆) δ 8.35 (d, J=7.8 Hz, 1H), 8.31 (t, J=2.2 Hz, 1H), 8.18-8.05 (m, 3H), 7.55-7.49 (m, 2H), 7.44-7.22 (m, 8H), 4.56 (d, J=1.8 Hz, 2H), 4.27 (dq, J=21.7, 6.8, 6.1 Hz, 3H), 3.87 (dt, J=6.1, 3.8 Hz, 1H), 3.43 (q, J=5.9 Hz, 2H), 2.92-2.83 (m, 1H), 2.76 (dt, J=15.8, 7.7 Hz, 1H), 2.34 (dd, J=15.1, 8.9 Hz, 8H), 2.11-1.42 (m, 10H). MS (ESI+) m/z 651 (M+H)⁺.

### Example 92

### 6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide

The title compound was prepared as described Step 8D substituting the product of Step 30C for Intermediate 33 and substituting the product of Step 34A for the product of Step 8C. ¹H NMR (400 MHz, DMSO-d₆) δ 8.49-8.40 (m, 2H), 8.17 (d, J=2.4 Hz, 1H), 7.67 (d, J=7.1 Hz, 1H), 7.56-7.46 (m, 2H), 7.45-7.29 (m, 6H), 7.26 (d, J=7.8 Hz, 1H), 6.77 (d, J=2.0 Hz, 1H), 6.60 (dd, J=7.2, 2.1 Hz, 1H), 4.56 (s, 2H), 4.37-4.19 (m, 3H), 4.06 (q, J=5.4 Hz, 2H), 3.98-3.80 (m, 1H), 3.41 (d, J=18.0 Hz, 5H), 2.87 (ddd, J=16.1, 8.1, 5.7 Hz, 1H), 2.74 (dd, J=15.9, 7.7 Hz, 1H), 2.35 (dt, J=12.4, 8.0 Hz, 8H), 2.06-1.84 (m, 4H), 1.75-1.49 (m, 4H). MS (ESI+) m/z 663 (M+H)⁺.

### Example 93

### 2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide Step 93A 1-(5-bromo-3,3-dimethyl-2,3-dihydro-1H-inden-1-yl)-4-methylpiperazine

The title compound was prepared as described in Step 6B substituting the product of Step 83B for the product of Step 6A. MS (APCI+) m/z 323.15 (M+H)⁺.

### Step 93B

### 2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 43D substituting the product of Step 93A for the product of Step 43C. ¹H NMR (400 MHz, DMSO-d₆, 120° C.) δ 8.23 (d, J=2.2 Hz, 1H), 7.98-7.88 (m, 2H), 7.83 (s, 1H), 7.67 (s, 1H), 7.57-7.51 (m, 2H), 7.40-7.33 (m, 4H), 7.28-7.22 (m, 1H), 6.53 (brs, 2H), 4.66-4.56 (m, 2H), 4.37-4.25 (m, 2H), 4.04-3.98 (m, 1H), 3.82 (s, 3H), 2.57-2.47 (m, 4H), 2.39-2.32 (m, 4H), 2.17 (s, 3H), 2.11-1.89 (m, 4H), 1.79-1.56 (m, 4H), 1.36 (s, 3H), 1.19 (s, 3H). MS (ESI+) m/z 634.2 (M+H)⁺.

### Example 94

### 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-6′-fluoro[3,3′-bipyridine]-5-carboxamide

The title compound was prepared as described in Example 19 substituting the product of Step 93A for the product of Step 8C. ¹H NMR (400 MHz, DMSO-d₆, 120° C.) δ 8.46 (d, J=2.9 Hz, 1H), 8.37 (d, J=2.4 Hz, 1H), 8.14 (td, J=8.2, 2.7 Hz, 1H), 8.09 (d, J=2.4 Hz, 1H), 8.06-7.96 (m, 1H), 7.57-7.48 (m, 2H), 7.39-7.32 (m, 4H), 7.25 (d, J=7.8 Hz, 1H), 7.10 (dd, J=8.5, 3.0 Hz, 1H), 6.84 (brs, 2H), 4.60 (d, J=3.0 Hz, 2H), 4.37-4.26 (m, 2H), 4.04-3.96 (m, 1H), 2.56-2.48 (m, 4H), 2.35 (t, J=4.8 Hz, 4H), 2.17 (s, 3H), 2.12-1.88 (m, 4H), 1.79-1.56 (m, 4H), 1.36 (d, J=1.3 Hz, 3H), 1.19 (d, J=1.4 Hz, 3H). MS (ESI+) m/z 649.3 (M+H)⁺.

### Example 95

### 2-amino-N-{(1S,2S)-2-[(4-{3-[4-(2-hydroxyethyl)piperazin-1-yl]-1-benzofuran-6-yl}phenyl)methoxy]cyclopentyl}-5-(1-methyl-1H-pyrazol-4-yl)pyridine-3-carboxamide

### Step 95A

### tert-butyl 4-(6-bromo-1-benzofuran-3-yl)piperazine-1-carboxylate

A mixture of 6-bromobenzofuran-3 (2H)-one (0.99 g, 4.65 mmol), triethylamine (1.943 mL, 13.94 mmol) and tert-butyl piperazine-1-carboxylate (1.731 g, 9.29 mmol) in dichloromethane (35 mL) was treated with 1 M titanium(IV) chloride in dichloromethane (2.324 mL, 2.324 mmol), and the solution was stirred at ambient temperature for 18 hours. Sodium cyanoborohydride (1.022 g, 16.27 mmol) was added, followed by methanol (11 mL), and the mixture was stirred at ambient temperature for 3 days. The mixture was partitioned in 5% aqueous Na₂CO₃ and dichloromethane and the aqueous layer was extracted with additional dichloromethane. The combined organic layers were washed with brine, dried over Na₂SO₄, filtered through diatomaceous earth and concentrated. The residue was purified by flash chromatography on silica gel eluting with 0-30% ethyl acetate/heptane to afford the title compound (465 mg). MS (ESI+) m/z 381.1 (M+H)⁺.

### Step 95B

### 1-(6-bromo-1-benzofuran-3-yl)piperazine

A solution of the product of Step 95A (0.46 g, 1.207 mmol) in methanol (8 mL) was treated with 4 M HCl in dioxane (3.02 mL, 12.07 mmol), and the mixture was stirred at ambient temperature for 16 hours. The mixture was diluted with ether and the resulting suspension was filtered. The solid was washed with ether and dried under vacuum. The solid was partitioned in dichloromethane and saturated Na₂CO₃ and the aqueous layer was extracted with additional dichloromethane. The combined organic layers were washed with brine, dried over Na₂SO₄, and concentrated to afford the title compound (59 mg). MS (APCI+) m/z 281.1 (M+H)⁺.

### Step 95C

### 2-[4-(6-bromo-1-benzofuran-3-yl)piperazin-1-yl]ethan-1-ol

A solution of the product of Step 95B (0.058 g, 0.206 mmol) in methanol (1 mL) was treated with K₂CO₃ (0.071 g, 0.516 mmol) followed by 2-bromoethanol (0.026 mL, 0.371 mmol), and the mixture was stirred at ambient temperature for 24 hours. Additional 2-bromoethanol (0.013 mL, 0.135 mmol) was added, and the mixture was stirred at ambient temperature for 3 days. The mixture was diluted with dichloromethane and filtered through diatomaceous earth with dichloromethane. The filtrate was washed with water and brine, dried over Na₂SO₄, and concentrated. The residue was purified by flash chromatography on silica gel eluting with 0-10% methanol/dichloromethane to afford the title compound (33 mg). MS (APCI+) m/z 325.10 (M+H)⁺.

### Step 95D

### 2-amino-N-{(1S,2S)-2-[(4-{3-[4-(2-hydroxyethyl)piperazin-1-yl]-1-benzofuran-6-yl}phenyl)methoxy]cyclopentyl}-5-(1-methyl-1H-pyrazol-4-yl)pyridine-3-carboxamide

The title compound was prepared as described in Step 8D substituting the product of Step 95C for the product of Step 8C. ¹H NMR (501 MHz, methanol-d₄) δ 8.43 (d, J=2.1 Hz, 1H), 8.19 (d, J=2.1 Hz, 1H), 7.95 (s, 1H), 7.82 (s, 1H), 7.67 (d, J=8.0 Hz, 1H), 7.65-7.59 (m, 2H), 7.49-7.44 (m, 2H), 7.36-7.29 (m, 2H), 4.76-4.61 (m, 4H), 4.47-4.38 (m, 1H), 4.03-3.97 (m, 1H), 3.91 (s, 3H), 3.81-3.74 (m, 2H), 3.42-3.35 (m, 4H), 3.20-3.13 (m, 3H), 2.94 (t, J=5.4 Hz, 2H), 2.23-2.13 (m, 1H), 2.11-2.02 (m, 1H), 1.90-1.75 (m, 3H), 1.68-1.58 (m, 1H). MS (ESI+) m/z 636.4 (M+H)⁺.

### Example 96

### 2-amino-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 43D substituting Intermediate 15 for the product of Step 43C. ¹H NMR (501 MHz, DMSO-d₆) δ 8.38-8.28 (m, 2H), 8.04-7.95 (m, 2H), 7.78 (d, J=0.8 Hz, 1H), 7.62-7.53 (m, 2H), 7.45-7.34 (m, 4H), 7.29-7.20 (m, 1H), 6.96 (s, 2H), 4.67-4.54 (m, 2H), 4.45-4.25 (m, 3H), 3.97-3.88 (m, 1H), 3.83 (s, 3H), 3.48 (q, J=6.0 Hz, 2H), 2.46-2.31 (m, 6H), 2.12-1.82 (m, 4H), 1.81-1.52 (m, 4H), 1.37 (s, 3H), 1.18 (s, 3H). MS (APCI+) m/z 664.46 (M+H)⁺.

### Example 97

### 6-amino-6′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Example 19 substituting Intermediate 15 for the product of Step 8C. ¹H NMR (501 MHz, DMSO-d₆) δ 8.59-8.51 (m, 1H), 8.47 (d, J=2.3 Hz, 1H), 8.40 (d, J=7.7 Hz, 1H), 8.30-8.20 (m, 1H), 8.17 (d, J=2.4 Hz, 1H), 7.60-7.52 (m, 2H), 7.44-7.34 (m, 4H), 7.30-7.17 (m, 4H), 4.67-4.55 (m, 2H), 4.40 (t, J=7.9 Hz, 1H), 4.36-4.27 (m, 2H), 3.97-3.87 (m, 1H), 3.53-3.44 (m, 2H), 2.49-2.31 (m, 9H), 2.12-1.82 (m, 4H), 1.81-1.51 (m, 4H), 1.36 (s, 3H), 1.17 (s, 3H). MS (APCI+) m/z 679.16 (M+H)⁺.

### Example 98

### 2-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 98A

### 3-[4-(5-bromo-2,3-dihydro-1H-inden-1-yl)piperazin-1-yl]propane-1,2-diol

A mixture of the product of Step 78A (1 g, 2.53 mmol), dichloromethane (10 mL) and trifluoroacetic acid (3.90 mL, 50.6 mmol) was stirred at ambient temperature overnight. The mixture was concentrated, and the residue was dissolved in dichloromethane, washed with saturated NaHCO₃, washed with brine, dried over MgSO₄, and concentrated to afford the title compound (899 mg). MS (ESI+) m/z 355 (M+H)⁺.

### Step 98B

### 3-{4-[(1S)-5-bromo-2,3-dihydro-1H-inden-1-yl]piperazin-1-yl}propane-1,2-diol

Chiral preparative super critical fluid chromatography was performed as described in Step 33A substituting the product of Step 98A for the product of Step 8C to afford the title compound as the second eluting peak.

### Step 98C

### 2-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 4C, substituting the product of Step 98B for the product of Step 4B. ¹H NMR (400 MHz, DMSO-d₆) δ 8.42-8.26 (m, 2H), 8.04-7.85 (m, 2H), 7.75 (s, 1H), 7.63-7.47 (m, 2H), 7.49-7.16 (m, 5H), 6.93 (s, 2H), 4.64-4.46 (m, 2H), 4.47-4.08 (m, 4H), 3.97-3.86 (m, 1H), 3.80 (s, 3H), 3.55 (t, J=5.9 Hz, 1H), 3.26 (s, 4H), 2.97-2.68 (m, 2H), 2.47-2.11 (m, 8H), 2.07-1.43 (m, 8H). MS (ESI+) m/z 666 (M+H)⁺.

### Example 99

### 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide

The title compound was prepared as described in Step 4C, substituting the product of Step 30C for Intermediate 33 and substituting the product of Step 98B for the product of Step 4B. ¹H NMR (400 MHz, DMSO-d₆) δ 8.47 (q, J=3.4, 2.7 Hz, 2H), 8.17 (d, J=2.4 Hz, 1H), 7.67 (d, J=7.2 Hz, 1H), 7.52 (d, J=8.1 Hz, 2H), 7.47-7.18 (m, 7H), 6.77 (d, J=2.0 Hz, 1H), 6.60 (dd, J=7.1, 2.1 Hz, 1H), 4.56 (s, 2H), 4.45-4.06 (m, 4H), 3.96-3.82 (m, 1H), 3.64-3.49 (m, 1H), 3.4 (s, 3H), 3.28 (t, J=4.8 Hz, 5H), 2.94-2.71 (m, 2H), 2.50 (s, 2H), 2.30 (ddd, J=50.7, 12.8, 7.3 Hz, 5H), 2.07-1.87 (m, 4H), 1.75-1.50 (m, 4H). MS (ESI+) m/z 693 (M+H)⁺.

### Example 100

### 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide

The title compound was prepared as described in Step 4C substituting Intermediate 32 for Example 33 and substituting the product of Step 98B for the product of Step 4B. ¹H NMR (501 MHz, DMSO-d₆) δ 8.53 (d, J=2.6 Hz, 1H), 8.44 (d, J=2.3 Hz, 1H), 8.37 (d, J=7.8 Hz, 1H), 8.23 (td, J=8.2, 2.7 Hz, 1H), 8.14 (d, J=2.4 Hz, 1H), 7.56-7.48 (m, 2H), 7.41-7.19 (m, 8H), 4.58 (s, 2H), 4.38-4.14 (m, 3H), 3.90 (dt, J=6.4, 4.0 Hz, 1H), 3.60-3.53 (m, 1H), 3.3-3.2 (m, 4H), 3.15 (d, J=5.0 Hz, 1H), 2.89 (ddd, J=15.9, 8.1, 5.5 Hz, 1H), 2.77 (dt, J=16.0, 7.7 Hz, 1H), 2.45-2.18 (m, 8H), 2.09-1.49 (m, 8H). MS (ESI+) m/z 681 (M+H)⁺.

### Example 101

### 2-amino-N-{(1S,2S)-2-[(4-{(1R)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 101A

### 3-{4-[(1R)-5-bromo-2,3-dihydro-1H-inden-1-yl]piperazin-1-yl}propane-1,2-diol

Chiral preparative super critical fluid chromatography was performed as described in Step 33A substituting the product of Step 98A for the product of Step 8C to afford the title compound as the first eluting peak. MS (ESI+) m/z 355 (M+H)⁺.

### Step 101B

### 2-amino-N-{(1S,2S)-2-[(4-{(1R)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 8D substituting the product of Step 101A for the product of Step 8C. ¹H NMR (500 MHz, DMSO-d₆) δ 8.51-8.27 (m, 2H), 8.22-7.91 (m, 2H), 7.79 (d, J=0.9 Hz, 1H), 7.66-7.54 (m, 2H), 7.51-7.16 (m, 5H), 6.99 (s, 2H), 4.67-4.54 (m, 2H), 4.53-4.23 (m, 3H), 4.11 (q, J=5.3 Hz, 1H), 3.93 (dt, J=6.4, 3.9 Hz, 1H), 3.84 (s, 3H), 3.61 (s, 1H), 3.33-3.27 (m, 3H), 3.18 (d, J=5.1 Hz, 2H), 2.93 (dt, J=14.4, 6.8 Hz, 1H), 2.81 (dt, J=15.7, 7.6 Hz, 1H), 2.61-2.53 (m, 2H), 2.36 (d, J=62.0 Hz, 5H), 2.12-1.87 (m, 4H), 1.81-1.53 (m, 4H). MS (ESI+) m/z 666 (M+H)⁺.

### Example 102

### 6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-2′-fluoro[3,3′-bipyridine]-5-carboxamide

The title compound was prepared as described in Step 8D substituting Intermediate 19 for Intermediate 33 and substituting the product of Step 98B for the product Step 8C. ¹H NMR (400 MHz, DMSO-d₆) δ 8.36 (d, J=7.8 Hz, 1H), 8.31 (t, J=2.2 Hz, 1H), 8.14 (dt, J=4.8, 1.6 Hz, 1H), 8.12-8.03 (m, 2H), 7.56-7.46 (m, 2H), 7.45-7.30 (m, 5H), 7.30-7.21 (m, 3H), 4.55 (s, 2H), 4.36-4.21 (m, 3H), 3.87 (dt, J=6.1, 3.8 Hz, 1H), 3.55 (s, 1H), 3.27 (d, J=11.7 Hz, 4H), 2.88 (ddd, J=14.5, 8.0, 5.8 Hz, 1H), 2.76 (dt, J=15.8, 7.7 Hz, 1H), 2.44-2.28 (m, 6H), 2.20 (dd, J=12.6, 6.9 Hz, 1H), 2.09-1.81 (m, 5H), 1.74-1.41 (m, 5H). MS (ESI+) m/z 681 (M+H)⁺.

### Example 103

### 2-amino-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 43D substituting Intermediate 14 for the product of Step 43C. ¹H NMR (400 MHz, DMSO-d₆, 120° C.) δ 8.24 (d, J=2.3 Hz, 1H), 7.99-7.88 (m, 2H), 7.84 (s, 1H), 7.67 (s, 1H), 7.57-7.51 (m, 2H), 7.41-7.34 (m, 4H), 7.29-7.24 (m, 1H), 6.54 (brs, 2H), 4.66-4.57 (m, 2H), 4.38-4.27 (m, 2H), 4.04-3.98 (m, 1H), 3.83 (s, 3H), 3.77-3.68 (m, 1H), 3.54-3.47 (m, 2H), 2.58-2.45 (m, 7H), 2.43 (t, J=6.1 Hz, 2H), 2.12-2.01 (m, 1H), 2.01-1.88 (m, 3H), 1.80-1.57 (m, 4H), 1.37 (s, 3H), 1.20 (s, 3H). MS (ESI+) m/z 664.3 (M+H)⁺.

### Example 104

### 6-amino-6′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Example 19 substituting Intermediate 14 for the product of Step 8C. ¹H NMR (400 MHz, DMSO-d₆, 120° C.) δ 8.48-8.44 (m, 1H), 8.38 (d, J=2.4 Hz, 1H), 8.15 (td, J=8.1, 2.6 Hz, 1H), 8.10 (d, J=2.4 Hz, 1H), 8.06-7.96 (m, 1H), 7.56-7.51 (m, 2H), 7.40-7.34 (m, 4H), 7.28-7.23 (m, 1H), 7.11 (dd, J=8.5, 3.0 Hz, 1H), 6.85 (brs, 2H), 4.66-4.56 (m, 2H), 4.38-4.29 (m, 2H), 4.04-3.98 (m, 1H), 3.77-3.69 (m, 1H), 3.50 (q, J=5.8 Hz, 2H), 2.57-2.45 (m, 7H), 2.43 (t, J=6.1 Hz, 2H), 2.13-2.02 (m, 1H), 2.01-1.87 (m, 3H), 1.79-1.57 (m, 4H), 1.37 (s, 3H), 1.20 (s, 3H). MS (ESI+) m/z 679.3 (M+H)⁺.

### Example 105

### 2-amino-N-{(1S,2S)-2-[(4-{1-[4-(2-hydroxyethyl)piperazin-1-yl]-2,2-dimethyl-2,3-dihydro-1H-inden-5-yl}phenyl)methoxy]cyclopentyl}-5-(1-methyl-1H-pyrazol-4-yl)pyridine-3-carboxamide

### Step 105A

### 5-bromo-2,2-dimethyl-2,3-dihydro-1H-inden-1-one

To a suspension of 5-bromo-1-indanone (0.5 g, 2.37 mmol) in N,N-dimethylformamide (5.9 mL) was added sodium hydride (0.208 g, 5.21 mmol), and the mixture was stirred for 1 hour. Iodomethane (1.037 mL, 16.58 mmol) was added, and the mixture was stirred for 1.5 hours. The mixture was quenched with water, extracted with dichloromethane and concentrated. Purification by flash chromatography on silica gel eluting with 0-50% ethyl acetate/heptanes afforded the title compound. ¹H NMR (501 MHz, CDCl₃) δ 7.64-7.58 (m, 2H), 7.54-7.49 (m, 1H), 2.98 (d, J=1.1 Hz, 2H), 1.23 (s, 6H). LCMS (ESI+) m/z 239.0 (M+H)⁺.

### Step 105B

### 5-bromo-2,2-dimethyl-2,3-dihydro-1H-inden-1-amine

A solution of the product of Step 105A (603 mg, 2.52 mmol) and ammonium acetate (1942 mg, 25.2 mmol) in methanol (12.6 mL) was stirred at ambient temperature for 2 hours. Sodium cyanoborohydride (633 mg, 10.08 mmol) and titanium tetrachloride (1.26 mL, 1.26 mmol) were added, and the mixture was stirred at 60° C. for 5 days. The mixture was quenched with water, extracted with dichloromethane and concentrated. Purification by flash chromatography on silica gel eluting with 0-15% methanol/dichloromethane with 3% NH₄OH afforded the title compound (762 mg). ¹H NMR (501 MHz, CDCl₃) δ 7.37-7.29 (m, 2H), 7.20 (d, J=7.9 Hz, 1H), 3.92 (s, 1H), 2.71 (d, J=3.4 Hz, 2H), 1.20 (s, 3H), 0.94 (s, 3H). LCMS (ESI+) m/z 239.98 (M+H)⁺.

### Step 105C

### 1-(5-bromo-2,2-dimethyl-2,3-dihydro-1H-inden-1-yl)-4-(4-nitrobenzene-1-sulfonyl)piperazine

To as solution of the product of Step 105B (30 mg, 0.125 mmol) in acetonitrile (1.2 mL) was added Intermediate 27 and N,N-diisopropylethylamine (0.07 mL, 0.375 mmol), and the mixture was stirred at 40° C. for 3 days. The mixture was concentrated and purified by flash chromatography on silica gel eluting with 5-70% ethyl acetate/heptanes to afford the title compound (50 mg). LCMS (ESI+) m/z 494.07 (M+H)⁺.

### Step 105D

### 1-(5-bromo-2,2-dimethyl-2,3-dihydro-1H-inden-1-yl)piperazine

Tetrahydrofuran (0.5 mL) was added to nitrogen sparged sodium hydride (6.07 mg, 0.25 mmol) followed by 1-decanethiol (0.06 mL, 0.30 mmol), and the mixture was stirred for 30 minutes. A solution of the product of Step 105C (50 mg, 0.10 mmol) in tetrahydrofuran (0.5 mL) was added, and the mixture was stirred at ambient temperature for 16 hours. The mixture was quenched with water, extracted with dichloromethane, concentrated, and purified by flash chromatography on silica gel eluting with 0-20% methanol/dichloromethane with 3% NH₄OH to afford the title compound (30 mg). LCMS (ESI+) m/z 309.07 (M+H)⁺.

### Step 105E

### 2-[4-(5-bromo-2,2-dimethyl-2,3-dihydro-1H-inden-1-yl)piperazin-1-yl]ethan-1-ol

The title compound was prepared from the product of Step 105D by following the procedures of Step 50E. LCMS (ESI+) m/z 353.11 (M+H)⁺.

### Step 105F

### 2-amino-N-{(1S,2S)-2-[(4-{1-[4-(2-hydroxyethyl)piperazin-1-yl]-2,2-dimethyl-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 105E in place of the product of Step 50E. ¹H NMR (501 MHz, methanol-d₄) δ 8.54 (d, J=2.1 Hz, 1H), 8.22 (t, J=2.5 Hz, 1H), 8.01 (d, J=3.1 Hz, 1H), 7.88 (s, 1H), 7.56 (d, J=8.1 Hz, 2H), 7.47-7.38 (m, 5H), 4.69 (d, J=3.4 Hz, 2H), 4.45 (td, J=7.3, 4.3 Hz, 1H), 4.04 (dt, J=6.5, 4.3 Hz, 1H), 3.93 (s, 3H), 3.85 (dd, J=6.1, 4.3 Hz, 2H), 3.78 (s, 1H), 3.23 (dd, J=6.3, 4.1 Hz, 3H), 2.97 (d, J=16.2 Hz, 4H), 2.67 (d, J=16.3 Hz, 1H), 2.27-2.14 (m, 1H), 2.14-2.01 (m, 1H), 1.84 (ttdd, J=13.2, 10.0, 6.6, 4.0 Hz, 3H), 1.67 (dq, J=14.6, 7.4 Hz, 1H), 1.30 (s, 3H), 1.11 (s, 3H). MS (ESI+) m/z 664.6 (M+H)⁺.

### Example 106

### 6-amino-6′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

### Step 106A

### 2-[4-(6-bromo-1,2,3,4-tetrahydronaphthalen-1-yl)piperazin-1-yl]ethan-1-ol

To a mixture of 6-bromo-3,4-dihydronaphthalen-1 (2H)-one (500 mg, 2.22 mmol) and N-(2-hydroxyethyl)piperazine (376 mg, 2.89 mmol) in anhydrous dioxane (8 mL) and was added titanium(IV) isopropoxide (1.263 g, 4.44 mmol). The mixture was heated at 80° C. for 6 hours and NaCNBH₃ (279 mg, 4.44 mmol) was added. The mixture was heated at 60° C. overnight and at 80° C. for 2 days. After cooling, the mixture was stirred with ethyl acetate and aqueous NaOH. The solid was filtered through diatornaceous earth and the organic phase was washed with brine and concentrated. The residue was purified by flash chromatography on silica gel using 15-60% ethyl acetate/heptane to provide the title compound (0.27 g).

### Step 106B

### 6-amino-6′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

A mixture of Intermediate 32 (149 mg, 0.28 mmol), the product of Step 106A (95 mg, 0.28 mmol), bis(di-tert-butyl(4-dimethylaminophenyl)phosphine) dichloropalladium(II) (9.91 mg, 0.014 mmol), and potassium phosphate tribasic (0.357 g, 1.68 mmol) was purged with nitrogen. Dioxane (5 mL) and water (1.56 mL) were added, and the mixture was purged with nitrogen and heated at 90° C. for 3 hours. After cooling, the mixture was partitioned between ethyl acetate and brine. The organic phase was concentrated, and the residue was purified by flash chromatography on silica gel using 0-20% (3% NH₄OH in methanol)/dichloromethane to provide the title compound (85 mg). ¹H NMR (400 MHz, DMSO-d₆) δ 8.55 (d, J=2.6 Hz, 1H), 8.46 (d, J=2.3 Hz, 1H), 8.40 (d, J=7.8 Hz, 1H), 8.31-8.20 (m, 1H), 8.16 (d, J=2.4 Hz, 1H), 7.62 (d, J=8.1 Hz, 1H), 7.54 (d, J=7.9 Hz, 2H), 7.36 (dd, J=8.2, 6.5 Hz, 3H), 7.31-7.16 (m, 4H), 4.60 (s, 2H), 4.44-4.24 (m, 2H), 3.92 (dt, J=6.3, 3.9 Hz, 1H), 3.77 (q, J=5.1 Hz, 1H), 3.49 (q, J=5.8 Hz, 3H), 2.80-2.63 (m, 2H), 2.40 (t, J=6.3 Hz, 2H), 2.19-1.83 (m, 8H), 1.84-1.44 (m, 8H). MS (ESI+) m/z 665 (M+H)⁺.

### Example 107

### 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-1′-methyl-2′-oxo-1′,2′-dihydro[3,4′-bipyridine]-5-carboxamide

The title compound was prepared as described in Step 12D substituting the product of Step 30C for Intermediate 33. ¹H NMR (501 MHz, DMSO-d₆) δ 8.49 (p, J=2.4 Hz, 2H), 8.20 (t, J=2.5 Hz, 1H), 7.69 (dd, J=7.2, 1.2 Hz, 1H), 7.60-7.50 (m, 2H), 7.44 (s, 2H), 7.41-7.28 (m, 4H), 7.21 (ddd, J=8.4, 2.4, 0.9 Hz, 1H), 6.79 (t, J=1.9 Hz, 1H), 6.62 (dt, J=7.2, 2.4 Hz, 1H), 4.65-4.51 (m, 2H), 4.34 (dt, J=28.7, 6.9 Hz, 2H), 3.92 (dtd, J=5.6, 4.0, 1.4 Hz, 1H), 3.40 (s, 3H), 2.48-2.24 (m, 7H), 2.13 (s, 3H), 2.09-1.50 (m, 9H), 1.33 (s, 3H), 1.14 (d, J=4.4 Hz, 3H). MS (ESI+) m/z 661 (M+H)⁺.

### Example 108

### 6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide

The title compound was prepared as described in Step 12D substituting Intermediate 19 for Intermediate 24. ¹H NMR (501 MHz, DMSO-d₆) δ 8.37 (dd, J=7.7, 2.2 Hz, 1H), 8.33 (t, J=2.1 Hz, 1H), 8.15 (dt, J=4.9, 1.6 Hz, 1H), 8.14-8.06 (m, 2H), 7.61-7.52 (m, 2H), 7.42-7.34 (m, 5H), 7.30 (s, 2H), 7.25-7.19 (m, 1H), 4.66-4.54 (m, 2H), 4.46-4.35 (m, 1H), 4.29 (qd, J=7.6, 4.0 Hz, 1H), 4.07 (q, J=5.3 Hz, 1H), 3.89 (dt, J=6.7, 3.9 Hz, 1H), 3.15 (d, J=4.8 Hz, 1H), 2.47-2.23 (m, 6H), 2.14 (s, 3H), 2.09-1.48 (m, 8H), 1.35 (d, J=2.2 Hz, 3H), 1.16 (t, J=2.5 Hz, 3H). MS (ESI+) m/z 649 (M+H)⁺.

### Example 109

### 2-amino-N-[(1S,2S)-2-({4-[1-(dimethylamino)-2,2-dimethyl-2,3-dihydro-1H-inden-5-yl]phenyl}methoxy)cyclopentyl]-5-(1-methyl-1H-pyrazol-4-yl)pyridine-3-carboxamide

### Step 109A

### 5-bromo-N,N, 2,2-tetramethyl-2,3-dihydro-1H-inden-1-amine

To a solution of the product of Step 105B (200 mg, 0.666 mmol) in 1,2-dichloroethane (4.4 mL) was added aqueous formaldehyde solution (0.15 mL, 1.999 mmol), followed by sodium triacetoxyborohydride (706 mg, 3.33 mmol), and the mixture was stirred at ambient temperature for 16 hours. The mixture was purified by flash chromatography on silica gel eluting with 0-10% methanol/dichloromethane to afford the title compound (46 mg). LCMS (ESI+) m/z 268.01 (M+H)⁺.

### Step 109B

### 2-amino-N-[(1S,2S)-2-({4-[1-(dimethylamino)-2,2-dimethyl-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 50F using the product of Step 109A in place of the product of Step 50E. ¹H NMR (501 MHz, methanol-d₄) δ 8.51 (dd, J=2.2, 1.1 Hz, 1H), 8.24 (dd, J=2.1, 1.2 Hz, 1H), 8.02 (d, J=1.9 Hz, 1H), 7.87 (s, 1H), 7.67 (d, J=7.8 Hz, 1H), 7.65-7.57 (m, 4H), 7.51-7.43 (m, 2H), 4.71 (s, 2H), 4.50-4.42 (m, 2H), 4.05 (dt, J=6.5, 4.2 Hz, 1H), 3.94 (s, 3H), 3.22 (d, J=16.9 Hz, 1H), 3.10 (s, 3H), 2.82 (dd, J=17.0, 1.7 Hz, 1H), 2.50 (s, 3H), 2.29-2.15 (m, 1H), 2.14-2.01 (m, 1H), 1.92-1.77 (m, 3H), 1.68 (dq, J=14.7, 7.6 Hz, 1H), 1.52 (s, 3H), 1.18 (s, 3H). MS (ESI+) m/z 578.8 (M+H)⁺.

### Example 110

### 2-amino-N-{(1S,2S)-2-[(4-{1-[1-(2-hydroxyethyl)piperidin-4-yl]-3-methyl-1H-pyrrolo[2,3-b]pyridin-5-yl}phenyl)methoxy]cyclopentyl}-5-(1-methyl-1H-pyrazol-4-yl)pyridine-3-carboxamide

### Step 110A

### tert-butyl 4-(5-bromo-3-methyl-1H-pyrrolo[2,3-b]pyridin-1-yl)piperidine-1-carboxylate

The title compound was prepared as described in Step 73A using 5-bromo-3-methyl-1H-pyrrolo[2,3-b]pyridine in place of 5-bromo-3,3-dimethyl-1H-pyrrolo[3,2-b]pyridin-2 (3H)-one. MS (ESI+) m/z 393.9 (M+H)⁺.

### Step 110B

### 5-bromo-3-methyl-1-(piperidin-4-yl)-1H-pyrrolo[2,3-b]pyridine

The title compound was prepared as described in Step 72A using the product of Step 110A in place of tert-butyl 6′-bromo-1′H-spiro[cyclopropane-1,4′-isoquinoline]-2′(3′H)-carboxylate. MS (ESI+) m/z 293.9 (M+H)⁺.

### Step 110C

### 2-[4-(5-bromo-3-methyl-1H-pyrrolo[2,3-b]pyridin-1-yl)piperidin-1-yl]ethan-1-ol

The title compound was prepared as described in Step 72D using the product of Step 110B in place of the product of Step 72C. ¹H NMR (400 MHz, methanol-d₄) δ 8.26 (d, J=2.2 Hz, 1H), 8.09 (d, J=2.1 Hz, 1H), 7.31 (s, 1H), 4.94 (ddt, J=12.1, 7.9, 4.1 Hz, 1H), 3.97-3.90 (m, 2H), 3.79 (t, J=8.6 Hz, 2H), 3.35 (d, J=10.1 Hz, 4H), 2.55 (dd, J=12.5, 3.6 Hz, 1H), 2.50 (s, 1H), 2.29 (d, J=1.1 Hz, 4H), 2.25 (s, 1H). MS (ESI+) m/z 338.4 (M+H)⁺.

### Step 110D

### 2-amino-N-{(1S,2S)-2-[(4-{1-[1-(2-hydroxyethyl)piperidin-4-yl]-3-methyl-1H-pyrrolo[2,3-b]pyridin-5-yl}phenyl)methoxy]cyclopentyl}-5-(1-methyl-1H-pyrazol-4-yl)pyridine-3-carboxamide

The title compound was prepared as described in Step 72E using the product of Step 110C in place of the product of Step 72D. ¹H NMR (400 MHz, methanol-d₄) δ 8.33 (d, J=2.1 Hz, 1H), 8.21 (d, J=2.3 Hz, 1H), 7.99 (d, J=2.1 Hz, 1H), 7.92 (d, J=2.3 Hz, 1H), 7.74 (s, 1H), 7.69 (d, J=0.9 Hz, 1H), 7.59-7.51 (m, 2H), 7.43 (d, J=8.2 Hz, 2H), 7.29 (d, J=1.3 Hz, 1H), 4.75-4.57 (m, 3H), 4.42 (td, J=7.6, 4.7 Hz, 1H), 3.98 (dt, J=6.7, 4.6 Hz, 1H), 3.78 (s, 3H), 3.73 (t, J=6.0 Hz, 2H), 3.21-3.11 (m, 2H), 2.62 (t, J=6.0 Hz, 2H), 2.33 (dd, J=8.7, 1.8 Hz, 5H), 2.23-1.92 (m, 7H), 1.87-1.47 (m, 4H). MS (ESI+) m/z 649.5 (M+H)⁺.

### Example III

### 2-amino-N-{(1S,2S)-2-[(4-{1-[1-(2-hydroxyethyl)piperidin-4-yl]-1H-indazol-5-yl}phenyl)methoxy]cyclopentyl}-5-(1-methyl-1H-pyrazol-4-yl)pyridine-3-carboxamide

### Step 111A

### tert-butyl 4-(5-bromo-1H-indazol-1-yl)piperidine-1-carboxylate

The title compound was prepared as described in Step 73A using 5-bromo-1H-indazole in place of 5-bromo-3,3-dimethyl-1H-pyrrolo[3,2-b]pyridin-2 (3H)-one. ¹H NMR (400 MHz, methanol-d₄) δ 8.02-7.96 (m, 1H), 7.91 (dd, J=1.8, 0.7 Hz, 1H), 7.59 (dt, J=9.0, 0.8 Hz, 1H), 7.48 (dd, J=8.9, 1.8 Hz, 1H), 4.24 (dp, J=13.6, 2.1 Hz, 2H), 3.04 (s, 2H), 2.19-1.91 (m, 5H), 1.49 (s, 9H).

### Step 111B

### 5-bromo-1-(piperidin-4-yl)-1H-indazole

The title compound was prepared as described in Step 72A using the product of Step 111A in place of tert-butyl 6′-bromo-1′H-spiro[cyclopropane-1,4′-isoquinoline]-2′(3′H)-carboxylate. MS (ESI+) m/z 279.9 (M+H)⁺.

### Step 111C

### 2-[4-(5-bromo-1H-indazol-1-yl)piperidin-1-yl]ethan-1-ol

The title compound was prepared as described in Step 72D using the product of Step 111B in place of the product of Step 72C. MS (ESI+) m/z 326.1 (M+H)⁺.

### Step 111D

### 2-amino-N-{(1S,2S)-2-[(4-{1-[1-(2-hydroxyethyl)piperidin-4-yl]-1H-indazol-5-yl}phenyl)methoxy]cyclopentyl}-5-(1-methyl-1H-pyrazol-4-yl)pyridine-3-carboxamide

The title compound was prepared as described in Step 72E using the product of Step 111C in place of the product of Step 72D. ¹H NMR (500 MHz, methanol-d₄) δ 8.23 (d, J=2.3 Hz, 1H), 8.02 (s, 1H), 7.95 (d, J=2.2 Hz, 1H), 7.84 (dd, J=1.7, 0.8 Hz, 1H), 7.78 (d, J=0.7 Hz, 1H), 7.72 (d, J=0.8 Hz, 1H), 7.65 (d, J=8.9 Hz, 1H), 7.60-7.53 (m, 3H), 7.44-7.39 (m, 2H), 4.70 (d, J=12.4 Hz, 1H), 4.67-4.57 (m, 2H), 4.42 (td, J=7.7, 4.8 Hz, 1H), 3.97 (dt, J=6.6, 4.7 Hz, 1H), 3.80 (s, 3H), 3.73 (t, J=6.1 Hz, 2H), 3.20-3.09 (m, 2H), 2.63 (t, J=6.1 Hz, 2H), 2.44-2.31 (m, 4H), 2.16 (tt, J=13.3, 6.4 Hz, 1H), 2.04 (ddt, J=15.4, 7.5, 5.9 Hz, 3H), 1.91-1.71 (m, 3H), 1.60 (dq, J=13.1, 7.8 Hz, 1H). MS (ESI+) m/z 635.3 (M+H)⁺.

### Example 112

### 2-amino-N-{(1S,2S)-2-[(4-{1-[1-(2-hydroxyethyl)piperidin-4-yl]-1,2,3,4-tetrahydroquinolin-6-yl}phenyl)methoxy]cyclopentyl}-5-(1-methyl-1H-pyrazol-4-yl)pyridine-3-carboxamide

### Step 112A

### tert-butyl 4-(6-bromo-3,4-dihydroquinolin-1 (2H)-yl)piperidine-1-carboxylate

The title compound was prepared as described in Step 72B using 6-bromo-1,2,3,4-tetrahydroquinoline in place of the product of Step 72A. MS (ESI+) m/z 395.2 (M+H)⁺.

### Step 112B

### 6-bromo-1-(piperidin-4-yl)-1,2,3,4-tetrahydroquinoline

The title compound was prepared as described in Step 72A using the product of Step 112A in place of tert-butyl 6′-bromo-1′H-spiro[cyclopropane-1,4′-isoquinoline]-2′(3′H)-carboxylate. MS (ESI+) m/z 293.7 (M+H)⁺.

### Step 112C

### 2-[4-(6-bromo-3,4-dihydroquinolin-1 (2H)-yl)piperidin-1-yl]ethan-1-ol

The title compound was prepared as described in Step 72D using the product of Step 112B in place of the product of Step 72C. ¹H NMR (501 MHz, methanol-d₄) δ 7.14-6.99 (m, 2H), 6.70 (d, J=8.9 Hz, 1H), 4.00 (ddd, J=11.8, 7.9, 3.9 Hz, 1H), 3.89 (dd, J=6.0, 4.5 Hz, 2H), 3.77-3.62 (m, 2H), 3.25-3.12 (m, 5H), 2.69 (t, J=6.3 Hz, 2H), 2.13 (qd, J=13.5, 12.9, 3.9 Hz, 2H), 2.02-1.80 (m, 5H). MS (ESI+) m/z 339.3 (M+H)⁺.

### Step 112D

### 2-amino-N-{(1S,2S)-2-[(4-{1-[1-(2-hydroxyethyl)piperidin-4-yl]-1,2,3,4-tetrahydroquinolin-6-yl}phenyl)methoxy]cyclopentyl}-5-(1-methyl-1H-pyrazol-4-yl)pyridine-3-carboxamide

The title compound was prepared as described in Step 72E using the product of Step 112C in place of the product of Step 72D. ¹H NMR (400 MHz, methanol-d₄) δ 8.23 (d, J=2.3 Hz, 1H), 7.93 (d, J=2.3 Hz, 1H), 7.78-7.70 (m, 2H), 7.45-7.38 (m, 2H), 7.30 (d, J=8.0 Hz, 2H), 7.18 (dd, J=8.6, 2.4 Hz, 1H), 7.08 (d, J=2.3 Hz, 1H), 6.70 (d, J=8.8 Hz, 1H), 4.68-4.54 (m, 2H), 4.40 (td, J=7.6, 4.8 Hz, 1H), 3.94 (dt, J=6.7, 4.7 Hz, 1H), 3.82 (s, 3H), 3.70 (t, J=6.1 Hz, 2H), 3.23 (t, J=5.8 Hz, 2H), 3.17-3.05 (m, 3H), 2.71 (t, J=6.4 Hz, 2H), 2.57 (t, J=6.1 Hz, 2H), 2.23 (td, J=12.1, 2.4 Hz, 2H), 2.13 (dt, J=13.3, 6.2 Hz, 1H), 2.09-1.96 (m, 1H), 1.96-1.69 (m, 9H), 1.57 (dq, J=13.0, 7.7 Hz, 1H). MS (ESI+) m/z 650.6 (M+H)⁺.

### Example 113

### 2-amino-N-{(1S,2S)-2-[(4-{1-[1-(2-hydroxyethyl)piperidin-4-yl]-3,3-dimethyl-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide

### Step 113A

### tert-butyl 4-(5-bromo-3,3-dimethyl-2-oxo-2,3-dihydro-1H-pyrrolo[3,2-b]pyridin-1-yl)piperidine-1-carboxylate

The title compound was prepared as described in Step 73A using 5-bromo-3,3-dimethyl-1H-pyrrolo[3,2-b]pyridin-2 (3H)-one in place of 5-bromo-3,3-dimethyl-1H-pyrrolo[3,2-b]pyridin-2 (3H)-one. MS (ESI+) m/z 426.0 (M+H)⁺.

### Step 113B

### 5-bromo-3,3-dimethyl-1-(piperidin-4-yl)-1,3-dihydro-2H-pyrrolo[3,2-b]pyridin-2-one

The title compound was prepared as described in Step 72A using the product of Step 113A in place of tert-butyl 6′-bromo-1′H-spiro[cyclopropane-1,4′-isoquinoline]-2′(3′H)-carboxylate. ¹H NMR (400 MHz, methanol-d₄) δ 7.47 (s, 2H), 4.37 (tt, J=12.2, 4.1 Hz, 1H), 3.56 (ddt, J=13.2, 4.3, 2.0 Hz, 2H), 3.17 (td, J=13.3, 3.0 Hz, 2H), 2.67 (tdd, J=13.5, 12.1, 4.3 Hz, 2H), 2.02 (ddt, J=14.1, 3.8, 1.6 Hz, 2H), 1.36 (s, 5H). MS (ESI+) m/z 324.3 (M+H)⁺.

### Step 113C

### 5-bromo-3,3-dimethyl-1-(piperidin-4-yl)-2,3-dihydro-1H-pyrrolo[3,2-b]pyridine

To a solution of the product of Step 113B (142 mg, 0.257 mmol) in tetrahydrofuran (2 mL) was added 1 M borane-tetrahydrofuran complex in tetrahydrofuran (3.6 mL, 3.60 mmol), and the mixture was heated at 80° C. under nitrogen for 18 hours. The mixture was cooled to ambient temperature, quenched with methanol/water and concentrated. The mixture was purified by flash chromatography on silica gel eluting with 0-10% 1 M NH₃/methanol in dichloromethane to afford the title compound (48 mg). MS (ESI+) m/z 312.0 (M+H)⁺.

### Step 113D

### 2-[4-(5-bromo-3,3-dimethyl-2,3-dihydro-1H-pyrrolo[3,2-b]pyridin-1-yl)piperidin-1-yl]ethan-1-ol

The title compound was prepared as described in Step 72D using the product of Step 113C in place of the product of Step 72C. MS (ESI+) m/z 354.2 (M+H)⁺.

### Step 113E

### 2-amino-N-{(1S,2S)-2-[(4-{1-[1-(2-hydroxyethyl)piperidin-4-yl]-3,3-dimethyl-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide

The title compound was prepared as described in Step 72E using the product of Step 113D in place of the product of Step 72D. ¹H NMR (400 MHz, methanol-d₄) δ 8.24 (d, J=2.2 Hz, 1H), 7.98 (d, J=2.2 Hz, 1H), 7.81 (s, 1H), 7.78-7.71 (m, 3H), 7.36 (d, J=8.0 Hz, 2H), 7.28 (d, J=8.3 Hz, 1H), 6.76 (d, J=8.3 Hz, 1H), 4.65 (s, 2H), 4.42 (td, J=7.5, 4.5 Hz, 1H), 3.97 (dt, J=6.6, 4.4 Hz, 1H), 3.85 (s, 3H), 3.74-3.61 (m, 2H), 3.44 (td, J=10.3, 9.7, 4.8 Hz, 1H), 3.29 (m, 2H), 3.16-3.05 (m, 3H), 2.68-2.53 (m, 2H), 2.24 (dd, J=11.0, 4.3 Hz, 1H), 2.23-2.09 (m, 1H), 2.09-1.96 (m, 1H), 1.91-1.68 (m, 7H), 1.60 (dq, J=13.0, 7.6 Hz, 1H), 1.32 (d, J=4.3 Hz, 6H). MS (ESI+) m/z 665.3 (M+H)⁺.

### Example 114

### 2-amino-N-{(1S,2S)-2-[(4-{1-[1-(2-hydroxyethyl)piperidin-4-yl]-4,4-dimethyl-1,2,3,4-tetrahydroquinolin-6-yl}phenyl)methoxy]cyclopentyl}-5-(1-methyl-1H-pyrazol-4-yl)pyridine-3-carboxamide

### Step 114A

### tert-butyl 4-(6-bromo-4,4-dimethyl-3,4-dihydroquinolin-1 (2H)-yl)piperidine-1-carboxylate

The title compound was prepared as described in Step 72B using 6-bromo-4,4-dimethyl-1,2,3,4-tetrahydroquinoline in place of the product of Step 72A. MS (ESI+) m/z 425.2 (M+H)⁺.

### Step 114B

### 6-bromo-4,4-dimethyl-1-(piperidin-4-yl)-1,2,3,4-tetrahydroquinoline

The title compound was prepared as described in Step 72A using the product of Step 114A in place of tert-butyl 6′-bromo-1′H-spiro[cyclopropane-1,4′-isoquinoline]-2′(3′H)-carboxylate. MS (ESI+) m/z 323.4 (M+H)⁺.

### Step 114C

### 2-[4-(6-bromo-4,4-dimethyl-3,4-dihydroquinolin-1 (2H)-yl)piperidin-1-yl]ethan-1-ol

The title compound was prepared as described in Step 72D using the product of Step 114B in place of the product of Step 72C. MS (ESI+) m/z 367.3 (M+H)⁺.

### Step 114D

### 2-amino-N-{(1S,2S)-2-[(4-{1-[1-(2-hydroxyethyl)piperidin-4-yl]-4,4-dimethyl-1,2,3,4-tetrahydroquinolin-6-yl}phenyl)methoxy]cyclopentyl}-5-(1-methyl-1H-pyrazol-4-yl)pyridine-3-carboxamide

The title compound was prepared as described in Step 72E using the product of Step 114C in place of the product of Step 72D. ¹H NMR (500 MHz, methanol-d₄) δ 8.24 (d, J=2.2 Hz, 1H), 7.96 (d, J=2.3 Hz, 1H), 7.79-7.72 (m, 2H), 7.49-7.41 (m, 2H), 7.38-7.30 (m, 3H), 7.19 (dd, J=8.6, 2.4 Hz, 1H), 6.72 (d, J=8.9 Hz, 1H), 4.67-4.57 (m, 2H), 4.41 (td, J=7.6, 4.8 Hz, 1H), 3.95 (dt, J=6.7, 4.7 Hz, 1H), 3.81 (s, 3H), 3.79-3.66 (m, 3H), 3.30-3.22 (m, 2H), 3.17-3.06 (m, 3H), 2.58 (t, J=6.2 Hz, 2H), 2.29-2.20 (m, 2H), 2.20-2.09 (m, 1H), 2.02 (ddd, J=12.7, 7.7, 6.1 Hz, 1H), 1.98-1.84 (m, 2H), 1.86-1.67 (m, 6H), 1.58 (dq, J=13.1, 7.8 Hz, 1H), 1.26 (d, J=8.7 Hz, 6H). MS (ESI+) m/z 678.1 (M+H)⁺.

### Example 115

### 6-amino-1′-methyl-N-[(1S,2S)-2-({4-[(1S)-1-(4-methylpiperazin-1-yl)-2,3-dihydro-1H-inden-5-yl]phenyl}methoxy)cyclopentyl]-2′-oxo-1′,2′-dihydro[3,4′-bipyridine]-5-carboxamide

The title compound was prepared as described in Step 12D substituting the product of Step 30C for Intermediate 24 and substituting the product of Step 139A for the product of Step 12C. ¹H NMR (501 MHz, DMSO-d₆) δ 8.53-8.45 (m, 2H), 8.19 (d, J=2.4 Hz, 1H), 7.69 (d, J=7.2 Hz, 1H), 7.56-7.50 (m, 2H), 7.49-7.30 (m, 6H), 7.26 (d, J=7.9 Hz, 1H), 6.79 (d, J=2.1 Hz, 1H), 6.62 (dd, J=7.1, 2.1 Hz, 1H), 4.65-4.47 (m, 2H), 4.39-4.20 (m, 2H), 3.92 (dt, J=6.3, 4.0 Hz, 1H), 3.40 (s, 3H), 2.88 (ddd, J=16.1, 8.3, 5.5 Hz, 1H), 2.77 (dt, J=15.7, 7.7 Hz, 1H), 2.40-2.21 (m, 6H), 2.12 (s, 3H), 2.08-1.86 (m, 5H), 1.78-1.37 (m, 5H). MS (ESI+) m/z 633 (M+H)⁺.

### Example 116

### 6-amino-2′-fluoro-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide

The title compound was prepared as described in Step 12D substituting Intermediate 19 for Intermediate 24 and substituting the product of Step 139A for the product of Step 12C. ¹H NMR (501 MHz, DMSO-d₆) δ 8.37 (d, J=7.7 Hz, 1H), 8.33 (t, J=2.2 Hz, 1H), 8.16 (dt, J=4.8, 1.6 Hz, 1H), 8.14-8.05 (m, 2H), 7.56-7.51 (m, 2H), 7.44-7.25 (m, 8H), 4.63-4.52 (m, 2H), 4.28 (dt, J=14.1, 5.4 Hz, 2H), 4.08 (s, 1H), 3.89 (dt, J=6.3, 3.9 Hz, 1H), 2.94-2.71 (m, 3H), 2.42-2.25 (m, 6H), 2.16 (s, 3H), 2.08-1.95 (m, 3H), 1.94-1.86 (m, 1H), 1.75-1.60 (m, 3H), 1.54 (dtd, J=15.0, 8.2, 7.6, 4.4 Hz, 1H). MS (ESI+) m/z 621 (M+H)⁺.

### Example 117

### 6-amino-6′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide

### Step 117A

### (7R,8aS)-2-[(1S)-5-bromo-2,3-dihydro-1H-inden-1-yl]octahydropyrrolo[1,2-a]pyrazin-7-ol

To a solution of (S)-5-bromo-2,3-dihydro-1H-inden-1-ol (0.36 g, 1.69 mmol) in dichloromethane (2.81 mL) was added N,N-diisopropylethylamine (0.305 mL, 1.74 mmol). The solution was cooled 0° C. and methanesulfonyl chloride (0.132 mL, 1.69 mmol) was added dropwise. The mixture was stirred for 1 hour at ambient temperature, water was added and extracted with dichloromethane. The organic layer was dried over anhydrous magnesium sulfate, filtered, and concentrated under reduced pressure. To a solution of the residue in N-methyl-2-pyrrolidinone (2.8 mL) was added (7R,8aS)-octahydropyrrolo[1,2-a]pyrazin-7-ol (0.16 g, 1.13 mmol) and N,N-diisopropylethylamine (0.59 mL, 3.38 mmol). The mixture was stirred overnight and diluted with ethyl acetate. 1 M LiCl was added, and the ethyl acetate layer was dried over MgSO₄, filtered, and concentrated. The crude material was purified by flash chromatography on silica gel eluting with 0-25% (9:1 methanol/NH₄OH)/dichloromethane to afford the title compound (0.156 g). ¹H NMR (400 MHz, CDCl₃) δ 7.36-7.27 (m, 2H), 7.21 (d, 1H), 4.47 (tdd, 1H), 4.30 (t, 1H), 3.46 (dd, 1H), 3.42-3.34 (m, 1H), 3.00-2.84 (m, 3H), 2.84 (s, 2H), 2.79 (dd, 1H), 2.59-2.45 (m, 2H), 2.43-2.34 (m, 2H), 2.30 (td, 1H), 2.19-2.10 (m, 3H), 2.13-2.06 (m, 1H), 2.10-1.95 (m, 2H), 1.83-1.64 (m, 2H). MS (ESI+) m/z 337.3 (M+H)⁺.

### Step 117B

### 6-amino-6′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide

A mixture of Step 117A (0.028 g, 0.083 mmol), Intermediate 32 (0.053 g, 0.10 mmol), tris(dibenzylideneacetone)dipalladium(0) (1.52 mg, 1.66 umol), (4-(N,N-dimethylamino)phenyl)di-tert-butyl phosphine (0.969 mg, 3.65 umol) and potassium phosphate, tribasic (0.037 g, 0.174 mmol) was purged with argon and a degassed mixture of dioxane (0.66 mL) and water (0.17 mL) was added. The mixture heated at 75° C. for 2 hours, cooled to ambient temperature, and ammonium pyrrolidine-1-carbodithioate (1.364 mg) was added. ethyl acetate and water were added, and the biphasic mixture was stirred for 20 minutes. The mixture was filtered through diatomaceous earth and treated with 1 M NaOH. The mixture was extracted with ethyl acetate and the organic layers were dried over MgSO₄, filtered, and concentrated. The residue was purified by flash chromatography on silica gel eluting with 0-60% methanol/ethyl acetate to afford the title compound (0.040 mg). ¹H NMR (400 MHz, methanol-d₄) δ 8.34 (dt, 1H), 8.30 (d, 1H), 8.05 (ddd, 1H), 7.98 (d, 1H), 7.51-7.43 (m, 2H), 7.42-7.35 (m, 3H), 7.34-7.27 (m, 2H), 7.02 (ddd, 1H), 4.69 (d, 1H), 4.61 (d, 1H), 4.56 (s, 1H), 4.39 (tdd, 3H), 3.95 (dt, 1H), 3.39 (dd, 1H), 3.06-2.77 (m, 4H), 2.65-2.55 (m, 2H), 2.48 (td, 1H), 2.32 (td, 1H), 2.25-2.09 (m, 5H), 2.05 (td, 1H), 1.86-1.75 (m, 2H), 1.75-1.68 (m, 2H), 1.57 (dq, 1H). MS (ESI+) m/z 663.1 (M+H)⁺.

### Example 118

### 6-amino-6′-fluoro-N-{(1S,2S)-2-[(4-{(1R)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide

### Step 118A

### (7R,8aS)-2-[(1R)-5-bromo-2,3-dihydro-1H-inden-1-yl]octahydropyrrolo[1,2-a]pyrazin-7-ol

The title compound was prepared as described in Step 117A using (R)-5-bromo-2,3-dihydro-1H-inden-1-ol in place of (S)-5-bromo-2,3-dihydro-1H-inden-1-ol. ¹H NMR (501 MHz, DMSO-d₆) δ 7.42 (d, 1H), 7.36 (dd, 1H), 7.19 (dd, 1H), 4.75 (d, 1H), 4.28 (dt, 1H), 4.17 (q, 1H), 3.25 (dd, 1H), 2.91-2.81 (m, 2H), 2.78 (t, 1H), 2.77-2.69 (m, 1H), 2.46-2.36 (m, 2H), 2.29-2.23 (m, 2H), 2.00 (tdd, 3H), 1.88 (t, 1H), 1.50 (s, 1H), 1.49-1.40 (m, 1H). MS (ESI+) m/z 337.3.

### Step 118B

### 6-amino-6′-fluoro-N-{(1S,2S)-2-[(4-{(1R)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide

The title compound was prepared as in described in Step 117B using Step 118A in place of Step 117A. ¹H NMR (501 MHz, methanol-d₄) δ 8.35 (d, 1H), 8.30 (d, 1H), 8.09-8.01 (m, 1H), 7.98 (d, 1H), 7.49-7.41 (m, 2H), 7.41-7.35 (m, 3H), 7.34-7.28 (m, 2H), 7.01 (dd, 1H), 4.69 (d, 1H), 4.61 (d, 1H), 4.56 (s, 1H), 4.41 (td, 2H), 4.38-4.31 (m, 1H), 3.95 (dt, 1H), 3.40 (dd, 1H), 3.04-2.93 (m, 2H), 2.85 (td, 2H), 2.75-2.69 (m, 1H), 2.56 (td, 1H), 2.45 (tdd, 2H), 2.22-2.09 (m, 4H), 2.09-1.99 (m, 2H), 1.87-1.69 (m, 2H), 1.69-1.51 (m, 2H). MS (ESI+) m/z 663.1 (M+H)⁺.

### Example 119

### 6-amino-2′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Step 12D substituting Intermediate 19 for Intermediate 24 and substituting Intermediate 13 for the product of Step 12C. ¹H NMR (501 MHz, DMSO-d₆) δ 8.52-8.28 (m, 2H), 8.24-8.01 (m, 3H), 7.66-7.48 (m, 2H), 7.48-7.12 (m, 8H), 4.66-4.51 (m, 2H), 4.43-4.23 (m, 3H), 3.89 (dt, J=6.8, 3.9 Hz, 1H), 3.46 (q, J=5.7 Hz, 2H), 2.76 (s, 1H), 2.45-2.25 (m, 9H), 2.09-1.50 (m, 8H), 1.34 (d, J=2.3 Hz, 3H), 1.16 (d, J=2.6 Hz, 3H). MS (ESI+) m/z 679 (M+H)⁺.

### Example 120

### 2-amino-N-[(1S,2S)-2-({4-[5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

### Step 120A

### 6-bromo-1,2,3,4-tetrahydronaphthalen-1-amine

A mixture of 6-bromo-3,4-dihydronaphthalen-1 (2H)-one (1 g, 4.4 mmol), NaCNBH₃ (0.33 g, 5.3 mmol) and ammonium acetate (3.4 g, 44 mmol) in ethanol (2 mL) was heated in a microwave under 130° C. for 3 minutes. The mixture was quenched with water and extracted with ethyl acetate. The organic phase dried over MgSO₄, concentrated and purified by flash chromatography on silica gel eluting with 10% methanol/ethyl acetate to afford the title compound (0.85 g). ¹H NMR (400 MHz, methanol-d₄) δ 7.33-7.22 (m, 3H), 4.02 (d, J=11.5 Hz, 1H), 2.85-2.62 (m, 2H), 2.10-1.85 (m, 2H), 1.82-1.68 (m, 2H). MS (DCI/NH₃) m/z 297 (M+H)⁺.

### Step 120B

### 1-(6-bromo-1,2,3,4-tetrahydronaphthalen-1-yl)-4-(4-methylbenzene-1-sulfonyl)piperazine

To a mixture of N,N-bis(2-chloroethyl)-4-methylbenzenesulfonamide (0.85 g, 2.87 mmol) in N-ethyl-N-isopropylpropan-2-amine (8 mL) was added the product of Step 120A (0.65 g, 2.87 mmol), and the mixture was stirred at 145° C. for 50 hours. The mixture was cooled to ambient temperature and partitioned between ethyl acetate and water. The organic phase was concentrated and purified by flash chromatography on silica gel eluting with 10% ethyl acetate/hexane to afford the title compound (0.7 g). MS (DCI/NH₃) m/z 450 (M+H)⁺.

### Step 120C

### 1-(6-bromo-1,2,3,4-tetrahydronaphthalen-1-yl)piperazine

To a solution of the product of Step 120B (700 mg, 1.56 mmol) in HBr/acetic acid (5 mL) was added 4-hydroxybenzoic acid (400 mg, 3 mmol), and the mixture was stirred at 90° C. for 1 hour. Water was added, and the solid was filtered and washed with water. The filtrate was adjusted to pH 9 with aqueous NaOH, extracted with ethyl acetate. The organic phase was concentrated to afford the title compound (0.28 g) which was used in the next step without further purification. MS (DCI/NH₃) m/z 296 (M+H)⁺.

### Step 120D

### 1-(6-bromo-1,2,3,4-tetrahydronaphthalen-1-yl)-4-methylpiperazine

To a solution of the product of Step 120C (100 mg, 0.34 mmol) in dichloromethane (5 mL) was added methyl iodide (95 mg, 0.68 mmol) and K₂CO₃ (93 mg, 0.68 mmol), and the mixture was stirred at ambient temperature overnight. The mixture was partitioned between ethyl acetate and water, and the organic phase was dried over MgSO₄ and concentrated to afford the title compound (80 mg) which was used without further purification. MS (DCI/NH₃) m/z 309 (M+H)⁺.

### Step 120E

### 2-amino-N-[(1S,2S)-2-({4-[5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

A mixture of Intermediate 3 (50 mg, 0.1 mmol), the product of Step 120D (31 mg, 0.1 mmol), bis(di-tert-butyl(4-dimethylaminophenyl)phosphine)dichloropalladium(II) (3 mg, 0.0048 mmol) and K₃PO₄ (40 mg, 0.19 mmol) was sparged with argon, and dioxane (5 mL) and water (1 mL) were added. The mixture was sparged with argon and stirred at 100° C. for 1 hour. The mixture was quenched with water and partitioned between water and ethyl acetate. The organic phase was dried over MgSO₄, concentrated and purified by flash chromatography on silica gel eluting with 30% methanol/ethyl acetate to afford the title compound (20 mg). ¹H NMR (400 MHz, DMSO-d₆) δ 8.34-8.27 (m, 2H), 7.96 (dd, J=11.6, 2.3 Hz, 2H), 7.75 (s, 1H), 7.58 (d, J=8.1 Hz, 1H), 7.55-7.49 (m, 2H), 7.34 (d, J=8.3 Hz, 3H), 7.26 (d, J=2.0 Hz, 1H), 6.94 (s, 2H), 4.64-4.51 (m, 2H), 4.28 (dq, J=7.8, 3.7 Hz, 1H), 3.89 (dt, J=6.3, 3.8 Hz, 1H), 3.80 (s, 3H), 3.75 (p, J=6.2, 5.4 Hz, 1H), 2.70 (t, J=5.0 Hz, 2H), 2.42 (s, 3H), 2.36-2.25 (m, 4H), 2.14 (m, 4H), 2.01 (ddd, J=15.4, 8.2, 5.3 Hz, 1H), 1.96-1.81 (m, 4H), 1.76-1.49 (m, 5H). MS (DCI/NH₃) m/z 620 (M+H)⁺.

### Example 121

### 2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(1-methyl-1H-pyrazol-4-yl)pyridine-3-carboxamide

### Step 121A

### 7-bromochroman-4-amine

A mixture of 7-bromochroman-4-one (1 g, 4.4 mmol), NaCNBH₃ (0.33 g, 5.3 mmol) and ammonium acetate (3.4 g, 44 mmol) in ethanol (2 mL) was heated in a microwave reactor at 130° C. for 3 minutes. The mixture was quenched with water and extracted with ethyl acetate. The organic phase was dried over MgS04, concentrated and purified by flash chromatography on silica gel eluting with 10% methanol/ethyl acetate to afford the title compound (0.77 g). ¹H NMR (400 MHz, CDCl₃) δ 7.18 (dd, J=8.2, 0.8 Hz, 1H), 7.06-6.96 (m, 2H), 4.33-4.17 (m, 2H), 4.00 (t, J=5.3 Hz, 1H), 2.14 (dddd, J=13.7, 8.6, 4.9, 3.6 Hz, 1H), 1.84 (dtd, J=14.0, 6.1, 3.0 Hz, 1H). MS (DCI/NH₃) m/z 229 (M+H)⁺.

### Step 121B

### 1-(7-bromochroman-4-yl)-4-tosylpiperazine

To a mixture of N,N-bis(2-chloroethyl)-4-methylbenzenesulfonamide (1 g, 3.38 mmol) in N-ethyl-N-isopropylpropan-2-amine (9 mL) was added the product of Step 121A (0.77 g, 3.38 mmol), and the mixture was stirred at 145° C. for 50 hours. The mixture was cooled to ambient temperature and partitioned between ethyl acetate and water. The organic phase was concentrated and purified by flash chromatography on silica gel eluting with 50% ethyl acetate/hexane to afford the title compound (1.3 g). MS (DCI/NH₃) m/z 452 (M+H)⁺.

### Step 121C

### 2-(4-(7-bromochroman-4-yl)piperazin-1-yl)ethanol

To a solution of the product of Step 121B (1.3 g, 2.88 mmol) in HBr/acetic acid (10 mL) was added 4-hydroxybenzoic acid (760 mg, 5.5 mmol), and the mixture was stirred at 90° C. for 1 h. Water was added, and the solid was filtered and washed with water. The aqueous phase was adjusted to pH 9 with aqueous NaOH and extracted with ethyl acetate. The organic phase was concentrated and stirred with 2-bromoethanol (540 mg, 4.3 mmol) and K₂CO₃ (600 mg, 4.3 mmol) in methanol (20 mL) at ambient temperature overnight. The mixture was concentrated and partitioned between ethyl acetate and water. Concentration of the organic layer provided the title compound which was used in the next step without further purification. MS (DCI/NH₃) m/z 342 (M+H)⁺.

### Step 121D

### 2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(1-methyl-1H-pyrazol-4-yl)pyridine-3-carboxamide

A mixture of Intermediate 33 (70 mg, 0.15 mmol), the product of Step 121C (46 mg, 0.135 mmol), bis(di-tert-butyl(4-dimethylaminophenyl)phosphine)dichloropalladium(II) (5 mg, 0.006 mmol) and K₃PO₄ (57 mg, 0.21 mmol) was sparged with argon. To this mixture was added dioxane (5 mL) and water (1 mL), and the mixture was sparged with argon and stirred at 100° C. overnight. The mixture was quenched with water and partitioned between water and ethyl acetate. The organic phase was dried over MgSO₄, concentrated and purified by flash chromatography on silica gel eluting with 30% methanol/ethyl acetate to afford the title compound (35 mg). ¹H NMR (501 MHz, DMSO-d₆) δ 8.34 (d, J=7.7 Hz, 1H), 8.29 (d, J=2.3 Hz, 1H), 7.98 (dd, J=11.4, 2.4 Hz, 2H), 7.76 (s, 1H), 7.53 (d, J=7.9 Hz, 2H), 7.42 (d, J=8.0 Hz, 1H), 7.34 (d, J=7.9 Hz, 2H), 7.09 (dt, J=8.0, 2.3 Hz, 1H), 6.94 (d, J=1.7 Hz, 3H), 4.62-4.51 (m, 2H), 4.35-4.23 (m, 3H), 4.10 (ddd, J=11.5, 9.2, 2.8 Hz, 1H), 3.90 (m, 1H), 3.82 (s, 3H), 3.81 (t, J=7.1 Hz, 1H), 3.46 (t, J=6.4 Hz, 2H), 2.53 (q, J=7.1, 6.5 Hz, 2H), 2.43-2.40 (m, 5H), 2.36 (t, J=6.3 Hz, 2H), 2.07-1.85 (m, 4H), 1.77-1.62 (m, 3H), 1.57 (ddd, J=15.1, 13.1, 7.2 Hz, 1H). MS (DCI/NH₃) m/z 652 (M+H)⁺.

### Example 122

### 2-amino-5-(1-methyl-1H-pyrazol-4-yl)-N-[(1S,2S)-2-({4-[5-(piperazin-1-yl)-5,6,7,8-tetrahydronaphthalen-2-yl]phenyl}methoxy)cyclopentyl]pyridine-3-carboxamide

A mixture of Intermediate 33 (50 mg, 0.1 mmol), the product of Step 120C (28 mg, 0.1 mmol), bis(di-tert-butyl(4-dimethylaminophenyl)phosphine)dichloropalladium(II) (5 mg, 0.006 mmol) and K₃PO₄ (57 mg, 0.21 mmol) was prepared. Dioxane (5 mL) and water (1 mL) were added, and the mixture was sparged with argon and stirred at 100° C. for 1 hour. The mixture was quenched with water and partitioned between water and ethyl acetate. The organic phase was dried over MgSO₄, concentrated and purified by flash chromatography on silica gel eluting with 30% methanol/ethyl acetate to afford the title compound (34 mg). ¹H NMR (400 MHz, DMSO-d₆) δ 8.34 (d, J=7.7 Hz, 1H), 8.29 (d, J=2.2 Hz, 1H), 7.97 (dd, J=11.2, 2.5 Hz, 2H), 7.75 (s, 1H), 7.60 (d, J=8.1 Hz, 1H), 7.57-7.50 (m, 2H), 7.40-7.31 (m, 3H), 7.29 (d, J=1.9 Hz, 1H), 6.93 (s, 2H), 4.62-4.50 (m, 2H), 4.28 (qd, J=7.5, 3.9 Hz, 1H), 4.08 (m, 1H), 3.94-3.87 (m, 2H), 3.82 (s, 3H), 3.16-3.01 (m, 4H), 2.73 (q, J=4.9 Hz, 1H), 2.70-2.59 (m, 4H), 2.07-1.94 (m, 1H), 1.91 (tdd, J=11.9, 6.8, 3.4 Hz, 3H), 1.78-1.49 (m, 6H). MS (DCI/NH₃) m/z 606 (M+H)⁺.

### Example 123

### 6-amino-5′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

### Step 123A

### 2-[4-(6-bromo-1,2,3,4-tetrahydronaphthalen-1-yl)piperazin-1-yl]ethan-1-ol

To a solution of the product of Step 122C (100 mg, 0.34 mmol) in methanol (5 mL) was added 2-bromoethanol (72 mg, 0.6 mmol) and K₂CO₃ (60 mg, 0.4 mmol), and the mixture was stirred at ambient temperature overnight. The mixture was concentrated and partitioned between water and ethyl acetate. The organic phase was dried over MgSO₄ and concentrated to afford the crude title compound (80 mg). MS (DCI/NH₃) m/z 339 (M+H)⁺.

### Step 123B

### 6-amino-N-{(1S,2S)-2-[(4-bromophenyl)methoxy]cyclopentyl}-5′-fluoro[3,3′-bipyridine]-5-carboxamide

To a mixture of Intermediate 1 (6.57 g, 21 mmol) and triethylamine (6.5 g, 64 mmol) in N,N-dimethylformamide (90 mL) was added 1-[bis(dimethylamino)methylene]-1H-1,2,3-triazolo[4,5-b]pyridinium 3-oxid hexafluorophosphate (HATU, 10 g, 26 mmol) followed by Intermediate 5 (5 g, 21 mmol), and the mixture was stirred at ambient temperature for 1 hour. The mixture was quenched with water and extracted with ethyl acetate. The organic phase was concentrated, and the residue was purified by flash chromatography on silica gel eluting with 0-10% methanol/ethyl acetate to afford the title compound (7.2 g). MS (ESI+) m/z 486 (M+H)⁺.

### Step 123C

### 6-amino-5′-fluoro-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

A mixture of the product of Step 123B (2.7 g, 5.5 mmol), 4,4,4′,4′,5,5,5′,5′-octamethyl-2,2′-bi(1,3,2-dioxaborolane) (2.8 g, 11 mmol), Pd(PPh₃)₄ (321 mg, 0.28 mmol) and potassium acetate (1 g, 11 mmol) was sparged with argon and dioxane (50 mL) was added. The mixture was stirred under argon at 110° C. overnight. The mixture was quenched with water and extracted with ethyl acetate. The organic phase was dried over MgSO₄, concentrated and purified by flash chromatography on silica gel eluting with 0-5% of methanol/ethyl acetate to afford the title compound (2.8 g). ¹H NMR (400 MHz, methanol-d₄) δ 8.67 (t, J=1.7 Hz, 1H), 8.40 (dd, J=8.5, 2.6 Hz, 2H), 8.14 (d, J=2.3 Hz, 1H), 7.89 (ddd, J=10.0, 2.7, 1.8 Hz, 1H), 7.69-7.59 (m, 2H), 7.32 (d, J=7.8 Hz, 2H), 4.64 (s, 2H), 4.41 (ddd, J=7.9, 6.7, 4.3 Hz, 1H), 4.05-3.89 (m, 1H), 2.28-2.06 (m, 1H), 2.02-1.90 (m, 1H), 1.92-1.72 (m, 3H), 1.60 (m, 1H), 1.30 (s, 12H). MS (ESI+) m/z 532 (M+H)⁺.

### Step 123D

### 6-amino-5′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

The title compound was prepared as described in Step 121D using the product of Step 123C in place of Intermediate 33 and the product of Step 123A in place of the product of Step 121C. ¹H NMR (400 MHz, DMSO-d₆) δ 8.80 (q, J=1.7 Hz, 1H), 8.52 (d, J=2.3 Hz, 1H), 8.47-8.40 (m, 2H), 8.23 (d, J=2.4 Hz, 1H), 8.04 (dq, J=10.6, 2.1 Hz, 1H), 7.57 (d, J=8.1 Hz, 1H), 7.50 (d, J=8.2 Hz, 2H), 7.39-7.27 (m, 5H), 7.23 (s, 1H), 4.56 (s, 2H), 4.30 (qd, J=7.5, 4.1 Hz, 1H), 3.91 (dt, J=6.5, 4.0 Hz, 1H), 3.72 (dq, J=11.7, 6.0, 5.4 Hz, 1H), 3.54-3.33 (m, 4H), 2.76-2.56 (m, 2H), 2.45-2.36 (m, 8H), 2.08-1.78 (m, 4H), 1.75-1.50 (m, 6H). MS (ESI+) m/z 665 (M+H)⁺.

### Example 124

### 2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(trifluoromethyl)pyridine-3-carboxamide

The title compound was prepared as described in Step 121D using Intermediate 25 in place of Intermediate 33 and using the product of Step 123A in place of the product of Step 121C. ¹H NMR (400 MHz, DMSO-d₆) δ 8.53 (d, J=7.6 Hz, 1H), 8.35 (d, J=2.3 Hz, 1H), 8.16 (d, J=2.4 Hz, 1H), 7.71 (s, 2H), 7.60 (d, J=8.1 Hz, 1H), 7.53 (d, J=8.1 Hz, 2H), 7.34 (dd, J=12.7, 8.0 Hz, 3H), 7.28 (d, J=2.0 Hz, 1H), 4.54 (d, J=2.4 Hz, 2H), 4.25 (qd, J=7.4, 3.8 Hz, 1H), 3.87 (dt, J=6.3, 3.7 Hz, 1H), 3.79 (m, 1H), 3.50 (m, 4H), 2.72 (q, J=4.9 Hz, 2H), 2.62-2.47 (m, 8H), 2.05-1.80 (m, 4H), 1.77-1.46 (m, 6H). MS (ESI+) m/z 638 (M+H)⁺.

### Example 125

### 2-amino-N-{(1S,2S)-2-[(4-{1-[(3,4-dihydroxybutyl)(methyl)amino]-2,3-dihydro-1H-inden-5-yl}phenyl)methoxy]cyclopentyl}-5-(1-methyl-1H-pyrazol-4-yl)pyridine-3-carboxamide

### Step 125A

### 5-bromo-N-methyl-2,3-dihydro-1H-inden-1-amine

To a solution of 5-bromo-1-indanone (0.5 g, 2.37 mmol) in 1,2-dichloroethane (15.8 mL) was added methylamine in ethanol (0.44 mL, 3.55 mmol), and the mixture was stirred for 1 hour. Sodium triacetoxyborohydride (1.0 g, 4.74 mmol) was added in 5 batches and the mixture was stirred at ambient temperature for 3 days. The mixture was purified by flash chromatography on silica gel eluting with 0-10% methanol/dichloromethane to provide the title compound (471 mg). ¹H NMR (501 MHz, methanol-d₄) δ 7.54-7.49 (m, 1H), 7.47-7.39 (m, 2H), 4.54 (dd, J=7.7, 4.5 Hz, 1H), 3.14 (ddd, J=15.4, 8.4, 6.3 Hz, 1H), 2.96 (ddd, J=16.5, 8.9, 5.3 Hz, 1H), 2.63 (d, J=0.7 Hz, 3H), 2.52 (dddd, J=14.0, 8.8, 7.8, 6.4 Hz, 1H), 2.15 (dddd, J=13.7, 8.3, 5.3, 4.5 Hz, 1H). LCMS (ESI+) m/z 226.04 (M+H)⁺.

### Step 125B

### (2,2-dimethyl-1,3-dioxolan-4-yl)acetaldehyde

To a solution of pyridinium chlorochromate (885 mg, 4.10 mmol) in dichloromethane (10.9 mL) was added 2-(2,2-dimethyl-1,3-dioxolan-4-yl)ethanol (0.4 mL, 2.74 mmol), and the mixture was stirred for 8 hours. The suspension was filtered through silica gel and the filtrate was concentrated to afford the title compound which was used without further purification.

### Step 125C

### 5-bromo-N-[2-(2,2-dimethyl-1,3-dioxolan-4-yl)ethyl]-N-methyl-2,3-dihydro-1H-inden-1-amine

To a solution of the product of Step 125A (470.7 mg, 2.082 mmol) in methanol (9.2 mL) was added the product of Step 125B (394 mg, 1.831 mmol), and the mixture was stirred for 30 minutes. Sodium borohydride (139 mg, 3.66 mmol) was added in 5 batches and the mixture was stirred at ambient temperature for 16 hours. The mixture was purified by flash chromatography on silica gel eluting with 0-10% methanol/dichloromethane) to provide the title compound (159 mg). ¹H NMR (400 MHz, CDCl₃) δ 7.39-7.31 (m, 3H), 4.47 (s, 1H), 4.22-3.99 (m, 2H), 3.64-3.49 (m, 1H), 2.98-2.86 (m, 1H), 2.83 (dt, J=16.4, 8.0 Hz, 1H), 2.60 (s, 2H), 2.32-2.21 (m, 3H), 2.12 (s, 3H), 1.87 (m, 2H), 1.50-1.23 (m, 6H). LCMS (ESI+) m/z 354.08 (M+H)⁺.

### Step 125D

### 4-[(5-bromo-2,3-dihydro-1H-inden-1-yl)(methyl)amino]butane-1,2-diol

To a solution of the product of Step 125C (159 mg, 0.45 mmol) in tetrahydrofuran (3 mL) was added 2 M HCl (4.5 mL, 9.0 mmol), and the mixture was stirred at ambient temperature for 3 days. The mixture was concentrated, and the residue de-salted with a Si-Carbonate cartridge to afford the title compound which was used without further purification. LCMS (ESI+) m/z 314.06 (M+H)⁺.

### Step 125E

### 2-amino-N-{(1S,2S)-2-[(4-{1-[(3,4-dihydroxybutyl)(methyl)amino]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 50F using the product of Step 125D in place of the product of Step 50E. ¹H NMR (400 MHz, methanol-d₄) δ 8.51 (t, J=1.9 Hz, 1H), 8.24 (d, J=2.1 Hz, 1H), 8.03-7.96 (m, 1H), 7.88 (d, J=0.8 Hz, 1H), 7.70-7.55 (m, 5H), 7.52-7.39 (m, 2H), 5.19 (s, 1H), 4.71 (d, J=2.5 Hz, 2H), 4.46 (ddd, J=8.0, 6.7, 4.3 Hz, 1H), 4.05 (dt, J=6.4, 4.2 Hz, 1H), 3.94 (d, J=1.7 Hz, 3H), 3.69 (d, J=114.7 Hz, 4H), 3.32-3.18 (m, 2H), 3.12 (ddd, J=16.9, 9.3, 3.7 Hz, 1H), 3.01-2.55 (m, 4H), 2.50 (dddt, J=15.0, 8.5, 6.5, 2.9 Hz, 1H), 2.27-2.15 (m, 1H), 2.15-1.75 (m, 6H), 1.68 (dq, J=14.6, 7.5 Hz, 1H). MS (ESI+) m/z 625.2 (M+H)⁺.

### Example 126

### 6-amino-N-{(1S,2S)-2-[(4-{1-[(3,4-dihydroxybutyl)(methyl)amino]-2,3-dihydro-1H-inden-5-yl}phenyl)methoxy]cyclopentyl}-6′-fluoro[3,3′-bipyridine]-5-carboxamide

The title compound as described in Step 50F using the product of Step 125D in place of the product of Step 50E and Intermediate 32 and Intermediate 33. ¹H NMR (500 MHz, methanol-d₄) δ 8.48-8.42 (m, 2H), 8.35 (d, J=2.2 Hz, 1H), 8.18 (ddt, J=10.0, 8.0, 2.6 Hz, 1H), 7.55 (t, J=8.1 Hz, 5H), 7.48-7.40 (m, 2H), 7.18-7.11 (m, 1H), 5.33-5.04 (m, 1H), 4.68 (q, J=12.4 Hz, 2H), 4.43 (td, J=7.3, 4.5 Hz, 1H), 4.01 (dt, J=6.5, 4.5 Hz, 1H), 3.73 (d, J=91.8 Hz, 1H), 3.53 (d, J=40.1 Hz, 2H), 3.29-3.16 (m, 2H), 3.16-3.03 (m, 1H), 2.97-2.53 (m, 4H), 2.52-2.42 (m, 1H), 2.26-2.14 (m, 1H), 2.14-1.87 (m, 3H), 1.82 (dddd, J=20.8, 13.0, 10.9, 5.6 Hz, 3H), 1.63 (dq, J=13.0, 7.6 Hz, 1H). MS (ESI+) m/z 640.3 (M+H)⁺.

### Example 127

### 6-amino-N-{(1S,2S)-2-[(4-{1-[(3,4-dihydroxybutyl)(methyl)amino]-2,3-dihydro-1H-inden-5-yl}phenyl)methoxy]cyclopentyl}-2′-fluoro[3,3′-bipyridine]-5-carboxamide

The title compound (19 mg) as described in Step 50F using the product of Step 125D in place of the product of Step 50E and Intermediate 19 in place of Intermediate 33. ¹H NMR (400 MHz, methanol-d₄) δ 8.40 (dd, J=2.2, 0.7 Hz, 1H), 8.35 (t, J=1.9 Hz, 1H), 8.29-8.20 (m, 1H), 8.15 (ddt, J=9.5, 7.5, 1.8 Hz, 1H), 7.71-7.54 (m, 5H), 7.53-7.39 (m, 3H), 5.17 (s, 1H), 4.76-4.65 (m, 2H), 4.45 (td, J=7.3, 4.4 Hz, 1H), 4.03 (dt, J=6.4, 4.3 Hz, 1H), 3.93-3.65 (m, 1H), 3.56 (d, J=28.0 Hz, 3H), 3.32-3.18 (m, 2H), 3.18-3.06 (m, 1H), 2.78 (dd, J=88.8, 32.1 Hz, 4H), 2.50 (ddd, J=15.2, 8.4, 3.9 Hz, 1H), 2.27-2.15 (m, 1H), 2.15-1.90 (m, 3H), 1.90-1.75 (m, 3H), 1.65 (dq, J=13.0, 7.5 Hz, 1H). MS (ESI+) m/z 640.3 (M+H)⁺.

### Example 128

### 2-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-5-(1-methyl-1H-pyrazol-4-yl)pyridine-3-carboxamide

### Step 128A

### 2-{4-[(1S)-5-bromo-2,3-dihydro-1H-inden-1-yl]piperazin-1-yl}propane-1,3-diol

To Intermediate 16 (300 mg, 0.944 mmol) and 2,2-dimethyl-1,3-dioxan-5-one (184 mg, 1.417 mmol) in dichloromethane (10 mL) was added sodium triacetoxyhydroborate (400 mg, 1.889 mmol), and the mixture was stirred at ambient temperature overnight. The mixture was quenched with saturated NaHCO₃, extracted with ethyl acetate and purified by flash chromatography on silica gel eluting with 0-10% methanol/dichloromethane to afford the protected diol. This was treated with trifluoroacetic acid (1455 μl, 18.89 mmol) in dichloromethane (3 mL) at ambient temperature overnight. The mixture was concentrated, and the residue was dissolved in dichloromethane, washed with NaHCO₃ and brine, dried over MgSO₄ and purified by flash chromatography on silica gel eluting with 0-10% methanol/dichloromethane to afford the title compound (290 mg). MS (ESI+) m/z 357.1 (M+H)⁺.

### Step 128B

### 2-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 12D substituting the product of Step 128A for the product of Step 12C. ¹H NMR (400 MHz, DMSO-d₆) δ 8.34-8.22 (m, 2H), 7.99-7.93 (m, 2H), 7.75 (d, J=0.8 Hz, 1H), 7.56-7.50 (m, 2H), 7.42-7.26 (m, 5H), 6.93 (s, 2H), 4.63-4.51 (m, 2H), 4.25 (dtd, J=28.2, 14.4, 12.9, 5.6 Hz, 4H), 3.89 (dt, J=6.1, 3.8 Hz, 1H), 3.80 (s, 3H), 3.48-3.37 (m, 3H), 2.88 (ddd, J=14.6, 8.3, 5.6 Hz, 1H), 2.76 (dt, J=15.8, 7.7 Hz, 1H), 2.65-2.55 (m, 4H), 2.38 (ddd, J=31.9, 9.7, 3.8 Hz, 4H), 2.09-1.48 (m, 10H). MS (ESI+) m/z 666 (M+H)⁺.

### Example 129

### 6-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-2′-fluoro[3,3′-bipyridine]-5-carboxamide

The title compound was prepared as described in Step 8D substituting Intermediate 19 for Intermediate 33 and substituting the product of Step 128A for the product of Step 8C. ¹H NMR (501 MHz, DMSO-d₆) δ 8.37 (d, J=7.7 Hz, 1H), 8.35-8.29 (m, 1H), 8.16 (dt, J=5.0, 1.5 Hz, 1H), 8.14-8.05 (m, 2H), 7.54 (d, J=8.1 Hz, 2H), 7.44-7.21 (m, 8H), 4.62-4.48 (m, 2H), 4.38-4.00 (m, 4H), 3.92-3.85 (m, 1H), 3.51-3.41 (m, 4H), 3.15 (d, J=4.7 Hz, 1H), 2.89 (ddd, J=15.9, 8.4, 5.4 Hz, 1H), 2.78 (dp, J=13.6, 6.7, 6.2 Hz, 1H), 2.68-2.55 (m, 4H), 2.47-2.27 (m, 4H), 2.08-1.87 (m, 4H), 1.75-1.50 (m, 4H). MS (ESI+) m/z 681 (M+H)⁺.

### Example 130

### 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(trifluoromethyl)pyridine-3-carboxamide

Intermediate 25 (0.03 g, 0.071 mmol), the product of Step 83C (0.027 g, 0.077 mmol), bis(di-tert-butyl(4-dimethylaminophenyl)phosphine)dichloropalladium(II) (2.51 mg, 3.54 μmol) and K₂CO₃ (0.029 g, 0.213 mmol) in degassed water (0.2 mL) and degassed dioxane (0.7 mL) were heated at 75° C. for 1 hour. The mixture was cooled to ambient temperature and partitioned in water and ethyl acetate. The organic layer was washed with brine, dried over Na₂SO₄, and concentrated. The residue was purified by flash chromatography on silica gel eluting with 0-80% (89:10:1 dichloromethane/methanol/NH₄OH) in dichloromethane to afford the title compound (34 mg). ¹H NMR (501 MHz, DMSO-d₆) δ 8.53 (d, J=7.6 Hz, 1H), 8.37 (d, J=2.2 Hz, 1H), 8.22-8.15 (m, 1H), 7.73 (brs, 2H), 7.61-7.52 (m, 2H), 7.46-7.32 (m, 4H), 7.24 (d, J=7.7 Hz, 1H), 4.64-4.52 (m, 2H), 4.44-4.22 (m, 3H), 3.94-3.85 (m, 1H), 3.46 (q, J=6.1 Hz, 2H), 2.47-2.29 (m, 9H), 2.09-1.81 (m, 4H), 1.79-1.48 (m, 4H), 1.35 (s, 3H), 1.16 (s, 3H). MS (ESI+) m/z 652.3 (M+H)⁺.

### Example 131

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(trifluoromethyl)pyridine-3-carboxamide

The title compound was prepared as described in Step 130 substituting the product of Step 34A for the product of Step 83C. ¹H NMR (400 MHz, DMSO-d₆) δ 8.55 (d, J=7.6 Hz, 1H), 8.44-8.35 (m, 1H), 8.19 (d, J=2.4 Hz, 1H), 7.85-7.68 (m, 2H), 7.62-7.52 (m, 2H), 7.49-7.26 (m, 5H), 4.66-4.51 (m, 2H), 4.38-4.22 (m, 3H), 3.97-3.85 (m, 1H), 3.46 (q, J=6.1 Hz, 2H), 3.00-2.72 (m, 2H), 2.44-2.28 (m, 8H), 2.15-1.85 (m, 4H), 1.82-1.48 (m, 4H). MS (ESI+) m/z 624.3 (M+H)⁺.

### Example 1326-amino-5′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide

The title compound was prepared as described in Step 121D using the product of Step 123C in place of Intermediate 33. ¹H NMR (500 MHz, DMSO-d₆) δ 8.84 (t, J=1.9 Hz, 1H), 8.56 (d, J=2.4 Hz, 1H), 8.49 (t, J=2.5 Hz, 1H), 8.42 (dd, J=7.8, 1.7 Hz, 1H), 8.24 (dd, J=2.4, 1.4 Hz, 1H), 8.10-8.02 (m, 1H), 7.56-7.51 (m, 2H), 7.43 (d, J=8.1 Hz, 1H), 7.37 (d, J=8.0 Hz, 4H), 7.10 (dd, J=8.0, 1.9 Hz, 1H), 6.95 (t, J=1.7 Hz, 1H), 4.60 (s, 2H), 4.34 (ddd, J=11.3, 6.1, 3.6 Hz, 2H), 4.14 (ddd, J=11.5, 9.0, 2.9 Hz, 1H), 3.98-3.84 (m, 2H), 3.60 (m, 2H), 3.02-2.53 (m, 10H), 2.15-1.88 (m, 4H), 1.85-1.64 (m, 3H), 1.59 (ddd, J=15.1, 13.2, 7.3 Hz, 1H). MS (ESI+) m/z 667 (M+H)⁺.

### Example 133

### 2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(trifluoromethyl)pyridine-3-carboxamide

The title compound was prepared as described in Step 121D using Intermediate 34 in place of Intermediate 33. ¹H NMR (501 MHz, DMSO-d₆) δ 8.55 (d, J=7.6 Hz, 1H), 8.37 (d, J=2.3 Hz, 1H), 8.18 (d, J=2.3 Hz, 1H), 7.73 (s, 2H), 7.53 (d, J=8.0 Hz, 2H), 7.43 (d, J=8.1 Hz, 1H), 7.35 (dd, J=12.6, 7.0 Hz, 3H), 7.11 (dt, J=8.1, 1.6 Hz, 1H), 4.65-4.49 (m, 2H), 4.49-4.35 (m, 1H), 4.28 (dddd, J=22.6, 10.7, 5.8, 3.4 Hz, 2H), 4.08 (dddd, J=22.6, 11.2, 9.0, 2.8 Hz, 2H), 3.89 (dt, J=6.5, 3.8 Hz, 1H), 3.81 (ddd, J=19.0, 8.5, 5.2 Hz, 1H), 3.48 (td, J=6.2, 3.7 Hz, 4H), 2.62-2.50 (m, 4H), 2.47-2.36 (m, 2H), 2.05-1.78 (m, 4H), 1.76-1.59 (m, 3H), 1.60-1.49 (m, 1H). MS (ESI+) m/z 640 (M+H)⁺.

### Example 134

### 6-amino-2′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide

The title compound was prepared as described in Step 121D using Intermediate 19 in place of Intermediate 33. ¹H NMR (400 MHz, DMSO-d₆) δ 8.54 (d, J=7.6 Hz, 1H), 8.36 (d, J=2.1 Hz, 1H), 8.26 (d, J=2.2 Hz, 1H), 8.19 (d, J=4.7 Hz, 1H), 8.18-8.11 (m, 1H), 7.53 (d, J=8.0 Hz, 2H), 7.43 (t, J=8.0 Hz, 2H), 7.34 (d, J=8.1 Hz, 2H), 7.11 (dd, J=8.2, 1.8 Hz, 1H), 6.97 (s, 1H), 4.71-4.51 (m, 2H), 4.28 (tdq, J=12.9, 5.9, 3.1, 2.6 Hz, 2H), 4.10 (ddd, J=11.2, 9.0, 2.7 Hz, 1H), 3.93-3.86 (m, 1H), 3.81 (dd, J=8.5, 5.3 Hz, 1H), 3.61-3.41 (m, 4H), 2.63-2.49 (m, 2H), 2.47-2.38 (m, 4H), 2.36 (d, J=6.5 Hz, 2H), 2.05-1.96 (m, 2H), 1.90 (dt, J=14.4, 6.1 Hz, 2H), 1.77-1.58 (m, 3H), 1.59-1.46 (m, 1H). MS (ESI+) m/z 667 (M+H)⁺.

### Example 135

### 6-amino-2′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Step 12D substituting Intermediate 19 for Intermediate 24 and substituting Intermediate 15 for the product of Step 12C. ¹H NMR (501 MHz, DMSO-d₆) δ 8.37 (d, J=7.7 Hz, 1H), 8.33 (t, J=2.2 Hz, 1H), 8.15 (dt, J=4.8, 1.5 Hz, 1H), 8.14-8.05 (m, 2H), 7.59-7.53 (m, 2H), 7.46-7.14 (m, 8H), 4.71-4.47 (m, 2H), 4.46-4.23 (m, 3H), 4.07 (q, J=5.3 Hz, 1H), 3.94-3.77 (m, 1H), 3.47 (q, J=5.6 Hz, 2H), 3.15 (d, J=4.5 Hz, 2H), 2.46-2.27 (m, 7H), 2.12-1.79 (m, 4H), 1.76-1.45 (m, 4H), 1.35 (s, 3H), 1.16 (d, J=3.0 Hz, 3H). MS (ESI+) m/z 679 (M+H)⁺.

### Example 136

### 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-4-methoxypyridine-3-carboxamide

### Step 136A

### 2-amino-N-{(1S,2S)-2-[(4-bromophenyl)methoxy]cyclopentyl}-4-methoxypyridine-3-carboxamide

A solution of 2-amino-4-methoxynicotinic acid (0.225 g, 1.338 mmol), 1-[bis(dimethylamino)methylene]-1H-1,2,3-triazolo[4,5-b]pyridinium 3-oxid hexafluorophosphate (HATU, 0.56 g, 1.472 mmol) and triethylamine (0.933 mL, 6.69 mmol) in N,N-dimethylformamide (4 mL) was stirred at ambient temperature for 1.5 hours. A separate solution of Intermediate 1 (free base, 0.398 g, 1.472 mmol) in N,N-dimethylformamide (1 mL) was added, and the mixture was stirred at ambient temperature for 16 hours. The mixture was partitioned in water and ethyl acetate and the organic layer was washed with brine, dried over Na₂SO₄, and concentrated. The residue was purified by flash chromatography on silica gel eluting with 0-8% methanol/dichloromethane to afford the title compound (340 mg). MS (ESI+) m/z 420.1 (M+H)⁺.

### Step 136B

### 2-amino-4-methoxy-N-[(1S,2S)-2-{[4-(4,4,5,5-tetramethyl-1,3,2-dioxaborolan-2-yl)phenyl]methoxy}cyclopentyl]pyridine-3-carboxamide

The product of Step 136A (0.335 g, 0.797 mmol) and 4,4,4′,4′,5,5,5′,5′-octamethyl-2,2′-bi(1,3,2-dioxaborolane) (0.385 g, 1.514 mmol) in degassed dioxane (5 mL) was treated with potassium acetate (0.235 g, 2.391 mmol) followed by [1,1′-bis(diphenylphosphino)ferrocene]dichloropalladium(II) (0.052 g, 0.064 mmol), and the mixture was heated under nitrogen at 100° C. for 2.5 hours. The mixture was cooled to ambient temperature and partitioned in ethyl acetate and water, and the organic layer was washed with brine, dried over Na₂SO₄, and concentrated. The residue was purified by flash chromatography on silica gel eluting with 0-10% methanol/ethyl acetate to afford the title compound (280 mg). MS (ESI+) m/z 468.5 (M+H)⁺.

### Step 136C

### 2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-4-methoxypyridine-3-carboxamide

A mixture of the product of Step 136B (0.026 g, 0.056 mmol), the product of Step 83C (0.021 g, 0.060 mmol), bis(di-tert-butyl(4-dimethylaminophenyl)phosphine)dichloropalladium (II) (1.970 mg, 2.78 μmol) and K₂CO₃ (0.023 g, 0.167 mmol) in degassed water (0.2 mL) and degassed dioxane (0.7 mL) was heated at 75° C. for 1 hour. The mixture was cooled to ambient temperature and partitioned in water and dichloromethane. The organic layer was dried over Na₂SO₄, concentrated and purified by flash chromatography on silica gel eluting with 0-10% methanol/dichloromethane followed by 0-100% (89:10:1 dichloromethane/methanol/NH₄OH) in dichloromethane to afford the title compound (16 mg). ¹H NMR (501 MHz, DMSO-d₆) δ 8.05 (d, J=7.6 Hz, 1H), 7.90 (d, J=5.8 Hz, 1H), 7.65-7.58 (m, 2H), 7.50-7.43 (m, 2H), 7.39 (d, J=8.1 Hz, 2H), 7.26 (d, J=7.6 Hz, 1H), 6.52 (s, 2H), 6.37 (d, J=5.9 Hz, 1H), 4.68-4.53 (m, 2H), 4.40 (t, J=7.9 Hz, 1H), 4.34 (t, J=5.4 Hz, 1H), 4.30-4.20 (m, 1H), 3.89 (dt, J=6.5, 3.7 Hz, 1H), 3.79 (s, 3H), 3.48 (q, J=6.0 Hz, 2H), 2.49-2.40 (m, 6H), 2.38 (t, J=6.4 Hz, 3H), 2.09-1.97 (m, 1H), 1.97-1.83 (m, 3H), 1.75-1.58 (m, 3H), 1.58-1.42 (m, 1H), 1.38 (s, 3H), 1.19 (s, 3H). MS (ESI+) m/z 614.4 (M+H)⁺.

### Example 137

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxypyridine-3-carboxamide

The title compound was prepared as described in Step 136C substituting the product of Step 34A for the product of Step 83C. ¹H NMR (501 MHz, DMSO-d₆) δ 8.05 (d, J=7.7 Hz, 1H), 7.90 (d, J=5.8 Hz, 1H), 7.64-7.52 (m, 2H), 7.51-7.42 (m, 2H), 7.42-7.34 (m, 2H), 7.32 (d, J=7.8 Hz, 1H), 6.53 (s, 2H), 6.37 (d, J=5.9 Hz, 1H), 4.68-4.52 (m, 2H), 4.37-4.17 (m, 3H), 3.92-3.85 (m, 1H), 3.79 (s, 3H), 3.46 (q, J=6.2 Hz, 2H), 2.99-2.88 (m, 1H), 2.88-2.72 (m, 1H), 2.47-2.30 (m, 8H), 2.15-1.97 (m, 3H), 1.97-1.83 (m, 1H), 1.75-1.58 (m, 3H), 1.58-1.45 (m, 1H). MS (ESI+) m/z 586.2 (M+H)⁺.

### Example 138

### 2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-4-methoxypyridine-3-carboxamide

The title compound was prepared as described in Step 136C substituting the product of Step 93A for the product of Step 83C. ¹H NMR (400 MHz, DMSO-d₆) δ 8.05 (d, J=7.6 Hz, 1H), 7.90 (d, J=5.8 Hz, 1H), 7.68-7.57 (m, 2H), 7.51-7.42 (m, 2H), 7.42-7.34 (m, 2H), 7.31-7.21 (m, 1H), 6.52 (s, 2H), 6.37 (d, J=5.8 Hz, 1H), 4.70-4.51 (m, 2H), 4.41 (t, J=7.9 Hz, 1H), 4.31-4.18 (m, 1H), 3.94-3.83 (m, 1H), 3.79 (s, 3H), 2.50-2.22 (m, 8H), 2.16 (s, 3H), 2.10-1.81 (m, 4H), 1.81-1.58 (m, 3H), 1.58-1.45 (m, 1H), 1.38 (s, 3H), 1.19 (s, 3H). MS (ESI+) m/z 584.2 (M+H)⁺.

### Example 139

### 2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]pyridine-3-carboxamide

### Step 139A

### 1-[(1S)-5-bromo-2,3-dihydro-1H-inden-1-yl]-4-methylpiperazine

A solution of Intermediate 16 (HCl salt, 400 mg, 1.259 mmol) in dichloromethane (5 mL) was treated with formaldehyde (151 mg, 2.52 mmol) followed by sodium triacetoxyhydroborate (534 mg, 2.52 mmol), and the mixture was stirred at ambient temperature 16 hours. The mixture was partitioned in saturated NaHCO₃ and ethyl acetate. The organic layer was washed with brine, dried over Na₂SO₄, and concentrated. The residue was purified by flash chromatography on silica gel eluting with 0-10% methanol/dichloromethane to afford the title compound (320 mg). MS (APCI+) m/z 295.06 (M+H)⁺.

### Step 139B

### 2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]pyridine-3-carboxamide

The title compound was prepared as described in Step 136C substituting the product of Step 139A for the product of Step 83C. ¹H NMR (400 MHz, DMSO-d₆) δ 8.05 (d, J=7.7 Hz, 1H), 7.90 (d, J=5.8 Hz, 1H), 7.65-7.54 (m, 2H), 7.52-7.42 (m, 2H), 7.42-7.35 (m, 2H), 7.31 (d, J=7.8 Hz, 1H), 6.52 (s, 2H), 6.37 (d, J=5.8 Hz, 1H), 4.69-4.51 (m, 2H), 4.35-4.18 (m, 2H), 3.94-3.83 (m, 1H), 3.79 (s, 3H), 3.01-2.73 (m, 2H), 2.44-2.20 (m, 6H), 2.14 (s, 3H), 2.11-1.82 (m, 4H), 1.77-1.58 (m, 3H), 1.58-1.43 (m, 1H). MS (ESI+) m/z 556.3 (M+H)⁺.

### Example 140

### 6-amino-N-{(1S,2S)-2-[(4-{1-[4-(1,3-dihydroxypropan-2-yl)piperazin-1-yl]-3,3-dimethyl-2,3-dihydro-1H-inden-5-yl}phenyl)methoxy]cyclopentyl}-2′-fluoro[3,3′-bipyridine]-5-carboxamide

### Step 140A

### 2-[4-(5-bromo-3,3-dimethyl-2,3-dihydro-1H-inden-1-yl)piperazin-1-yl]propane-1,3-diol

The title compound was prepared as described in Step 128A substituting Intermediate 9 for Intermediate 16.

### Step 140B

### 6-amino-N-{(1S,2S)-2-[(4-{1-[4-(1,3-dihydroxypropan-2-yl)piperazin-1-yl]-3,3-dimethyl-2,3-dihydro-1H-inden-5-yl}phenyl)methoxy]cyclopentyl}-2′-fluoro[3,3′-bipyridine]-5-carboxamide

The title compound was prepared as described in Step 12D substituting Intermediate 19 for Intermediate 24 and substituting the product of Step 140A for the product of Step 12C. ¹H NMR (400 MHz, DMSO-d₆) δ 8.41-8.34 (m, 1H), 8.32 (t, J=2.2 Hz, 1H), 8.18-8.04 (m, 3H), 7.58-7.50 (m, 2H), 7.43-7.14 (m, 8H), 4.65-4.49 (m, 2H), 4.43-4.17 (m, 3H), 3.88 (dt, J=6.1, 3.7 Hz, 1H), 3.60-3.36 (m, 4H), 2.79-2.56 (m, 4H), 2.40 (ddd, J=20.3, 10.7, 5.2 Hz, 6H), 2.09-1.76 (m, 4H), 1.74-1.44 (m, 4H), 1.33 (d, J=1.9 Hz, 3H), 1.15 (d, J=2.1 Hz, 3H). MS (ESI+) m/z 709 (M+H)⁺.

### Example 141

### 2-amino-5-(1-methyl-1H-pyrazol-4-yl)-N-[(1S,2S)-2-({4-[(5S)-5-(piperazin-1-yl)-5,6,7,8-tetrahydronaphthalen-2-yl]phenyl}methoxy)cyclopentyl]pyridine-3-carboxamide

### Step 141A

### 1-[(1S)-6-chloro-1,2,3,4-tetrahydronaphthalen-1-yl]-4-(2-nitrobenzene-1-sulfonyl)piperazine

To (S)-6-chloro-1,2,3,4-tetrahydronaphthalen-1-amine (ASTATech, 0.801 g, 4.41 mmol) purged with nitrogen was added acetonitrile (15 mL), N,N-diisopropylethylamine (2.31 mL, 13.23 mmol) and a solution of Intermediate 27 (3 g, 4.54 mmol) in acetonitrile (15 mL). After stirring at ambient temperature under nitrogen for 2.5 days, the mixture was partitioned between ethyl acetate and brine. The organic phase was washed with brine, concentrated and purified by flash chromatography on silica gel eluting with 20-70% ethyl acetate/heptane to provide the title compound (1.05 g). MS (ESI+) m/z 436 (M+H)⁺.

### Step 141B

### 1-[(1S)-6-chloro-1,2,3,4-tetrahydronaphthalen-1-yl]piperazine

To a solution of the product of Step 141A (1.05 g, 2.4 mmol) in tetrahydrofuran (10 mL) was added sodium methanethiolate (0.203 g, 2.89 mmol), and the mixture was stirred at ambient temperature for 2.5 days. Additional sodium methanethiolate (0.100 g) was added, and the mixture was stirred for another 1 days. The mixture was partitioned between ethyl acetate and brine, and the organic phase was washed with brine and concentrated. The residue was purified by flash chromatography on silica gel eluting with 5-20% methanol (containing 3% NH₄OH) in dichloromethane) to provide the title compound (313 mg). MS (ESI+) m/z 251 (M+H)⁺.

### Step 141C

### 2-amino-5-(1-methyl-1H-pyrazol-4-yl)-N-[(1S,2S)-2-({4-[(5S)-5-(piperazin-1-yl)-5,6,7,8-tetrahydronaphthalen-2-yl]phenyl}methoxy)cyclopentyl]pyridine-3-carboxamide

A mixture of Intermediate 33 (512 mg, 0.989 mmol), the product of Step 141B (250 mg, 0.989 mmol), bis(di-tert-butyl(4-dimethylaminophenyl)phosphine)dichloropalladium(II) (35 mg, 0.049 mmol), and potassium phosphate tribasic (1.26 g, 5.93 mmol) was purged with nitrogen and dioxane (15 mL) and water (4.7 mL) were added. The mixture was purged with nitrogen and heated at 92.5° C. for 3 hours. After cooling, the mixture was partitioned between ethyl acetate and brine, and the organic phase was washed with brine and concentrated. The residue was purified by flash chromatography on silica gel eluting with 5-20% methanol (containing 3% NH₄OH) in dichloromethane to provide the title compound (449 mg). ¹H NMR (501 MHz, DMSO-d₆) δ 8.37-8.30 (m, 2H), 8.03-7.95 (m, 2H), 7.78 (s, 1H), 7.65 (d, J=8.1 Hz, 1H), 7.59-7.54 (m, 2H), 7.38 (td, J=5.7, 2.7 Hz, 3H), 7.29 (d, J=2.0 Hz, 1H), 6.97 (s, 2H), 4.65-4.54 (m, 2H), 4.31 (qd, J=7.5, 3.9 Hz, 1H), 3.92 (dt, J=6.6, 3.9 Hz, 1H), 3.74 (dt, J=6.2, 3.3 Hz, 1H), 2.74 (tt, J=7.0, 2.8 Hz, 6H), 2.54-2.43 (m, 6H), 2.39 (dt, J=11.2, 4.7 Hz, 2H), 2.05 (dtt, J=12.6, 7.8, 3.9 Hz, 1H), 1.92 (ddd, J=20.8, 9.3, 5.3 Hz, 3H), 1.80-1.53 (m, 5H). MS (ESI+) m/z 606 (M+H)⁺.

### Example 142

### 2-amino-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide

### Step 142A

### 6-bromo-1,2,3,4-tetrahydronaphthalen-1-ol

To a solution of 6-bromo-3,4-dihydronaphthalen-1 (2H)-one (4 g, 17.77 mmol) in methanol (50 mL) at 10° C. was added sodium borohydride (0.672 g, 17.77 mmol) portionwise over 5 minutes. The mixture was stirred in an ice bath for 10 minutes and then overnight at ambient temperature. The reaction was quenched with water and stirred at ambient temperature for 1 hour. The mixture was concentrated and partitioned between water and ethyl acetate. The organic extracts were washed with water and brine, dried over Na₂SO₄, and concentrated to provide the title compound (3.87 g). MS (ESI−) m/z 224.8, 226.8 (M−H)⁻.

### Step 142B

### ethyl 4-(6-bromo-1,2,3,4-tetrahydronaphthalen-1-yl)piperazine-1-carboxylate

Stage 1: To as suspension of the product of Step 142A (1.89 g, 8.32 mmol) in toluene (11.89 mL) at 0° C. was added thionyl chloride (0.911 mL, 12.48 mmol) dropwise. The ice bath was removed, and the mixture was stirred at ambient temperature for 30 minutes then heated at 55° C. for 1 hour. The mixture was cooled to ambient temperature, washed with ice-water, and the organic phase was dried over Na₂SO₄, and concentrated to afford Stage 1 product.

Stage 2: To a solution of the Stage 1 product in acetonitrile (15.34 mL) was added K₂CO₃ (2.332 g, 16.87 mmol), sodium iodide (0.115 g, 0.767 mmol) and ethyl piperazine-1-carboxylate (1.348 mL, 9.20 mmol), and the mixture was heated at reflux overnight. The mixture was diluted with ethyl acetate and filtered through diatomaceous earth, rinsing with additional ethyl acetate. The filtrate was concentrated, and the residue was purified by flash chromatography on silica gel eluting with 0-25% ethyl acetate/heptane to afford the title compound (2.4 g). MS (ESI+) m/z 367, 369 (M+H)⁺.

### Step 142C

### 1-(6-bromo-1,2,3,4-tetrahydronaphthalen-1-yl)piperazine

To as suspension of the product of Step 142B (500 mg, 1.361 mmol) in methanol (1.2 mL) and water (0.3 mL) was added KOH (764 mg, 13.61 mmol), and the mixture was heated at reflux overnight. The mixture was cooled and partitioned between water and dichloromethane. The organic extracts were washed with brine, dried over Na₂SO₄, and concentrated to afford the title compound (386 mg). MS (ESI+) m/z 295.1, 297.1 (M+H)⁺.

### Step 142D

### 1-[(1S)-6-bromo-1,2,3,4-tetrahydronaphthalen-1-yl]piperazine

The enantiomers of the product of Step 142C were separated by chiral preparative super critical fluid chromatography on a Waters SFC 80Q system (Column: Chiralpak AD-H; Mobile phase: A: CO₂ and B: methanol (0.2% diethylamine), 20% B in A). The S-isomer, was isolated as the later-eluting peak. [α]²⁵_{D}=+85° (c=1.0, methanol). The absolute configuration was confirmed by X-ray crystallography.

### Step 142E

### 2-{4-[(1S)-6-bromo-1,2,3,4-tetrahydronaphthalen-1-yl]piperazin-1-yl}ethan-1-ol

To a solution of the product of Step 142D (1.0 g, 3.39 mmol) in acetonitrile (20 mL) was added K₂CO₃ (0.936 g, 6.77 mmol) followed by 2-bromoethanol (0.480 mL, 6.77 mmol), and the mixture was heated at 45° C. for 24 hours. The mixture was concentrated and partitioned between water and ethyl acetate. The organic extracts were washed with water and brine, dried over Na₂SO₄, and concentrated. The residue was purified by flash chromatography on silica gel eluting with 0-10% methanol containing 1% NH₄OH/dichloromethane to afford the title compound (0.66 g). MS (ESI+) m/z 339, 341 (M+H)⁺.

### Step 142F

### 2-amino-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide

A mixture of Intermediate 24 (145 mg, 0.333 mmol), the product of Step 142E (100 mg, 0.295 mmol), tris(dibenzylideneacetone)dipalladium(0) (2.70 mg, 2.95 μmol), 4-(di-tert-butylphosphino)-N,N-dimethylaniline (1.721 mg, 6.48 μmol) and potassium phosphate tribasic (131 mg, 0.619 mmol) was flushed with nitrogen for 30 minutes. Anhydrous dioxane (1.5 mL) was added, followed by nitrogen-sparged water (0.5 mL), and the sealed reaction was heated at a 70° C. overnight. The mixture was cooled to ambient temperature and diluted with ethyl acetate and water. The mixture was treated with ammonium pyrrolidinedithiocarbamate (5 mg), stirred for 30 minutes and filtered through diatomaceous earth, rinsing with additional ethyl acetate. The organic phase was separated, and the aqueous phase was extracted with additional ethyl acetate. The organic extracts were dried over Na₂SO₄, and concentrated, and the residue was purified by flash chromatography on silica gel eluting with 0-12% methanol with 5% NH₄OH/dichloromethane) to afford the title compound (107 mg). ¹H NMR (400 MHz, DMSO-d₆) δ 8.33-8.27 (m, 2H), 7.97 (d, J=2.3 Hz, 1H), 7.95 (s, 1H), 7.75 (s, 1H), 7.59 (d, J=8.1 Hz, 1H), 7.53 (d, J=8.2 Hz, 2H), 7.34 (d, J=8.1 Hz, 3H), 7.25 (d, J=2.0 Hz, 1H), 6.93 (s, 2H), 4.61-4.52 (m, 2H), 4.37-4.24 (m, 2H), 3.91-3.87 (m, 1H), 3.80 (s, 3H), 3.73 (q, J=5.1 Hz, 1H), 3.45 (t, J=6.3 Hz, 2H), 2.73-2.67 (m, 2H), 2.53-2.37 (m, 8H), 2.35 (t, J=6.3 Hz, 2H), 2.07-1.81 (m, 4H), 1.78-1.50 (m, 6H). MS (ESI+) m/z 650.3 (M+H)⁺.

### Example 143

### 2-amino-N-[(1S,2S)-2-({4-[(1S)-1-(4-methylpiperazin-1-yl)-2,3-dihydro-1H-inden-5-yl]phenyl}methoxy)cyclopentyl]-5-(trifluoromethyl)pyridine-3-carboxamide

The title compound was prepared as described in Example 130 substituting the product of Step 139A for the product of Step 83C. ¹H NMR (400 MHz, DMSO-d₆) δ 8.55 (d, J=7.6 Hz, 1H), 8.43-8.34 (m, 1H), 8.23-8.14 (m, 1H), 7.75 (brs, 2H), 7.61-7.53 (m, 2H), 7.48-7.39 (m, 2H), 7.37 (d, J=8.1 Hz, 2H), 7.31 (d, J=7.8 Hz, 1H), 4.65-4.53 (m, 2H), 4.36-4.23 (m, 2H), 3.97-3.87 (m, 1H), 3.00-2.86 (m, 1H), 2.86-2.74 (m, 1H), 2.53-2.44 (m, 2H), 2.43-2.20 (m, 6H), 2.14 (s, 3H), 2.10-1.85 (m, 4H), 1.80-1.50 (m, 4H). MS (ESI+) m/z 594.2 (M+H)⁺.

### Example 144

### 2-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 43D substituting Intermediate 11 for the product of Step 43C. ¹H NMR (500 MHz, DMSO-d₆) δ 8.35 (d, J=7.7 Hz, 1H), 8.32 (d, J=2.2 Hz, 1H), 8.03-7.96 (m, 2H), 7.78 (d, J=0.8 Hz, 1H), 7.61-7.55 (m, 2H), 7.45-7.35 (m, 4H), 7.28-7.21 (m, 1H), 6.98 (brs, 2H), 4.66-4.56 (m, 2H), 4.41 (t, J=7.9 Hz, 1H), 4.35-4.27 (m, 1H), 3.95-3.89 (m, 1H), 3.83 (s, 3H), 2.52-2.46 (m, 2H), 2.46-2.22 (m, 6H), 2.16 (s, 3H), 2.09-2.00 (m, 1H), 1.99-1.83 (m, 3H), 1.80-1.53 (m, 4H), 1.36 (s, 3H), 1.18 (s, 3H). MS (ESI+) m/z 634.2 (M+H)⁺.

### Example 145

### 2-amino-N-[(1S,2S)-2-({4-[(1R)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 43D substituting Intermediate 12 for the product of Step 43C. ¹H NMR (501 MHz, DMSO-d₆) δ 8.37-8.30 (m, 2H), 8.02-7.97 (m, 2H), 7.78 (d, J=0.8 Hz, 1H), 7.60-7.55 (m, 2H), 7.44-7.36 (m, 4H), 7.27-7.22 (m, 1H), 6.96 (s, 2H), 4.66-4.56 (m, 2H), 4.41 (t, J=7.9 Hz, 1H), 4.36-4.27 (m, 1H), 3.96-3.90 (m, 1H), 3.83 (s, 3H), 2.53-2.46 (m, 2H), 2.46-2.24 (m, 6H), 2.17 (s, 3H), 2.10-2.00 (m, 1H), 1.99-1.83 (m, 3H), 1.80-1.53 (m, 4H), 1.37 (s, 3H), 1.17 (s, 3H). MS (ESI+) m/z 634.1 (M+H)⁺.

### Example 146

### 2-amino-N-[(1S,2S)-2-({4-[8,8-dimethyl-5-(piperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

### Step 146A

### tert-butyl 4-(6-bromo-4,4-dimethyl-1,2,3,4-tetrahydronaphthalen-1-yl)piperazine-1-carboxylate

A mixture of 6-bromo-4,4-dimethyl-3,4-dihydronaphthalen-1 (2H)-one (0.98 g, 3.87 mmol), triethylamine (1.619 mL, 11.61 mmol) and tert-butyl piperazine-1-carboxylate (1.442 g, 7.74 mmol) in dichloromethane (30 mL) was treated with 1 M titanium(IV) chloride in dichloromethane (1.936 mL, 1.936 mmol), and the solution was stirred at ambient temperature for 20 hours. Methanol (10 mL) was added, followed by sodium cyanoborohydride (0.852 g, 13.55 mmol), and the mixture was stirred at ambient temperature for 2 days. The mixture was partitioned in saturated NaHCO₃ and dichloromethane and the aqueous layer was extracted with additional dichloromethane. The combined organic layers were washed with brine, dried over Na₂SO₄, and concentrated. The residue was purified by flash chromatography on silica gel eluting with 0-10% ethyl acetate/heptane to afford the title compound (520 mg). MS (ESI+) m/z 423.0 (M+H)⁺.

### Step 146B

### 1-(6-bromo-4,4-dimethyl-1,2,3,4-tetrahydronaphthalen-1-yl)piperazine

A solution of the product of Step 146A (0.53 g, 1.252 mmol) in methanol (8 mL) was treated with 4 M HCl in dioxane (3.13 mL, 12.52 mmol), and the mixture was stirred at ambient temperature for 16 hours. The mixture was cooled to 0° C. and ethyl acetate and saturated Na₂CO₃ were added. The bilayer was stirred vigorously for 5 minutes and the aqueous layer was extracted with additional ethyl acetate. The combined organic layers were washed with brine, dried over Na₂SO₄, and concentrated to afford the title compound (417 mg). MS (APCI+) m/z 325.07 (M+H)⁺.

### Step 146C

### 2-amino-N-[(1S,2S)-2-({4-[8,8-dimethyl-5-(piperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

A mixture of Intermediate 33 (0.03 g, 0.055 mmol), the product of Step 146B (0.020 g, 0.063 mmol), bis(di-tert-butyl(4-dimethylaminophenyl)phosphine)dichloropalladium(II) (1.950 mg, 2.75 μmol) and K₂CO₃ (0.023 g, 0.165 mmol) in degassed water (0.2 mL) and degassed dioxane (0.7 mL) was heated at 75° C. for 1 hour. The mixture was cooled to ambient temperature and partitioned in water and dichloromethane. The organic layer was washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (18 mg). ¹H NMR (500 MHz, DMSO-d₆) δ 8.36 (d, J=7.7 Hz, 1H), 8.32 (d, J=2.2 Hz, 1H), 8.03-7.96 (m, 2H), 7.78 (s, 1H), 7.72-7.66 (m, 1H), 7.60-7.55 (m, 2H), 7.52 (t, J=1.8 Hz, 1H), 7.42-7.33 (m, 3H), 6.98 (s, 2H), 4.66-4.56 (m, 2H), 4.35-4.26 (m, 1H), 3.95-3.89 (m, 1H), 3.83 (d, J=1.9 Hz, 3H), 3.78-3.67 (m, 1H), 2.83-2.70 (m, 3H), 2.50-2.37 (m, 9H), 2.09-1.88 (m, 2H), 1.85-1.53 (m, 5H), 1.30 (d, J=4.3 Hz, 3H), 1.24 (d, J=3.2 Hz, 3H). MS (ESI+) m/z 634.3 (M+H)⁺.

### Example 147

### 2-amino-N-[(1S,2S)-2-({4-[8,8-dimethyl-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

### Step 147A

### 1-(6-bromo-4,4-dimethyl-1,2,3,4-tetrahydronaphthalen-1-yl)-4-methylpiperazine

The title compound was prepared as described in Step 139A substituting the product of Step 146B for Intermediate 16. MS (ESI+) m/z 339.1 (M+H)⁺.

### Step 147B

### 2-amino-N-[(1S,2S)-2-({4-[8,8-dimethyl-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

The title compound was prepared as described in Step 146C substituting the product of Step 147A for the product of Step 146B. ¹H NMR (501 MHz, DMSO-d₆) δ 8.37-8.30 (m, 2H), 8.03-7.96 (m, 2H), 7.78 (d, J=0.9 Hz, 1H), 7.66 (dd, J=8.2, 1.0 Hz, 1H), 7.61-7.50 (m, 3H), 7.41-7.33 (m, 3H), 6.97 (s, 2H), 4.66-4.56 (m, 2H), 4.36-4.27 (m, 1H), 3.96-3.89 (m, 1H), 3.83 (d, J=1.8 Hz, 3H), 3.76 (dd, J=9.9, 5.4 Hz, 1H), 2.56-2.41 (m, 4H), 2.40-2.24 (m, 4H), 2.17 (s, 3H), 2.10-2.00 (m, 1H), 1.98-1.90 (m, 1H), 1.84-1.54 (m, 8H), 1.27 (dd, J=29.6, 3.7 Hz, 6H). MS (ESI+) m/z 648.2 (M+H)⁺.

### Example 148

### 2-amino-N-{(1S,2S)-2-[(4-{5-[4-(2-hydroxyethyl)piperazin-1-yl]-8,8-dimethyl-5,6,7,8-tetrahydronaphthalen-2-yl}phenyl)methoxy]cyclopentyl}-5-(1-methyl-1H-pyrazol-4-yl)pyridine-3-carboxamide

### Step 148A

### 2-(4-(6-bromo-4,4-dimethyl-1,2,3,4-tetrahydronaphthalen-1-yl)piperazin-1-yl)ethanol

The title compound was prepared as described in Step 8C substituting the product of Step 146B for the product of Step 8B. MS (ESI+) m/z 367.2 (M+H)⁺.

### Step 148B

### 2-amino-N-{(1S,2S)-2-[(4-{5-[4-(2-hydroxyethyl)piperazin-1-yl]-8,8-dimethyl-5,6,7,8-tetrahydronaphthalen-2-yl}phenyl)methoxy]cyclopentyl}-5-(1-methyl-1H-pyrazol-4-yl)pyridine-3-carboxamide

The title compound was prepared as described in Step 146C substituting the product of Step 148A for the product of Step 146B. ¹H NMR (501 MHz, DMSO-d₆) δ 8.37-8.30 (m, 2H), 8.03-7.96 (m, 2H), 7.78 (s, 1H), 7.70-7.64 (m, 1H), 7.60-7.50 (m, 3H), 7.41-7.33 (m, 3H), 6.97 (s, 2H), 4.66-4.56 (m, 2H), 4.38-4.27 (m, 2H), 3.96-3.90 (m, 1H), 3.83 (d, J=1.3 Hz, 3H), 3.75 (dd, J=10.1, 5.4 Hz, 1H), 3.49 (q, J=6.0 Hz, 2H), 2.56-2.36 (m, 9H), 2.10-2.00 (m, 1H), 1.98-1.89 (m, 1H), 1.86-1.54 (m, 8H), 1.30 (d, J=4.1 Hz, 3H), 1.24 (d, J=3.2 Hz, 3H). MS (ESI+) m/z 678.2 (M+H)⁺.

### Example 149

### 6-amino-2′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

The title compound was prepared as described in Step 12D substituting Intermediate 19 for Intermediate 24 and substituting the product of Step 88C for the product of Step 12C. ¹H NMR (400 MHz, DMSO-d₆) δ 8.46-8.32 (m, 2H), 8.23-8.05 (m, 3H), 7.62 (d, J=8.1 Hz, 1H), 7.56 (d, J=8.1 Hz, 2H), 7.48-7.26 (m, 6H), 7.19-6.98 (m, 1H), 4.68-4.52 (m, 2H), 4.33 (dt, J=16.1, 6.2 Hz, 3H), 3.90 (dt, J=6.5, 3.9 Hz, 1H), 3.77 (q, J=5.2 Hz, 2H), 3.48 (q, J=6.1 Hz, 3H), 2.74 (t, J=5.1 Hz, 3H), 2.46-2.35 (m, 6H), 2.09-1.50 (m, 10H). MS (ESI+) m/z 665 (M+H)⁺.

### Example 150

### 6-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide

The title compound was prepared as described in Step 12D substituting Intermediate 19 for Intermediate 24 and substituting Intermediate 11 for the product of Step 12C. ¹H NMR (501 MHz, DMSO-d₆) δ 8.38 (d, J=7.7 Hz, 1H), 8.33 (t, J=2.2 Hz, 1H), 8.18-8.03 (m, 3H), 7.60-7.50 (m, 2H), 7.42-7.19 (m, 8H), 4.63-4.52 (m, 2H), 4.39 (t, J=7.9 Hz, 1H), 4.29 (qd, J=7.8, 4.0 Hz, 1H), 4.15-3.93 (m, 1H), 3.89 (dt, J=6.3, 3.9 Hz, 1H), 3.15 (d, J=3.0 Hz, 1H), 2.45-2.24 (m, 6H), 2.15 (s, 3H), 2.02 (ddd, J=15.4, 8.1, 5.4 Hz, 1H), 1.95-1.83 (m, 3H), 1.74-1.50 (m, 4H), 1.35 (s, 3H), 1.16 (s, 3H). MS (ESI+) m/z 649 (M+H)⁺.

### Example 151

### 6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6′-methoxy[3,3′-bipyridine]-5-carboxamide

A mixture of Intermediate 20 (0.25 g, 0.459 mmol), the product of Step 34A (0.164 g, 0.505 mmol), K₂CO₃ (0.159 g, 1.148 mmol) and bis(di-tert-butyl(4-dimethylaminophenyl)phosphine) dichloropalladium(II) (0.016 g, 0.023 mmol) in degassed water (1.5 mL) and degassed dioxane (4.7 mL) was heated under nitrogen at 75° C. for 1 hour. The mixture was cooled to ambient temperature and partitioned in water and dichloromethane. The organic layer was washed with brine, dried over Na₂SO₄, and concentrated. The residue was purified by flash chromatography on silica gel eluting with 0-10% methanol/dichloromethane followed by 0-100% (89:10:1 dichloromethane/methanol/NH₄OH) in dichloromethane to afford the title compound (142 mg). ¹H NMR (501 MHz, DMSO-d₆) δ 8.47 (d, J=2.6 Hz, 1H), 8.44-8.34 (m, 2H), 8.11 (d, J=2.4 Hz, 1H), 7.98 (dd, J=8.5, 2.6 Hz, 1H), 7.62-7.50 (m, 2H), 7.46-7.33 (m, 4H), 7.29 (d, J=7.8 Hz, 1H), 7.16 (s, 2H), 6.87 (d, J=8.6 Hz, 1H), 4.67-4.56 (m, 2H), 4.39-4.21 (m, 3H), 3.96-3.81 (m, 4H), 3.56-3.45 (m, 2H), 2.98-2.73 (m, 2H), 2.45-2.28 (m, 7H), 2.13-1.86 (m, 4H), 1.80-1.50 (m, 4H). MS (ESI+) m/z 663.6 (M+H)⁺.

### Example 152

### 6-amino-6′-methoxy-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide

The title compound was prepared as described in Example 151 substituting the product of Step 139A for the product of Step 34A. ¹HNMR (400 MHz, DMSO-d₆) δ 8.47 (d, J=2.5 Hz, 1H), 8.43-8.36 (m, 2H), 8.12 (d, J=2.3 Hz, 1H), 7.98 (dd, J=8.6, 2.6 Hz, 1H), 7.58-7.52 (m, 2H), 7.45-7.34 (m, 4H), 7.29 (d, J=7.8 Hz, 1H), 7.16 (s, 2H), 6.87 (d, J=8.6 Hz, 1H), 4.65-4.55 (m, 2H), 4.38-4.24 (m, 2H), 3.96-3.85 (m, 4H), 2.96-2.73 (m, 2H), 2.50-2.21 (m, 8H), 2.15 (s, 3H), 2.11-1.89 (m, 4H), 1.80-1.51 (m, 4H). MS (APCI+) m/z 633.47 (M+H)⁺.

### Example 1536-amino-6′-cyano-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide

### Step 153A

### 2-amino-N-{(1S,2S)-2-[(4-bromophenyl)methoxy]cyclopentyl}-5-iodopyridine-3-carboxamide

A solution of 2-amino-5-iodonicotinic acid (500 mg, 1.894 mmol), I-[bis(dimethylamino)methylene]-1H-1,2,3-triazolo[4,5-b]pyridinium 3-oxid hexafluorophosphate (HATU, 756 mg, 1.988 mmol) and triethylamine (0.528 mL, 3.79 mmol) in N,N-dimethylformamide (5 mL) was stirred at ambient temperature for 10 minutes. A separate solution of Intermediate 1 (free base, 610 mg, 1.988 mmol) and triethylamine (1.056 mL, 7.58 mmol) in N,N-dimethylformamide (5 mL) was added, and the mixture was stirred at ambient temperature for 6 hours. The mixture was poured into water, and the resulting suspension was filtered, and the solid was washed with water and dried under vacuum to afford the title compound (930 mg). MS (APCI+) m/z 516.06 (M+H)⁺.

### Step 153B

### 6-amino-N-{(1S,2S)-2-[(4-bromophenyl)methoxy]cyclopentyl}-6′-cyano[3,3′-bipyridine]-5-carboxamide

A mixture of the product of Step 153A (300 mg, 0.558 mmol), 5-(4,4,5,5-tetramethyl-1,3,2-dioxaborolan-2-yl)picolinonitrile (134 mg, 0.558 mmol), [1,1′-bis(diphenylphosphino)ferrocene]palladiurm(II) dichloride (23 mg, 0.028 mmol) and K₂CO₃ (193 mg, 1.395 mmol) in degassed dioxane (9 mL) and degassed water (2 mL) was heated under nitrogen at 80° C. for 2 hours. The mixture was cooled to ambient temperature and partitioned in dichloromethane and water. The organic layer was washed with brine, dried over Na₂SO₄, and concentrated. The residue was purified by flash chromatography on silica gel eluting with 0-6% methanol/dichloromethane to afford the title compound (200 mg). MS (ESI+) m/z 494.3 (M+H)⁺.

### Step 153C

### 6-amino-6′-cyano-N-[(1S,2S)-2-{[4-(4,4,5,5-tetramethyl-1,3,2-dioxaborolan-2-yl)phenyl]methoxy}cyclopentyl][3,3′-bipyridine]-5-carboxamide

A mixture of the product of Step 153B (200 mg, 0.406 mmol), 4,4,4′,4′,5,5,5′,5′-octamethyl-2,2′-bi(1,3,2-dioxaborolane) (196 mg, 0.772 mmol), potassium acetate (120 mg, 1.219 mmol) and [1,1′-bis(diphenylphosphino)ferrocene]palladium(II) dichloride (27 mg, 0.032 mmol) in dioxane (2.6 mL) was heated under nitrogen at 100° C. for 3.5 hours. The mixture was cooled to ambient temperature and partitioned in ethyl acetate and water. The organic layer was washed with brine, dried over Na₂SO₄, and concentrated. The residue was purified by flash chromatography on silica gel eluting with 0-100% ethyl acetate/heptane to afford the title compound (144 mg). MS (ESI+) m/z 540.0 (M+H)⁺.

### Step 153D

### 6-amino-6′-cyano-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide

A mixture of the product of Step 153C (30 mg, 0.056 mmol), the product of Step 34A (20 mg, 0.061 mmol), bis(di-tert-butyl(4-dimethylaminophenyl)phosphine)dichloropalladium (II) (1.969 mg, 2.78 μmol) and K₂CO₃ (19 mg, 0.139 mmol) in degassed water (0.2 mL) and degassed dioxane (0.7 mL) was heated at 70° C. for 2 hours. The mixture was cooled to ambient temperature and partitioned in water and dichloromethane. The organic layer was washed with brine, dried over Na₂SO₄, and concentrated. The residue was purified by flash chromatography on silica gel eluting with 0-15% methanol/dichloromethane to afford the title compound (11 mg). ¹HNMR (501 MHz, DMSO-d₆) δ 9.12 (dd, J=2.4, 0.9 Hz, 1H), 8.62 (d, J=2.4 Hz, 1H), 8.43 (d, J=7.8 Hz, 1H), 8.34-8.25 (m, 2H), 8.05 (dd, J=8.3, 0.8 Hz, 1H), 7.57-7.51 (m, 2H), 7.48 (brs, 2H), 7.43-7.35 (m, 4H), 7.31-7.26 (m, 1H), 4.60 (s, 2H), 4.37-4.25 (m, 3H), 3.96-3.90 (m, 1H), 3.50-3.43 (m, 2H), 2.95-2.86 (m, 1H), 2.84-2.75 (m, 1H), 2.48-2.32 (m, 9H), 2.11-1.92 (m, 4H), 1.80-1.52 (m, 4H). MS (ESI+) m/z 658.4 (M+H)⁺.

### Example 154

### 6-amino-6′-cyano-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide

The title compound was prepared as described in Step 153D substituting the product of Step 139A for the product of Step 34A. ¹H NMR (400 MHz, DMSO-d₆) δ 9.12 (d, J=2.3 Hz, 1H), 8.62 (d, J=2.4 Hz, 1H), 8.43 (d, J=7.8 Hz, 1H), 8.35-8.25 (m, 2H), 8.05 (d, J=8.2 Hz, 1H), 7.57-7.46 (m, 4H), 7.43-7.34 (m, 4H), 7.28 (d, J=7.8 Hz, 1H), 4.60 (s, 2H), 4.38-4.25 (m, 2H), 3.97-3.89 (m, 1H), 2.96-2.73 (m, 2H), 2.50-2.23 (m, 8H), 2.15 (s, 3H), 2.12-1.89 (m, 4H), 1.80-1.51 (m, 4H). MS (ESI+) m/z 628.3 (M+H)⁺.

### Example 155

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxy-5-(1-methyl-1H-pyrazol-4-yl)pyridine-3-carboxamide

### Step 155A

### 2-amino-N-{(1S,2S)-2-[(4-bromophenyl)methoxy]cyclopentyl}-4-methoxypyridine-3-carboxamide

A solution of 2-amino-4-methoxynicotinic acid (0.52 g, 3.09 mmol), 1-[bis(dimethylamino)methylene]-1H-1,2,3-triazolo[4,5-b]pyridinium 3-oxid hexafluorophosphate (HATU, 1.235 g, 3.25 mmol) and triethylamine (2.155 mL, 15.46 mmol) in N,N-dimethylformamide (10 mL) was stirred at ambient temperature for 1.5 hours. A solution of Intermediate 1 (free base, 0.877 g, 3.25 mmol) in N,N-dimethylformamide (2 mL) was added, and the mixture was stirred at ambient temperature for 16 hours. The mixture was partitioned in water and ethyl acetate, and the organic layer was washed with brine, dried over Na₂SO₄, and concentrated. The residue was purified by flash chromatography on silica gel eluting with 0-8% methanol/dichloromethane to afford the title compound (789 mg). MS (ESI+) m/z 420.19 (M+H)⁺.

### Step 155B

### 2-amino-N-{(1S,2S)-2-[(4-bromophenyl)methoxy]cyclopentyl}-5-iodo-4-methoxypyridine-3-carboxamide

A solution of the product of Step 155A (0.785 g, 1.868 mmol) in N,N-dimethylformamide (15 mL) at 0° C. was treated with N-iodosuccinimide (0.840 g, 3.74 mmol) in a single portion. After stirring at 0° C. for 15 minutes, the mixture was stirred at ambient temperature for 40 hours. The mixture was quenched with 5% sodium sulfite and extracted with ethyl acetate. The aqueous layer was extracted with additional ethyl acetate and the combined organic layers were washed with brine, dried over Na₂SO₄, and concentrated. The residue was purified by flash chromatography on silica gel eluting with 0-5% methanol/dichloromethane to afford the title compound (860 mg). MS (ESI+) m/z 546.2 (M+H)⁺.

### Step 155C

### 2-amino-N-{(1S,2S)-2-[(4-bromophenyl)methoxy]cyclopentyl}-4-methoxy-5-(1-methyl-1H-pyrazol-4-yl)pyridine-3-carboxamide

A mixture of the product of Step 155B (0.773 g, 1.415 mmol), (1-methyl-1H-pyrazol-4-yl)boronic acid (0.232 g, 1.84 mmol) and K₂CO₃ (0.488 g, 3.538 mmol) in dioxane (19 mL) and water (5 mL) was degassed with nitrogen for 30 minutes. Bis(di-tert-butyl(4-dimethylaminophenyl)phosphine)dichloropalladium(II) (0.050 g, 0.071 mmol) was added, and the mixture was stirred under nitrogen at ambient temperature for 60 hours. The mixture was partitioned in dichloromethane and water, and the organic layer was washed with brine, dried over Na₂SO₄, and concentrated. The residue was purified by flash chromatography on silica gel eluting with 0-7% methanol/dichloromethane to afford the title compound (252 mg). MS (ESI+) m/z 500.4 (M+H)⁺.

### Step 155D

### 2-amino-4-methoxy-5-(1-methyl-1H-pyrazol-4-yl)-N-[(1S,2S)-2-{[4-(4,4,5,5-tetramethyl-1,3,2-dioxaborolan-2-yl)phenyl]methoxy}cyclopentyl]pyridine-3-carboxamide

A mixture of the product of Step 155C (0.125 g, 0.250 mmol), 4,4,4′,4′,5,5,5′,5′-octamethyl-2,2′-bi(1,3,2-dioxaborolane) (0.095 g, 0.375 mmol), and potassium acetate (0.074 g, 0.749 mmol) in degassed dioxane (1.5 mL) was treated with tris(dibenzylideneacetone)dipalladium(0) (2.288 mg, 2.498 μmol) and 2-dicyclohexylphosphino-2′,4′,6′-triisopropylbiphenyl (4.76 mg, 9.99 μmol), and the mixture was heated under nitrogen at 80° C. for 16 hours. The mixture was cooled to ambient temperature and partitioned in ethyl acetate and water. The organic layer was washed with brine, dried over Na₂SO₄, and concentrated. The residue was purified by flash chromatography on silica gel eluting with 0-8% methanol/ethyl acetate to afford the title compound (90 mg). MS (ESI+) m/z 548.3 (M+H)⁺.

### Step 155E

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxy-5-(1-methyl-1H-pyrazol-4-yl)pyridine-3-carboxamide

A mixture of the product of Step 155D (0.03 g, 0.055 mmol), the product of Step 34A (0.020 g, 0.060 mmol), bis(di-tert-butyl(4-dimethylaminophenyl)phosphine)dichloropalladium (II) (1.940 mg, 2.74 μmol) and K₂CO₃ (0.019 g, 0.137 mmol) in degassed water (0.2 mL) and degassed dioxane (0.7 mL) was heated at 75° C. for 1 hour. The mixture was cooled to ambient temperature and partitioned in water and dichloromethane. The organic layer was washed with brine, dried over Na₂SO₄, and concentrated. The residue was purified by flash chromatography on silica gel eluting with 0-15% methanol/dichloromethane to afford the title compound (20 mg). ¹H NMR (400 MHz, DMSO-d₆) δ 8.44 (d, J=7.8 Hz, 1H), 8.19 (s, 1H), 7.95 (s, 1H), 7.74 (s, 1H), 7.64-7.57 (m, 2H), 7.51-7.37 (m, 4H), 7.32 (d, J=7.8 Hz, 1H), 6.04 (s, 2H), 4.67-4.55 (m, 2H), 4.37-4.24 (m, 3H), 3.97-3.89 (m, 1H), 3.85 (s, 3H), 3.65 (s, 3H), 3.46 (q, J=6.0 Hz, 2H), 2.99-2.75 (m, 2H), 2.54-2.32 (m, 9H), 2.14-1.85 (m, 4H), 1.76-1.49 (m, 4H). MS (APCI+) m/z 666.48 (M+H)⁺.

### Example 156

### 2-amino-N-{(1S,2S)-2-[(4-{1-[1-(2-hydroxyethyl)piperidin-4-yl]-1H-benzimidazol-5-yl}phenyl)methoxy]cyclopentyl}-5-(1-methyl-1H-pyrazol-4-yl)pyridine-3-carboxamide

### Step 156A

### tert-butyl 4-(4-bromo-2-nitroanilino)piperidine-1-carboxylate

A solution of 4-bromo-1-fluoro-2-nitrobenzene (1.03 g, 4.68 mmol), tert-butyl 4-aminopiperidine-1-carboxylate (939.6 mg, 4.69 mmol) and N,N-diisopropylethylamine (0.8 mL, 4.62 mmol) in N,N-dimethylformamide (15 mL) was heated at 80° C. for 18 hours. The mixture was cooled to ambient temperature and partitioned between ethyl acetate and water. The organic phase was washed with brine, dried over MgSO₄, filtered, and concentrated. Purification by flash chromatography on silica gel eluting with 0-50% ethyl acetate/hexanes gave the title compound (1.45 g). ¹H NMR (400 MHz, DMSO-d₆) δ 8.09 (d, J=2.5 Hz, 1H), 7.82 (d, J=7.8 Hz, 1H), 7.57 (dd, J=9.3, 2.5 Hz, 1H), 7.10 (d, J=9.5 Hz, 1H), 3.81 (d, J=14.1 Hz, 2H), 2.87 (s, 2H), 1.84 (d, J=12.5 Hz, 2H), 1.38 (t, J=13.1 Hz, 2H), 1.32 (s, 9H). MS (ESI−) m/z 397.8 (M−H)⁻.

### Step 156B

### tert-butyl 4-(2-amino-4-bromoanilino)piperidine-1-carboxylate

To a suspension of the product of Step 156A (1.45 g, 3.62 mmol) and zinc (1.692 g, 25.9 mmol) in methanol was added NH₄Cl (0.934 g, 17.46 mmol) in water (0.5 mL), and the mixture was stirred at ambient temperature for 18 hours, filtered through diatomaceous earth and concentrated. Purification by flash chromatography on silica gel eluting with 0-50% ethyl acetate/hexanes gave the title compound (0.89 g). ¹H NMR (400 MHz, DMSO-d₆) δ 6.67 (d, J=2.3 Hz, 1H), 6.56 (dd, J=8.3, 2.4 Hz, 1H), 6.41 (d, J=8.5 Hz, 1H), 4.83 (s, 2H), 4.31 (d, J=7.7 Hz, 1H), 3.88 (d, J=13.3 Hz, 2H), 2.89 (s, 2H), 1.93-1.84 (m, 2H), 1.40 (s, 9H), 1.24 (qd, J=12.4, 4.1 Hz, 2H). MS (ESI−) m/z 368.0 (M−H)⁻.

### Step 156C

### tert-butyl 4-(5-bromo-1H-benzimidazol-1-yl)piperidine-1-carboxylate

To a solution of the product of Step 156B (0.898 g, 2.425 mmol) and 4-methylbenzenesulfonic acid hydrate (14.8 mg, 0.078 mmol) in toluene (10 mL) was added trimethyl orthoformate (0.5 mL, 4.56 mmol), and the mixture was heated at 110° C. for 2 hours, cooled to ambient temperature and concentrated. The residue was dissolved in ethyl acetate, washed with saturated NaHCO₃ and brine, dried over MgSO₄, filtered, and concentrated to afford the title compound (889 mg) which was used without further purification. ¹H NMR (400 MHz, methanol-d₄) δ 8.32 (s, 1H), 7.82 (d, J=1.8 Hz, 1H), 7.59 (d, J=8.7 Hz, 1H), 7.44 (dd, J=8.7, 1.8 Hz, 1H), 4.59 (tt, J=11.9, 4.0 Hz, 1H), 4.30 (ddd, J=13.6, 4.6, 2.4 Hz, 2H), 3.03 (s, 2H), 2.14 (ddt, J=11.5, 4.7, 2.4 Hz, 2H), 2.09-1.94 (m, 2H), 1.49 (s, 9H). MS (ESI+) m/z 382.1 (M+H)⁺.

### Step 156D

### 5-bromo-1-(piperidin-4-yl)-1H-benzimidazole

The title compound was prepared as described in Step 72A using the product of Step 156C in place of tert-butyl 6′-bromo-1′H-spiro[cyclopropane-1,4′-isoquinoline]-2′(3′H)-carboxylate. ¹H NMR (400 MHz, methanol-d₄) δ 8.19 (s, 1H), 7.71 (d, J=1.8 Hz, 1H), 7.50 (d, J=8.6 Hz, 1H), 7.32 (dd, J=8.7, 1.8 Hz, 1H), 4.41 (tt, J=11.9, 4.3 Hz, 1H), 3.10 (q, J=2.2 Hz, 1H), 2.72 (ddd, J=13.0, 11.9, 2.8 Hz, 2H), 2.07-1.86 (m, 5H).

### Step 156E

### 2-[4-(5-bromo-1H-benzimidazol-1-yl)piperidin-1-yl]ethan-1-ol

The title compound was prepared as described in Step 72D using the product of Step 156D in place of the product of Step 72C. ¹H NMR (400 MHz, methanol-d₄) δ 8.30 (s, 1H), 7.82 (d, J=1.9 Hz, 1H), 7.62 (d, J=8.6 Hz, 1H), 7.43 (dd, J=8.7, 1.9 Hz, 1H), 4.42 (tt, J=11.7, 4.4 Hz, 1H), 3.73 (t, J=6.0 Hz, 2H), 3.25-3.12 (m, 2H), 2.63 (t, J=6.0 Hz, 2H), 2.36 (td, J=12.0, 2.6 Hz, 2H), 2.29-2.08 (m, 4H). MS (ESI+) m/z 324.3 (M+H)⁺.

### Step 156F

### 2-amino-N-{(1S,2S)-2-[(4-{1-[1-(2-hydroxyethyl)piperidin-4-yl]-1H-benzimidazol-5-yl}phenyl)methoxy]cyclopentyl}-5-(1-methyl-1H-pyrazol-4-yl)pyridine-3-carboxamide

The title compound was prepared as described in Step 88D using the product of Step 156E in place of the product of Step 88C. ¹H NMR (400 MHz, methanol-d₄) δ 8.28 (s, 1H), 8.23 (d, J=2.3 Hz, 1H), 7.96 (d, J=2.3 Hz, 1H), 7.81 (d, J=1.6 Hz, 1H), 7.78 (s, 1H), 7.72 (s, 1H), 7.66 (d, J=8.6 Hz, 1H), 7.56 (d, J=8.1 Hz, 2H), 7.48 (dd, J=8.5, 1.6 Hz, 1H), 7.41 (d, J=8.0 Hz, 2H), 4.66 (d, J=3.8 Hz, 2H), 4.42 (td, J=7.9, 4.8 Hz, 2H), 4.04-3.89 (m, 1H), 3.79 (s, 3H), 3.73 (t, J=5.9 Hz, 2H), 3.18 (dd, J=9.3, 6.3 Hz, 2H), 2.63 (t, J=6.0 Hz, 2H), 2.46-1.94 (m, 8H), 1.89-1.49 (m, 4H). MS (ESI+) m/z 635.3 (M+H)⁺.

### Example 157

### 2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 155E substituting Step 139A for the product of Step 34A. ¹H NMR (400 MHz, DMSO-d₆) δ 8.45 (d, J=7.8 Hz, 1H), 8.19 (s, 1H), 7.95 (d, J=0.7 Hz, 1H), 7.74 (d, J=0.7 Hz, 1H), 7.65-7.57 (m, 2H), 7.51-7.43 (m, 2H), 7.43-7.37 (m, 2H), 7.32 (d, J=7.8 Hz, 1H), 6.03 (s, 2H), 4.67-4.55 (m, 2H), 4.37-4.25 (m, 2H), 3.97-3.89 (m, 1H), 3.85 (s, 3H), 3.65 (s, 3H), 2.99-2.87 (m, 1H), 2.87-2.75 (m, 1H), 2.52-2.35 (m, 4H), 2.35-2.30 (m, 4H), 2.14 (s, 3H), 2.12-1.98 (m, 3H), 1.98-1.86 (m, 1H), 1.76-1.59 (m, 3H), 1.62-1.49 (m, 1H). MS (ESI+) m/z 636.3 (M+H)⁺.

### Example 158

### 2-amino-N-{(1S,2S)-2-[(4-{8-[1-(2-hydroxyethyl)piperidin-4-yl]-5,5-dimethyl-5,6,7,8-tetrahydro-1,8-naphthyridin-3-yl}phenyl)methoxy]cyclopentyl}-5-(1-methyl-1H-pyrazol-4-yl)pyridine-3-carboxamide

### Step 158A

### tert-butyl 4-(6-bromo-4,4-dimethyl-2-oxo-3,4-dihydro-1,8-naphthyridin-1 (2H)-yl)piperidine-1-carboxylate

The title compound was prepared as described in Step 73A using 6-bromo-4,4-dimethyl-3,4-dihydro-1,8-naphthyridin-2 (1H)-one in place of 5-bromo-3,3-dimethyl-1H-pyrrolo[3,2-b]pyridin-2 (3H)-one. MS (ESI+) m/z 382.2 (M-tBu)⁺.

### Step 158B

### 6-bromo-4,4-dimethyl-1-(piperidin-4-yl)-3,4-dihydro-1,8-naphthyridin-2 (1H)-one

The title compound was prepared as described in Step 72A using the product of Step 158A in place of of tert-butyl 6′-bromo-1′H-spiro[cyclopropane-1,4′-isoquinoline]-2′(3′H)-carboxylate. MS (ESI+) m/z 338.0 (M+H)⁺.

### Step 158C

### 6-bromo-4,4-dimethyl-1-(piperidin-4-yl)-1,2,3,4-tetrahydro-1,8-naphthyridine

The title compound was prepared as described in Step 113C using the product of Step 158B in place of the product of Step 113B. MS (ESI+) m/z 324.1 (M+H)⁺.

### Step 158D

### 2-[4-(6-bromo-4,4-dimethyl-3,4-dihydro-1,8-naphthyridin-1 (2H)-yl)piperidin-1-yl]ethan-1-ol

The title compound was prepared as described in Step 72D using the product of Step 158C in place of the product of Step 72C. MS (ESI+) m/z 368.3 (M+H)⁺.

### Step 158E

### 2-amino-N-{(1S,2S)-2-[(4-{8-[1-(2-hydroxyethyl)piperidin-4-yl]-5,5-dimethyl-5,6,7,8-tetrahydro-1,8-naphthyridin-3-yl}phenyl)methoxy]cyclopentyl}-5-(1-methyl-1H-pyrazol-4-yl)pyridine-3-carboxamide

The title compound was prepared as described in Step 88D using the product of Step 158D in place of the product of Step 88C. ¹H NMR (400 MHz, methanol-d₄) δ 8.14 (d, J=2.3 Hz, 1H), 7.94 (d, J=2.4 Hz, 1H), 7.86 (d, J=2.3 Hz, 1H), 7.71 (s, 1H), 7.64 (s, 1H), 7.48 (d, J=2.4 Hz, 1H), 7.34 (d, J=8.2 Hz, 2H), 7.28 (d, J=8.3 Hz, 2H), 4.57 (dd, J=16.7, 4.3 Hz, 2H), 4.32 (td, J=7.5, 4.6 Hz, 1H), 3.87 (dt, J=6.4, 4.5 Hz, 1H), 3.76 (s, 3H), 3.62 (t, J=6.1 Hz, 1H), 3.29 (t, J=6.0 Hz, 2H), 3.01 (d, J=11.5 Hz, 1H), 2.90 (d, J=11.4 Hz, 1H), 2.49 (t, J=6.1 Hz, 1H), 2.23 (s, 2H), 2.24-2.15 (m, 1H), 2.12 (dd, J=12.1, 2.5 Hz, 1H), 2.05 (dt, J=13.8, 6.9 Hz, 1H), 2.00-1.80 (m, 2H), 1.84 (s, 1H), 1.77-1.65 (m, 2H), 1.62 (q, J=7.2, 6.5 Hz, 4H), 1.50 (dq, J=13.0, 7.7 Hz, 1H), 1.17 (d, J=4.6 Hz, 7H). MS (ESI+) m/z 679.5 (M+H)⁺.

### Example 159

### 6-amino-2′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide

The title compound was prepared as described in Step 12D substituting Intermediate 19 for Intermediate 24 and substituting Intermediate 14 for the product of Step 12C. ¹H NMR (400 MHz, DMSO-d₆) δ 8.36 (d, J=7.7 Hz, 1H), 8.32 (t, J=2.3 Hz, 1H), 8.17-8.04 (m, 3H), 7.60-7.51 (m, 2H), 7.42-7.10 (m, 8H), 4.56 (d, J=2.5 Hz, 2H), 4.40-4.23 (m, 3H), 3.94-3.82 (m, 1H), 3.44 (q, J=6.2 Hz, 2H), 2.44-2.30 (m, 8H), 2.09-1.77 (m, 5H), 1.74-1.43 (m, 5H), 1.33 (s, 3H), 1.15 (s, 3H). MS (ESI+) m/z 679 (M+H)⁺.

### Example 160

### 6-amino-2′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide

The title compound was prepared as described in Step 142F using Intermediate 19 in place of Intermediate 24 and (7R,8aS)-2-((S)-5-bromo-2,3-dihydro-1H-inden-1-yl)octahydropyrrolo[1,2-a]pyrazin-7-ol in place of Step 142E. ¹H NMR (400 MHz, methanol-d₄) δ 8.27 (t, J=2.1 Hz, 1H), 8.11-8.05 (m, 1H), 7.99-7.94 (m, 2H), 7.47 (d, J=8.3 Hz, 2H), 7.38-7.26 (m, 6H), 4.62 (dd, J=22.3, 12.2 Hz, 2H), 4.42-4.30 (m, 3H), 3.93 (dt, J=6.7, 4.6 Hz, 1H), 3.36 (dd, J=9.7, 6.8 Hz, 1H), 3.01-2.78 (m, 5H), 2.60-2.49 (m, 2H), 2.45 (td, J=11.2, 2.9 Hz, 1H), 2.29 (td, J=10.9, 3.0 Hz, 1H), 2.19-2.06 (m, 5H), 2.05-1.94 (m, 1H), 1.83-1.66 (m, 4H), 1.60-1.49 (m, 1H). MS (ESI+) m/z 663.1 (M+H)⁺.

### Example 161

### 2-amino-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}-5-(1-methyl-1H-pyrazol-4-yl)pyridine-3-carboxamide

The title compound was prepared as described in Step 142F using the product of Step 117A in place of Step 142E. ¹H NMR (501 MHz, methanol-d₄) δ 8.23 (d, J=2.3 Hz, 1H), 7.95 (d, J=2.3 Hz, 1H), 7.81 (d, J=0.8 Hz, 1H), 7.73 (d, J=0.8 Hz, 1H), 7.49 (d, J=8.3 Hz, 2H), 7.41-7.32 (m, 5H), 4.67 (d, J=12.4 Hz, 1H), 4.63 (d, J=12.4 Hz, 1H), 4.44-4.33 (m, 3H), 3.96 (dt, J=6.7, 4.6 Hz, 1H), 3.85 (s, 3H), 3.38 (dd, J=9.8, 6.8 Hz, 1H), 3.02-2.81 (m, 5H), 2.63-2.52 (m, 2H), 2.48 (td, J=11.3, 3.0 Hz, 1H), 2.32 (td, J=11.0, 3.0 Hz, 1H), 2.21-2.08 (m, 5H), 2.07-1.98 (m, 1H), 1.86-1.68 (m, 4H), 1.64-1.54 (m, 1H). MS (ESI+) m/z 648.2 (M+H)⁺.

### Example 162

### 2-amino-N-{(1S,2S)-2-[(4-{8-[1-(2-hydroxyethyl)piperidin-4-yl]-5,6,7,8-tetrahydro-1,8-naphthyridin-3-yl}phenyl)methoxy]cyclopentyl}-5-(1-methyl-1H-pyrazol-4-yl)pyridine-3-carboxamide

### Step 162A

### tert-butyl 4-(6-bromo-3,4-dihydro-1,8-naphthyridin-1 (2H)-yl)piperidine-1-carboxylate

A mixture of 6-bromo-1,2,3,4-tetrahydro-1,8-naphthyridine (336 mg, 1.578 mmol), tert-butyl 4-oxopiperidine-1-carboxylate (629 mg, 3.16 mmol), 1 M titanium(IV) chloride in dichloromethane (0.8 mL, 0.80 mmol) and triethylamine (0.7 mL, 5.0 mmol) were stirred in dichloromethane (5 mL) for 24 hours. Sodium cyanotrihydroborate (399 mg, 6.35 mmol) in methanol (2 mL) was added, and the mixture was stirred for 18 hours. The mixture was concentrated, and the residue purified by flash chromatography on silica gel eluting with 0-50% ethyl acetate/hexanes to afford the title compound (185 mg). ¹H NMR (400 MHz, DMSO-d₆) δ 7.90 (d, J=2.5 Hz, 1H), 7.33 (dd, J=2.4, 1.3 Hz, 1H), 4.74 (tt, J=10.7, 5.3 Hz, 1H), 4.04 (dd, J=10.2, 6.3 Hz, 2H), 3.23 (dd, J=6.6, 4.6 Hz, 2H), 2.79 (s, 2H), 2.66 (t, J=6.3 Hz, 2H), 1.81-1.71 (m, 2H), 1.65-1.49 (m, 4H), 1.41 (s, 9H). MS (ESI+) m/z 396.1 (M+H)⁺.

### Step 162B

### 6-bromo-1-(piperidin-4-yl)-1,2,3,4-tetrahydro-1,8-naphthyridine

The title compound was prepared as described in Step 72A using the product of Step 162A in place of tert-butyl 6′-bromo-1′H-spiro[cyclopropane-1,4′-isoquinoline]-2′(3′H)-carboxylate. ¹H NMR (501 MHz, methanol-d₄) δ 7.87 (d, J=2.4 Hz, 1H), 7.29 (d, J=2.4 Hz, 1H), 3.55-3.20 (m, 4H), 3.00 (td, J=12.8, 2.9 Hz, 2H), 2.71 (t, J=6.3 Hz, 2H), 2.06-1.63 (m, 7H). MS (ESI+) m/z 296.2 (M+H)⁺.

### Step 162C

### 2-[4-(6-bromo-3,4-dihydro-1,8-naphthyridin-1 (2H)-yl)piperidin-1-yl]ethan-1-ol

The title compound was prepared as described in Step 72D using the product of Step 162B in place of the product of Step 72C. ¹H NMR (501 MHz, methanol-d₄) δ 7.87 (d, J=2.5 Hz, 1H), 7.30 (d, J=2.3 Hz, 1H), 4.57 (s, 1H), 3.90-3.84 (m, 2H), 3.62 (d, J=10.0 Hz, 2H), 3.60 (s, 2H), 3.17 (t, J=5.4 Hz, 2H), 3.05 (t, J=11.9 Hz, 2H), 2.72 (t, J=6.3 Hz, 2H), 2.17 (qd, J=13.3, 4.0 Hz, 2H), 1.93-1.82 (m, 4H). MS (ESI+) m/z 340.2 (M+H)⁺.

### Step 162D

### 2-amino-N-{(1S,2S)-2-[(4-{8-[1-(2-hydroxyethyl)piperidin-4-yl]-5,6,7,8-tetrahydro-1,8-naphthyridin-3-yl}phenyl)methoxy]cyclopentyl}-5-(1-methyl-1H-pyrazol-4-yl)pyridine-3-carboxamide

The title compound was prepared as described in Step 88D using the product of Step 162C in place of the product of Step 88C. ¹H NMR (400 MHz, DMSO-d₆) δ 8.38 (d, J=7.7 Hz, 1H), 8.32 (d, J=2.2 Hz, 1H), 8.16 (d, J=2.5 Hz, 1H), 8.02 (d, J=2.3 Hz, 1H), 7.99 (s, 1H), 7.79 (s, 1H), 7.52-7.44 (m, 2H), 7.42 (d, J=2.4 Hz, 1H), 7.32 (d, J=8.0 Hz, 2H), 6.97 (s, 2H), 4.71 (tt, J=12.0, 4.0 Hz, 1H), 4.63-4.51 (m, 2H), 4.31 (qd, J=7.5, 3.9 Hz, 1H), 3.92 (dt, J=6.5, 3.8 Hz, 1H), 3.84 (s, 3H), 3.50 (t, J=6.3 Hz, 2H), 3.30 (t, J=5.7 Hz, 3H), 2.95 (dd, J=8.9, 6.1 Hz, 2H), 2.71 (t, J=6.2 Hz, 2H), 2.40 (t, J=6.4 Hz, 2H), 2.12-1.47 (m, 12H). MS (ESI+) m/z 651.6 (M+H)⁺.

### Example 163

### 6-amino-2′-fluoro-N-[(1S,2S)-2-({4-[(5S)-5-(4-methylpiperazin-1-yl)-5,6,7,8-tetrahydronaphthalen-2-yl]phenyl}methoxy)cyclopentyl][3,3′-bipyridine]-5-carboxamide

### Step 163A

### 1-[(1S)-6-bromo-1,2,3,4-tetrahydronaphthalen-1-yl]-4-methylpiperazine

To a solution of the product of Step 142D (250 mg, 0.847 mmol) in methanol (5 mL) was added 37% aqueous formaldehyde solution (0.126 mL, 1.694 mmol) and acetic acid (0.097 mL, 1.694 mmol) dropwise, and the mixture was stirred for 15 minutes. Sodium triacetoxyborohydride (538 mg, 2.54 mmol) was added in portions over 5 minutes, and the mixture was stirred at ambient temperature for 2 hours. The mixture was concentrated, and the residue was dissolved in 6 N NaOH (20 mL). The solution was extracted with dichloromethane and the extracts were washed with water and brine, dried over Na₂SO₄, and concentrated. The residue was purified by flash chromatography on silica gel eluting with 0-7% methanol containing 1% NH₄OH/dichloromethane to afford the title compound (150 mg).

¹H NMR (400 MHz, DMSO-d₆) δ 8.35 (d, J=7.7 Hz, 1H), 8.31 (t, J=2.3 Hz, 1H), 8.16-8.13 (m, 1H), 8.13-8.05 (m, 2H), 7.58 (d, J=8.1 Hz, 1H), 7.54-7.50 (m, 2H), 7.40 (ddd, J=7.1, 4.8, 2.0 Hz, 1H), 7.33 (d, J=8.2 Hz, 3H), 7.29-7.25 (m, 3H), 4.59-4.51 (m, 2H), 4.27 (dt, J=11.6, 7.3 Hz, 1H), 3.89-3.84 (m, 1H), 3.78-3.72 (m, 1H), 2.78-2.66 (m, 2H), 2.53-2.22 (m, 8H), 2.13 (s, 3H), 2.06-1.83 (m, 4H), 1.74-1.46 (m, 6H). MS (ESI+) m/z 309, 311 (M+H)⁺, Br pattern.

### Step 163B

### 6-amino-2′-fluoro-N-[(1S,2S)-2-({4-[(5S)-5-(4-methylpiperazin-1-yl)-5,6,7,8-tetrahydronaphthalen-2-yl]phenyl}methoxy)cyclopentyl][3,3′-bipyridine]-5-carboxamide

The title compound was prepared as described in Step 142F using Intermediate 19 in place of Intermediate 24 and the product of Step 163A in place of the product of Step 142E. ¹H NMR (400 MHz, DMSO-d₆) δ 8.35 (d, J=7.7 Hz, 1H), 8.31 (t, J=2.3 Hz, 1H), 8.16-8.13 (m, 1H), 8.13-8.05 (m, 2H), 7.58 (d, J=8.1 Hz, 1H), 7.54-7.50 (m, 2H), 7.40 (ddd, J=7.1, 4.8, 2.0 Hz, 1H), 7.33 (d, J=8.2 Hz, 3H), 7.29-7.25 (m, 3H), 4.59-4.51 (m, 2H), 4.27 (dt, J=11.6, 7.3 Hz, 1H), 3.89-3.84 (m, 1H), 3.78-3.72 (m, 1H), 2.78-2.66 (m, 2H), 2.53-2.22 (m, 8H), 2.13 (s, 3H), 2.06-1.83 (m, 4H), 1.74-1.46 (m, 6H). MS (ESI+) m/z 635.3 (M+H)⁺.

### Example 164

### 2-amino-N-[(1S,2S)-2-({4-[(5S)-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide

The title compound was prepared as described in Step 142F using the product of Step 163A in place of the product of Step 142E. ¹H NMR (400 MHz, DMSO-d₆) δ 8.31 (d, J=7.8 Hz, 1H), 8.28 (d, J=2.2 Hz, 1H), 7.96 (d, J=2.3 Hz, 1H), 7.94 (d, J=0.8 Hz, 1H), 7.74 (d, J=0.8 Hz, 1H), 7.58 (d, J=8.3 Hz, 1H), 7.52 (d, J=8.3 Hz, 2H), 7.34 (dd, J=8.3, 2.6 Hz, 3H), 7.25 (d, J=2.0 Hz, 1H), 6.92 (s, 2H), 4.61-4.51 (m, 2H), 4.27 (qd, J=7.5, 4.0 Hz, 1H), 3.88 (dt, J=6.3, 3.8 Hz, 1H), 3.80 (s, 3H), 3.77-3.71 (m, 1H), 2.78-2.63 (m, 2H), 2.51-2.22 (m, 8H), 2.12 (s, 3H), 2.06-1.81 (m, 4H), 1.76-1.49 (m, 6H). MS (ESI+) m/z 620.3 (M+H)⁺.

### Example 165

### 6-amino-2′-fluoro-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide

The title compound was prepared as described in Step 142F using Intermediate 19 in place of Intermediate 24. ¹H NMR (400 MHz, DMSO-d₆) δ 8.36 (d, J=7.7 Hz, 1H), 8.31 (t, J=2.2 Hz, 1H), 8.14 (dt, J=4.8, 1.6 Hz, 1H), 8.12-8.05 (m, 2H), 7.59 (d, J=8.2 Hz, 1H), 7.52 (d, J=8.2 Hz, 2H), 7.39 (ddd, J=7.1, 4.8, 2.0 Hz, 1H), 7.33 (dd, J=8.3, 2.2 Hz, 3H), 7.29-7.23 (m, 3H), 4.59-4.51 (m, 2H), 4.34 (t, J=5.4 Hz, 1H), 4.27 (dt, J=11.7, 7.5 Hz, 1H), 3.89-3.84 (m, 1H), 3.74 (s, 1H), 3.45 (q, J=6.3 Hz, 2H), 2.77-2.66 (m, 2H), 2.52-2.37 (m, 8H), 2.34 (t, J=6.4 Hz, 2H), 2.05-1.83 (m, 4H), 1.72-1.46 (m, 6H). MS (ESI+) m/z 665.4 (M+H)⁺.

### Example 166

### 6-amino-N-[(1S,2S)-2-({4-[(1S)-1-{4-[(2S)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide

### Step 166A

### 1-[(1S)-5-bromo-2,3-dihydro-1H-inden-1-yl]-4-{[(4S)-2,2-dimethyl-1,3-dioxolan-4-yl]methyl}piperazine

A mixture of Intermediate 16 (568 mg, 2.02 mmol), (R)-(2,2-dimethyl-1,3-dioxolan-4-yl)methyl 4-methylbenzenesulfonate (878 mg, 3.07 mmol) and K₂CO₃ (1.40 g, 10.13 mmol) in acetonitrile (5 mL) was heated at 70° C. for 18 hours. After cooling to ambient temperature, the mixture was filtered through diatomaceous earth and purified by flash chromatography on silica gel eluting with 0-100% ethyl acetate/hexanes to afford the title compound (341 mg). ¹H NMR (501 MHz, DMSO-d₆) δ 7.42 (d, J=1.8 Hz, 1H), 7.38-7.32 (m, 1H), 7.19 (d, J=8.0 Hz, 1H), 4.24-4.11 (m, 2H), 3.97 (dd, J=8.0, 6.2 Hz, 1H), 3.50 (dd, J=8.1, 7.0 Hz, 1H), 2.87 (dt, J=14.7, 6.9 Hz, 1H), 2.76 (dt, J=16.1, 7.7 Hz, 1H), 2.54-2.27 (m, 9H), 2.07-1.94 (m, 2H), 1.29 (d, J=0.7 Hz, 3H), 1.26-1.20 (m, 3H). MS (ESI+) m/z 395.3 (M+H)⁺.

### Step 166B

### (2S)-3-{4-[(1S)-5-bromo-2,3-dihydro-1H-inden-1-yl]piperazin-1-yl}propane-1,2-diol

To a solution of the product of Step 166A (341 mg, 0.863 mmol) in dichloromethane (4 mL) at 0° C. was added water (0.1 mL, 5.55 mmol) followed by trifluoroacetic acid (0.4 mL, 5.19 mmol), and the mixture was stirred at ambient temperature for 18 hours and concentrated. The residue was dissolved in ethyl acetate and stir with NaHCO₃ for 18 hours. The mixture was filtered through diatomaceous earth, concentrated, and the residue dissolved in methanol, filtered through a Silicycle Carbonate column and concentrated to afford the title compound (314 mg). ¹H NMR (400 MHz, methanol-d₄) δ 7.38 (d, J=1.8 Hz, 1H), 7.35-7.22 (m, 2H), 4.26 (dd, J=7.8, 5.4 Hz, 1H), 3.78 (dq, J=7.6, 5.1 Hz, 1H), 3.58-3.43 (m, 2H), 3.02-2.76 (m, 2H), 2.67-2.36 (m, 10H), 2.24-2.05 (m, 2H). MS (ESI+) m/z 357.0 (M+H)⁺.

### Step 166C

### 6-amino-N-[(1S,2S)-2-({4-[(1S)-1-{4-[(2S)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide

The title compound was prepared as described in Step 88D using the product of Step 166B in place of the product of Step 88C and Intermediate 19 in place of Intermediate 33. ¹H NMR (400 MHz, methanol-d₄) δ 8.29 (t, J=2.2 Hz, 1H), 8.10 (dt, J=4.7, 1.6 Hz, 1H), 8.05-7.94 (m, 2H), 7.52-7.43 (m, 2H), 7.42-7.25 (m, 7H), 4.67 (d, J=12.4 Hz, 1H), 4.61 (d, J=12.4 Hz, 1H), 4.40 (td, J=7.6, 4.7 Hz, 1H), 4.32 (dd, J=7.7, 5.3 Hz, 1H), 3.95 (dt, J=6.5, 4.5 Hz, 1H), 3.79 (dq, J=7.5, 5.1 Hz, 1H), 3.56-3.42 (m, 2H), 2.98 (dt, J=15.2, 7.4 Hz, 1H), 2.84 (ddd, J=15.7, 8.7, 5.8 Hz, 1H), 2.70-2.34 (m, 11H), 2.15 (ddt, J=20.3, 10.6, 6.6 Hz, 3H), 2.08-1.95 (m, 1H), 1.88-1.65 (m, 3H), 1.57 (dq, J=13.0, 7.7 Hz, 1H). MS (ESI+) m/z 681.4 (M+H)⁺.

### Example 1676-amino-N-[(1S,2S)-2-({4-[(1S)-1-{4-[(2R)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide

### Step 167A

### 1-[(1S)-5-bromo-2,3-dihydro-1H-inden-1-yl]-4-{[(4R)-2,2-dimethyl-1,3-dioxolan-4-yl]methyl}piperazine

The title compound was prepared as described in Step 166A using (S)-2,2-dimethyl-1,3-dioxolane-4-methanol p-toluenesulfonate in place of (R)-(2,2-dimethyl-1,3-dioxolan-4-yl)methyl 4-methylbenzenesulfonate. ¹H NMR (500 MHz, methanol-d₄) δ 7.38 (d, J=1.8 Hz, 1H), 7.35-7.30 (m, 1H), 7.33-7.25 (m, 1H), 4.33-4.24 (m, 2H), 4.04 (ddd, J=13.0, 8.2, 6.4 Hz, 1H), 3.57-3.50 (m, 1H), 2.95 (ddd, J=15.3, 8.6, 6.1 Hz, 1H), 2.82 (ddd, J=15.9, 8.8, 5.9 Hz, 1H), 2.65-2.59 (m, 6H), 2.56-2.45 (m, 5H), 2.22-2.06 (m, 2H), 1.36 (d, J=0.8 Hz, 3H), 1.31 (d, J=0.7 Hz, 3H). MS (ESI+) m/z 395.3 (M+H)⁺.

### Step 167B

### (2R)-3-{4-[(1S)-5-bromo-2,3-dihydro-1H-inden-1-yl]piperazin-1-yl}propane-1,2-diol

The title compound was prepared as described in Step 166B using the product of Step 167A in place of the product of Step 166A. ¹H NMR (501 MHz, methanol-d₄) δ 7.40 (d, J=1.7 Hz, 1H), 7.34 (dd, J=8.1, 1.8 Hz, 1H), 7.28 (d, J=8.1 Hz, 1H), 4.33 (t, J=6.6 Hz, 1H), 3.88-3.78 (m, 1H), 3.56-3.44 (m, 2H), 2.96 (dt, J=15.2, 7.3 Hz, 1H), 2.84 (dt, J=16.0, 7.3 Hz, 6H), 2.78-2.54 (m, 5H), 2.16 (dtd, J=8.0, 6.2, 3.1 Hz, 2H). MS (ESI+) m/z 355.2 (M+H)⁺.

### Step 167C

### 6-amino-N-[(1S,2S)-2-({4-[(1S)-1-{4-[(2R)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide

The title compound was prepared as described in Step 88D using the product of Step 167B in place of the product of Step 88C and Intermediate 19 in place of Intermediate 33. ¹H NMR (400 MHz, methanol-d₄) δ 8.29 (t, J=2.2 Hz, 1H), 8.09 (dt, J=4.9, 1.5 Hz, 1H), 8.05-7.93 (m, 2H), 7.52-7.45 (m, 2H), 7.45-7.26 (m, 7H), 4.67 (d, J=12.4 Hz, 1H), 4.61 (d, J=12.4 Hz, 1H), 4.40 (td, J=7.5, 4.7 Hz, 1H), 4.31 (dd, J=7.7, 5.3 Hz, 1H), 3.95 (dt, J=6.5, 4.5 Hz, 1H), 3.78 (dt, J=7.3, 5.1 Hz, 1H), 3.56-3.43 (m, 2H), 2.97 (dt, J=15.3, 7.4 Hz, 1H), 2.83 (ddd, J=15.7, 8.6, 5.8 Hz, 1H), 2.58 (td, J=16.7, 14.0, 7.3 Hz, 9H), 2.44 (dd, J=8.2, 6.1 Hz, 2H), 2.16 (dddd, J=19.4, 11.8, 9.1, 4.4 Hz, 3H), 2.12-1.95 (m, 1H), 1.88-1.70 (m, 3H), 1.57 (dq, J=13.0, 7.7 Hz, 1H). MS (ESI+) m/z 681.4 (M+H)⁺.

### Example 168

### 6-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-2′-fluoro[3,3′-bipyridine]-5-carboxamide

### Step 168A

### (SR)—N-[(1S)-5-bromo-3,3-dimethyl-2,3-dihydro-1H-inden-1-yl]-2-methylpropane-2-sulfinamide

To a mixture of 5-bromo-3,3-dimethyl-2,3-dihydro-1H-inden-1-one (1.0 g, 4.18 mmol) and (R)-(+)-2-methyl-2-propanesulfinamide (1.014 g, 8.36 mmol) was added tetrahydrofuran (5 mL) followed by addition of tetraethoxytitanium (1.754 mL, 8.36 mmol), and the mixture was heated under nitrogen at 75° C. for 10 hours. The mixture was cooled to ambient temperature and diluted with tetrahydrofuran (10 mL). The mixture was cooled to −70° C. under nitrogen and 1 M L-Selectride in tetrahydrofuran (20.91 mL, 20.91 mmol) was added dropwise over 1.5 hours. After addition, the mixture was slowly warmed to ambient temperature and was stirred at ambient temperature for 3 hours. The mixture was cooled to 0° C. and methanol was added dropwise until gas evolution was no longer observed. Brine (25 mL) was added, and the mixture was filtered through diatomaceous earth with ethyl acetate washes. The filtrate was washed with brine, dried over Na₂SO₄, and concentrated. The crude product was purified by flash chromatography on silica gel eluting with 0-70% ethyl acetate/heptane to afford the title compound (860 mg). ¹H NMR (501 MHz, CDCl₃) δ 7.34 (dd, J=8.0, 1.8 Hz, 1H), 7.30 (d, J=1.8 Hz, 1H), 7.20-7.15 (m, 1H), 4.85-4.76 (m, 1H), 3.28 (d, J=9.8 Hz, 1H), 2.61 (dd, J=12.9, 7.2 Hz, 1H), 1.85 (dd, J=12.9, 8.5 Hz, 1H), 1.36 (s, 3H), 1.27 (s, 9H), 1.19 (s, 3H). MS (ESI+) m/z 344.2 (M+H)⁺.

### Step 168B

### (1S)-5-bromo-3,3-dimethyl-2,3-dihydro-1H-inden-1-amine

The product of Step 168A (0.85 g, 2.469 mmol) in methanol (8 mL) was treated with 4 M HCl in dioxane (3.09 mL, 12.34 mmol), and the mixture was stirred at ambient temperature for 3 hours. The mixture was concentrated, and the residue was triturated with methyl tert-butyl ether (25 mL). The solid was collected by filtration and dried in vacuo. The solid was dissolved in water (15 mL) and brought to pH 10 with 6 N NaOH. The mixture was extracted with ethyl acetate and the organic layers were washed with brine, dried over Na₂SO₄, and concentrated. The residue was dried in vacuo to afford the title compound (518 mg). MS (ESI+) m/z 240.13 (M+H)⁺.

### Step 168C

### 1-[(1S)-5-bromo-3,3-dimethyl-2,3-dihydro-1H-inden-1-yl]-4-(2-nitrobenzene-1-sulfonyl)piperazine

A solution of the product of Step 168B (0.518 g, 2.157 mmol) in acetonitrile (3 mL) under nitrogen was treated with N-ethyl-N-isopropylpropan-2-amine (1.13 mL, 6.47 mmol) followed by Intermediate 27 (1.468 g, 2.222 mmol) in acetonitrile (8 mL). The mixture was stirred at ambient temperature for 1 hour and at 40° C. for 17 hours. The mixture was concentrated, and the residue was partitioned in ethyl acetate and water. The aqueous layer was extracted with additional ethyl acetate and the combined organic layers were washed with brine, dried over Na₂SO₄, and concentrated. The residue was purified by flash chromatography on silica gel eluting with 5-50% ethyl acetate/heptane to afford the title compound (948 mg). ¹H NMR (400 MHz, DMSO-d₆) δ 8.03-7.84 (m, 4H), 7.40 (d, J=1.9 Hz, 1H), 7.33 (dd, J=8.0, 1.9 Hz, 1H), 7.11 (dd, J=8.0, 1.1 Hz, 1H), 4.39 (t, J=8.1 Hz, 1H), 3.26-3.13 (m, 4H), 2.62-2.52 (m, 2H), 2.49-2.39 (m, 2H), 1.90-1.75 (m, 2H), 1.30 (s, 3H), 1.13 (s, 3H). MS (ESI+) m/z 494.2 (M+H)⁺.

### Step 168D

### 1-[(1S)-5-bromo-3,3-dimethyl-2,3-dihydro-1H-inden-1-yl]piperazine

To a mixture of sodium hydride (0.094 g, 2.339 mmol) in tetrahydrofuran (12 mL) under nitrogen at 0° C. was added decane-1-thiol (0.515 mL, 2.432 mmol) dropwise over 3 minutes. The mixture was stirred until gas evolution had ceased (˜30 minutes). A solution of the product of Step 168C (0.925 g, 1.871 mmol) in tetrahydrofuran (6 mL) was added over 2 minutes and the mixture was stirred at ambient temperature for 30 minutes. The mixture was cooled to 0° C. and quenched by slow addition of water. The mixture was extracted with ethyl acetate and the aqueous layer was extracted with additional ethyl acetate. The combined organic layers were washed with brine, dried over Na₂SO₄, and concentrated. The residue was purified by flash chromatography on silica gel eluting with 10% methanol/ethyl acetate with 1% NH₄OH to afford the title compound (568 mg). ¹H NMR (400 MHz, DMSO-d₆) δ 7.42-7.32 (m, 2H), 7.15 (d, J=8.0 Hz, 1H), 4.30 (t, J=8.0 Hz, 1H), 2.76-2.63 (m, 4H), 2.43-2.24 (m, 4H), 1.93-1.78 (m, 2H), 1.31 (s, 3H), 1.14 (s, 3H). MS (ESI+) m/z 309.3 (M+H)⁺.

### Step 168E

### 1-[(1S)-5-bromo-3,3-dimethyl-2,3-dihydro-1H-inden-1-yl]-4-{[(4S)-2,2-dimethyl-1,3-dioxolan-4-yl]methyl}piperazine

A mixture of the product of Step 168D (0.508 g, 1.643 mmol), (R)-(2,2-dimethyl-1,3-dioxolan-4-yl)methyl 4-methylbenzenesulfonate (0.753 g, 2.63 mmol) and K₂CO₃ (0.908 g, 6.57 mmol) in acetonitrile (5 mL) and methanol (1 mL) was heated at 75° C. for 46 hours. The mixture was cooled to ambient temperature and partitioned in ethyl acetate and water. The aqueous layer was extracted with additional ethyl acetate and the combined organic layer was washed with brine, dried over Na₂SO₄, and concentrated. The residue was purified by flash chromatography on silica gel eluting with 0-5% methanol/dichloromethane to afford the title compound (446 mg). ¹H NMR (400 MHz, DMSO-d₆) δ 7.40 (d, J=1.9 Hz, 1H), 7.35 (dd, J=8.0, 1.9 Hz, 1H), 7.17-7.10 (m, 1H), 4.33 (t, J=7.9 Hz, 1H), 4.17 (p, J=6.2 Hz, 1H), 3.98 (dd, J=8.1, 6.3 Hz, 1H), 3.51 (dd, J=8.0, 7.0 Hz, 1H), 2.49-2.31 (m, 10H), 1.93-1.79 (m, 2H), 1.30 (d, J=6.5 Hz, 6H), 1.25 (s, 3H), 1.14 (s, 3H). MS (ESI+) m/z 424.9 (M+H)⁺.

### Step 168F

### (2S)-3-{4-[(1S)-5-bromo-3,3-dimethyl-2,3-dihydro-1H-inden-1-yl]piperazin-1-yl}propane-1,2-diol

A mixture of the product of Step 168E (0.508 g, 1.200 mmol) in dichloromethane (6 mL) and water (0.15 mL) was cooled in an ice bath and trifluoroacetic acid (0.555 mL, 7.20 mmol) was added. The mixture was stirred at ambient temperature for 28 hours and was concentrated. The residue was partitioned in dichloromethane and saturated NaHCO₃ and the aqueous layer was extracted with additional dichloromethane. The combined organic layer was washed with brine, dried over Na₂SO₄, and concentrated. The residue was dried in vacuo to afford the title compound (440 mg). ¹H NMR (400 MHz, DMSO-d₆) δ 7.42-7.32 (m, 2H), 7.14 (dd, J=8.1, 1.0 Hz, 1H), 4.53 (brs, 1H), 4.38-4.29 (m, 2H), 3.63-3.54 (m, 1H), 3.35-3.26 (m, 3H), 2.48-2.31 (m, 8H), 2.23 (dd, J=12.6, 6.9 Hz, 1H), 1.93-1.79 (m, 2H), 1.31 (s, 3H), 1.14 (s, 3H). MS (ESI+) m/z 383.3 (M+H)⁺.

### Step 168G

### 6-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-2′-fluoro[3,3′-bipyridine]-5-carboxamide

A mixture of Intermediate 19 (0.19 g, 0.357 mmol), the product of Step 168F (0.137 g, 0.357 mmol), potassium phosphate, tribasic (0.159 g, 0.749 mmol), water (0.7 mL) and dioxane (3 mL) was sparged with nitrogen for 45 minutes and tris(dibenzylideneacetone)dipalladium(0) (3.27 mg, 3.57 μmol) and 4-(di-tert-butylphosphino)-N,N-dimethylaniline (3.79 mg, 0.014 mmol) were added. The mixture was heated at 75° C. for 4 hours, cooled to ambient temperature and partitioned in saturated NaHCO₃ and dichloromethane. The aqueous layer was extracted with additional dichloromethane and the combined organic layer was dried over Na₂SO₄, and concentrated. The residue was purified by flash chromatography on silica gel eluting with 0-15% methanol in dichloromethane to afford the title compound (110 mg). ¹H NMR (400 MHz, methanol-d₄) δ 8.31 (t, J=2.1 Hz, 1H), 8.13-8.07 (m, 1H), 8.05-7.97 (m, 2H), 7.55-7.48 (m, 2H), 7.45-7.26 (m, 6H), 4.71-4.47 (m, 3H), 4.41 (td, J=7.6, 4.7 Hz, 1H), 4.00-3.93 (m, 1H), 3.87-3.77 (m, 1H), 3.52 (h, J=5.3 Hz, 2H), 2.75-2.42 (m, 10H), 2.23-2.09 (m, 1H), 2.08-1.94 (m, 3H), 1.87-1.69 (m, 3H), 1.64-1.52 (m, 1H), 1.40 (s, 3H), 1.21 (s, 3H). MS (ESI+) m/z 709.3 (M+H)⁺.

### Example 169

### 2-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-5-(1-methyl-1H-pyrazol-4-yl)pyridine-3-carboxamide

The title compound was prepared as described in Step 168G substituting Intermediate 24 for Intermediate 19. ¹H NMR (501 MHz, methanol-d₄) δ 8.25 (d, J=2.3 Hz, 1H), 7.97 (d, J=2.3 Hz, 1H), 7.83 (s, 1H), 7.75 (s, 1H), 7.55-7.49 (m, 2H), 7.43-7.30 (m, 5H), 4.71-4.62 (m, 2H), 4.50 (t, J=8.1 Hz, 1H), 4.46-4.38 (m, 1H), 4.01-3.94 (m, 1H), 3.87-3.77 (m, 4H), 3.56-3.46 (m, 2H), 2.76-2.55 (m, 8H), 2.54-2.41 (m, 2H), 2.21-2.11 (m, 1H), 2.07-1.94 (m, 3H), 1.86-1.71 (m, 3H), 1.66-1.55 (m, 1H), 1.38 (s, 3H), 1.21 (s, 3H). MS (APCI+) m/z 694.3 (M+H)⁺.

### Example 170

### 2-amino-N-[(1S,2S)-2-({4-[(5R)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide

### Step 170A

### 1-[(1R)-6-bromo-1,2,3,4-tetrahydronaphthalen-1-yl]-4-{[(4S)-2,2-dimethyl-1,3-dioxolan-4-yl]methyl}piperazine

The title compound was prepared as described in Step 166A using Intermediate 17 in place of Intermediate 16. ¹H NMR (400 MHz, methanol-d₄) δ 7.53 (dd, J=8.4, 1.0 Hz, 1H), 7.23 (dd, J=8.3, 2.2 Hz, 1H), 7.20 (dd, J=2.0, 1.0 Hz, 1H), 4.40-4.20 (m, 1H), 4.07 (dd, J=8.1, 6.3 Hz, 1H), 3.75 (dd, J=9.2, 4.9 Hz, 1H), 3.54 (dd, J=8.1, 7.4 Hz, 1H), 2.88-2.36 (m, 11H), 2.09-1.84 (m, 2H), 1.84-1.57 (m, 2H), 1.37 (d, J=0.8 Hz, 3H), 1.32 (d, J=0.7 Hz, 3H). MS (ESI+) m/z 409.3 (M+H)⁺.

### Step 170B

### (2S)-3-{4-[(1R)-6-bromo-1,2,3,4-tetrahydronaphthalen-1-yl]piperazin-1-yl}propane-1,2-diol

The title compound was prepared as described in Step 166B using the product of Step 170A in place of the product of Step 166A. ¹H NMR (400 MHz, methanol-d₄) δ 7.43 (dd, J=8.3, 1.0 Hz, 1H), 7.14 (dd, J=8.3, 2.2 Hz, 1H), 7.10 (dd, J=2.1, 1.0 Hz, 1H), 3.76-3.60 (m, 2H), 3.40 (h, J=5.8 Hz, 2H), 2.71-2.28 (m, 12H), 1.93-1.80 (m, 2H), 1.70-1.48 (m, 2H). MS (ESI+) m/z 371.3 (M+H)⁺.

### Step 170C

### 2-amino-N-[(1S,2S)-2-({4-[(5R)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide

The title compound was prepared as described in Step 88D using the product of Step 170B in place of the product of Step 88C. ¹H NMR (400 MHz, methanol-d₄) δ 8.22 (d, J=2.2 Hz, 1H), 7.93 (d, J=2.2 Hz, 1H), 7.77 (d, J=0.8 Hz, 1H), 7.72 (d, J=0.9 Hz, 1H), 7.61 (d, J=8.1 Hz, 1H), 7.51-7.44 (m, 2H), 7.40-7.33 (m, 2H), 7.26 (dd, J=8.1, 2.0 Hz, 1H), 7.17 (d, J=2.0 Hz, 1H), 4.70-4.57 (m, 2H), 4.41 (td, J=7.5, 4.7 Hz, 1H), 3.95 (dt, J=6.6, 4.6 Hz, 1H), 3.86-3.75 (m, 5H), 3.51 (h, J=5.3 Hz, 2H), 2.83-2.39 (m, 13H), 2.21-1.90 (m, 4H), 1.87-1.52 (m, 5H). MS (ESI+) m/z 680.2 (M+H)⁺.

### Example 171

### 2-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide

### Step 171A

### 1-[(1S)-6-bromo-1,2,3,4-tetrahydronaphthalen-1-yl]-4-{[(4S)-2,2-dimethyl-1,3-dioxolan-4-yl]methyl}piperazine

The title compound was prepared as described in Step 166A using Intermediate 18 in place of Intermediate 16. ¹H NMR (400 MHz, methanol-d₄) δ 7.53 (dd, J=8.4, 0.9 Hz, 1H), 7.24 (dd, J=8.4, 2.2 Hz, 1H), 7.20 (d, J=2.1 Hz, 1H), 4.39-4.21 (m, 1H), 4.07 (dd, J=8.1, 6.2 Hz, 1H), 3.74 (dd, J=9.2, 4.8 Hz, 1H), 3.61-3.47 (m, 1H), 2.87-2.37 (m, 12H), 1.97 (dddd, J=14.8, 6.7, 5.6, 3.7 Hz, 2H), 1.84-1.55 (m, 2H), 1.37 (s, 3H), 1.32 (s, 3H). MS (ESI+) m/z 409.1 (M+H)⁺.

### Step 171B

### (2S)-3-{4-[(1S)-6-bromo-1,2,3,4-tetrahydronaphthalen-1-yl]piperazin-1-yl}propane-1,2-diol

The title compound was prepared as described in Step 166B using the product of Step 171A in place of the product of Step 166A. ¹H NMR (501 MHz, methanol-d₄) δ 7.53 (dd, J=8.3, 1.0 Hz, 1H), 7.23 (dd, J=8.4, 2.2 Hz, 1H), 7.20 (dd, J=2.1, 1.0 Hz, 1H), 3.85-3.70 (m, 2H), 3.56-3.45 (m, 2H), 2.80-2.62 (m, 2H), 2.60-2.38 (m, 10H), 2.01-1.90 (m, 1H), 1.78-1.59 (m, 2H). MS (ESI+) m/z 369.2 (M+H)⁺.

### Step 171C

### 2-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide

The title compound was prepared as described in Step 88D using the product of Step 171B in place of the product of Step 88C. ¹H NMR (500 MHz, methanol-d₄) δ 8.23 (d, J=2.2 Hz, 1H), 7.95 (d, J=2.3 Hz, 1H), 7.79 (d, J=0.8 Hz, 1H), 7.73 (d, J=0.8 Hz, 1H), 7.63-7.58 (m, 1H), 7.51-7.44 (m, 2H), 7.40-7.31 (m, 2H), 7.25 (dd, J=8.1, 2.0 Hz, 1H), 7.17 (d, J=2.0 Hz, 1H), 4.69-4.58 (m, 2H), 4.41 (td, J=7.6, 4.7 Hz, 1H), 3.96 (dt, J=6.7, 4.6 Hz, 1H), 3.86-3.76 (m, 5H), 3.56-3.46 (m, 2H), 2.81-2.62 (m, 2H), 2.59 (s, 10H), 2.52-2.39 (m, 2H), 2.14 (dtd, J=13.1, 7.7, 5.6 Hz, 1H), 2.08-1.90 (m, 3H), 1.81-1.53 (m, 4H). MS (ESI+) m/z 680.3 (M+H)⁺.

### Example 172

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-5-yl)pyridine-3-carboxamide

### Step 172A

### 2-amino-N-{(1S,2S)-2-[(4-bromophenyl)methoxy]cyclopentyl}-5-(2-methyl-1,3-thiazol-5-yl)pyridine-3-carboxamide

A mixture of the product of Step 153A (0.11 g, 0.205 mmol), 2-methyl-5-(4,4,5,5-tetramethyl-1,3,2-dioxaborolan-2-yl)thiazole (0.092 g, 0.409 mmol), 1,1-bis(diphenylphosphino) ferrocene]palladium(II) dichloride (8.35 mg, 10.23 μmol) and K₂CO₃ (0.071 g, 0.511 mmol) in degassed dioxane (2 mL) and degassed water (0.6 mL) was heated under nitrogen at 110° C. for 6 hours. The mixture was partitioned in dichloromethane and water, and the organic layer was washed with brine, dried over Na₂SO₄, and concentrated. The residue was purified by flash chromatography on silica gel eluting with 0-10% methanol/dichloromethane to afford the title compound (59 mg). MS (ESI+) m/z 489.3 (M+H)⁺.

### Step 172B

### 2-amino-5-(2-methyl-1,3-thiazol-5-yl)-N-[(1S,2S)-2-{[4-(4,4,5,5-tetramethyl-1,3,2-dioxaborolan-2-yl)phenyl]methoxy}cyclopentyl]pyridine-3-carboxamide

The title compound was prepared as described in Step 153C substituting the product of Step 172A for the product of Step 153B. MS (ESI+) m/z 535.4 (M+H)⁺.

### Step 172C

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-5-yl)pyridine-3-carboxamide

The title compound was prepared as described in Step 155E substituting the product of Step 172B for the product of Step 155D. ¹H NMR (501 MHz, DMSO-d₆) δ 8.45 (d, J=7.8 Hz, 1H), 8.32 (d, J=2.3 Hz, 1H), 8.03 (d, J=2.4 Hz, 1H), 7.87 (s, 1H), 7.59-7.54 (m, 2H), 7.46-7.35 (m, 4H), 7.30 (d, J=7.8 Hz, 1H), 7.25 (s, 2H), 4.63-4.57 (m, 2H), 4.36 (t, J=5.4 Hz, 1H), 4.34-4.24 (m, 2H), 3.95-3.89 (m, 1H), 3.52-3.44 (m, 3H), 2.96-2.86 (m, 1H), 2.85-2.75 (m, 1H), 2.64 (s, 3H), 2.46-2.31 (m, 8H), 2.11-1.90 (m, 4H), 1.79-1.52 (m, 4H). MS (ESI+) m/z 653.2 (M+H)⁺.

### Example 173

### 6-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-2′-fluoro[3,3′-bipyridine]-5-carboxamide

The title compound was prepared as described in Step 88D using the product of Step 171B in place of the product of Step 88C and Intermediate 19 in place of Intermediate 33. ¹H NMR (501 MHz, methanol-d₄) δ 8.29 (t, J=2.1 Hz, 1H), 8.09 (dt, J=4.8, 1.5 Hz, 1H), 8.02-7.93 (m, 2H), 7.61 (d, J=8.1 Hz, 1H), 7.51-7.43 (m, 2H), 7.41-7.33 (m, 2H), 7.32-7.22 (m, 2H), 7.18 (d, J=2.0 Hz, 1H), 4.74-4.53 (m, 2H), 4.40 (td, J=7.6, 4.7 Hz, 1H), 3.95 (dt, J=6.7, 4.7 Hz, 1H), 3.80 (td, J=9.3, 8.5, 4.7 Hz, 2H), 3.59-3.44 (m, 2H), 2.87-2.34 (m, 13H), 2.14 (dtd, J=13.2, 7.7, 5.2 Hz, 1H), 1.98 (dddd, J=27.4, 14.4, 5.0, 3.2 Hz, 3H), 1.87-1.63 (m, 4H), 1.63-1.44 (m, 1H). MS (ESI+) m/z 695.4 (M+H)⁺.

### Example 174

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,2-thiazol-5-yl)pyridine-3-carboxamide

### Step 174A

### 2-amino-N-{(1S,2S)-2-[(4-bromophenyl)methoxy]cyclopentyl}-5-(1,2-thiazol-5-yl)pyridine-3-carboxamide

The title compound was prepared as described in Step 172A substituting 5-(4,4,5,5-tetramethyl-1,3,2-dioxaborolan-2-yl)isothiazole for 2-methyl-5-(4,4,5,5-tetramethyl-1,3,2-dioxaborolan-2-yl)thiazole. MS (ESI+) m/z 474 (M+H)⁺.

### Step 174B

### 2-{4-[(1S)-5-(4,4,5,5-tetramethyl-1,3,2-dioxaborolan-2-yl)-2,3-dihydro-1H-inden-1-yl]piperazin-1-yl}ethan-1-ol

The title compound was prepared as described as in Intermediate 19B substituting the product of Step 34A for Intermediate 19A. MS (ESI+) m/z 373 (M+H)⁺.

### Step 174C

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,2-thiazol-5-yl)pyridine-3-carboxamide

A mixture of the product of Step 174A (38 mg, 0.08 mmol), the product of Step 174B (34.4 mg, 0.092 mmol), bis(di-tert-butyl(4-dimethylaminophenyl)phosphine)dichloropalladium (II) (2.84 mg, 4.01 μmol), K₂CO₃ (33.3 mg, 0.241 mmol) in dioxane (1.6 mL) and water (0.4 mL) was degassed and heated at 80° C. for 4 hours. Water was added, and the mixture was extracted with ethyl acetate and the organic was washed with brine, dried over MgSO₄ and purified by flash chromatography on silica gel eluting with 0-12% methanol/dichloromethane containing 1% NH₄OH to afford the title compound (24 mg). ¹H NMR (501 MHz, DMSO-d₆) δ 8.51-8.45 (m, 3H), 8.12 (d, J=2.4 Hz, 1H), 7.64 (d, J=1.8 Hz, 1H), 7.57-7.51 (m, 2H), 7.45 (s, 2H), 7.42-7.27 (m, 5H), 4.58 (s, 2H), 4.51 (d, J=12.8 Hz, 1H), 4.29 (dt, J=11.8, 7.2 Hz, 2H), 4.10 (d, J=5.4 Hz, 1H), 3.91 (dt, J=6.4, 4.1 Hz, 1H), 3.49 (d, J=6.3 Hz, 2H), 3.15 (d, J=3.3 Hz, 1H), 2.90 (dd, J=15.8, 7.3 Hz, 1H), 2.78 (dt, J=15.7, 7.5 Hz, 1H), 2.53 (s, 4H), 2.44-2.39 (m, 2H), 2.08-1.87 (m, 5H), 1.77-1.52 (m, 5H). MS (ESI+) m/z 639 (M+H)⁺.

### Example 175

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(3-methyl-1,2-thiazol-5-yl)pyridine-3-carboxamide

### Step 175A

### 2-amino-N-{(1S,2S)-2-[(4-bromophenyl)methoxy]cyclopentyl}-5-(3-methyl-1,2-thiazol-5-yl)pyridine-3-carboxamide

The title compound was prepared as described in Step 172A substituting 3-methyl-5-(4,4,5,5-tetramethyl-1,3,2-dioxaborolan-2-yl)isothiazole for 2-methyl-5-(4,4,5,5-tetramethyl-1,3,2-dioxaborolan-2-yl)thiazole. MS (ESI+) m/z 488 (M+H)⁺.

### Step 175B

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(3-methyl-1,2-thiazol-5-yl)pyridine-3-carboxamide

The title compound was prepared as described in Step 174C substituting the product of Step 175A for the product of Step 174A. ¹H NMR (400 MHz, DMSO-d₆) δ 8.45 (d, J=7.8 Hz, 1H), 8.39 (d, J=2.3 Hz, 1H), 8.07 (d, J=2.3 Hz, 1H), 7.55-7.49 (m, 2H), 7.42 (s, 2H), 7.40-7.23 (m, 6H), 4.56 (s, 2H), 4.36-4.16 (m, 3H), 4.07 (d, J=5.5 Hz, 1H), 3.89 (dt, J=6.2, 4.0 Hz, 1H), 3.43 (d, J=5.2 Hz, 3H), 3.14 (d, J=4.6 Hz, 1H), 2.87 (ddd, J=14.5, 8.2, 5.7 Hz, 1H), 2.76 (q, J=8.0 Hz, 1H), 2.44 (s, 6H), 2.32 (d, J=6.4 Hz, 2H), 2.13-1.82 (m, 5H), 1.78-1.45 (m, 5H). MS (ESI+) m/z 653 (M+H)⁺.

### Example 176

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,3-thiazol-5-yl)pyridine-3-carboxamide

### Step 176A

### 2-amino-N-{(1S,2S)-2-[(4-bromophenyl)methoxy]cyclopentyl}-5-(1,3-thiazol-5-yl)pyridine-3-carboxamide

The title compound was prepared as described in Step 172A substituting 5-(4,4,5,5-tetramethyl-1,3,2-dioxaborolan-2-yl)thiazole for 2-methyl-5-(4,4,5,5-tetramethyl-1,3,2-dioxaborolan-2-yl)thiazole. MS (ESI+) m/z 474 (M+H)⁺.

### Step 176B

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,3-thiazol-5-yl)pyridine-3-carboxamide

The title compound was prepared as described in Step 174C substituting the product of Step 176A for the product of Step 174A. ¹H NMR (501 MHz, DMSO-d₆) δ 8.97 (s, 1H), 8.44 (d, J=7.7 Hz, 1H), 8.38 (d, J=2.4 Hz, 1H), 8.15 (s, 1H), 8.08 (d, J=2.4 Hz, 1H), 7.57-7.51 (m, 2H), 7.49-7.12 (m, 7H), 4.58 (s, 2H), 4.40-4.16 (m, 3H), 4.09 (s, 1H), 3.91 (dt, J=6.2, 3.9 Hz, 1H), 3.44 (t, J=6.5 Hz, 2H), 3.15 (s, 2H), 2.96-2.71 (m, 2H), 2.44-2.24 (m, 7H), 2.09-1.84 (m, 4H), 1.77-1.47 (m, 4H). MS (ESI+) m/z 639 (M+H)⁺.

### Example 177

### 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[2,3′-bipyridine]-5′-carboxamide

### Step 177A

### 6′-amino-N-{(1S,2S)-2-[(4-bromophenyl)methoxy]cyclopentyl}[2,3′-bipyridine]-5′-carboxamide

A mixture of 2-(tributylstannyl)pyridine (219 mg, 0.595 mmol), the product of Step 153A (209 mg, 0.405 mmol), bis(triphenylphosphine)palladium(II) dichloride (17 mg, 0.024 mmol) and triethylamine (0.06 mL, 0.430 mmol) in dioxane (2 mL) was sparged with nitrogen for 10 minutes and heated at 100° C. for 18 hours. The mixture was cooled to ambient temperature and 3-mercaptopropyl-functionalized silica gel was added. The mixture was filtered through diatomaceous earth, concentrated and purified by flash chromatography on silica gel eluting with 0-60% ethyl acetate/hexanes gave the title compound (59 mg). ¹H NMR (400 MHz, DMSO-d₆) δ 8.82 (d, J=2.3 Hz, 1H), 8.49 (d, J=2.3 Hz, 1H), 8.00-7.72 (m, 2H), 7.55-7.45 (m, 2H), 7.33-7.18 (m, 4H), 4.61-4.38 (m, 2H), 4.30 (qd, J=7.5, 4.0 Hz, 1H), 3.95-3.74 (m, 1H), 2.10-1.87 (m, 1H), 1.82-1.68 (m, 1H), 1.65-1.39 (m, 4H). MS (ESI+) m/z 467.3 (M+H)⁺.

### Step 177B

### 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[2,3′-bipyridine]-5′-carboxamide

To a mixture of the product of Step 174B (43 mg, 0.11 mmol), the product of Step 177A (54 mg, 0.11 mmol), 4-(di-tert-butylphosphino)-N,N-dimethylaniline (1.7 mg, 0.006 mmol), and tris(dibenzylideneacetone)dipalladium(0) (2.4 mg, 0.002 mmol) in dioxane (2 mL) was added potassium phosphate (49 mg, 0.23 mmol) in water (0.5 mL), and the mixture was sparged with nitrogen for 20 minutes. After heating at 75° C. for 5 hours, the mixture was cooled, 3-mercaptopropyl-functionalized silica gel was added, and the solution was filtered through diatomaceous earth and concentrated. Purification by reverse phase-HPLC eluting with 5/95 to 90/10 acetonitrile/0.10% trifluoroacetic acid in water provided the title compound as a trifluoroacetic acid salt. The salt was dissolved in methanol and eluted through a Silicycle carbonate column with methanol to generate the free base of the title compound (11 mg). ¹H NMR (400 MHz, DMSO-d₆) δ 8.82 (d, J=2.3 Hz, 1H), 8.62-8.55 (m, 2H), 8.51 (d, J=2.3 Hz, 1H), 7.93-7.86 (m, 1H), 7.81 (td, J=7.7, 1.9 Hz, 1H), 7.60-7.53 (m, 2H), 7.46-7.34 (m, 4H), 7.34-7.20 (m, 4H), 4.61 (d, J=2.0 Hz, 2H), 4.39-4.23 (m, 2H), 4.18 (s, 1H), 3.95 (dt, J=6.2, 3.9 Hz, 1H), 3.47 (t, J=6.3 Hz, 3H), 2.96-2.73 (m, 2H), 2.50 (d, J=1.8 Hz, 2H), 2.43-2.32 (m, 7H), 2.13-1.90 (m, 4H), 1.81-1.53 (m, 3H). MS (ESI+) m/z 633.1 (M+H)⁺.

### Example 178

### 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6-methoxy[2,3′-bipyridine]-5′-carboxamide Step 178A 6′-amino-N-{(1S,2S)-2-[(4-bromophenyl)methoxy]cyclopentyl}-6-methoxy[2,3′-bipyridine]-5′-carboxamide

A mixture of 6-methoxypyridine-2-boronic acid pinacol ester (165 mg, 0.70 mmol), the product of Step 153A (350 mg, 0.68 mmol), copper(I) chloride (7.8 mg, 0.08 mmol), diacetoxypalladium (17 mg, 0.08 mmol), 1,1′-bis(diphenylphosphino)ferrocene (79 mg, 0.14 mmol) and cesium carbonate (268 mg, 0.82 mmol) in N,N-dimethylformamide (3 mL) was sparged with nitrogen for 10 minutes and heated at 100° C. for 18 hours. After cooling to ambient temperature, 3-mercaptopropyl-functionalized silica gel was added, and the mixture was filtered through diatomaceous earth and concentrated. Purification by flash chromatography on silica gel eluting with 0-50% ethyl acetate/hexanes gave the title compound which was used without any further purification. MS (ESI+) m/z 495.5 (M+H)⁺.

### Step 178B

### 6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6-methoxy[2,3′-bipyridine]-5′-carboxamide

The title compound was prepared as described in Step 177B using the product of Step 178A in place of the product of Step 177A. ¹H NMR (400 MHz, DMSO-d₆) δ 8.87 (d, J=2.3 Hz, 1H), 8.58-8.39 (m, 2H), 7.70 (t, J=7.8 Hz, 1H), 7.59-7.52 (m, 2H), 7.50-7.25 (m, 8H), 6.68 (d, J=8.1 Hz, 1H), 4.61 (s, 2H), 4.38-4.23 (m, 2H), 3.93 (s, 4H), 3.47 (t, J=6.4 Hz, 2H), 2.95-2.72 (m, 2H), 2.50 (d, J=2.0 Hz, 3H), 2.37 (dd, J=14.8, 8.5 Hz, 8H), 2.12-1.88 (m, 4H), 1.79-1.52 (m, 3H). MS (ESI+) m/z 663.2 (M+H)⁺.

### Example 179

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methoxy-1,3-thiazol-4-yl)pyridine-3-carboxamide

### Step 179A

### methyl 2-amino-5-(4,4,5,5-tetramethyl-1,3,2-dioxaborolan-2-yl)nicotinate

The title compound was prepared as described in Intermediate 19B substituting methyl 2-amino-5-bromonicotinate for Intermediate 19A. MS (ESI+) m/z 279 (M+H)⁺.

### Step 179B

### methyl 2-amino-5-(2-methoxy-1,3-thiazol-4-yl)pyridine-3-carboxylate

A mixture of the product of Step 179A (200 mg, 0.719 mmol), 4-bromo-2-methoxythiazole (181 mg, 0.935 mmol), bis(di-tert-butyl(4-dimethylaminophenyl)phosphine)dichloropalladium (II) (25.5 mg, 0.036 mmol), K₂CO₃ (298 mg, 2.157 mmol) in dioxane (3 mL) and water (0.6 mL) was degassed and heated at 80° C. for 4 hours. Water was added, and the mixture was extracted with ethyl acetate. The organic was washed with brine, dried over MgSO₄ and purified by flash chromatography on silica gel eluting with 0-90% ethyl acetate/heptane to afford the title compound (150 mg). MS (ESI+) m/z 266 (M+H)⁺.

### Step 179C

### 2-amino-5-(2-methoxy-1,3-thiazol-4-yl)pyridine-3-carboxylic acid

The product of Step 179B (150 mg, 0.565 mmol) in methanol (3 mL) and tetrahydrofuran (3 mL) was added 2 M LiOH (848 μl, 1.696 mmol), and the mixture was stirred at ambient temperature overnight. The mixture was neutralized with 2 N HCl and filtered. The solid was rinsed with water and dried in vacuo to afford the title compound (130 mg). MS (ESI+) m/z 252 (M+H)⁺.

### Step 179D

### 2-amino-N-{(1S,2S)-2-[(4-bromophenyl)methoxy]cyclopentyl}-5-(2-methoxy-1,3-thiazol-4-yl)pyridine-3-carboxamide

The title compound was prepared as described in Intermediate 3 substituting the product of Step 179C for Intermediate 2. MS (ESI+) m/z 504 (M+H)⁺.

### Step 179E

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methoxy-1,3-thiazol-4-yl)pyridine-3-carboxamide

The title compound was prepared as described in Step 174C substituting the product of Step 179D for the product of Step 174A. ¹H NMR (501 MHz, DMSO-d₆) δ 8.56 (d, J=2.2 Hz, 1H), 8.45 (d, J=7.8 Hz, 1H), 8.19 (d, J=2.3 Hz, 1H), 7.57-7.50 (m, 2H), 7.43-7.32 (m, 4H), 7.28 (d, J=7.8 Hz, 1H), 7.17 (s, 1H), 7.13 (s, 2H), 4.58 (d, J=1.6 Hz, 2H), 4.37-4.22 (m, 3H), 4.05 (s, 3H), 3.91 (dt, J=6.3, 4.0 Hz, 1H), 3.44 (q, J=6.2 Hz, 2H), 3.15 (d, J=5.1 Hz, 1H), 2.88 (ddd, J=16.6, 8.4, 5.5 Hz, 1H), 2.77 (dt, J=15.8, 7.7 Hz, 1H), 2.43-2.30 (m, 7H), 2.10-1.87 (m, 5H), 1.76-1.48 (m, 5H). MS (ESI+) m/z 669 (M+H)⁺.

### Example 180

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-4-yl)pyridine-3-carboxamide

### Step 180A

### methyl 2-amino-5-(2-methyl-1,3-thiazol-4-yl)pyridine-3-carboxylate

The title compound was prepared as described in Step 179B substituting 4-bromo-2-methylthiazole for 4-bromo-2-methoxythiazole. MS (ESI+) m/z 250 (M+H)⁺.

### Step 180B

### 2-amino-5-(2-methyl-1,3-thiazol-4-yl)pyridine-3-carboxylic acid

The title compound was prepared as described in Step 179C substituting the product of Step 180A for the product of Step 179B. MS (ESI+) m/z 236 (M+H)⁺.

### Step 180C

### 2-amino-N-{(1S,2S)-2-[(4-bromophenyl)methoxy]cyclopentyl}-5-(2-methyl-1,3-thiazol-4-yl)pyridine-3-carboxamide

The title compound was prepared as described in Intermediate 3 substituting the product of Step 180B for Intermediate 2. MS (ESI+) m/z 488 (M+H)⁺.

### Step 180D

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-4-yl)pyridine-3-carboxamide

The title compound was prepared as described in Step 174C substituting the product of Step 180C for the product of Step 174A. ¹H NMR (400 MHz, DMSO-d₆) δ 8.88 (s, 1H), 8.43 (d, J=7.8 Hz, 1H), 8.33 (d, J=2.2 Hz, 1H), 8.13 (d, J=2.3 Hz, 1H), 7.56-7.49 (m, 2H), 7.45-7.23 (m, 5H), 7.13 (s, 2H), 4.63-4.45 (m, 2H), 4.38-4.17 (m, 3H), 3.87 (dt, J=6.0, 3.8 Hz, 1H), 3.43 (q, J=6.1 Hz, 2H), 2.94-2.83 (m, 1H), 2.76 (dt, J=15.7, 7.7 Hz, 1H), 2.49 (s, 3H), 2.45-2.27 (m, 8H), 2.10-1.79 (m, 5H), 1.74-1.36 (m, 5H). MS (ESI+) m/z 653 (M+H)⁺.

### Example 181

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(4-methyl-1,3-thiazol-2-yl)pyridine-3-carboxamide

### Step 181A

### methyl 2-amino-5-(4-methyl-1,3-thiazol-2-yl)pyridine-3-carboxylate

The title compound was prepared as described in Step 179B substituting 2-bromo-4-methylthiazole for 4-bromo-2-methoxythiazole. MS (ESI+) m/z 250 (M+H)⁺.

### Step 181B

### 2-amino-5-(4-methyl-1,3-thiazol-2-yl)pyridine-3-carboxylic acid

The title compound was prepared as described in Step 179C substituting the product of Step 181A for the product of Step 179B. MS (ESI+) m/z 236 (M+H)⁺.

### Step 181C

### 2-amino-N-{(1S,2S)-2-[(4-bromophenyl)methoxy]cyclopentyl}-5-(4-methyl-1,3-thiazol-2-yl)pyridine-3-carboxamide

The title compound was prepared as described in Intermediate 3 substituting the product of Step 181B for Intermediate 2. MS (ESI+) m/z 488 (M+H)⁺.

### Step 181D

### 2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(4-methyl-1,3-thiazol-2-yl)pyridine-3-carboxamide

The title compound was prepared as described in Step 174C substituting the product of Step 181C for the product of Step 174A. ¹H NMR (400 MHz, DMSO-d₆) δ 8.66 (d, J=7.8 Hz, 1H), 8.61 (d, J=2.3 Hz, 1H), 8.29 (d, J=2.3 Hz, 1H), 7.61-7.53 (m, 2H), 7.52 7.32 (m, 6H), 7.30 (d, J=7.8 Hz, 1H), 7.18 (q, J=1.0 Hz, 1H), 4.60 (s, 2H), 4.45-4.21 (m, 3H), 4.13 (s, 1H), 3.94 (dt, J=6.2, 4.0 Hz, 1H), 3.48 (s, 3H), 3.18 (d, J=3.5 Hz, 1H), 2.91 (ddd, J=14.6, 7.9, 5.4 Hz, 1H), 2.79 (dt, J=15.9, 7.6 Hz, 1H), 2.45 (d, J=31.0 Hz, 8H), 2.17-1.84 (m, 5H), 1.81-1.48 (m, 5H). MS (API+) m/z 653 (M+H)⁺.

### Comparative Example AA

### 2-amino-N-{(1S,2S)-2-[(4′-{2-[4-(2-hydroxyethyl)piperazin-1-yl]propan-2-yl}[1,1′-biphenyl]-4-yl)methoxy]cyclopentyl}-5-(1-methyl-1H-pyrazol-4-yl)pyridine-3-carboxamide

The title compound was synthesized as described in Example 221, U.S. Pat. No. 10,125,118.

### Comparative Example BB

### 2-amino-N-{(1S,2S)-2-[(4′-{(1S)-1-[4-(2-hydroxyethyl)piperazin-1-yl]ethyl}[1,1′-biphenyl]-4-yl)methoxy]cyclopentyl}-5-(1-methyl-1H-pyrazol-4-yl)pyridine-3-carboxamide

The title compound was synthesized as described in Example 219, U.S. Pat. No. 10,125,118.

### Determination of Biological Activity

### HTRF Kinase Activity Biochemical Assay

For the SAR (structure-activity relationship) and compound screening, HTRF (Homogeneous Time Resolved Fluorescence) kinase activity assay was employed for all MER, AXL and TYRO3 kinases using Cisbio HTRF® KinEASE™-TK kit (Cisbio, USA). The kit included biotin-labeled TK substrate, streptavidin-XL665, Eu3+-cryptate-labeled TK antibody and HTRF® Detection buffer. There are two main steps in the kinase assay: kinase reaction and detection of phosphorylated substrate. The reaction was carried out in white low volume 384-well plate (Corning, USA) with 25 nL compound in dimethyl sulfoxide in each well. To measure the compound mediated inhibition of kinase activity, 2.5 μL of the recombinant kinases were pre-incubated with test compounds for 30 minutes in the kinase reaction buffer (20 mM HEPES pH 7.4, 2 mM MnCl₂, 10 mM MgCl₂, 100 μM Na₃VO₄, 0.0075% Triton X 100, 0.005% BSA and 1 mM DTT) prior to the addition of 2.5 μL of 1 μM biotin-labeled TK substrates and 10 μM ATP. Then the reaction was stopped after 1 hour incubation at room temperature by adding 5 μL of HTRF® Detection buffer which also contains 0.375 nM Eu3+-cryptate-labeled TK antibody and 0.062 μM streptavidin-XL665(SA-XL665) to allow for detection of the phosphorylated peptide product. After 1 hour incubation at room temperature, the fluorescence intensity was measured with Envision□ plate reader (PerkinElmer, USA). Upon excitation at 340 nm by UV, the energy from Eu3+ donor of the antibody is transferred to the FRET acceptor XL665, and XL655 emits light at 665 nm. The level of kinase activity was quantified by the HTRF ratio that calculated from the intensity of emission at 665 nm and emission at 620 nm (fluorescence intensity @665 nm/fluorescence intensity @620 nm×10,000). The recombinant protein of human MER (528-end) was purchased from Camnabio, Japan. The recombinant human AXL (473-end) and TYRO3 (455-end) were purchased from SignalChem, Canada.

TABLE 1
HTRF Kinase Activity Data

| | HTRF | HTRF | HTRF |
|---|---|---|---|
| | MERTK | AXL | TYRO |
| EXAMPLE | IC₅₀^{a} | IC₅₀^{a} | IC₅₀^{a} |
| 1 | ++ | + | + |
| 2 | ++ | ++ | + |
| 3 | +++ | ++ | + |
| 4 | + | ++ | + |
| 5 | ++++ | ++++ | ++ |
| 6 | ++++ | ++++ | ++ |
| 7 | ++++ | ++++ | ++ |
| 8 | ++++ | ++++ | +++ |
| 9 | ++++ | +++ | + |
| 10 | ++++ | +++ | + |
| 11 | ++++ | +++ | + |
| 12 | ++++ | ++++ | +++ |
| 13 | ++++ | ++++ | +++ |
| 14 | ++++ | +++ | + |
| 15 | +++ | ++ | + |
| 16 | +++ | +++ | + |
| 17 | +++ | +++ | + |
| 18 | ++++ | +++ | ++ |
| 19 | ++++ | ++++ | ++ |
| 20 | ++++ | +++ | + |
| 21 | ++++ | +++ | + |
| 22 | ++++ | +++ | ++ |
| 23 | ++++ | ++++ | ++++ |
| 24 | +++ | ++ | + |
| 25 | ++++ | +++ | + |
| 26 | ++++ | +++ | + |
| 27 | ++++ | +++ | ++ |
| 28 | +++ | +++ | +++ |
| 29 | +++ | ++ | + |
| 30 | ++++ | ++++ | +++ |
| 31 | ++++ | ++++ | +++ |
| 32 | ++++ | ++++ | +++ |
| 33 | ++ | ++ | + |
| 34 | ++++ | ++++ | +++ |
| 35 | ++++ | +++ | ++ |
| 36 | ++++ | ++++ | +++ |
| 37 | ++++ | +++ | ++ |
| 38 | ++++ | ++++ | +++ |
| 39 | ++++ | ++++ | ++++ |
| 40 | ++++ | ++++ | ++++ |
| 41 | ++++ | +++ | ++ |
| 42 | ++++ | ++++ | +++ |
| 43 | ++++ | ++ | ++ |
| 44 | ++++ | ++ | ++ |
| 45 | ++++ | ++++ | ++ |
| 46 | ++++ | ++++ | +++ |
| 47 | ++++ | ++++ | +++ |
| 48 | ++++ | ++++ | +++ |
| 49 | ++++ | ++++ | ++ |
| 50 | ++++ | ++++ | ++ |
| 51 | ++++ | ++++ | ++++ |
| 52 | ++++ | +++ | ++ |
| 53 | ++ | ++ | + |
| 54 | +++ | +++ | + |
| 55 | +++ | +++ | + |
| 56 | +++ | +++ | + |
| 57 | ++++ | +++ | + |
| 58 | +++ | ++ | + |
| 59 | ++++ | ++++ | ++ |
| 60 | ++++ | +++ | + |
| 61 | +++ | +++ | + |
| 62 | ++++ | ++++ | + |
| 63 | ++++ | ++++ | ++ |
| 64 | ++++ | +++ | + |
| 65 | ++++ | ++++ | ++ |
| 66 | +++ | +++ | + |
| 67 | ++++ | ++++ | ++ |
| 68 | ++++ | ++++ | ++ |
| 69 | ++ | ++ | + |
| 70 | ++++ | ++++ | ++ |
| 71 | ++++ | +++ | ++ |
| 72 | ++ | + | + |
| 73 | ++ | + | ++ |
| 74 | ++ | + | ++ |
| 75 | + | + | + |
| 76 | +++ | ++ | ++ |
| 77 | ++++ | ++++ | +++ |
| 78 | ++++ | +++ | + |
| 79 | ++++ | ++++ | + |
| 80 | ++++ | ++++ | ++ |
| 81 | ++++ | ++++ | +++ |
| 82 | ++++ | ++++ | ++ |
| 83 | ++++ | ++++ | ++++ |
| 84 | ++++ | ++++ | +++ |
| 85 | ++++ | ++++ | ++ |
| 86 | ++++ | ++++ | ++ |
| 87 | ++++ | ++++ | ++ |
| 88 | ++++ | ++++ | ++++ |
| 89 | ++++ | ++++ | + |
| 90 | ++++ | ++++ | + |
| 91 | ++++ | ++++ | +++ |
| | HTRF | HTRF | HTRF |
| | MERTK | AXL | TYRO |
| EXAMPLE | IC₅₀^{a}) | IC₅₀^{a} | IC₅₀^{a} |
| 92 | ++++ | ++++ | +++ |
| 93 | ++++ | ++++ | ++++ |
| 94 | ++++ | ++++ | +++ |
| 95 | +++ | ++ | + |
| 96 | +++ | +++ | + |
| 97 | ++ | ++ | + |
| 98 | ++++ | ++++ | +++ |
| 99 | ++++ | ++++ | ++ |
| 100 | ++++ | ++++ | ++ |
| 101 | +++ | +++ | + |
| 102 | ++++ | ++++ | ++ |
| 103 | ++++ | ++++ | ++++ |
| 104 | ++++ | ++++ | ++++ |
| 105 | ++++ | ++++ | ++ |
| 106 | ++++ | ++++ | +++ |
| 107 | ++++ | ++++ | +++ |
| 108 | ++++ | ++++ | +++ |
| 109 | +++ | ++++ | + |
| 110 | ++++ | ++++ | ++ |
| 111 | +++ | ++ | + |
| 112 | ++++ | ++++ | +++ |
| 113 | ++++ | +++ | ++ |
| 114 | ++++ | ++++ | +++ |
| 115 | ++++ | ++++ | ++ |
| 116 | ++++ | ++++ | ++ |
| 117 | ++++ | ++++ | ++ |
| 118 | ++++ | +++ | + |
| 119 | ++++ | ++++ | +++ |
| 120 | ++++ | ++++ | +++ |
| 121 | ++++ | ++++ | +++ |
| 122 | ++++ | ++++ | +++ |
| 123 | ++++ | ++++ | +++ |
| 124 | ++++ | ++++ | ++ |
| 125 | +++ | +++ | + |
| 126 | +++ | ++ | + |
| 127 | +++ | ++ | + |
| 128 | ++++ | ++++ | ++ |
| 129 | ++++ | ++++ | + |
| 130 | ++++ | ++++ | ++ |
| 131 | ++++ | ++++ | ++ |
| 132 | ++++ | ++++ | ++ |
| 133 | ++++ | ++++ | + |
| 134 | ++++ | ++++ | ++ |
| 135 | ++++ | ++++ | ++ |
| 136 | ++++ | ++++ | ++ |
| 137 | ++++ | ++++ | ++ |
| 138 | ++++ | ++++ | ++ |
| 139 | ++++ | ++++ | ++ |
| 140 | ++++ | ++++ | ++ |
| 141 | ++++ | ++++ | +++ |
| 142 | ++++ | ++++ | ++++ |
| 143 | ++++ | ++++ | ++ |
| 144 | ++++ | ++++ | ++++ |
| 145 | +++ | +++ | + |
| 146 | ++++ | ++++ | +++ |
| 147 | ++++ | ++++ | +++ |
| 148 | ++++ | ++++ | +++ |
| 149 | ++++ | ++++ | +++ |
| 150 | ++++ | ++++ | +++ |
| 151 | ++++ | ++++ | +++ |
| 152 | ++++ | ++++ | ++ |
| 153 | ++++ | ++++ | ++ |
| 154 | ++++ | ++++ | ++ |
| 155 | ++++ | ++++ | ++ |
| 156 | ++ | + | + |
| 157 | ++++ | ++++ | ++ |
| 158 | ++++ | ++++ | +++ |
| 159 | ++++ | ++++ | +++ |
| 160 | ++++ | ++++ | +++ |
| 161 | ++++ | ++++ | +++ |
| 162 | ++++ | ++++ | +++ |
| 163 | ++++ | ++++ | +++ |
| 164 | ++++ | ++++ | ++++ |
| 165 | ++++ | ++++ | ++++ |
| 166 | ++++ | ++++ | ++ |
| 167 | ++++ | ++++ | ++ |
| 168 | ++++ | ++++ | +++ |
| 169 | ++++ | ++++ | ++++ |
| 170 | +++ | +++ | + |
| 171 | ++++ | ++++ | +++ |
| 172 | ++++ | ++++ | +++ |
| 173 | ++++ | ++++ | +++ |
| 174 | ++++ | ++++ | +++ |
| 175 | ++++ | ++++ | +++ |
| 176 | ++++ | ++++ | +++ |
| 177 | ++++ | ++++ | +++ |
| 178 | ++++ | ++++ | +++ |
| 179 | ++++ | ++++ | +++ |
| 180 | ++++ | ++++ | +++ |
| 181 | ++++ | ++++ | +++ |

^{a}++++ for IC₅₀ < 0.010; +++ for 0.010 ≤ IC₅₀ < 0.100; ++ for 0.100 ≤ IC₅₀ < 1.00; + for IC₅₀ ≥ 1.0 μM

TABLE 2
Comparative HTRF Kinase Activity Data

| | HTRF MERTK | HTRF AXL | HTRF TYRO |
|---|---|---|---|
| EXAMPLE | IC₅₀^{a} | IC₅₀^{a} | IC₅₀^{a} |
| AA | ++++ | ++++ | ++ |
| BB | ++++ | ++++ | ++ |

^{a}++++ for IC₅₀ < 0.010; ++ for 0.100 ≤ IC₅₀ < 1.00 μM

In Vivo Ba/F3 pMER Assay

BaF3.MerTK tumor cells were mixed with 50% Matrigel (BD, Franklin Lakes, NJ). A cell suspension of 5.0E+04 cells in 0.1 mL, per mouse, were inoculated subcutaneously into the hind right flank of 6-8 week old CD-1 Nude female mice (Charles River Laboratories, Wilmington, MA). Tumors were allocated into dose groups at 10 to 11 days post tumor cell inoculation, when they were roughly 250-400 mm³ in size. Test compounds were formulated in 10% ethanol, 30% PEG-400, and 60% Phosal-50PG, for oral dosing at 0.2 mL per dose per mouse. Plasma and tumors were harvested 4 hours after a single dose. Ba/F3 tumors were harvested into 2 mL Precellys CKmix tubes, snap frozen in liquid nitrogen, and stored at −80° C. overnight. Then, 600 μL tumor lysis buffer [RIPA (Sigma R0278) with 1×HALT protease and phosphatase inhibitors (Thermo 78440) and Benzonase (Millipore Sigma 712063)]was added on ice and tumors were homogenized in a pre-cooled Bertin Precellys 24 at 5,500 RPM for 2 cycles of 50 sec with 1 minute break between cycles. Precellys tubes were then centrifuged at >18,000 g for 20 minutes at 4° C. and the supernatant was collected on ice. The lysate was then centrifuged again at >18,000 g for 10 minutes at 4° C. to remove any residual debris. The protein concentration was quantified (Thermo BSA Standards 23208 and Pierce 660 1861426) and normalized, and 15 μg of total protein in 12 μL per well was aliquoted into clear bottom white 384 well plates (Corning 3765) in duplicate, one well for phospho-MerTK ALISA and another for total MerTK ALISA. Plates were then frozen at −80° C. overnight. For ALISA assays, 5 μL of phospho-MerTK [0.3 nM mouse monoclonal TEL/ETV6 antibody (Abnova H00002120-MO1) and 2 nM rabbit polyclonal phospho-MERTK antibody (Fabgennix PMKT-140AP)] or total MerTK [0.5 nM mouse monoclonal TEL/ETV6 antibody (Abnova H00002120-M01) and 0.4 nM rabbit monoclonal Myc-Tag (71D10) antibody (CST 2278)]antibody mix in 1× ALISA Immunoassay assay buffer (Perkin Elmer ALOOOF) was added to each well and incubated overnight at 4° C. in a humidified chamber. Then, 10 μL of AlphaLISA donor and acceptor bead mix [40 μg/mL of both anti-rabbit IgG Alpha Acceptor Beads (Perkin Elmer cat. no. AL 104) and anti-mouse IgG Alpha Donor Beads ((Perkin Elmer cat. no. AS104)] in 1× AlphaLISA Immunoassay assay buffer was added to each well and incubated for 5 hours at room temperature in a humidified chamber. Plates were then read on a Perkin Elmer Envision using the 615 nm AlphaLISA protocol. After background subtraction, the phospho-MerTK signal was normalized to total MerTK signal and percent inhibition was calculated as 1−(normalized phospho-MerTK compound/mean normalized phospho-MerTK vehicle control)*100.

TABLE 3
In Vivo Ba/F3 pMer ALISA Assay Data

| Example | % inhibition^{a} |
|---|---|
| 46 | 37 |
| 83 | 58 |
| 88 | 74 |
| 91 | 84 |
| 93 | 53 |
| 102 | 74 |
| 103 | 74 |
| 117 | 64 |
| 120 | 67 |
| 142 | 86 |
| 144 | 93 |
| 151 | 66 |
| 159 | 89 |
| 160 | 75 |
| 161 | 85 |
| 164 | 89 |
| 165 | 92 |
| 166 | 74 |
| 167 | 68 |
| 168 | 74 |
| 169 | 76 |
| 171 | 94 |
| 173 | 94 |

^{a}% inhibition at 50 milligrams per kilogram, orally

TABLE 4
Comparative In Vivo Ba/F3 pMer ALISA Assay Data

| Example | % inhibition^{a} |
|---|---|
| AA | 50 |
| BB | 51 |

^{a}% inhibition at 50 milligrams per kilogram, orally

The compounds of formula (I) of the present disclosure comprise a series of compounds that demonstrate unexpected properties with respect to their improved in vivo Ba/F3 pMER inhibition. Comparative Examples AA and BB which are disclosed in U.S. Pat. No. 10,125,118, have a monocyclic phenyl at the position corresponding to Ring A of the compounds of the present disclosure. Comparative Examples AA and BB inhibit HTRF MerTK and HTRF Axl in vitro with an IC₅₀ of less than 0.010 μM as shown in Table 2. Comparative Examples AA and BB exhibit 50 and 51% inhibition in the in vivo Ba/F3 pMer ALISA Assay as shown in Table 4.

Examples 83, 88, 103, and 142 of the present disclosure inhibit HTRF MerTK and HTRF Axl in vitro with an IC₅₀ of less than 0.010 μM as shown in Table 1. Examples 83, 88, 103, and 142 exhibit 58, 74, 74 and 86% inhibition, respectively, in the in vivo Ba/F3 pMer ALISA Assay as shown in Table 3.

Comparative Examples AA and BB are similar to Examples 83, 88, 103 and 142 of the present disclosure in that the left hand and right hand portions of the molecules comprise 1-methyl-1H-pyrazol-4-yl corresponding to R¹, CH₂ corresponding to X, and (2-hydroxyethyl)piperazin-1-yl corresponding to Y-L¹-R³. Examples 83, 88, 103 and 142 differ from Comparative Examples AA and BB in the position corresponding to Ring A. Examples 83, 88, 103 and 142 have fused bicylic ring corresponding to Ring A. Comparative Examples AA and BB have a monocyclic ring corresponding to Ring A.

Comparative Examples AA and BB and Examples 83, 88, 103 and 142 all have in vitro inhibition of HTRF MerTK and HTRF Axl with an IC₅₀ of less than 0.010 μM. Unexpectedly, Examples 83, 88, 103 and 142 show improved in vivo activity as compared to Comparative Examples AA and BB. Examples 83, 88, 103 and 142 have superior Ba/F3 tumor inhibition of 58, 74, 74 and 86%, respectively, as compared to Comparative Examples AA and BB with 50 and 51% inhibition.

Further benefits of Applicants' invention will be apparent to one skilled in the art from reading this patent application. It is understood that the foregoing detailed description and accompanying examples are merely illustrative and are not to be taken as limitations upon the scope of the present disclosure, which is defined by the appended claims and their equivalents. Various changes and modifications to the described embodiments will be apparent to those skilled in the art. Such changes and modifications, including without limitation those relating to the chemical structures, substituents, derivatives, intermediates, syntheses, formulations, or methods, or any combination of such changes and modifications of use of the present disclosure, may be made without departing from the spirit and scope thereof.

1. A compound of formula (I) or a pharmaceutically acceptable salt thereof: 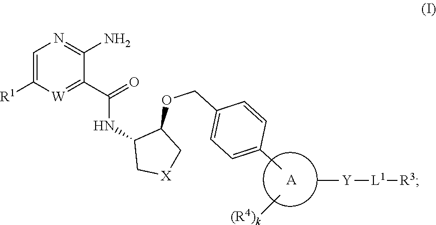
wherein,
Ring A is selected from the group consisting of benzofuran, benzoimidazole, chromane, dihydroindene, dihydropyrrolopyridine, indazole, indole, indoline, isoindoline, pyrrolopyridine, tetrahydroisoquinoline, tetrahydronaphthalene, tetrahydronaphthyridine, and tetrahydroquinoline;
W is selected from the group consisting of N and C—R²;
X is selected from the group consisting of —CR⁶R⁷—, —O—, and —NR⁸—;
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)˜-, —N(R^{a}), and —NHCO₂—(CH₂)ₚ—;
R^{a} is C₁-C₄ alkyl;
R¹ is selected from the group consisting of hydrogen, C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R² is hydrogen or C₁-C₄ alkoxy;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR^{3c}, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁴ is selected from the group consisting of F, Cl, Br, I, ═O, C₁-C₄ alkyl, C₁-C₄ alkoxyalkyl, and C₁-C₄ hydroxyalkyl;
wherein two R⁴ substituents together may be combined with the atoms to which they are attached to form a cycloalkyl ring or a heterocycloalkyl ring;
R⁶ is selected from the group consisting of hydrogen, C₁-C₄ alkyl, and —OH;
R⁷ is selected from the group consisting of hydrogen, and C₁-C₄ alkyl;
R⁸ is C₁-C₄ alkyl;
k is 1, 2, 3, or 4;
m is 1, 2, or 3;
n is 0, 1, 2, or 3; and
p is 0, 1, 2, or 3.

2. The compound of claim 1, or a pharmaceutically acceptable salt thereof,
wherein
W is C—R².

3. The compound of claim 2, or a pharmaceutically acceptable salt thereof,
wherein
X is —CR⁶R⁷—;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I; and
R², R⁶, and R⁷ are each independently hydrogen.

4. The compound of claim 1, or a pharmaceutically acceptable salt thereof, wherein Ring A is selected from the group consisting of dihydroindene, and tetrahydronaphthalene.

5. The compound of claim 4, or a pharmaceutically acceptable salt thereof, of formula (II): 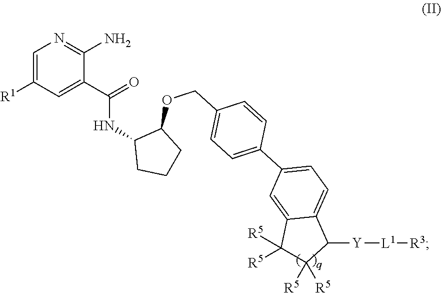
wherein,
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)ₙ—, —N(R^{a})—, and —NHCO₂—(CH₂)ₚ—;
R^{a} is C₁-C₄ alkyl;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR³⁰, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein no more than three R⁵ are other than hydrogen;
wherein two R⁵ substituents together may be combined with the atoms to which they are attached to form a cycloalkyl ring or a heterocycloalkyl ring;
m is 1, 2, or 3;
n is 0, 1, 2, or 3;
p is 0, 1, 2, or 3; and
q is 1 or 2.

6. The compound of claim 5, or a pharmaceutically acceptable salt thereof, wherein
L¹ is a bond;
R³ is selected from the group consisting of hydrogen, C₁-C₄ alkyl, C₁-C₄ hydroxyalkyl, —OH, —NR^{3a}R^{3b}; and
R⁵ is selected from the group consisting of F, and C₁-C₄ alkyl.

7. The compound of claim 4, or a pharmaceutically acceptable salt thereof, of formula (III): 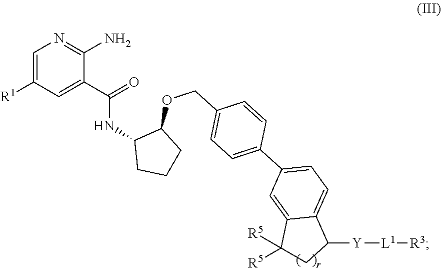
wherein
Y is selected from the group consisting of a bond, piperidinediyl, piperazinediyl and octahydropyrrolopyrazinediyl;
L¹ is selected from the group consisting of a bond, —(CH₂)ₘ—, —C(O)—(CH₂)ₙ—, —N(R^{a})—, and —NHCO₂—(CH₂)ₚ—;
R^{a} is C₁-C₄ alkyl;
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, C₁-C₄ alkyl, C₁-C₄ haloalkyl, C₁-C₄ hydroxyalkyl, azetidinyl, dioxolanyl, —OH, —NR^{3a}R^{3b}, —C(O)OH, C(O)OR³⁰, and —C(O)NH₂; wherein each R³ azetidinyl and dioxolanyl is optionally substituted with one or more C₁-C₄ alkyl substituents;
R^{3a}, and R^{3b} are each independently selected from the group consisting of hydrogen and C₁-C₄ alkyl;
R^{3c} is C₁-C₄ alkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein no more than three R⁵ are other than hydrogen;
wherein two R⁵ substituents together may be combined with the atoms to which they are attached to form a cycloalkyl ring or a heterocycloalkyl ring;
m is 1, 2, or 3;
n is 0, 1, 2, or 3;
p is 0, 1, 2, or 3; and
r is 1 or 2.

8. The compound of claim 7, or a pharmaceutically acceptable salt thereof,
wherein
Y is piperazinediyl; and
Li is a bond.

9. The compound of claim 8, or a pharmaceutically acceptable salt thereof, of formula (IV): 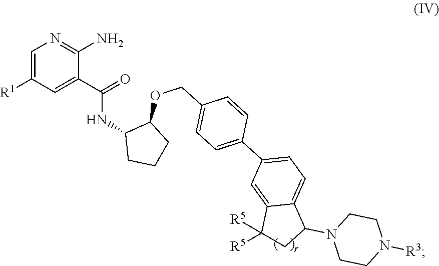
wherein
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R³ is selected from the group consisting of hydrogen, C₁-C₄ alkyl, and C₁-C₄ hydroxyalkyl;
R⁵ is selected from the group consisting of hydrogen, F, Cl, Br, I, ═O, C₁-C₄ alkyl, C₁-C₄ alkoxyalkyl, and C₁-C₄ hydroxyalkyl;
wherein two R⁵ substituents together may be combined with the atoms to which they are attached to form a cycloalkyl ring or a heterocycloalkyl ring; and
r is 1 or 2.

10. The compound of claim 9, or a pharmaceutically acceptable salt thereof, wherein R³ is C₁-C₄ hydroxyalkyl.

11. The compound of claim 9, or a pharmaceutically acceptable salt thereof, wherein R¹ is selected from the group consisting of pyrazolyl, pyridinyl, isothiazol, and thiazolyl; wherein each R¹ pyrazolyl, pyridinyl, isothiazol, and thiazolyl is optionally substituted with one or more substituents independently selected from the group consisting of C₁-C₄ alkyl, C₁-C₄ haloalkyl, C₁-C₄ alkoxy, ═O, CN, F, Cl, Br, and I.

12. The compound of claim 11, or a pharmaceutically acceptable salt thereof,
wherein R¹ is selected from the group consisting of

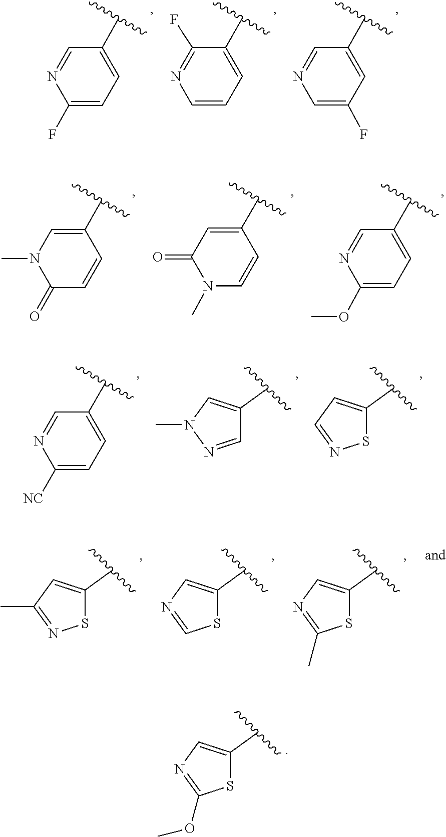

13. The compound of claim 7, or a pharmaceutically acceptable salt thereof, wherein
Y is octahydropyrrolopyrazinediyl; and
L¹ is a bond.

14. The compound of claim 13, or a pharmaceutically acceptable salt thereof, of formula (V): 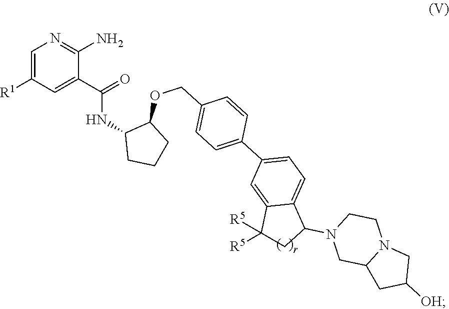
wherein
R¹ is selected from the group consisting of C₁-C₈ alkyl, C₁-C₈ haloalkyl, C₂-C₈ alkenyl, pyrazolyl, pyridinyl, pyridinonyl, and thiazolyl; wherein each R¹ pyrazolyl, pyridinyl, pyridinonyl, and thiazolyl is optionally substituted with one or more substituents independently selected from the group consisting of C₁-C₄ alkyl, C₁-C₄ haloalkyl, C₁-C₄ alkoxy, CN, F, Cl, Br, and I;
R⁵ is selected from the group consisting of hydrogen, F, Cl, Br, I, ═O, C₁-C₄ alkyl, C₁-C₄ alkoxyalkyl, and C₁-C₄ hydroxyalkyl; and
r is 1 or 2.

15. The compound of claim 3, or a pharmaceutically acceptable salt thereof, wherein Ring A is indole.

16. The compound of claim 15, or a pharmaceutically acceptable salt thereof, wherein
Y is piperidinediyl; and
L¹ is a bond.

17. The compound of claim 2, or a pharmaceutically acceptable salt thereof, wherein Ring A is indoline.

18. The compound of claim 1, selected from the group consisting of
2-amino-N-{(1S,2S)-2-[(4-{2-[3-(dimethylamino)propanoyl]-2,3-dihydro-H-isoindol-5-yl}phenyl)methoxy]cyclopentyl}-5-(1-methyl-1H-pyrazol-4-yl)pyridine-3-carboxamide;
2-amino-N-{(1S,2S)-2-[(4-{1-[(dimethylamino)acetyl]-2,3-dihydro-H-indol-5-yl}phenyl)methoxy]cyclopentyl}-5-(1-methyl-1H-pyrazol-4-yl)pyridine-3-carboxamide;
2-amino-N-[(1S,2S)-2-{[4-(1-methyl-2-oxo-2,3-dihydro-1H-indol-5-yl)phenyl]methoxy}cyclopentyl]-5-(1-methyl-1H-pyrazol-4-yl)pyridine-3-carboxamide;
tert-butyl 4-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}piperidine-1-carboxylate;
2-amino-5-(1-methyl-1H-pyrazol-4-yl)-N-[(1S,2S)-2-({4-[1-(piperidin-4-yl)-1H-indol-5-yl]phenyl}methoxy)cyclopentyl]pyridine-3-carboxamide;
2-amino-N-[(1S,2S)-2-({4-[1-(1-methylpiperidin-4-yl)-1H-indol-5-yl]phenyl}methoxy)cyclopentyl]-5-(1-methyl-1H-pyrazol-4-yl)pyridine-3-carboxamide;
2-amino-N-{(1S,2S)-2-[(4-{1-[1-(2-hydroxyethyl)piperidin-4-yl]-1H-indol-5-yl}phenyl)methoxy]cyclopentyl}-5-(1-methyl-1H-pyrazol-4-yl)pyridine-3-carboxamide;
2-amino-N-{(1S,2S)-2-[(4-{1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{2-[2-(dimethylamino)acetamido]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{2-[(1-methylpiperidine-4-carbonyl)amino]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{2-[(1-methylazetidine-3-carbonyl)amino]-2,3-dihydro-1H-inden-5-yl}phenyl)methoxy]cyclopentyl}-5-(1-methyl-1H-pyrazol-4-yl)pyridine-3-carboxamide;
2-amino-N-[(1S,2S)-2-({4-[3,3-dimethyl-1-(1-methylpiperidin-4-yl)-2,3-dihydro-1H-indol-5-yl]phenyl}methoxy)cyclopentyl]-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide;
2-amino-N-{(3S,4R)-4-[(4-{1-[1-(2-hydroxyethyl)piperidin-4-yl]-1H-indol-5-yl}phenyl)methoxy]oxolan-3-yl}-5-(1-methyl-1H-pyrazol-4-yl)pyridine-3-carboxamide;
2-amino-N-[(1S,2S)-2-({4-[3-(1-methylpiperidin-4-yl)-1H-indol-6-yl]phenyl}methoxy)cyclopentyl]-5-(1-methyl-1H-pyrazol-4-yl)pyridine-3-carboxamide;
2-amino-N-[(3S,4R)-4-{[4-(1-methyl-1H-indol-5-yl)phenyl]methoxy}oxolan-3-yl]-5-(1-methyl-1H-pyrazol-4-yl)pyridine-3-carboxamide;
2-amino-5-(1-methyl-1H-pyrazol-4-yl)-N-[(1S,2S)-2-{[4-(1,3,3-trimethyl-2,3-dihydro-1H-indol-5-yl)phenyl]methoxy}cyclopentyl]pyridine-3-carboxamide;
2-amino-N-{(3S,4R)-4-[(4-{1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide;
6-amino-6′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide;
6-amino-6′-fluoro-N-{(3S,4R)-4-[(4-{1-[4-(2-hydroxyethyl)piperazin-1-yl]-2,3-dihydro-1H-inden-5-yl}phenyl)methoxy]oxolan-3-yl}[3,3′-bipyridine]-5-carboxamide;
2-amino-N-{(1S,2S)-2-[(4-{1-[1-(2-hydroxyethyl)piperidin-4-yl]-1H-indol-5-yl}phenyl)methoxy]cyclopentyl}-5-(trifluoromethyl)pyridine-3-carboxamide;
6-amino-6′-fluoro-N-{(1S,2S)-2-[(4-{1-[1-(2-hydroxyethyl)piperidin-4-yl]-1H-indol-5-yl}phenyl)methoxy]cyclopentyl}[3,3′-bipyridine]-5-carboxamide;
2-amino-N-{(1S,2S)-2-[(4-{1-[1-(2,3-dihydroxypropyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide;
2-amino-N-{(3S,4R)-4-[(4-{1-[1-(2-hydroxyethyl)piperidin-4-yl]-1H-indol-5-yl}phenyl)methoxy]oxolan-3-yl}-5-(trifluoromethyl)pyridine-3-carboxamide;
6-amino-6′-fluoro-N-{(3S,4R)-4-[(4-{1-[1-(2-hydroxyethyl)piperidin-4-yl]-1H-indol-5-yl}phenyl)methoxy]oxolan-3-yl}[3,3′-bipyridine]-5-carboxamide;
2-amino-N-{(1S,2S)-2-[(4-{3-[1-(2-hydroxyethyl)piperidin-4-yl]-1-methyl-1H-indol-6-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-amino-2-oxoethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide;
2-amino-N-{(1S,2S)-2-[(4-{1′-[1-(2-hydroxyethyl)piperidin-4-yl]-1′,2′-dihydrospiro[cyclopropane-1,3′-indol]-5′-yl}phenyl)methoxy]cyclopentyl}-5-(1-methyl-1H-pyrazol-4-yl)pyridine-3-carboxamide;
2-amino-N-[(1S,2S)-2-{[4-(1H-indol-7-yl)phenyl]methoxy}cyclopentyl]-5-(1-methyl-1H-pyrazol-4-yl)pyridine-3-carboxamide;
6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-2′-oxo-1′,2′-dihydro[3,4′-bipyridine]-5-carboxamide;
6-amino-6′-fluoro-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}[3,3′-bipyridine]-5-carboxamide;
2-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-5-(1-methyl-1H-pyrazol-4-yl)pyridine-3-carboxamide;
2-amino-N-{(1S,2S)-2-[(4-{(1R)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-6′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide;
6-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-1′-methyl-6′-oxo-1′,6′-dihydro[3,3′-bipyridine]-5-carboxamide;
6-amino-6′-fluoro-N-{(3S,4S)-4-[(4-{1-[1-(2-hydroxyethyl)piperidin-4-yl]-3,3-dimethyl-2,3-dihydro-1H-indol-5-yl}phenyl)methoxy]-1-methylpyrrolidin-3-yl}[3,3′-bipyridine]-5-carboxamide;
rac-2-amino-N-{(1S,2S,4S)-4-hydroxy-2-[(4-{1-[1-(2-hydroxyethyl)piperidin-4-yl]-3,3-dimethyl-2,3-dihydro-1H-indol-5-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3-methyl-3-propyl-2,3-dihydro-1H-indol-5-yl}phenyl)methoxy]cyclopentyl}-5-(1-methyl-1H-pyrazol-4-yl)pyridine-3-carboxamide;
2-amino-N-{(1S,2S)-2-[(4-{3-ethyl-1-[1-(2-hydroxyethyl)piperidin-4-yl]-3-methyl-2,3-dihydro-1H-indol-5-yl}phenyl)methoxy]cyclopentyl}-5-(1-methyl-1H-pyrazol-4-yl)pyridine-3-carboxamide;
3-amino-6-(6-fluoropyridin-3-yl)-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}pyrazine-2-carboxamide;
2-amino-5-(1,3-dimethyl-1H-pyrazol-4-yl)-N-{(3S,4R)-4-[(4-{1-I[1-(2-hydroxyethyl)piperidin-4-yl]-3,3-dimethyl-2,3-dihydro-1H-indol-5-yl}phenyl)methoxy]oxolan-3-yl}pyridine-3-carboxamide;
2-amino-N-{(1S,2S)-2-[(4-{1-[1-(2-hydroxyethyl)piperidin-4-yl]-1,2-dihydrospiro[indole-3,3′-oxolan]-5-yl}phenyl)methoxy]cyclopentyl}-5-(1-methyl-1H-pyrazol-4-yl)pyridine-3-carboxamide;
6-amino-5′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide;
2-amino-N-{(3S,4R)-4-[(4-{(1S)-1-[4-(2-hydroxyethyl)piperazin-1-yl]-2,3-dihydro-1H-inden-5-yl}phenyl)methoxy]oxolan-3-yl}-5-(1-methyl-1H-pyrazol-4-yl)pyridine-3-carboxamide;
6-amino-6′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide;
2-amino-5-(1,5-dimethyl-1H-pyrazol-4-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyridine-3-carboxamide;
6-amino-2′-fluoro-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}[3,3′-bipyridine]-5-carboxamide;
2-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3,3-dimethyl-2,3-dihydro-1H-indol-5-yl}phenyl)methoxy]cyclopentyl}-5-(prop-1-en-2-yl)pyridine-3-carboxamide;
3-amino-6-(6-fluoropyridin-3-yl)-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}pyrazine-2-carboxamide;
2-amino-5-(1-methyl-1H-pyrazol-4-yl)-N-[(1S,2S)-2-({4-[2-(trifluoromethyl)-1H-indol-5-yl]phenyl}methoxy)cyclopentyl]pyridine-3-carboxamide;
2-amino-N-[(1S,2S)-2-{[4-(1,3-dimethyl-1H-indol-5-yl)phenyl]methoxy}cyclopentyl]-5-(1-methyl-1H-pyrazol-4-yl)pyridine-3-carboxamide;
2-amino-N-[(1S,2S)-2-{[4-(2,3-dimethyl-1H-indol-5-yl)phenyl]methoxy}cyclopentyl]-5-(1-methyl-1H-pyrazol-4-yl)pyridine-3-carboxamide;
2-amino-N-[(1S,2S)-2-{[4-(2-methyl-1H-indol-5-yl)phenyl]methoxy}cyclopentyl]-5-(1-methyl-1H-pyrazol-4-yl)pyridine-3-carboxamide;
2-amino-N-[(1S,2S)-2-({4-[1-(2-hydroxyethyl)-1H-indol-5-yl]phenyl}methoxy)cyclopentyl]-5-(1-methyl-1H-pyrazol-4-yl)pyridine-3-carboxamide;
3-{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}propanoic acid;
2-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-5-(1-methyl-1H-pyrazol-4-yl)pyridine-3-carboxamide;
2-amino-5-(1-methyl-1H-pyrazol-4-yl)-N-[(1S,2S)-2-({4-[1-(propan-2-yl)-1H-indol-5-yl]phenyl}methoxy)cyclopentyl]pyridine-3-carboxamide;
{5-[4-({[(1S,2S)-2-{[2-amino-5-(1-methyl-1H-pyrazol-4-yl)pyridine-3-carbonyl]amino}cyclopentyl]oxy}methyl)phenyl]-1H-indol-1-yl}acetic acid;
6-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-6′-fluoro[3,3′-bipyridine]-5-carboxamide;
6-amino-N-[(1S,2S)-2-{[4-(1-{4-[(2S)-2,3-dihydroxypropyl]piperazin-1-yl}-2,2-difluoro-2,3-dihydro-1H-inden-5-yl)phenyl]methoxy}cyclopentyl]-1′-methyl-2′-oxo-1′,2′-dihydro[3,4′-bipyridine]-5-carboxamide;
2-amino-N-[(1S,2S)-2-({4-[1-(difluoromethyl)-1H-indol-5-yl]phenyl}methoxy)cyclopentyl]-5-(1-methyl-1H-pyrazol-4-yl)pyridine-3-carboxamide;
2-amino-N-{(1S,2S)-2-[(4-{(1R)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{(1S)-2,2-difluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
2-amino-N-[(1S,2S)-2-({4-[1-(2-aminoethyl)-1H-indol-5-yl]phenyl}methoxy)cyclopentyl]-5-(1-methyl-1H-pyrazol-4-yl)pyridine-3-carboxamide;
2-amino-N-[(1S,2S)-2-{[4-(1H-indol-5-yl)phenyl]methoxy}cyclopentyl]-5-(1-methyl-1H-pyrazol-4-yl)pyridine-3-carboxamide;
2-amino-N-{(1S,2S)-2-[(4-{3-[4-(2-hydroxyethyl)piperazin-1-yl]-2-oxo-2,3-dihydro-1H-indol-6-yl}phenyl)methoxy]cyclopentyl}-5-(1-methyl-1H-pyrazol-4-yl)pyridine-3-carboxamide;
2-amino-N-{(1S,2S)-2-[(4-{1-[2-(dimethylamino)ethyl]-1H-indol-5-yl}phenyl)methoxy]cyclopentyl}-5-(1-methyl-1H-pyrazol-4-yl)pyridine-3-carboxamide;
3-amino-N-{(3S,4R)-4-[(4-{1-[1-(2-hydroxyethyl)piperidin-4-yl]-3,3-dimethyl-2,3-dihydro-1H-indol-5-yl}phenyl)methoxy]oxolan-3-yl}-6-(1-methyl-1H-pyrazol-4-yl)pyrazine-2-carboxamide;
2-amino-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}-5-(1-methyl-H-pyrazol-4-yl)pyridine-3-carboxamide;
6-amino-6′-fluoro-N-{(1S,2S)-2-[(4-{1-[1-(2-hydroxyethyl)piperidin-4-yl]-3,3-dimethyl-2-oxo-2,3-dihydro-1H-pyrrolo[3,2-b]pyridin-5-yl}phenyl)methoxy]cyclopentyl}[3,3′-bipyridine]-5-carboxamide;
6-amino-6′-fluoro-N-{(1S,2S)-2-[(4-{2′-[1-(2-hydroxyethyl)piperidin-4-yl]-2′,3′-dihydro-1′H-spiro[cyclopropane-1,4′-isoquinolin]-6′-yl}phenyl)methoxy]cyclopentyl}[3,3′-bipyridine]-5-carboxamide;
2-amino-N-{(1S,2S)-2-[(4-{1-[1-(2-hydroxyethyl)piperidin-4-yl]-3,3-dimethyl-2-oxo-2,3-dihydro-1H-pyrrolo[2,3-b]pyridin-5-yl}phenyl)methoxy]cyclopentyl}-5-(1-methyl-H-pyrazol-4-yl)pyridine-3-carboxamide;
2-amino-N-{(1S,2S)-2-[(4-{1-[1-(2-hydroxyethyl)piperidin-4-yl]-3,3-dimethyl-2,3-dihydro-1H-pyrrolo[2,3-b]pyridin-5-yl}phenyl)methoxy]cyclopentyl}-5-(1-methyl-H-pyrazol-4-yl)pyridine-3-carboxamide;
6-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-6′-fluoro[3,3′-bipyridine]-5-carboxamide;
2-amino-N-[(1S,2S)-2-{[4-(1-{4-[(2,2-dimethyl-1,3-dioxolan-4-yl)methyl]piperazin-1-yl}-2,3-dihydro-1H-inden-5-yl)phenyl]methoxy}cyclopentyl]-5-(1-methyl-1H-pyrazol-4-yl)pyridine-3-carboxamide;
6-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide;
2-amino-N-{(1S,2S)-2-[(4-{1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-2′-fluoro-N-{(1S,2S)-2-[(4-{1-[4-(2-hydroxyethyl)piperazin-1-yl]-2,3-dihydro-1H-inden-5-yl}phenyl)methoxy]cyclopentyl}[3,3′-bipyridine]-5-carboxamide;
2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide;
6-amino-6′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
2-amino-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-6′-fluoro[3,3′-bipyridine]-5-carboxamide;
6-amino-N-{(1S,2S)-2-[(4-{2,2-difluoro-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide;
2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide;
6-amino-6′-fluoro-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}[3,3′-bipyridine]-5-carboxamide;
6-amino-N-{(1S,2S)-2-[(4-{(1R,2S)-2-fluoro-1-[4-(2-hydroxyethyl)piperazin-1-yl]-2,3-dihydro-1H-inden-5-yl}phenyl)methoxy]cyclopentyl}-1′-methyl-6′-oxo-1′,6′-dihydro[3,3′-bipyridine]-5-carboxamide;
6-amino-2′-fluoro-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide;
6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide;
2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide;
6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-6′-fluoro[3,3′-bipyridine]-5-carboxamide;
2-amino-N-{(1S,2S)-2-[(4-{3-[4-(2-hydroxyethyl)piperazin-1-yl]-1-benzofuran-6-yl}phenyl)methoxy]cyclopentyl}-5-(1-methyl-1H-pyrazol-4-yl)pyridine-3-carboxamide;
2-amino-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide;
6-amino-6′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
2-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-1′-methyl-2′-oxo-1′,2′-dihydro[3,4′-bipyridine]-5-carboxamide;
6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-6′-fluoro[3,3′-bipyridine]-5-carboxamide;
2-amino-N-{(1S,2S)-2-[(4-{(1R)-1-[4-(2,3-dihydroxypropyl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-N-{(1S,2S)-2-[(4-{(1S)-1-[4-(2,3-dihydroxypropyl)piperazin-1-yl]-2,3-dihydro-1H-inden-5-yl}phenyl)methoxy]cyclopentyl}-2′-fluoro[3,3′-bipyridine]-5-carboxamide;
2-amino-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}-5-(1-methyl-1H-pyrazol-4-yl)pyridine-3-carboxamide;
6-amino-6′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
2-amino-N-{(1S,2S)-2-[(4-{1-[4-(2-hydroxyethyl)piperazin-1-yl]-2,2-dimethyl-2,3-dihydro-1H-inden-5-yl}phenyl)methoxy]cyclopentyl}-5-(1-methyl-1H-pyrazol-4-yl)pyridine-3-carboxamide;
rac-6-amino-6′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide;
6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-1′-methyl-2′-oxo-1′,2′-dihydro[3,4′-bipyridine]-5-carboxamide;
6-amino-N-[(1S,2S)-2-({4-[3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide;
2-amino-N-[(1S,2S)-2-({4-[1-(dimethylamino)-2,2-dimethyl-2,3-dihydro-1H-inden-5-yl]phenyl}methoxy)cyclopentyl]-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3-methyl-1H-pyrrolo[2,3-b]pyridin-5-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-1H-indazol-5-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-1,2,3,4-tetrahydroquinolin-6-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-3,3-dimethyl-2,3-dihydro-1H-pyrrolo[3,2-b]pyridin-5-yl}phenyl)methoxy]cyclopentyl}-5-(1-methyl-1H-pyrazol-4-yl)pyridine-3-carboxamide;
2-amino-N-{(1S,2S)-2-[(4-{1-[1-(2-hydroxyethyl)piperidin-4-yl]-4,4-dimethyl-1,2,3,4-tetrahydroquinolin-6-yl}phenyl)methoxy]cyclopentyl}-5-(1-methyl-1H-pyrazol-4-yl)pyridine-3-carboxamide;
6-amino-1′-methyl-N-[(1S,2S)-2-({4-[(1S)-1-(4-methylpiperazin-1-yl)-2,3-dihydro-1H-inden-5-yl]phenyl}methoxy)cyclopentyl]-2′-oxo-1′,2′-dihydro[3,4′-bipyridine]-5-carboxamide;
6-amino-2′-fluoro-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide;
6-amino-6′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide;
6-amino-6′-fluoro-N-{(1S,2S)-2-[(4-{(1R)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide;
6-amino-2′-fluoro-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
2-amino-N-[(1S,2S)-2-({4-[5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide;
2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(1-methyl-1H-pyrazol-4-yl)pyridine-3-carboxamide;
2-amino-5-(1-methyl-1H-pyrazol-4-yl)-N-[(1S,2S)-2-({4-[5-(piperazin-1-yl)-5,6,7,8-tetrahydronaphthalen-2-yl]phenyl}methoxy)cyclopentyl]pyridine-3-carboxamide;
6-amino-5′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide;
2-amino-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}-5-(trifluoromethyl)pyridine-3-carboxamide;
2-amino-N-{(1S,2S)-2-[(4-{1-[(3,4-dihydroxybutyl)(methyl)amino]-2,3-dihydro-1H-inden-5-yl}phenyl)methoxy]cyclopentyl}-5-(1-methyl-1H-pyrazol-4-yl)pyridine-3-carboxamide;
6-amino-N-{(1S,2S)-2-[(4-{1-[(3,4-dihydroxybutyl)(methyl)amino]-2,3-dihydro-1H-inden-5-yl}phenyl)methoxy]cyclopentyl}-6′-fluoro[3,3′-bipyridine]-5-carboxamide;
6-amino-N-{(1S,2S)-2-[(4-{1-[(3,4-dihydroxybutyl)(methyl)amino]-2,3-dihydro-1H-inden-5-yl}phenyl)methoxy]cyclopentyl}-2′-fluoro[3,3′-bipyridine]-5-carboxamide;
2-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-5-(1-methyl-1H-pyrazol-4-yl)pyridine-3-carboxamide;
6-amino-N-{(1S,2S)-2-[(4-{(1S)-1-[4-(1,3-dihydroxypropan-2-yl)piperazin-1-yl]-2,3-dihydro-1H-inden-5-yl}phenyl)methoxy]cyclopentyl}-2′-fluoro[3,3′-bipyridine]-5-carboxamide;
2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-5-(trifluoromethyl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(trifluoromethyl)pyridine-3-carboxamide;
6-amino-5′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide;
2-amino-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}-5-(trifluoromethyl)pyridine-3-carboxamide;
6-amino-2′-fluoro-N-{(1S,2S)-2-[(4-{4-[4-(2-hydroxyethyl)piperazin-1-yl]-3,4-dihydro-2H-1-benzopyran-7-yl}phenyl)methoxy]cyclopentyl}[3,3′-bipyridine]-5-carboxamide;
6-amino-2′-fluoro-N-{(1S,2S)-2-[(4-{(1R)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
2-amino-N-{(1S,2S)-2-[(4-{1-[4-(2-hydroxyethyl)piperazin-1-yl]-3,3-dimethyl-2,3-dihydro-1H-inden-5-yl}phenyl)methoxy]cyclopentyl}-4-methoxypyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxypyridine-3-carboxamide;
2-amino-N-[(1S,2S)-2-({4-[3,3-dimethyl-1-(4-methylpiperazin-1-yl)-2,3-dihydro-1H-inden-5-yl]phenyl}methoxy)cyclopentyl]-4-methoxypyridine-3-carboxamide;
2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]pyridine-3-carboxamide;
6-amino-N-{(1S,2S)-2-[(4-{1-[4-(1,3-dihydroxypropan-2-yl)piperazin-1-yl]-3,3-dimethyl-2,3-dihydro-1H-inden-5-yl}phenyl)methoxy]cyclopentyl}-2′-fluoro[3,3′-bipyridine]-5-carboxamide;
2-amino-5-(1-methyl-1H-pyrazol-4-yl)-N-[(1S,2S)-2-({4-[(5S)-5-(piperazin-1-yl)-5,6,7,8-tetrahydronaphthalen-2-yl]phenyl}methoxy)cyclopentyl]pyridine-3-carboxamide;
2-amino-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}-5-(1-methyl-1H-pyrazol-4-yl)pyridine-3-carboxamide;
2-amino-N-[(1S,2S)-2-({4-[(1S)-1-(4-methylpiperazin-1-yl)-2,3-dihydro-1H-inden-5-yl]phenyl}methoxy)cyclopentyl]-5-(trifluoromethyl)pyridine-3-carboxamide;
2-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide;
2-amino-N-[(1S,2S)-2-({4-[(1R)-3,3-dimethyl-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide;
2-amino-N-[(1S,2S)-2-({4-[8,8-dimethyl-5-(piperazin-1-yl)-5,6,7,8-tetrahydronaphthalen-2-yl]phenyl}methoxy)cyclopentyl]-5-(1-methyl-1H-pyrazol-4-yl)pyridine-3-carboxamide;
2-amino-N-[(1S,2S)-2-({4-[8,8-dimethyl-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide;
2-amino-N-{(1S,2S)-2-[(4-{5-[4-(2-hydroxyethyl)piperazin-1-yl]-8,8-dimethyl-5,6,7,8-tetrahydronaphthalen-2-yl}phenyl)methoxy]cyclopentyl}-5-(1-methyl-1H-pyrazol-4-yl)pyridine-3-carboxamide;
6-amino-2′-fluoro-N-{(1S,2S)-2-[(4-{5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide;
6-amino-N-[(1S,2S)-2-({4-[(1S)-3,3-dimethyl-1-(4-methylpiperazin-1-yl)-2,3-dihydro-1H-inden-5-yl]phenyl}methoxy)cyclopentyl]-2′-fluoro[3,3′-bipyridine]-5-carboxamide;
6-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-6′-methoxy[3,3′-bipyridine]-5-carboxamide;
6-amino-6′-methoxy-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide;
6-amino-6′-cyano-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[3,3′-bipyridine]-5-carboxamide;
6-amino-6′-cyano-N-[(1S,2S)-2-({4-[(1S)-1-(4-methylpiperazin-1-yl)-2,3-dihydro-1H-inden-5-yl]phenyl}methoxy)cyclopentyl][3,3′-bipyridine]-5-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-4-methoxy-5-(1-methyl-1H-pyrazol-4-yl)pyridine-3-carboxamide;
2-amino-N-{(1S,2S)-2-[(4-{1-[1-(2-hydroxyethyl)piperidin-4-yl]-1H-benzimidazol-5-yl}phenyl)methoxy]cyclopentyl}-5-(1-methyl-1H-pyrazol-4-yl)pyridine-3-carboxamide;
2-amino-4-methoxy-N-[(1S,2S)-2-({4-[(1S)-1-(4-methylpiperazin-1-yl)-2,3-dihydro-1H-inden-5-yl]phenyl}methoxy)cyclopentyl]-5-(1-methyl-1H-pyrazol-4-yl)pyridine-3-carboxamide;
2-amino-N-{(1S,2S)-2-[(4-{8-[1-(2-hydroxyethyl)piperidin-4-yl]-5,5-dimethyl-5,6,7,8-tetrahydro-1,8-naphthyridin-3-yl}phenyl)methoxy]cyclopentyl}-5-(1-methyl-1H-pyrazol-4-yl)pyridine-3-carboxamide;
6-amino-2′-fluoro-N-{(1S,2S)-2-[(4-{(1S)-1-[4-(2-hydroxyethyl)piperazin-1-yl]-3,3-dimethyl-2,3-dihydro-1H-inden-5-yl}phenyl)methoxy]cyclopentyl}[3,3′-bipyridine]-5-carboxamide;
6-amino-2′-fluoro-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}[3,3′-bipyridine]-5-carboxamide;
2-amino-N-{(1S,2S)-2-[(4-{(1S)-1-[(7R,8aS)-7-hydroxyhexahydropyrrolo[1,2-a]pyrazin-2 (1H)-yl]-2,3-dihydro-1H-inden-5-yl}phenyl)methoxy]cyclopentyl}-5-(1-methyl-1H-pyrazol-4-yl)pyridine-3-carboxamide;
2-amino-N-{(1S,2S)-2-[(4-{8-[1-(2-hydroxyethyl)piperidin-4-yl]-5,6,7,8-tetrahydro-1,8-naphthyridin-3-yl}phenyl)methoxy]cyclopentyl}-5-(1-methyl-1H-pyrazol-4-yl)pyridine-3-carboxamide;
6-amino-2′-fluoro-N-[(1S,2S)-2-({4-[(5S)-5-(4-methylpiperazin-1-yl)-5,6,7,8-tetrahydronaphthalen-2-yl]phenyl}methoxy)cyclopentyl][3,3′-bipyridine]-5-carboxamide;
2-amino-N-[(1S,2S)-2-({4-[(5S)-5-(4-methylpiperazin-1-yl)-5,6,7,8-tetrahydronaphthalen-2-yl]phenyl}methoxy)cyclopentyl]-5-(1-methyl-1H-pyrazol-4-yl)pyridine-3-carboxamide;
6-amino-2′-fluoro-N-{(1S,2S)-2-[(4-{(5S)-5-[4-(2-hydroxyethyl)piperazin-1-yl]-5,6,7,8-tetrahydronaphthalen-2-yl}phenyl)methoxy]cyclopentyl}[3,3′-bipyridine]-5-carboxamide;
6-amino-N-[(1S,2S)-2-({4-[(1S)-1-{4-[(2S)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide;
6-amino-N-[(1S,2S)-2-({4-[(1S)-1-{4-[(2R)-2,3-dihydroxypropyl]piperazin-1-yl}-2,3-dihydro-1H-inden-5-yl]phenyl}methoxy)cyclopentyl]-2′-fluoro[3,3′-bipyridine]-5-carboxamide;
6-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-2′-fluoro[3,3′-bipyridine]-5-carboxamide;
2-amino-N-[(1S,2S)-2-({4-[(1S)-1-{4-[(2S)-2,3-dihydroxypropyl]piperazin-1-yl}-3,3-dimethyl-2,3-dihydro-1H-inden-5-yl]phenyl}methoxy)cyclopentyl]-5-(1-methyl-1H-pyrazol-4-yl)pyridine-3-carboxamide;
2-amino-N-[(1S,2S)-2-({4-[(5R)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide;
2-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-5-(1-methyl-1H-pyrazol-4-yl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-5-yl)pyridine-3-carboxamide;
6-amino-N-[(1S,2S)-2-({4-[(5S)-5-{4-[(2S)-2,3-dihydroxypropyl]piperazin-1-yl}-5,6,7,8-tetrahydronaphthalen-2-yl]phenyl}methoxy)cyclopentyl]-2′-fluoro[3,3′-bipyridine]-5-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,2-thiazol-5-yl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(3-methyl-1,2-thiazol-5-yl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(1,3-thiazol-5-yl)pyridine-3-carboxamide;
6′-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}[2,3′-bipyridine]-5′-carboxamide;
6′-amino-N-{(1S,2S)-2-[(4-{(1S)-1-[4-(2-hydroxyethyl)piperazin--yl]-2,3-dihydro-1H-inden-5-yl}phenyl)methoxy]cyclopentyl}-6-methoxy[2,3′-bipyridine]-5′-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methoxy-1,3-thiazol-4-yl)pyridine-3-carboxamide;
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(2-methyl-1,3-thiazol-4-yl)pyridine-3-carboxamide; and
2-amino-N-{(1S,2S)-2-[(4-{(1S)-1-[4-(2-hydroxyethyl)piperazin-1-yl]-2,3-dihydro-1H-inden-5-yl}phenyl)methoxy]cyclopentyl}-5-(4-methyl-1,3-thiazol-2-yl)pyridine-3-carboxamide.

19. A pharmaceutical composition comprising a compound of claim 1, or a pharmaceutically acceptable salt thereof, and a pharmaceutically acceptable excipient.

20. A method of treating a disease, disorder, or condition, the method comprising administering an effective amount of claim 1, or pharmaceutically acceptable salt thereof, to a subject in need thereof, wherein the disease, disorder, or condition is cancer or an immune-related disease.
